SEQUENCE LISTING (208 sequences)

SEQ ID NO: 1 - protein, 50 aa, Arabidopsis thaliana
MMSFVANLAIKRFDHASTVYVEDVVDSSRVAYSENGGDDDDSGYDYAPAA

SEQ ID NO: 2 - protein, 50 aa, Arabidopsis thaliana
MMSYVANLVIKSFDRASVVYVEDVVDSSRATCVENGGDDDDSGYDYAPAA

SEQ ID NO: 3 - protein, 47 aa, Arabidopsis thaliana
MAVVSHNNAEGRLYESTQTWPIAYLQIGGQENGGDDDDDDCDVAPAA

SEQ ID NO: 4 - protein, 56 aa, Arabidopsis thaliana
MISVSEFVLCIDDNVSGTCMRGKVVISDQAFVYAQSVYVEDGDNDDDDIYDYAPAA

SEQ ID NO: 5 - protein, 56 aa, Arabidopsis thaliana
MFSIYKFVLCKWDQVGETFIRGDVTYNNGEFEYPQVAYVENGDDDDDIIXDYAPAA

SEQ ID NO: 6 - protein, 56 aa, Arabidopsis thaliana
MVSVSELVLYVHENVYETCIGVNIANNDQVFEYAQTAFVENGDNDDDVIYDYAPAA

SEQ ID NO: 7 - protein, 40 aa, Arabidopsis thaliana
MSSSLEFDFVLYYNVHYAFASFKNEGDDDDDVVYDYAPAA

SEQ ID NO: 8 - protein, 63 aa, Arabidopsis thaliana
MFSLSEFVFRIYDHISESCVGGDTTSYDKEIKYRQAAYAEIGDQNEDDIYDYAPAAGVHPSYE

SEQ ID NO: 9 - protein, 49 aa, Brassica napus
MSFAANLVIINFYCASAVCVEELLDNSLGSYTENGGDDDDSGYDYAPAA

SEQ ID NO: 10 - protein, 57 aa, Brassica napus
MISVREFVFCASNNNICEMCSGGVMANNDKRFEYAQAAYVENGDNDDDVIYDYAPAA

SEQ ID NO: 11 - protein, 52 aa, Brassica napus
MFSVSEFLFCTYDNVYGGDITNNDEAIQYAQAVFSENGDDDDDVIYDYAPAA

SEQ ID NO: 12 - protein, 47 aa, Brassica napus
MAVMSYNKAEGRLYESTQTRPVPYIQTVGQESGGDDDDDDSDVAPAA

SEQ ID NO: 13 - protein, 48 aa, Thlaspi arvense
MSFVANLVIKSFLHASAEFEYVVDSSLGAYVENGGDDDDSGYDYAPAA

SEQ ID NO: 14 - protein, 48 aa, Thlaspi arvense
MSSIANFVIKSFNRASAELEDVVDSSLGAYVENGGDDDDSGYDYAPAA

SEQ ID NO: 15 - protein, 50 aa, Glycine max
MVVFICKEEYGVPLSNDWAATHEFGHKFCISNEGDDDDDDNGVIDVAPAA

SEQ ID NO: 16 - protein, 51 aa, Glycine max
MVVFVSCTKSGLPLFSKGNDWPATRFPIHQETDDDDDDDDDDGGIDIAPAA

SEQ ID NO: 17 - protein, 50 aa, Glycine max
MVVFLCKEEYGVLLGNDWAATHEFGHNFCISNEGDDDDDDNGVIDVAPAA

SEQ ID NO: 18 - protein, 66 aa, Glycine max
MSFTSKVIAPWCKKHGNDDVVDAPAATTFIGGNVCNWHGDFVSFVPIAYMEGDDDDDGGYDYAPAA

SEQ ID NO: 19 - protein, 70 aa, Glycine max
MSFTSKVIALWCKKHGNDDGVDVYDAPAATACIEGNVCNWHGDFVSFVPVALVEGDDDDDDGGYDYAPAA

SEQ ID NO: 20 - protein, 68 aa, Glycine max
MALTSKAINQECKKHACGNKDGDWYLYYAPTACTEGDDHKGNRDSCFGHIAYMKGDDDGDIYDYAPAA

SEQ ID NO: 21 - protein, 64 aa, Glycine max
MAFISMAINLIDCMTHACGNKDNDWYLYAPTACTEGDDPNGDVDSWFAYMEGDDDDGYDYAPAA

SEQ ID NO: 22 - protein, 58 aa, Glycine max
MTPEIKKHACDKKDGVLYHYDPTACAEGDDYNGNINYVFAYMEGDDDDDGGYDYAPAA

SEQ ID NO: 23 - protein, 65 aa, Glycine max
MASMSKAMTPEIKKHACDKKDGVSYHYDPTACAEGDDYNGNINYVFAYMEGDDDDDGGYDYAPAA

SEQ ID NO: 24 - protein, 50 aa, Glycine max
MVVFVSCTKSGLPLFSKGNDWQATRLSIHQEADDDDDDDDDGGIDIAPAA

SEQ ID NO: 25 - protein, 50 aa, Glycine max
MVVFICKEEYGVPLSNGWAATHEFGHKFCISNEGDDDDDDNGVIDVAPAA

SEQ ID NO: 26 - protein, 68 aa, Glycine max
MALTSKAINQECKKHACGNKDGDWYLYYAPTACTEGDDHKGNGDSCFGHIAYMKGDDDSDIYDYAPAA

SEQ ID NO: 27 - protein, 65 aa, Glycine max
MASMSKAMTPEIKKHACDKKDGVLYHYDPTACAEGDDYNGNINYVFAYMEGDDDDDGGYDYAPAA

SEQ ID NO: 28 - protein, 45 aa, Solanum lycopersicum
MTEIYSFHLYNKIELTRPVISFCLNRELENDDDDDDDGKKVAPAA

SEQ ID NO: 29 - protein, 50 aa, Solanum lycopersicum
MVIVRGNTTPFLPGRIEARPVINFGLNREFEADDDDDDDDDDGKKVAPAA

SEQ ID NO: 30 - protein, 51 aa, Solanum lycopersicum
MVIVRGNTSRFHPYEIEARLVISFYLNRELENDVDDDDDDDDDGAKVAPAA

SEQ ID NO: 31 - protein, 66 aa, Solanum lycopersicum
MSQISTILMNSICNFNHDVGSHVYESRSMMDRHATGCIYVSATWFEDDGDDDDDDDDADYDYAPAA

SEQ ID NO: 32 - protein, 78 aa, Solanum lycopersicum
MSTIFMIIGFEKRRSCADGDYDYTSAASLEGDDDDGDYDYAPAASLEGNDDDGDYDYAPAASLEGDDDDGDYDYAPAA

SEQ ID NO: 33 - protein, 83 aa, Solanum lycopersicum
MFGIFKIIGFEKIRRSCLDGDDDGDYDYAPAACLKRNGDDDGDYDYAPAAFLEGDDDDRDYDCAPAATIDGDDDGDYDYAPAA

SEQ ID NO: 34 - protein, 86 aa, Solanum lycopersicum
MSGILKIIGFEKIRRSCLDGDDDSDYDYAPAACLERDGDDDGDYDYAPAASLEGDDDDRDYDYVPAASLEGDDDGDYDYAPSGCMK

SEQ ID NO: 35 - protein, 83 aa, Solanum lycopersicum
MSGIFKIIGFEKIKRSCLDGDDDGDYDFAPAACLERDGDDDGDYDYAPAASLEGDDDDRDYDYVPAASLEGDDDGDYDYAPAA

SEQ ID NO: 36 - protein, 84 aa, Solanum lycopersicum
MSSIFKIIGFQKRRSCSDGDDDGDYDYAPSACLEGGGDGDDGDYDYTPAASLEGDCNDQDYDYAPAVSFEGHDVDGDYDYAPAA

SEQ ID NO: 37 - protein, 54 aa, Oryza sativa
MAPVSEASPLVHQDGGIIASFAVYAGAPCCSARGRMAETDGDDDDDDYDCAPAA

SEQ ID NO: 38 - protein, 43 aa, Oryza sativa
MAIAKSECERLAWALLLESNLLVGNRRSNGDDDDDDVDVAPAA

SEQ ID NO: 39 - protein, 57 aa, Zea mays
MAPVSSEAASYLVLIKGGSIAASSRAVYPWDGCSARGRMTETDSDDDDDDYDCAPAA

SEQ ID NO: 40 - protein, 56 aa, Aegilops tauschii
MAPASKVMSHIVQDGGIADYAVYAAAPCDAWCGGRHRKAESDGDDDDDDYDCAPAA

SEQ ID NO: 41 - protein, 36 aa, Amborella trichopoda
MLQRYDAPFVGQKWHQKRIGEGDDDDDDDYDCAPAA

SEQ ID NO: 42 - protein, 36 aa, Amborella trichopoda
MLQRYDAPFVGQKWHQKRIGEGDDDDDDDYDFAPAA

SEQ ID NO: 43 - protein, 39 aa, Amborella trichopoda
MLDRHDAHLGCQKWHQKKILRTEGDDDDDDDDYDCAPAT

SEQ ID NO: 44 - protein, 39 aa, Amborella trichopoda
MLDRHDAHLCCLKWHQKKILRTEGDDDDDDDDYDCAPAA

SEQ ID NO: 45 - protein, 26 aa, Amborella trichopoda
MASEENPPNRRDDDDDDDDYDCAPAT

SEQ ID NO: 46 - protein, 23 aa, Amborella trichopoda
MASEENREGDDDDDDDYDCAPAA

SEQ ID NO: 47 - protein, 50 aa, Arabidopsis lyrata
MMSFVANLAIKSLDRASAVYVEDVVDSSRVAYGENGGDDDDSGYDYAPAA

SEQ ID NO: 48 - protein, 50 aa, Arabidopsis lyrata
MMSFVANLVIKSFYRASAMYVEDMVDSSRATCLENGGDDDDSGYDYAPAA

SEQ ID NO: 49 - protein, 50 aa, Arabidopsis lyrata
MMYFFANLVSKSFDRASAVYVEDVVDCSRATCVENGGDDDDSGYDYAPAA

SEQ ID NO: 50 - protein, 56 aa, Arabidopsis lyrata
MISVTEFILCIDDNVGGTCIGGEVVISGQAFVYAQSVYVEDGDNDDDDIYDYAPAA

SEQ ID NO: 51 - protein, 56 aa, Arabidopsis lyrata
MISVSEIVLYIHENVYETSIGVNIANNDKVFEYAQATFVENGDNDDDVIYDYAPAA

SEQ ID NO: 52 - protein, 56 aa, Arabidopsis lyrata
MSSLSEFVLSIYDHVSESCVGSDTTSYDQEIKSRQAAYAENGDQDDDDIYDYAPAA

SEQ ID NO: 53 - protein, 56 aa, Arabidopsis lyrata
MVSIYKFVLCKCDQVRETCIRGDVTYNNGEFEYHQVAFIENGDDDDDIIYDYAPAA

SEQ ID NO: 54 - protein, 53 aa, Arabis alpina
MISVTEFVLCIHENVYDKCNGDGIVNNNGASDSATVENGDNDDDDIYDYAPAA

SEQ ID NO: 55 - protein, 53 aa, Arabis alpina
MISVTEFVLCIDDIVYEKCIAVSGAKSIQASEFTSVENGDNDDDVIYDYAPAA

SEQ ID NO: 56 - protein, 56 aa, Arabis alpina
MVFVFHYVLCKYDEVCETFIEGNAIKNCAELEYSQAGYVENGDDDDDNVYDYAPAA

SEQ ID NO: 57 - protein, 47 aa, Arabis alpina
MAVMSHNKAESRLHESTQACPSPYSVTRAHENGGDDDDDDCDVAPAA

SEQ ID NO: 58 - protein, 52 aa, Brassica rapa
MFSVSEFLFCTYDNVYGGDITNNDEAVQYAQAVFSENGDDDDDVIYDYAPAA

SEQ ID NO: 59 - protein, 57 aa, Brassica rapa
MINVCDFFPNNNISETCIIGGVMASNDQAFEYAQVQATYVENGDNDDDIIYDYAPAA

SEQ ID NO: 60 - protein, 49 aa, Brassica rapa
MYLVAHLVIKSFDGDYAVSAEDVVDTSRAAYIENGGDDDDGGYDYAPAA

SEQ ID NO: 61 - protein, 54 aa, Brassica rapa
MYAANLVIMNFDRSSAVCVEDMGDSSIGAFIENGGDDDDIGHGYACMIHLTHQM

SEQ ID NO: 62 - protein, 49 aa, Brassica rapa
MSFAANLVIINFYCASAVCVEELLDNSRGSYTENGGDDDDSGYDYAPAA

SEQ ID NO: 63 - protein, 47 aa, Brassica rapa
MAVMSHNKAEGRLYESTQTRLVPYIQTLGQESGGDDDDDDSDVAPAA

SEQ ID NO: 64 - protein, 48 aa, Brassica rapa
MAVMSHDKAEDRLYESAHTRPIPYNSQIVGQESGGDDDDDDSDVAPAA

SEQ ID NO: 65 - protein, 63 aa, Brassica rapa
MCYIKPSFIFFKASFQLSRVSKRMSAIEFDLVLHCNGHTQFARSFKNEGDDDDDVVYDYAPAA

SEQ ID NO: 66 - protein, 41 aa, Brassica rapa
MYALVEFDLVLHYNGHTAFAISFKNEGDDDDDVVYDYAPAA

SEQ ID NO: 67 - protein, 50 aa, Camelina sativa
MMTFVANLLSKSLDRASSVYVEDVVDSSRVAYGENGGDDDDSGYDYAPAA

SEQ ID NO: 68 - protein, 50 aa, Camelina sativa
MMTFVANLLSKSLDRASSAYVEDVVDSSRVAYGENGGDDDDSGYDYAPAA

SEQ ID NO: 69 - protein, 46 aa, Camelina sativa
MMTLLSKSLDRASSVYVEDVVDSSRVAYGENGGDDDDSGYDYAPAA

SEQ ID NO: 70 - protein, 49 aa, Camelina sativa
MSFVANLVIKSFDRASTVCVEDVVDSFRAAYVENGGDDDDSGYDYAPAA

SEQ ID NO: 71 - protein, 50 aa, Camelina sativa
MMSSVANLVIKSFDYASTVCVEDVVDSSRAAYVENGGDDDDSGYDYAPAA

SEQ ID NO: 72 - protein, 49 aa, Camelina sativa
MSFVANLVIKSFDRASTVCVEDVVDSFRVAYVENGGDDDDSGYDYAPAA

SEQ ID NO: 73 - protein, 49 aa, Camelina sativa
MSFVANLVIKSFDRASTVCVEDVVDSFRVAYVENGGDDDDSGYDYAPVA

SEQ ID NO: 74 - protein, 50 aa, Camelina sativa
MMSSVANLVIKSFDYASTVCLEDVVDSSRAAYVENGGDDDDSGYDYAPAA

SEQ ID NO: 75 - protein, 49 aa, Camelina sativa
MSSVADLVIKSFNHASTVCDEDVVDTFRAAYVESGGDDDDSGYDYAPAA

SEQ ID NO: 76 - protein, 49 aa, Camelina sativa
MSSVADLVIKSFNHASTACDEDVVDSFRAAYVENGGDDDDSGYDYAPAA

SEQ ID NO: 77 - protein, 50 aa, Camelina sativa
MMPSVANLVIKSFEYVSTVCLEDVKDSSRVAYVENGGDDDDSGYDYAPAA

SEQ ID NO: 78 - protein, 50 aa, Camelina sativa
MAVLIVSRNNNGEGRLYESTRTQPIPYLQNGGQENGGDDDDDDCDVAPAA

SEQ ID NO: 79 - protein, 67 aa, Cicer arietinum
MASISMIIAPRCEKHAYGEGDRFCYISTACVELEDYHSGGGDFVSPQVTYNEGDDDDDGGYDYAPAA

SEQ ID NO: 80 - protein, 79 aa, Citrus clementina
MAPMSSSLEGITHGNVHHRDDDSIHVYGCPYYYRNEPFEGDGDDDDDDDDGCDLAPAASMEGDGDDDDDDGGYDYAPAA

SEQ ID NO: 81 - protein, 74 aa, Citrus clementina
MSLVSKSVMPSSSWTWCKKHGDGDDDDDDGYDYAPAACIEGYGDDDDDDGDYDYAPAASMEGDDDGSYDYAPAA

SEQ ID NO: 82 - protein, 76 aa, Citrus clementina
MSSSLEGITHGNVHHRDDDSIHVYGCPYYYRNEPFEGDGDDDDDDDDGCDLAPAASMEGDGDDDDDDGGYDYAPAA

SEQ ID NO: 83 - protein, 57 aa, Cucumis melo
MDVSIFEPVASTMIKNIIAYKDVKCGRQFSTNLTTTIIRREGDDDDDDGCYDYAPAA

SEQ ID NO: 84 - protein, 56 aa, Cucumis sativua
MAPISRLPCVLGLKNLGGDGGHGYREGCDCGYTTLVSMAEGDSDDDDGGYDFAPAA

SEQ ID NO: 85 - protein, 55 aa, Cucumis sativua
MVSTSKSVASMMIKNNVCEDVKCSRSFPIDLTKTIIRREGDDDDDDGCYDYAPAA

SEQ ID NO: 86 - protein, 58 aa, Erythranthe guttata
PNHTHSNSWNLCSKRSELTSEEIRVSPNFGILITQLHDREGDDDDDDDDGGTFVAPAA

SEQ ID NO: 87 - protein, 69 aa, Gossypium hirsutum
MSPFSKVVASSCKKHVDGDYDDNDGFDYAPIACMEGNGDDDDDDDYDYAPAASLDGDDDDDSYDYAPAA

SEQ ID NO: 88 - protein, 72 aa, Jatropha curcas
MSSVLLKAIASSWCNNQNLIIYDDGFDYASVVPSIDGDGGDDDDGDYDYAPAASMEGDDDDDDDDGDCAPAA

SEQ ID NO: 89 - protein, 50 aa, Jatropha curcas
MVIVDSKKLGFFRLVAGEGQAMACFCMSKQNDGDDDDDDDDGGADVAPAA

SEQ ID NO: 90 - protein, 48 aa, Lotus japonicus
MVVLVCKESRLPKFFMAPPELQSFCIQNESDSDDDDDGDNDIDIAPAA

SEQ ID NO: 91 - protein, 62 aa, Medicago truncatula
MVFISMVIALNCKQHAYGEGEVDWFGCTSVSCIEEDYHNTDHDSYWEGDDDDDGGYDYAPAA

SEQ ID NO: 92 - protein, 60 aa, Medicago truncatula
MASISMVIALNCKQHAYGEGNWFDYTSVSCIEEDYHNGDRDSYQEGDDDDDGGYDYAPAA

SEQ ID NO: 93 - protein, 62 aa, Medicago truncatula
MASIFTVIAPLCKQNACGEGNGDWFGYTSVSCIVEDYRNGDQDSYKEGDDDDDGGYDYAPAA

SEQ ID NO: 94 - protein, 63 aa, Medicago truncatula
MTFISTVIAPKCKQYAYNGEGDGDWFGYTCVSCIEEDYTNGDRNLYREGDDDDDGGYDYAPAA

SEQ ID NO: 95 - protein, 62 aa, Medicago truncatula
MASISLAIIPKCEQHGYGEGNGDWISYTCVSCIEENYHNGDRDSCKEGDDDDDGGYDYAPAA

SEQ ID NO: 96 - protein, 62 aa, Medicago truncatula
MASISLAITPKCKHHGYSEGNGDWFGYTSVSCIKEDNRNGDRDSCKEGDDDDDGGYDYAPTA

SEQ ID NO: 97 - protein, 62 aa, Medicago truncatula
MASISMAITPKCKEHGYDEGNGDWFGYTYVSCIEEDYRNGDRDSYMEGDDDDDGGYDYAPAA

SEQ ID NO: 98 - protein, 58 aa, Medicago truncatula
MASISMVIAPKCKQHAFNGEGDCDWFGYTNISCIEEDYYNGEGDDDDDDGGYDYAPAA

SEQ ID NO: 99 - protein, 59 aa, Medicago truncatula
MASISIAIATKSIKNVYDGGEWFGYASVACIEDYHIGDVDSYKEGDDDDDGGYDYAPAA

SEQ ID NO: 100 - protein, 61 aa, Medicago truncatula
MASISIAIATRSIKHVYDEDEWFGYAASVACIEDYHTEDVDSSYKEGDDDDDGGYDYAPAA

SEQ ID NO: 101 - protein, 58 aa, Medicago truncatula
MASISIPTRSIKNACGEGEWFGFASVSCNDEDYHTGDVDSYREGDDDDDGDYDYAPAA

SEQ ID NO: 102 - protein, 60 aa, Medicago truncatula
MAFISISIATRSFQNACDEGEWFCYASVGCIEQDNHIGEEDSYREGDDDDDGGYDYAPAA

SEQ ID NO: 103 - protein, 70 aa, Medicago truncatula
MTSISMVNISPKCNHAAYGECDGDWFGYASTICIKGNYYFRNEDSGSAHLIAYIEGDDDDDGGYDYAPAA

SEQ ID NO: 104 - protein, 70 aa, Medicago truncatula
MASISIVNTAPKCNHAAYGECDVDSFGYASTVCIKGNYYIRNEDSGSADLVTYMEGDDDDDGGYDYAPAA

SEQ ID NO: 105 - protein, 50 aa, Medicago truncatula
MSSISNVVAPWCKKHGNDHDGCVVWYDYPPTVCDEGDDDDDGGYDYAPAA

SEQ ID NO: 106 - protein, 60 aa, Morus notabilis
MPHITFMNMVTARNGKDGDNGDHCYDHYFQYYNLSAPGEGDGDDDNDDDDDSGYDYAPAA

SEQ ID NO: 107 - protein, 79 aa, Morus notabilis
MFPAVDKLIYSESLSKKREDGGNHEDGNDGISRRYAPTQVMENIYGASDRDYNYLPNATMDGDDDDDDDDSSYDYAPAA

SEQ ID NO: 108 - protein, 62 aa, Morus notabilis
MSPVSEKNVAILAIMLCKKQNITYGDVTNGDFEYNLDPVTRIEGDDDDDDDDDGGYDYAPAA

SEQ ID NO: 109 - protein, 49 aa, Pisum sativum
MVIITSMESTLPMFLMDNHLSATEFICFCNQSKSEGDDDDGDIDIAPAA

SEQ ID NO: 110 - protein, 66 aa, Phaseolus vulgaris
MAFTSKLIAPCCNNHHALHQNHHAPPTFIEESVFNGHGDSVSFVPAASMEGDDDDDDGSYDYAPAA

SEQ ID NO: 111 - protein, 49 aa, Phaseolus vulgaris
MLLFISCTKSGLPLFIKGNDWPETIFPLHHEGDDDDDDDDGGIDVAPAA

SEQ ID NO: 112 - protein, 44 aa, Phaseolus vulgaris
MMFFVCKEYGLVLSNDWPATHDFHNFHEGDDDDDNGVIDVAPAA

SEQ ID NO: 113 - protein, 71 aa, Populus trichocarpa
MSPLSKTIIAFCTIHRHADGDDDGEYGYDYAPAACMEGDGDDDDSDYDYAPAAPMEGDDDDDGDYDYAPAA

SEQ ID NO: 114 - protein, 84 aa, Ricinus communis
MVHMAFLTARSSGKCSSTNLADQNKDIDVLFGYNEFSMEAPLIEDDGDGDDDDDDGGYDFAPAATLEGDGDDDDDGDYDYAPAA

SEQ ID NO: 115 - protein, 72 aa, Ricinus communis
MSLFAISKAITISCCNKLADDCDNGDGCYFAPPPCIEGDGDDDDGDYDYAPAASSEGDDDDDDDGYDCAPAA

SEQ ID NO: 116 - protein, 55 aa, Ricinus communis
MSPLSKVIASWCNKPVVEEEMGNDDDRVYEYAPATSTEGDDDDDDDDDKDCAPAA

SEQ ID NO: 117 - protein, 81 aa, Solanum tuberosum
MAGKSGRKVVRGVSKSSKAVYFWKIRHGCGIIFGKSKKYKSCSYGSEDDDDDEHDYAPATYLERDDDDDDGNYDYAPAALT

SEQ ID NO: 118 - protein, 55 aa, Solanum tuberosum
MVVIMNANNKKVSLGCPDKFMATEAKLGSTICIDKECEADDDYNDDDASKIAPAA

SEQ ID NO: 119 - protein, 83 aa, Solanum tuberosum
MSGTFKIIGFQKRRSSSDGDDEGYNYAPVTCLEGDGDDNDAYYDYASAPFLEGDDDDGDYDYAPATSLEGEDNDGDYDYAPAA

SEQ ID NO: 120 - protein, 86 aa, Solanum tuberosum
MSTIFKIIGFQKRRSCSDGDDDSDDGYDYAPAACLEGDGDDNDGDYDYAPAASLEGDDDDGDYDYAPAASLEGDDDDGDYDYAPAA

SEQ ID NO: 121 - protein, 78 aa, Solanum tuberosum
MSTIFKIIGFEKRRSCPDGNYNYTLVASLEGDDDDGDYDYAPAASLEGDDDDGDYDYAPAASLEGDDDDGNYDYAPAA

SEQ ID NO: 122 - protein, 82 aa, Solanum tuberosum
MSSIFKIIGFQNKRSYSDGDDDGDYDFAPAAFLEGDDDDGDYDYAPAASLNGDDDDGDYDYVPAASLDGDDDDGDYDYAPAA

SEQ ID NO: 123 - protein, 82 aa, Solanum tuberosum
MSSIFKIIGFHNRRSYSDGDDDGDYDYAPAAYLEGDDDDEDYDYAPAASLNGDDDDGDYDYAPAASLEGDDDDGDYDYAPAA

SEQ ID NO: 124 - protein, 82 aa, Solanum tuberosum
MSSIFKIIGFQNRRSYSDGDDDGDYDYAPTAYLEGDDDDGDYDYAPVASLNGDDDDGDYDYAPATSLEGDDDDGDYDYAPAA

SEQ ID NO: 125 - protein, 82 aa, Solanum tuberosum
MSSIFKIIGFEKRRSCLDGDDDGDYDYAPAACLERDGDDDGDYDYAPAASLEGDDDDRDYDYAPAASLEGDDDGDYDYAPAA

SEQ ID NO: 126 - protein, 84 aa, Solanum tuberosum
MSSIFKIIGFQKRRLCLDGDDDGDYDYAPAACLEGGGDRDDGDYYYALAASLEGDDDDRDYDYAPAASLGGDGDDGDYDYAPVA

SEQ ID NO: 127 - protein, 78 aa, Solanum tuberosum
MSQISTILMNSICNLTFFDYHVERGNHDIGSHVYESTSMMDRHVIGCIYVSATWFEDDGDDDDDDDDDDADYDYAPAA

SEQ ID NO: 128 - protein, 83 aa, Solanum tuberosum
MSEIFTIIGFEKIRRSCLDGDDDGDYDYAPASCLERDGDDDGDYDYAPAASLEGDDDDRDYDYVPAASLEGDDDGDYDYAPAA

SEQ ID NO: 129 - protein, 83 aa, Solanum tuberosum
MSSGIFKILGFEKRRLCSYGDDDGDYVYAPAACLNRDGDDDGEYDYAPAASLEGDDDDRDYDYAPATNLEGDDDRDYDYASAA

SEQ ID NO: 130 - protein, 79 aa, Theobroma cacao
MSSSKCIMHDEDNIKKIGSSSKNIMNDDVHDDKGRRDGYVSNSKSLVQGGNSYTHVPSASVDGDGDDDDDDDYDFAPAA

SEQ ID NO: 131 - protein, 56 aa, Triticum urartu
MAPASKVMSHVVQDGGIADYAVYAAAPCDAWCGGRHRKAESDGDDDDDDYDCAPAA

SEQ ID NO: 132 - protein, 57 aa, Triticum urartu
MAPASKIMSHIVVQDGGIAAYAVYAAAPCDAWCGGRHRKAESDGDDDDDDYDCAPAA

SEQ ID NO: 133 - protein, 55 aa, Triticum urartu
MAPASKAMSHIVQDGGIATYAVYAAPCDAWCGGRHRKAETDGDDDDDDYDCAPAA

SEQ ID NO: 134 - protein, 65 aa, Vitis vinifera
MSSISMAIDSQSMMHDGHVRGEHDKHGHVYCTNDNGGCYYALTAPREGDGDDDDGDGGYDFAPAA

SEQ ID NO: 135 - DNA, 153 bp, Arabidopsis thaliana
  source 1..153: mol_type = other DNA
atgatgtcttttgtcgcaaacttggccatcaagagatttgaccatgcttccaccgtgtatgttgaagatgtggtagatagttctcgagtggcatatagtgagaatggtggtgatgacgatgacagtggctatgattatgctcctgctgcgtga

SEQ ID NO: 136 - DNA, 153 bp, Arabidopsis thaliana
  source 1..153: mol_type = other DNA
atgatgtcttacgttgctaacttggtcatcaagagttttgaccgtgcttccgtggtgtatgttgaagatgtggtggatagctctcgagcgacatgtgttgagaatggtggtgatgacgatgacagtggctatgattatgctcctgctgcgtga

SEQ ID NO: 137 - DNA, 144 bp, Arabidopsis thaliana
  source 1..144: mol_type = other DNA
atggcagtggtgagtcacaacaacgcagaaggcaggctatacgaatcaactcagacttggccaattgcttacttacaaattggtggccaagagaacggaggagacgatgacgacgatgactgtgacgttgcaccggcggcttga

SEQ ID NO: 138 - DNA, 171 bp, Arabidopsis thaliana
  source 1..171: mol_type = other DNA
atgatttctgtttctgaatttgttctctgcatagatgacaatgttagtggtacgtgcatgagaggtaaggtcgttatcagtgatcaagcatttgtgtatgctcaatctgtttatgtagaagacggtgacaacgatgatgacgatatctatgattatgctccagcagcatga

SEQ ID NO: 139 - protein, 1259 aa, Arabidopsis thaliana
MGGGNLHSLPPENASVSASYAVTVGNTKLSDAPVLFFVYCHKAFRAQLVELRRFATDAAEADSFSGDLAVELSRKFEFLKLVYKYHSAAEDEVIFLALDKRVKNIVSNYSLEHAGTDDLFTSIFHWLHVLEEEIGSRSDVLREVILCIGTIQSSICQHMLKEERQVFPLLIEKFSFREQASLVWQFICSVPVMVLEDFLPWMISHLSHEEKIEVENCIKDVAPNEDSLQQVISSWLLDDSQSSCGTPTEIMKGVQYVNVSKSLKKSPESHPSSGCFQRFWEWSKKSLSIPNVGRSPIHGLRLFQNAIEKDLRDIQEGLCQAKFQTLILDLDVLMARLNFLADVLVSYSNAFKKFFHPVLEEMTARRSSTAKQFNIDDCLENFQRLLYKSADDKTKTDNFLLQLQEELESLIIQVTKQFAIQRTEVFPIISKNCNHEMQKQLLYTSIHVLPLGLLKCVILWFSAHLSEEESQSILHFLSLEDSSPKKSFPRLLLQWLRFGYSGKTSVERFWKQLDVMFKVRCSCQKEHTEEASGSFSNQTQLQLCKVSKDVYPRKKDKSSTCFMSMDLAVGDMYETPYSSRMNQQMTFSGKLKPPLHLPDFFGEKNMDDPMIMDVKPIDLLFFFHKAMKMDLDYLVCGSTRLAADFRFLAEFQQRFHMIKFLYQIHSDAEDEIAFPALEAKGQLKNISHSFSIDHELETKHFDKVSFILNEMSELNMLVSTINTTAADHDRKMKYERLCLSLREICKSMHKLLSEHIQHEETELWGLFRNCFSIEEQEKIIGCMLGRISGEILQDMIPWLMESLTSDEQLAAMSLWRQATRKTMFVEWLTEWYNGHVLQEEAGEANNDPFGDSDPLEIVWKYLFEASADGEKGSMRSSLLKLPKTNFTGIMNQPPPNYKVEVGKKEEKDLERSESKKICRGSNQEGDKEQTDKMSQKVSQFGPSKKYEQLLTMSEEELVVVIKKISCDSSLDPQKKDYIKQNLLMSRWNISQRTYNLEPSSLSSNMETVHGQHPSYRDPHSLIFGCNHYKRNCKLLAPCCDKLFTCIRCHDEEADHSVDRKQITKMMCMKCLLIQPIGANCSNTSCKSSMGKYFCKICKLYDDERKIYHCPYCNLCRVGKGLGIDYFHCMKCNACMSRTLVEHVCREKCLEDNCPICHEYIFTSSSPVKALPCGHLMHSTCFQEYTCSHYTCPVCSKSLGDMQVYFKMLDALLAEEKMPDEYSNKTQVILCNDCGRKGNAPYHWLYHKCTTCGSYNSRLL

SEQ ID NO: 140 - protein, 1254 aa, Arabidopsis thaliana
MGVGDPLPLPPEKNRREVNKPPDIASTSSSSASAVNNARLSDAPILLFVYFHKAFRAQLAELQFLAGDTVRSGSDLAVELRSKFEFLKLVYKYHSAAEDEVIFSALDTRVKNIVFNYSLEHDATDDLFTSVFHWLNVLEEEQGNRADVLREVVLCIGTIQSSICQHMLKEERQVFPLMIENFSFEEQASLVWQFICSVPVMVLEEIFPWMTSLLSPKEKSEVETCFKEVVPNELSLQLVINSWLIDDSQSSLTALTKIMKGVQSVEVSENMTNSQTNSSSSGVFQRFWQWSKKMSFSSPNTGHILVHGIHLWHNAIRKDLVDIQKGLCQLTFPSLSLDLNVLVVRLNFLADVLIFYSNAFKTFFYPVFEDMVDQQHSSSSKQFTIDGHVENFKKSLDLETRAGSDNFVITLQEKLESLILTVAKQFSIEETEVFPIISKNCNIEMQRQLLYRSIHFLPLGLLKCVIMWFSAQLPEDECQSIIHYLSSEDSFPNKPFAHLLLQWFRFGYSGKTPVESFWNELSFMFKPRCSFEEELTEEASGSFFQQSPQKLFKVSDPYSMDPPAGYMNETPYSSAMNQQILIPGKLRPLLHLPDLFGDKTIGEHLTMDLKPIDLIFYFHKAMKKDLDYLVRGSARLATDYSFLGEFQQRFHLIKFLYQIHSDAEDEIAFPALEAKGKLQNISQSYSIDHELEVEHLNKVSFLLNELAELNMLVLDHKNVKYEKLCMSLQDICKSIHKLLSEHLHREETELWCLFRDCFTIEEQEKIIACMLGRISGEILQDMIPWLMESLIPDEQHAVMSLWRQATRKTMFGEWLTEWYNSHAVEEETEEANKDPSENSDPLDVVWSYLFEGAADEYKGSICSKPLEETELKGIMNKPLGKAAPNNKVEFGNKEENHLEISGSKKVCTGADETKYKEQTDSNAQAFQMSHNTSQSGQDSRYECLLSMSQEDVEATIRRISRDSSLDPQKKSYIIQNLLMSRWIATQRIYNLEPSILSSNREAVPGQNPSYRDPHKLIFGCKHYKRSCKLLAPCCNKLYTCIRCHDEEVDHLLDRKQITKMMCMKCMIIQPVGASCSNISCSSSMGKYYCKICKLFDDDREIYHCPYCNLCRLGKGLSIDYFHCMKCNACMSRLIVEHVCREKCLEDNCPICHEYIFTSNSPVKALPCGHVMHSTCFQEYTCSHYTCPICSKSLGDMQVYFRMLDALLAEQKMPDEYLNQTQVILCNDCGRKGNAPYHWLYHKCSSCASYNTRLF

SEQ ID NO: 141 - protein, 1266 aa, Thlaspi arvense
MGDGKLHRLPPENASTSGSAVSVENTKLADAPVLFFVYFHKAFLAQLVELRRFATDAAAKAGSFSGDLAGDLRRKFEFLKLVYKYHSAAEDEVIFLALDERVKNIVSNYSLEHAGTDDLFTSVFHWLQVLEEEVGSKSDVLRQVILCIGTIQSSISQHMLKEERQVFPLLIEKFSFKEQASLVWQFLCSVPVMVLEDFLPWMMSYLSHEEKTEVESCIKDVVPAEDSLQQVISSWLLDDSQSTKILKEDVSKNMKKSSHTHQSSGCFQRFWQWSKSPIFIPNVGHNPIHGLQLFQNAVEKDLRDIQKDLCQAKFPSLILDIDVLMARLNFLADVLVSYSNAFKKFFHPVLEDIADGRSSTPKHFTIDVCLESFQRLLYKSADDQVRTDNFILKLQEELESLIVQVTKQFSAQRREVFPIISKNCNHEMRRQLLYTSIHVLPLGLLKCVILWFSAHLSEEESQSILQFLSLEYSSPKKSFPRLLLQWLRFGYSGKTSVESFWKQLSVMFKIRCFCQKEHTEEASGSFSHHQAQLQLCQGSRVDDDSLVCPGKRNKSSTCFLAAGDVYETPYSSRMNQQMLFSGKLRPPLHLPDFFGGNNMDDPFVMDVKPIDLLFFFHKAMKADLDYLVCGSARLAADFGFLGEFQQRFRLIKFLYQIHSDAEDEIAFPALEAKGKLQNISHSFSIDHELEIKHFDKVSFILNEMSELNMLVSSIKFSASDHRKMKYERLCLSLQEICKSMHKILSQHFKHEETELWGLFRDCFAIEEQEKIIGCMLGRISGEILQDMIPWLMDSLTYDEQHVVMSLWRQATRKTMFVEWLTEWYNGHFIQEKTGDQANNNDPFGDSDPLEIVWNYFFEGGSDGDRGSIGIKLVELSETDLADIMKKPLEKVAPYNNLEAGNKGGEYEQLAERKKICREADKKEDKEQTDSNSQIMNLAPMSHKVSQFGQSGKYGHLLTLSQEELAMMIKKLSCDASLDPQKKYYIRQNLLMSRWIISQRLYNLEPSTLPSNGKTVPGQHPSYRDPQGLIFGCMHYKRNCKILAPCCDQLFTCIRCHDEEADHSVDRKQIKKMMCMKCLLIQPIGENCSNTSCKISMGKYFCKICKLHDDERKIYHCPYCNLCRVGKGLGIDYFHCMKCNACMSRLLVEHVCREKCLEDNCPICNEYIFTSSSPVKALPCGHLMHSTCFQEYTCSHYTCPVCSKSLGDMQVYFSMLDALLAEEKMPDEYSNKTQVILCNDCGRKGNAPYHWLYHKCTSCGSYNSRVL

SEQ ID NO: 142 - protein, 1139 aa, Thlaspi arvense
MVVDGSRRSSSTFPGQEPPDIASTSSSSVVNARLSDSPILLFVYFHKAFRAQLAELQSLAGDAARANSDLALELRRKLDFLKLVYKYHSAAEDEVIFSALDTRVKNIAFNYSLEHDDTEDLFTSVFHWLHVLEDEERGNVADVLREVVLCIGTIQSSICQHMLKEERQVFPLIIENFSFQEQSSLVWQFISSVPVMALEEIFPWMTSLLSPKEKSEVENCVKQVVPNEVSLQLVINSWLIEDSRSSSFPALTKIMKGVQCVEVSENMSNSSHQADSSSGGLFQRFWQWSKTSFSSPNTGGGHTLAHGIQIWHTAIKRDLLEIQKGLYQLKFPSFSLELNVLVVRLNFLADVLIFYSNAFKKFFYPVFEEMVDQHSSTSKQFTIDVESFKKSLNLETRTASDNFVITLQEKLESLILTVAKQFSVEETEVFPIISKNCNVEMQRQLLYRSLHVLPLGLLKCVTMWFSAQLSEDESQSIIHFLSLEDSFPNKSFSRLLLQWFRFGYSGKTSIESFWNELSFMFKPIFPFGDEHTEEASGSLCKGSDSHLLVRQGNKDKSSTCIESKDLHAGYMIETPYSSAMNQKILIPERIKPLQQLPGFFSDKNIGNHLTMDLKPIDLLFLFHKAMKKDLDYLVCGSARLASDCSFLGEFHQRFHLIKFLYQIHSDAEDEIAFPALEAKGKLQNISQSYSIDHELEVEHLNKVSFLLKEMAELNMLVSTTDSNSLDHHRDVKYEKLCSSLQDVCKSIHKLLSEHLHREETELWWLFRDFTVEEQEKIIACMLGRMSGEILQDVIPWLMESLIPEEQHAVMSLWSQATRKTMFAEWLTEWYNSHVIGEETKEANKNSYEDSDPLDIVWKYLFEGADHENGGSIGRKPLKLSEKDLKLIMSKPLGNDAPNNKAEIGNKKENHQEISENKKVCVGAEEKRCIEQSDDINFQQTKPAHQVFQMVTKSNQCGEMSKYECLLSMSQEDVDATIRRISCDAVLDSQKKSYIMQNLLTSRWIATQRIHNLEPSILLSSNKEAIPGQHPSYRDPHKLIFGCKHYKRSCKLLASCCNQLFTCIRCHDEETDHSVDRKHITKMMCMKCLLIQPVGANCSNSSCSSSMGKYYCKICKLFDDEREIYHCPYCNLCRGKVLRR

SEQ ID NO: 143 - protein, 1262 aa, Glycine max
MDDGDPSLSDKEEGENDEEDTPLLRVPLVDAPILLFVCFHKAFRSELDHLRRLAETASSLEDEPRRCRQIVLQLQRRFQFLKLAHKYHCAAEDEVIFLALDTHVKNVICTYSLEHRSTNGLFGSVFHFLDELMVPKENISKLFQELVYCIGILQTSIYQHMLKEEEQVFPLLIQKLSNKEQASLVWQFICSVPIMLLEEVLPWMVSFLSANKQSEVTQCLNEIAPMEKAMQEVLVSWLRSSKQTCTETCFQSGEFQGVDGFLHIERSLELSYCNRNSEEISSPMKVNGKEIEDGANQVNVLHLWHNAIKKDLKDILEELHLLRKSSCFQNLDSILIQLKFFADVLIFYSDAQKKFFHPVLNKHAYGWLSKSIEQFLGESNIEDIQQLLFYNSESGILLSKFIEKLCQTLESFVSGVNKQFAFQENEVFPIFRKNCRNGMQERLLSLSLYMMPLGLLRCVITWFSVRLSEKESSSILYCIKKGNNSVCKAFSSLLHEWFRIGYSGKTSIEKFRQELQHMFKRRCSLLPEQIKEAHEFSFLNSEKQPHKVSGQNCLSYSSSSGSNNVNKYETPYSTGINLHIFFPSTVAKLHQHPTLHAEERSSISFLDDPKPIDLIFFFHKAIKKDLEYLVLGSTQLEKNDKLLMDFHKRFHLIYFLHQIHSDAEDEIVFPAMEARGKLKNISHAYTFDHKHEVDHFNKISHILDKMSGLHLSVSTIDPNVKEKGILRYHHLCRKLQEMCKSMHKSLSDHINREEIEIWPIIRKFFSNHEQGRIIGCMLGRIRAEILQDMIPWLMASLTQEEQHVLMFLWSMATKNTMFDEWLGEWWDGYSLTKVTEGSNVAPLQPVEPLEIISKYLSEEILDELQEESSANKSINFLQKDHNGDNVVLSNYNFDDKVKVHNAEQNNNQCSKLTNQFHDHNKHACNEVTNIINPVNNEGKYSQLCDKSGRYDRLLKLSQDDLETVIRRVSRDSCLDPQKKSYIIQNLLMSRWIIRQQISSTEANIKNDELEFPGKHPSYRDPLKLIYGCKHYKRNCKLFAPCCNQLHTCIHCHNEESDHSVDRKSITKMMCMKCLVIQPISATCSTISCNLSMAKYYCRICKLFDDEREIYHCPYCNLCRVGKGLGVDYFHCMNCNACMSRSLMTHTCREKHLEDNCPICHEYIFTSCSPVKALPCGHVMHSTCFQEYTCFNYTCPICSKSLGDMQVYFRMLDALLAEERISDEISSQTQVLLCNDCEKKGETPFHWLYHKCPSCGSYNTRVL

SEQ ID NO: 144 - protein, 1236 aa, Glycine max
MASPLDGGGVAVLPNSVNKVDSSSVLNGGLKCSKPESPILIFLFFHKAIRNELDALHRLAVAFATGNRSDIKPLSGRYHFLSSMYRHHCNAEDEVIFPALDIRVKNVAQTYSLEHKGESNLFDHLFELLNSSINNVESFPKELASCTGALQTSVSQHMAKEEEQVFPLLIEKFSLEEQASLVWQFLCSIPVNMMAEFLPWLSASISPDESQDLRNCLIKIVPEEKLLQKVVFTWMEGRSSINTVETCADHSQVQCSSRALTHQLEKVNCACESTTTGKRKHSGSMIDVSDTTGTHPIDEILLWHSAIKKELSEIAVETRKIQHSEDFTNLSAFNERFQFIAEVCIFHSIAEDKVIFPAVDGEFSFFQEHAEEESQFNDFRRLIESIQSEGASSNSDVEFYSKLCIHADHIMETIQRHFHNEEVQVLPLARKHFSFRRQCELLYQSLCMMPLKLIERVLPWLVGSLTEDEAKTFQRNMQLAAPATDSALVTLFCGWACKARNEGLCLSSSASGCCPAQRLSDIEENIVRPSCACASALSNRHCSVLAESGGNKRSVKRNILESHKNEDLPETSETENIQKQCCSARSCCVPGLGVSSNNLGLSSLSTAKSLRSLSFCSSAPSLNSSLFIWETESSSCNVGSTQRPIDTIFKFHKAIRKDLEYLDVESGKLSDGDETILRQFNGRFRLLWGLYRAHSNAEDDIVFPALESKEALHNVSHSYMLDHKQEEQLFEDISCVLSEFSVLHEALQMTHMSDNLSESNFGTSDANTSDDIKKYNELATKLQGMCKSIRVTLDQHLFREECELWPLFGRHFTVEEQDKIVGRIIGTTGAEVLQSMLPWVTSALTQDEQNKMMDIWKQATKNTMFNEWLSECWKESRVSTAQTETSDHSTSRRGAEYQESLDHNDQMFKPGWKDIFRMNQNELESEIRKVYRDSTLDPRRKAYLVQNLLTSRWIAAQQKSPKALSEGSSNSVEIEGLSPSFQDPEEHVFGCEHYKRNCKLRAACCGKLFTCRFCHDNVSDHSMDRKATSEIMCMRCLNIQPIGPICMTPSCNGFSMAKYYCNICKFFDDERNVYHCPFCNLCRVGRGLGIDYFHCMKCNCCLGIKSASHKCLEKGLEMNCPICCDDLFTSSATVRALPCGHYMHSACFQAYTCNHYTCPICSKSLGDMAVYFGMLDALLAAEELPEEYKDRCQDILCHDCNRKGTSRFHWLYHKCGFCGSYNTRVIKCETSNSSCS

SEQ ID NO: 145 - protein, 1242 aa, Glycine max
MATPLTGLNGVGGGGGVAVLANPVSKVDSSANGGGGFGRSLSESPILIFSFFHKAIRNELDALHRLAMAFATGNCSDIQPLFQRYHFLTSMYRHHSNAEDEVIFPALDIRVKNVAQTYSLEHQGESDLFDHLFELLNSSIHNDESFPKELASCTGALQTSVSQHMAKEEEQVFPLLLEKFSLEEQASLVWQFLCSIPVNMMTEFLPWLSTSISPDESQDLRKCLSKIVPEEKLLQKVVFTWMEGGSSANTVENCLDHSQVRCSLNPLTHQNGKIKCACESTATGKRKYSGSIIDVSDTMRTHPIDEILLWHNAIKKELNEIAAQTRKIQLSGDFTNLSAFNERLQFIAEVCIFHSIAEDKVIFPAVDGKFSFFQEHAEEESQFNEFRSLIESIQSEGATSSSETEFYSTLCSHADHILETIQRHFHNEEVQVLPLARKHFSFKRQRELLYQSLCMMPLKLIERVLPWLIRSLTEDEAQMFLKNMQSTAPAIDSALVTLFCGWACKARKDGLCLSSSVSGCCPAQRFTDIEENTVHSSCTPASALSGRVCSVLAESDGTQQRSVKRNISEVHKNEDVSKTSESESFQKQCCSAQSCCVPALGVNKNNLGLGSLSTTKSLRSLSFTASAPSLNSSLFIWETDNSSCEVGSTERPIDTIFKFHKAIRKDLEYLDIESGKLCDGDETIIRQFSGRFRLLWGLYRAHSNAEDDIVFPALESKEALHNVSHSYTLDHKQEEKLFEDISCVLSELSVLHENLQRAHMSVDLSENDFGISDANDDDNIKKYNELATKLQGMCKSIRVTLDQHIFREELELWPLFGKHFTVEEQDKIVGRIIGTTGAEVLQSMLPWVTSALTQDEQNKMMDTWKQATKNTMFNEWLNECLKESPVSTSQTEASERSTSQRGGDYQESLNLNEQMFKPGWKDIFRMNQNELESEIRKVYRDSTLDPRRKAYLVQNLMTSRWIASQQKLPKAPSGESSKQIEGCSPSFRDPEKQIFGCEHYKRNCKLRAACCGKLFTCRFCHDNASDHSMDRKATLEMMCMQCLTIQPVGPICMSPSCNGLTMAKYYCNICKFFDDERNVYHCPFCNICRVGQGLGIDYFHCMKCNCCLGIKSASHKCLEKGLEMNCPICCDDLFTSSATVRALPCGHYMHSSCFQAYTCSHYTCPICSKSLGDMAVYFGMLDALLAAEELPEEYRDRYQDILCHDCDRKGTSRFHWLYHKCGSCGSYNTRVIKSEAANSSCL

SEQ ID NO: 146 - protein, 1238 aa, Glycine max
MATPLTGLNGGGGGVAVLTNPVNKVDSSANGGGGFGRSLSESPILIFSFFHKAIRNELDALHRLAMAFATGNCSDIQPLFQRYRFLRSMYSHHSNAEDEVIFPALDMRVKNVAQTYSLEHQGESDLFDHLFELLNSSIHNDESFPKELASCTGALQTSVSQHMAKEEEQVFPLLLEKFSLEEQASLVWRFLCSIPVNMMTEFLPWLSSSISPDESQDLQKCLSKIVPEEKLLQKVIFTWMEGRSSANTVENCLDHSQVRCSPNPLTHQNGKIKCACESTATGKRKYSGSSIDVSDTMRTHPIDEILLWHNAIKKELNEIAAQSRKIQLSGDFTNLSAFNERLQFIAEVCIFHSIAEDKVIFPAVDGKFSFYQEHAEEESQFNEFRSLIESIQSEEATSSSETEFYSTLCSHADHILEMIQRHFHNEEVQVLPLARKHFSFKRQRELLYQSLCMMPLKLIERVLPWLIRSLTEDEAQMFLKNMQLAAPAIDSALVTLFCGWACKARKDGLCLSSSVSGCCPAQRFTDIEENTVQSSCTSASALSGRVCSVLAESDGTQQRSVKRNISEVHKNEDVSKTSEIESIQKQCCSARSCCVPALGVNKNNLGLGSLSTTKSLRSLSFTASAPSLNSSLFIWETDNSSCDVGSTERPIDTIFKFHKAIRKDLEYLDIESGKLCDGDETIIRQFSGRFRLLWGLYRAHSNAEDDIVFPALESKEALHNVSHSYTLDHKQEEKLFEDISCVLSELSVLHENMQMTHMSVDLSENDFGISDANDNIKEYNELATKLQGMCKSIRVTLDQHIFREELELWPLFGKHFTVEEQDKIVGRIIGTTGAEVLQSMLPWVTSALTQDEQSKMMDTWKQATKNTMFNEWLNECLKETPVSTSQTEASERSTSQRGGDYQENLNLNEQMFKPGWKDIFRMNQNELESEIRKVYRDSTLDPRRKAYLVQNLMTSRWIAAQQKLPKALSGESSKQIEGCSPSFRDPEKEIFGCEHYKRNCKLRAACCGKLFTCRFCHDNASDHSMDRKATLEMMCMQCLTIQPVGPICMSPSCNGLTMAKYYCNICKFFDDERNVYHCPFCNICRVGQGLGIDYIHCMKCNCCLGIKSASHKCLEKGLEMNCPICCDDLFTSSATVRALPCGHYMHSSCFQAYTCSHYTCPICSKSLGDMAVYFGMLDALLAAEELPEEYRDRYQDILCHDCDRKGTSRFHWLYHKCGSCGSYNTRVIKSEATNSSCL

SEQ ID NO: 147 - protein, 1234 aa, Glycine max
MATPLDGGGVAVLPNSVNKVDSSSALIGGLKCSKPESPILIFLFFHKAIRNELDALHRLAIAFATGNRSDIKPLSERYHFLSSMYRHHCNAEDEVIFPALDIRVKNVAQTYSLEHKGESNLFDHLFELLNSSINNDESFPRELASCTGALQTSVSQHMAKEEEQVFPLLIEKFSLEEQASLVWQFLCSIPVNMMAEFLPWLSTSISPDESQDMQNCLIKIVPQEKLLQKVVFSWMEGRSSINTIETCVNHSQVQCSSRSLTHQVEKVNCACESTTTGKRKHSESMIDVSDTTGTHPIDEILLWHNAIKKELSEIAVEARNIQHSGDFTNLSAFNERFQFIAEVCIFHSIAEDKVIFSAVDGEFSFFQEHAEEESQFKDFRHLIESIQSEGASSNSDVEFYSKLCTHADHIMETIQRHFHNEEVQVLPLARKHFSFRRQCELLYQSLCMMPLKLIERVLPWLVGSLTQDEAKMFQRNMQLAAPATDSALVTLFCGWACKARNEGLCLSSGASGCCPAQRLSDIEENIGWPSCACASALSNSHVLAESGGNNRPVKRNISELHKNEDLPETSEAEDIQKQCCSARPCCVPGLGVSSNNLGLSSLSTAKSLRSLSFSSSAPSLNSSLFIWETESSSCNVGSTQRPIDTIFKFHKAIRKDLEYLDVESGKLSDGDETILRQFNGRFRLLWGLYRAHSNAEDEIVFPALESKEALHNVSHSYMLDHKQEEQLFEDISCVLSEFSVLHEALQMTHMSDNLTESNFGTSDANNSDDIKKYNELATKLQGMCKSIRVTLDQHLFREECELWPLFGRHFTVEEQDKIVGRIIGTTGAEVLQSMLPWVTSALTQDEQNKMMDTWKQATKNTMFNEWLSECWKESPVSTAQTETSDHITSQRGAEYQESLDHNDQMFKPGWKDIFRMNQNELESEIRKVYRDSTLDPRRKAYLVQNLMTSRWIAAQQKSPKALSEGSSNSVEIEGLSPSFRDPGKHVFGCEHYKRNCKLRAACCGKLFTCRFCHDNVRDHSMDRKATSEMMCMRCLNIQPIGPLCITPSCNGFSMAKYYCNICKFFDDERNVYHCPFCNLCRVGQGLGIDYFHCMKCNCCLGIKSSSHKCLEKGLEMNCPICCDDLFTSSATVRALPCGHYMHSACFQAYTCSHYTCPICSKSLGDMAVYFGMLDALLAAEELPEEYKDRCQDILCHDCDRKGTSRFHWLYHKCGFCGSYNTRVIKCETSNSSCS

SEQ ID NO: 148 - protein, 1233 aa, Glycine max
MATAGGGGGGGCVAVMAGSCTVSTPSSSYGGGACSSNSKDTLIESPILIFCLFHKAISSELQSLHATAFDFVSNRRHSQPHSPLKIMSFSHRCHFLRTLYKHHCNAEDQVIFPALDKRVKNVAHTYFLEHEGEGLLFDQLFKLPNSNLLNEESYGRELASCIGALRTSICQHMFKEKEQVFPLVIEKFSSEEQGSLVWQFLCSIPVKMMAEFLPWLASYISSDEYQGLLSCLCTIIPKEKLLHQVIFGWMEGLKIKHRKCTHDTKVQWKDVGMSNLLSHNEKVYSICGSSKTVKRKRVGLNEDPTNSNISCPLDELLLWHKAIKQELSDLAETARKIQLSEEFSNLSSFSGRLQFITEVCISHSIAEDRVIFPAIKAELHFLQDHTDEELQFDKLRCLIDSIQSAGADSSSAEFYFKLSSHTEQITDTILKHFEDEEAQVLPLARKLFSPQRQRELLYESLCSMPLKLIECVLPWLVGSLNQTEVRSFLQNMYMAAPATDHALVTLFSGWACNGYSRNSCFYSSTTGLCPDRRLMEIPFCMCEPSFGLNEKSSAIQQEDENGCIGPVKHGKPESKQDNDVANLMVTGKTLSSSLNQSCCVPELRVNASNLGLGSLALTKSLRELSDYPSAPSLNSSLFMWETNLVSADNQCIARPIDNIFKFHKAIRKDLEYLDVESVKLNDCDEIFIQQFTGRFCLLWGLYRAHSNAEDDIVFPALESKDNLHNVSHSYTLDHQQEEKLFLDISSGLTQLTQLHELLYKKNWSDHITNCFSNYAGCYDIDTVQNYNELSTKIQGMCKSIRVTLDQHILREELELWPLFDRHFSLEEQDKIVGHIIGTTGAEVLQSMLPWVTSALAQDEQNKMIDTLKQATKNTMFCEWLNEWWKGPASSLNITTPGDYSLGTDAYEDSEHSFLAFRPGWKDIFRMNQNELESEIRKVSQDSTLDPRRKAYLIQNLMTSRWIASQQKSSQSLGVESSKGDILETSLSFHDPEKKIFGCEHYKRNCKLRAACCGKLFTCQFCHDKVSDHLMDRKATTEMMCMQCQKIQPAGPVCATPSCGSLLMAKYYCSICKLFDDERTVYHCPFCNLCRLGKGLGVDFFHCMQCNCCMSKKLVDHICREKGLETNCPICCDFLFTSSESVRALPCGHFMHSACFQAYTCSHYICPICSKSMGDMSVYFGMLDALLASEELPEEYRNQCQDILCNDCHEKGTAPFHWLYHKCGFCGSYNTRVI

SEQ ID NO: 149 - protein, 1247 aa, Solanum lycopersicum
MDDLGELEKDADMVDLTSSLAGVKLVDAPILFFVISHKAIILELADIHRVAVEASETGSQHVELVRDLSRRLEFLKIVYKYHCAAEDEVVFLALDAQVKNVVYTYSLEHKSIDDLFVSIFKCIDCLQKETEEFAKLFNGLTCYIGTIETIISQHMLKEEEQIFPSLMKRFSSKEQARLVWQYLCSVPLLLLEDFLPWVITSLSSVRKTDFLNFIHVVLPEETLIQEVFISWLDNNELSAQSCLEHGKGANSHYGIANMKHMLKERTIVVHSSEMKLPTKKNPIDGFHLWHAAIRRDLKEILEELHHLRNSFCLSTLMSLVAQLKFFAEVLNFYSIALDQTFYPLIDELNKSDASASHEQFIEGSQIEELQRLLYCKLQDVIQLNVLVEMLCQEVESFVGRISKKLHFLETKVFLVIRENCSHELQLWLLYRSLQMLPLGLLKCMIIWFSAHLSEDESKLILNNVMLGSPVVNRSFTSLLYEWVRTGYSGKISLEKFRKDLEEMFSSRSSLLEKSFNNSGSSFLQSDMQSFDQSNNLLFQSASAVTSKNSVSYHPSPLGIIEKLDTSYSYGINTQIFSSDSQKSLSFFPGTSSRSSNDLKVPIREFIPIDFVHFFHKALINDIQDVVSLSVKLAEDVGVLAEFHRRFHHLHFLLRIHSNSEDEVAFPALESRVTLQNVSHSYSLDHNLEVENFNKISVILNKFISLQGDEVVDGKKFKYKRLCLKLHNACISMQRTLTDHINHEEIELWPLFREHFSVEEQEKIVGNMLGRTKTEILQEMIPWLMASLTPDEQRGMMMLWRKVTKHTKFFEWLGEWWEPVKREESLNAEMEPKIAPFLSIDPLEAVSTYLSRNGVKQGIWNEKGSEFVNHDIYQHGSFTEDKAHNAKGKQNIDLSEDMARYSTVVDKNKYTETVDSIAQKETTCQDIESCDKSRLQEHHLLTQEELVSVIRKISCDSSLDSEMRSHLMQSLLMSQWILTQKLSDSEVAAANDTEKFPGQCPSYRDEQEIIFGCSHYKRNCKLLAPCCKKLFPCIRCHDETTDHTLDRKTITQMMCMKCLKIQPIGPSCSTPSCDGFSMGRYYCKICKLFDDERQIYHCPFCNLCRLGKGLGVGYFHCMNCNACMSKSLSVHVCREKCLEENCPICHEYIFTSTNPVKALPCGHLMHSKCFQEYTCSHYTCPICSKSLGDMQVYFEMLDALLSEQKIPEEYAGQTQAILCNDCEKRGTASFHWLYHKCSSCGSYNTRLV

SEQ ID NO: 150 - protein, 1263 aa, Solanum lycopersicum
MGGKEDDDDTLALPLPSCSDSVVVVDDEFVKQLVDSPILFFVLSHKAVEIELHQIRCVAVEALDSGGEVVDELCKRLHFLKIVYKYHCVAEDEVLFQALDAQVKNVVFTYSLEHNSIDVLFSSIFDCLDRLQEEKDEISVLFNELTCSIGTIQTTISQHMLKEEEQIFPLMMEKFSSEEQARLIWQYLCSVPLMILEDFMRWLTASLSSHERAYFLKFIHIVLPEEKLIQEVFISWIDENKEASSRSCIEDGKGAKFHYGKANMKYIFEMDVLMVQCKEMQHQKASEEHNPIDGFHIWHAAITQDLRVIMDELYQIRNTLCVSTLLSVITQLKFFADVFTFYSNALDQIYYPLVNQLNKDSPSPFYEQFIERSQIEELQKLLYYKLHEEIQIKVFVDMLCQEVELFVGRMNKKLQFLETEVFVFIRKTCSYELQLWLLYISLHMLPLGLLKCMIIWFSAHLSENESKMMLNNIKLGSSVVSKSFSTLLYEWVRMGYSGKISVEKFRKDLEEMFSSGTYLFEKWCKNSGSSSSHSEIHSPDRPYHPSTLDNIGKHDTPYSNGINLRIFFSDSLNGLFCHPETAVDGMRLSRLDVKPIDFFHFFHKALKKDLQYALSLSVKLAEDVGLLAEFERHFHHVRFLYQLHSKSEDEIAFPALESKGQLRNVSHSYGIDHKLEVEQFDRISIVLNEITSLQGCVDMIDSNKLKYKKLCLNLHDTCISMHKTLTDHIYREEVELWPLFKEHFSVEEQEKIIGDMLGRTKAEILREMIPWLMASLTPEEEHGIMSIWRKVTKNTKFFEWLGEWWEGIKRDESVNAEKGSKLSLALAVDPLEVVSTYLPRDDFWSSSVCHEKGENFLSTESADCDLDQSGSFAADKSQKAKGNKNVDRSTDITQHSTEVDKKICNDTIDIADKKEITCQDIKLYEQSRQKNHKEHHLILTQDKLVDAIRRVSRDFSLDSEKKSHLMQSLLMRQIHFQLLFFDLFLRKNQWILTQEKFHSEVATAKDKEKITGQCPSFRDKTESVFGCKHYKRNCKLLAPCCNELFPCIRCHDEISDHFLDRKSITQMMCMKCLKIQPICPSCLTLTCNNFSMAKYYCRICIVFDDDRQIYHCPFCNLCRVGEGLGVGVFHCMTCNACMLSKSLSIHTCRENCLEDNCPICREDIFTSATPVKQLPCGHLMHSTCFQDYTFTHYTCPICSKTIGDMKVLFELLDAFLSEEKIPEEYAGQIQVILCNDCQKRGTASFHWHYHKCPYCGSYNTRLI

SEQ ID NO: 151 - protein, 1236 aa, Solanum lycopersicum
MATQGREGGGGVAVLCGGGVNAVDSSASSSNGVLEKETGGKQESPILFFLFFHKAIRLELDALHHSALAYATGQLEDIQPLLKRYRFLRSVYKHHCHAEDEVIFPALDIRVKNVAPTYSLEHKGENDLFDHLFEILNSEKQNCERFPRELASCTGALQTSVSQHMSKEEEQVFPLLTEKFSMDEQASLVWQFLCSIPVNMMKKFLPWLSSSISPDEHKDMQKCLSMIIPKEKLLQQVIFSWMEGGKCVTAVGGHDVDADPPGSVDFNSVTETYASGNEKCVCESSSPGKRKFRLKGDSFDTDSGNPIDEVLHWHNAIKRELDEIAAEARRIELAGELSSLTAFYARLQFIAQVCIFHSIAEDKVIFPAVDGGLSFFQEHAEEEIQFNELRCLIESIQCTEVNSTSAAEFFSKLYSQADLIIETIKQHFHNEEVQVLPLARKHFTRDRQRKVLYQSLCLMPLKLMEQVLPWLVGALSEDEARSFLKNLQLAAPEADTALVTLLSGWACKGRTDGVCLSSSVTGCCAVKRFADIEEYYTGAPCPCFLSVHSDDSKRPFKRNLNSLCSKDDTLDLSKGVNACNISCNDQSCRVPGLGVSDNNLVLTTISTPKSLRSLTFSSAAPSLESSLFVWETDCTSSQPNHKVHPIDTIFKFHKAIQKDLEYLDVESGKLSDCPETFLRQFIGRFRLLWGLYRAHSNAEDEIVFPELESKEALHNVSHSYMLDHKQEEKLFEDISSALTNLSELHKGLKEAYQKESGSSILESTGLYDRDCKRKYNELATKVQGMCKSIRVSLDQHIFREEHELWPLFGKHFSMEEQDIIVGRIIGSTGAEVLQSMLPWVTSALTQDEQNKMMDTLKQATRNTMFSEWLNDCWRRNPEVSSQSEALQNSYTNRGVDSHEGLDQSDHMFKPGWKDIFRMNQTELESEIRKVHWDSTLDPRRKSYLIQNLMTSRWIASQQKSQASTEEISRSEDVVGYSPSFRDKEKQIFGCEHYKRNCKLLAACCGKLFACRFCHDEVSDHSMDRKATLEMMCMRCLKVQPIRPSCTTPSCNGFSMAKYYCSICKFFDDERPIYHCPSCNLCRVGHGLGIDFYHCMKCNCCLGKGLVDHKCLEKALETNCPICCEFLFTSSATVRPLPCGHYMHSACFQEYASSNYICPICSKSMGNMAVYFGMLDALLANEVLPEEYRNHWQDILCNDCEQKCRTPFHWLYHKCGFCGSYNTRVITLPTTASDCPT

SEQ ID NO: 152 - DNA, 3780 bp, Arabidopsis thaliana
  source 1..3780: mol_type = other DNA
atgggaggcggaaatcttcattcccttccgccggaaaacgcttccgtttctgcatcttatgcggtgaccgttggaaataccaagctttctgatgctccggttcttttcttcgtttactgccacaaggctttccgtgctcagctggtggagctccggcgtttcgctactgacgctgctgaagccgattcctttagtggggatctggcggtggagctctcccggaagtttgagttcttgaagcttgtatataagtatcatagcgcagctgaagatgaggttatatttttggcgctagataaacgagtgaaaaacatagtttctaattactcacttgagcatgctggcacagatgatctctttacctcaattttccactggctacatgttcttgaagaagaaataggaagtagaagcgatgtacttcgtgaagttattttatgcataggcaccatccagtcatctatatgccaacatatgcttaaagaagagcggcaggtgtttcctttgttaatagagaagttttctttccgagaacaagcatcacttgtgtggcaattcatttgtagtgttccagtgatggtgcttgaagactttttgccatggatgatctctcatctctctcacgaggagaaaattgaagttgagaattgtataaaagatgttgccccaaatgaagactcattgcaacaggtcataagctcttggctgcttgacgatagtcaatcttcttgtgggactcctacagagatcatgaaaggagtccagtatgttaatgtgtctaaaagtttgaagaaatcacctgagtcacatccgtcaagcggatgttttcaacgcttttgggagtggagtaaaaagtccctttcaattccaaatgtaggacggagcccaattcatggtcttagactttttcaaaatgcaattgaaaaagatttgagagatattcaagaaggattatgccaagcaaaattccaaacccttattttggatcttgatgtactgatggctagattaaacttccttgctgatgtccttgtttcttatagcaatgcatttaagaagttctttcacccagtgttagaagaaatgacagctcgccgctcttcaactgccaaacagttcaacatagatgattgtcttgagaattttcagagattgttatacaaaagtgctgatgataaaacaaagacggacaattttctattgcagcttcaggaggaacttgagtcccttataatccaagtaacaaagcaatttgccatacagagaacagaggtattcccaatcatcagcaagaattgcaaccatgaaatgcagaagcagctcctatacacaagcattcatgtcttacctcttggattactaaagtgtgtcatactttggttttctgctcatttatcagaagaagaatctcaatccattcttcatttcttaagtttggaagattcttctcccaagaaatcatttccacgcctcttgctacagtggcttcgttttggctattcaggcaaaacctctgttgaaagattttggaaacagttggatgttatgttcaaagtaagatgttcctgccaaaaggagcacactgaagaagcatcaggatcattttccaaccaaacacagctgcaactatgtaaagtatctaaagatgtttatcctagaaagaaagataagtcttcaacctgcttcatgtcaatggatctagctgttggagacatgtatgagacaccttactcaagtcgaatgaatcaacaaatgactttctcaggaaagcttaagcctcctctccatctcccagatttttttggtgagaagaatatggatgatcctatgattatggatgttaaaccaattgacctccttttcttcttccacaaggcaatgaagatggatttggactaccttgtttgtggatcaacaagattggcagccgactttcgtttcctcgcggagtttcagcaacggttccatatgataaagtttttgtatcagatacattctgatgcagaagatgagattgcatttccagcattggaagccaagggacagctaaaaaacattagtcactcgtttagcattgatcatgagctagaaactaaacacttcgacaaagtttcgttcattttgaatgagatgtcagagctgaacatgttggtttctaccatcaataccactgcagctgaccacgaccgaaaaatgaagtatgagcggttatgtctcagtctccgagagatttgcaaatcaatgcacaagctattgtctgagcatatccaacatgaagaaactgagctttggggtctattcaggaactgcttctctattgaagaacaagagaaaatcataggatgcatgctcgggagaataagtggggaaatattgcaggatatgattccttggttaatggaatctttaacctctgatgaacagcttgccgcgatgtccttgtggcgccaagcaacaaggaaaacaatgtttgtcgaatggctaacagaatggtataacggtcatgttttacaagaggaagcaggagaagcaaacaacgatccatttggggattcagatccactagagattgtctggaagtatctctttgaagcatctgctgatggggaaaaagggagcatgcgcagcagccttctcaaactcccgaagacgaattttacaggtattatgaatcagcctcctcctaattacaaggttgaagttggcaaaaaagaggaaaaagatctagaacggtcagaaagtaaaaagatatgcagaggatctaaccaagagggggacaaagaacaaactgacaaaatgtctcagaaggttagccagtttggtccatccaagaaatatgaacaactgttaacaatgagtgaagaagaacttgtggttgtgattaagaaaatatcatgcgactcttccctggatcctcagaagaaagactatatcaagcagaacttgttaatgagtcggtggaatatctcacaacgtacatataatttggaaccatcctctctctcgagcaacatggaaacagttcatggtcaacatccatcatatcgagatcctcacagtttgatctttggttgcaatcactacaagagaaattgtaaacttcttgctccttgttgtgacaagctcttcacatgtatacgatgccatgatgaagaagctgatcactcggtggataggaaacagattacaaagatgatgtgcatgaaatgcctcttgattcaaccgattggtgcaaattgctcaaatacttcgtgcaagtcgtcaatgggaaaatacttctgcaaaatatgcaaattatatgacgatgaaaggaaaatttatcactgtccttactgcaacctctgtcgagtagggaaaggattgggtattgactacttccactgcatgaaatgcaatgcttgtatgtcacgtaccttagtagagcatgtttgcagggaaaagtgcttagaagataattgtccgatttgccatgagtatatcttcacctccagctccccagtgaaggctcttccttgtggtcacttgatgcactccacatgctttcaggagtacacatgttcacattacacatgccctgtctgcagcaagtcgcttggagatatgcaggtctactttaagatgttagacgcattgcttgcagaagagaagatgcctgatgaatactccaacaaaactcaggtgatcttgtgtaatgattgtggaagaaaaggaaatgctccttaccattggctttaccacaaatgcacaacctgtggctcctacaactcaaggctcctttag

SEQ ID NO: 153 - DNA, 3765 bp, Arabidopsis thaliana
  source 1..3765: mol_type = other DNA
atgggagtcggagatcctcttccacttccgccggaaaagaaccgtcgggaggttaataaaccgccggatattgcgtcgacttcctcctcctccgcttcggcggtgaataatgctcggttgtccgatgctccgattcttttgtttgtttatttccataaggctttccgtgcgcagctagcggagctccagtttctggccggtgatacagtccggtccggttctgatctggccgtggagctccgctctaagtttgagtttcttaagcttgtatataagtatcattccgcagctgaagatgaggttatattttcagcgttggacactcgtgtgaagaacattgtttttaattactcgcttgaacatgatgccacagatgatcttttcacttcagtttttcattggctgaatgttcttgaagaggaacaagggaacagagccgatgtacttcgtgaagttgttttatgcattggaaccattcagtcatccatatgtcaacatatgcttaaagaagaacgacaggtgtttcctttgatgattgagaacttctcctttgaagaacaggcatcgttagtgtggcaattcatttgtagtgttccagtgatggtgctggaagagattttcccatggatgacgtctttactctctcccaaggagaaatctgaagttgagacttgcttcaaagaagttgtcccaaatgaactctctttgcaactggtcataaactcttggctgattgatgacagtcaatcttctttgacggctcttacaaagattatgaaaggagttcagtctgtagaagtgtctgaaaatatgaccaactctcagacaaattcatcatcaagtggtgtgtttcaacgtttttggcagtggagtaaaaaaatgtctttttctagtccaaatacaggacacattttggttcatggtatccatctttggcataatgcaattagaaaagatttggtagacattcaaaaaggactatgccaattgacatttccaagcctttcattggacctcaatgttctggtggttcggctaaacttccttgctgatgtccttatcttttatagcaatgcatttaagacgttcttttacccagtgtttgaagatatggtggatcaacaacactcttcctcttccaaacaattcaccatagatggacatgtggaaaacttcaagaagtcactagaccttgaaactagagcagggagtgacaattttgtcataacccttcaggagaaactggagtctcttatattgacagtagctaagcagttctctatagaggaaacagaggtattcccgatcatcagcaagaactgcaacattgaaatgcagaggcaactactatatagaagcatccatttcctgcctcttggattgcttaagtgtgtcataatgtggttctctgctcagttaccagaagatgaatgtcaatcaattattcactacttaagctctgaagattcctttcccaacaaaccttttgcgcacctcttgttgcagtggtttcgcttcggttattcaggcaaaacaccagttgaaagtttctggaatgagctgtcctttatgttcaaaccaagatgttcttttgaagaggaactcactgaagaagcttctggatctttctttcaacaatcaccgcagaaactattcaaagtatccgatccctattctatggatccacctgctggttacatgaatgagacaccttactcaagtgcgatgaatcagcaaatacttatcccaggaaagctcaggccacttctgcatcttcctgatctttttggtgacaagactattggtgaacacttaactatggacttgaaaccaatcgatctgattttctacttccacaaggcaatgaagaaagatctagactaccttgtacgtggttcagctcggctcgcaacagattatagcttccttggagaatttcaacagcggtttcatcttataaagttcttgtatcagatacattcagatgcagaagatgagattgcatttccagctttggaggcaaagggtaaactacaaaacattagtcagtcatacagtatcgatcacgaactcgaagttgagcacctcaataaagtatcattccttttgaatgagttggcagaattgaacatgctggtactagaccacaagaatgtgaagtatgagaagttgtgtatgagcctccaggatatatgcaaatccattcacaagctattgtctgagcatctccaccgagaagaaactgagctttggtgtttattcagagactgcttcactattgaagaacaagaaaagatcatagcatgcatgcttggaagaataagtggagaaatattgcaagatatgattccatggctaatggaatctttgatccctgatgaacaacatgctgtgatgtccttgtggagacaagcaacaaggaaaactatgtttggtgaatggctaacagaatggtacaattctcatgctgtcgaggaggaaacagaggaagcaaataaggatccatctgagaattcagatccattagacgttgtctggagttatctctttgaaggagctgctgatgagtacaaagggagcatttgcagcaaacctctggaagagacagagttaaaaggtatcatgaataagcctcttggaaaggctgctcctaataacaaggtagaatttggaaacaaagaggagaatcatcttgagatctcaggaagtaaaaaggtgtgtacaggagctgatgaaacgaagtacaaagaacaaactgacagcaacgcgcaagcttttcagatgtcgcataatactagccaatctggccaagacagtagatatgagtgtctgttatcaatgagtcaagaggatgtggaagctacaattcgaagaatatctcgagattcttccctggatcctcagaagaaatcgtatatcattcaaaacttgttaatgagccgttggattgctacacaacggatatataatcttgaaccatccattctctcaagcaatagggaagcagttccggggcagaatccatcttatcgagatcctcacaaactgatctttggttgcaaacactacaagagaagttgcaaacttcttgctccttgttgcaacaagctctacacatgtatacgatgccatgatgaagaggttgatcacttattggatagaaaacagatcacaaagatgatgtgcatgaaatgcatgataattcagccagtaggtgctagttgctcaaatatttcatgtagttcatcaatgggaaaatattactgtaaaatatgcaagttgtttgacgatgatagggaaatctatcattgtccatactgcaatctctgccggctaggaaaaggactgagcatagactactttcattgcatgaaatgtaatgcttgtatgtcgcgactcatagtagaacatgtatgcagagaaaagtgcttagaagataattgtccgatttgtcatgaatacatatttacctcaaattctccggtgaaggctcttccatgcggtcacgtgatgcattcaacatgctttcaggagtacacatgttcgcattacacatgccctatttgcagcaagtcgctcggggatatgcaggtttactttagaatgttagacgcactgcttgcagaacaaaagatgccagatgaatacttaaaccaaactcaggtaatactgtgtaatgactgtggaagaaaggggaatgctccttaccattggctctatcacaagtgttcttcttgtgcctcctacaacaccagacttttctag

SEQ ID NO: 154 - DNA, 1989 bp, Arabidopsis thaliana
  source 1..1989: mol_type = other DNA
ctagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagg

SEQ ID NO: 155 - DNA, 1992 bp, Zea mays
  source 1..1992: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 156 - DNA, 1346 bp, Cauliflower mosaic virus
  source 1..1346: mol_type = other DNA
tcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacg

SEQ ID NO: 157 - DNA, 49 bp, synthetic construct
  source 1..49: mol_type = other DNA
ggggacaactttgtatagaaaagttgtaaatttgagttattattgttca

SEQ ID NO: 158 - DNA, 48 bp, synthetic construct
  source 1..48: mol_type = other DNA
ggggactgcttttttgtacaaacttgatttaattggcctgcttaatct

SEQ ID NO: 159 - DNA, 50 bp, synthetic construct
  source 1..50: mol_type = other DNA
ggggacaactttgtatagaaaagttgtagaaattcgaaagcgacgatgca

SEQ ID NO: 160 - DNA, 48 bp, synthetic construct
  source 1..48: mol_type = other DNA
ggggactgcttttttgtacaaacttgacggcgaaacgaacaaaaaagt

SEQ ID NO: 161 - DNA, 50 bp, synthetic construct
  source 1..50: mol_type = other DNA
ggggacaactttgtatagaaaagttgtataaaggatttgggtctattcga

SEQ ID NO: 162 - DNA, 49 bp, synthetic construct
  source 1..49: mol_type = other DNA
ggggactgcttttttgtacaaacttgaagatacctcacgatcctaagca

SEQ ID NO: 163 - DNA, 50 bp, synthetic construct
  source 1..50: mol_type = other DNA
ggggacagctttcttgtacaaagtggtaatgatgtcttttgtcgcaaact

SEQ ID NO: 164 - DNA, 46 bp, synthetic construct
  source 1..46: mol_type = other DNA
ggggacaactttgtataataaagttgatcacgcagcaggagcataa

SEQ ID NO: 165 - DNA, 54 bp, synthetic construct
  source 1..54: mol_type = other DNA
ggggacaactttgtatagaaaagttgtagtgtcttagtccttaaccacaatcac

SEQ ID NO: 166 - DNA, 56 bp, synthetic construct
  source 1..56: mol_type = other DNA
ggggactgcttttttgtacaaacttgatgatattttttgttggatatgaatgaaag

SEQ ID NO: 167 - DNA, 21 bp, synthetic construct
  source 1..21: mol_type = other DNA
acatgtccggtactatcgcag

SEQ ID NO: 168 - DNA, 22 bp, synthetic construct
  source 1..22: mol_type = other DNA
tccatcaagacagctaatgagc

SEQ ID NO: 169 - DNA, 21 bp, synthetic construct
  source 1..21: mol_type = other DNA
ccaaatgcgttcgtaggtaag

SEQ ID NO: 170 - DNA, 23 bp, synthetic construct
  source 1..23: mol_type = other DNA
tcagatttacacaaatttgcagc

SEQ ID NO: 171 - DNA, 21 bp, synthetic construct
  source 1..21: mol_type = other DNA
caaaatccattcggacatgtc

SEQ ID NO: 172 - DNA, 21 bp, synthetic construct
  source 1..21: mol_type = other DNA
gaggctaaaactccacccaag

SEQ ID NO: 173 - DNA, 21 bp, synthetic construct
  source 1..21: mol_type = other DNA
tgcttggcataatccttcttg

SEQ ID NO: 174 - DNA, 22 bp, synthetic construct
  source 1..22: mol_type = other DNA
gaactctcttgcttcctgaagc

SEQ ID NO: 175 - DNA, 21 bp, synthetic construct
  source 1..21: mol_type = other DNA
tcggttattcaggcaaaacac

SEQ ID NO: 176 - DNA, 21 bp, synthetic construct
  source 1..21: mol_type = other DNA
ccctttgtactcatcagcagc

SEQ ID NO: 177 - DNA, 20 bp, synthetic construct
  source 1..20: mol_type = other DNA
acggtgccggagataaccta

SEQ ID NO: 178 - DNA, 20 bp, synthetic construct
  source 1..20: mol_type = other DNA
gtcggtcacgttcactagca

SEQ ID NO: 179 - DNA, 20 bp, synthetic construct
  source 1..20: mol_type = other DNA
ccgttcatgtcttcctgcct

SEQ ID NO: 180 - DNA, 20 bp, synthetic construct
  source 1..20: mol_type = other DNA
gcctttggtggctgcttaac

SEQ ID NO: 181 - DNA, 21 bp, synthetic construct
  source 1..21: mol_type = other DNA
gccacatctgcgtatcaagtt

SEQ ID NO: 182 - DNA, 20 bp, synthetic construct
  source 1..20: mol_type = other DNA
tcccaaacaagctacgacca

SEQ ID NO: 183 - DNA, 22 bp, synthetic construct
  source 1..22: mol_type = other DNA
gaatgtggaagcgagtcagcga

SEQ ID NO: 184 - DNA, 22 bp, synthetic construct
  source 1..22: mol_type = other DNA
gatcccggaggcgaaacactta

SEQ ID NO: 185 - DNA, 21 bp, synthetic construct
  source 1..21: mol_type = other DNA
attggtaacctccaccagctt

SEQ ID NO: 186 - DNA, 21 bp, synthetic construct
  source 1..21: mol_type = other DNA
catagggaccaaacaccacat

SEQ ID NO: 187 - DNA, 20 bp, synthetic construct
  source 1..20: mol_type = other DNA
ttcttccctcgaaagctcaa

SEQ ID NO: 188 - DNA, 20 bp, synthetic construct
  source 1..20: mol_type = other DNA
aaggcccaccagagtgtatg

SEQ ID NO: 189 - DNA, 20 bp, synthetic construct
  source 1..20: mol_type = other DNA
gtaccaccgcggactactgt

SEQ ID NO: 190 - DNA, 20 bp, synthetic construct
  source 1..20: mol_type = other DNA
cgtggagcaataagcactca

SEQ ID NO: 191 - DNA, 20 bp, synthetic construct
  source 1..20: mol_type = other DNA
catccttaagcgaagccttg

SEQ ID NO: 192 - DNA, 20 bp, synthetic construct
  source 1..20: mol_type = other DNA
tcgatgcgtgcatctgtaat

SEQ ID NO: 193 - DNA, 20 bp, synthetic construct
  source 1..20: mol_type = other DNA
caggatgttgatgccatgag

SEQ ID NO: 194 - DNA, 20 bp, synthetic construct
  source 1..20: mol_type = other DNA
aaggttggagaaggctggat

SEQ ID NO: 195 - DNA, 20 bp, synthetic construct
  source 1..20: mol_type = other DNA
ctcgagggaacatcaacgat

SEQ ID NO: 196 - DNA, 20 bp, synthetic construct
  source 1..20: mol_type = other DNA
actttcactgcccattttgg

SEQ ID NO: 197 - DNA, 21 bp, synthetic construct
  source 1..21: mol_type = other DNA
tgtgccaatctacgagggttt

SEQ ID NO: 198 - DNA, 19 bp, synthetic construct
  source 1..19: mol_type = other DNA
tttcccgctctgctgttgt

SEQ ID NO: 199 - DNA, 20 bp, synthetic construct
  source 1..20: mol_type = other DNA
ggcggagagagttcaagagt

SEQ ID NO: 200 - DNA, 20 bp, synthetic construct
  source 1..20: mol_type = other DNA
agaccccaaaagctcctcaa

SEQ ID NO: 201 - DNA, 20 bp, synthetic construct
  source 1..20: mol_type = other DNA
gccgggcaagtgattgtatt

SEQ ID NO: 202 - DNA, 20 bp, synthetic construct
  source 1..20: mol_type = other DNA
tccaagaaccggtgaagtga

SEQ ID NO: 203 - DNA, 20 bp, synthetic construct
  source 1..20: mol_type = other DNA
aatgtcaagtttcggccttg

SEQ ID NO: 204 - DNA, 20 bp, synthetic construct
  source 1..20: mol_type = other DNA
aggtctcatgtcgacccaac

SEQ ID NO: 205 - DNA, 20 bp, synthetic construct
  source 1..20: mol_type = other DNA
cgacgttagttgtgccttca

SEQ ID NO: 206 - DNA, 20 bp, synthetic construct
  source 1..20: mol_type = other DNA
tctaaacgccgatgtcacag

SEQ ID NO: 207 - DNA, 20 bp, synthetic construct
  source 1..20: mol_type = other DNA
caccgaaagagaccttcagc

SEQ ID NO: 208 - DNA, 20 bp, synthetic construct
  source 1..20: mol_type = other DNA
aactaacgtaacccgcatcg

(19) United States
(12) Patent Application Publication
(10) Pub. No.: US 2025/0163450 A1
(43) Pub. Date: May 22, 2025

(54) IRON MAN (IMA) OVEREXPRESSION INCREASES DISEASE RESISTANCE

(71) Applicant: Salk Institute for Biological Studies, La Jolla, CA (US)

(72) Inventors: Min Cao, La Jolla, CA (US); Wolfgang Busch, La Jolla, CA (US)

(73) Assignee: Salk Institute for Biological Studies, La Jolla, CA (US)

(21) Appl. No.: 18/954,772

(22) Filed: Nov. 21, 2024

Related U.S. Application Data
(60) Provisional application No. 63/601,364, filed on Nov. 21, 2023.

(51) Int. Cl. C12N 15/82; C07K 14/415; C12N 9/22

(52) U.S. Cl. CPC C12N 15/8281; C07K 14/415; C12N 9/22; C12N 15/8201; C12N 15/8223; C12N 2310/20

(57) ABSTRACT

The present disclosure provides compositions and methods for increasing disease resistance in plants, for example by increasing IRON MAN (IMA) expression and/or activity, reducing BRUTUS-LIKE E3 ligase (BTSL) expression and/or activity, or both.

### CROSS REFERENCE TO RELATED APPLICATION

This application claims priority to U.S. Provisional Application No. 63/601,364, filed Nov. 21, 2023, which is herein incorporated by reference in its entirety.

### ACKNOWLEDGMENT OF GOVERNMENT SUPPORT

This invention was made with government support under R01GM127759 awarded by the National Institutes of Health. The government has certain rights in the invention.

### FIELD

The present disclosure generally relates to the field of increasing resistance to disease in plants. More particularly, the present disclosure relates to compositions and methods for generating plants that possess increased disease resistance, for example a disease that infects or diseases plant shoots.

### INCORPORATION OF SEQUENCE LISTING

The Sequence Listing is submitted as an XML file in the form of the file named “111144-02_Sequence Listing.xml” (˜217,872 bytes), which was created on Nov. 3, 2024 and is incorporated by reference herein.

### BACKGROUND

Iron is critical during host-microbe interactions. Restriction of available iron by the host during infection is a defense strategy, described as nutritional immunity. However, this poses a conundrum for externally facing, absorptive tissues like the gut epithelium or the plant root epidermis that generate environments favoring iron bioavailability. For instance, plant roots acquire iron mostly from the soil and when iron deficient, increase iron availability through mechanisms that include rhizosphere acidification and secretion of iron chelators. Yet, the elevated iron bioavailability would also be beneficial for the growth of bacteria which threaten plant health.

Iron is an essential nutrient for organismal growth throughout all branches of life. Although iron is among the most common elements on our planet, its bioavailability in most environments is low and is a limiting factor for growth. Consequently, strong competition for iron is common between organisms. In mammals, iron levels have a direct impact on the composition of gut microbiota and can modulate host inflammatory responses restricting iron availability for pathogenic microbes. In plants, iron-limited soil environments trigger the activation of coumarin secretion in roots, which contributes to the alteration of the root-associated microbiota. Conversely, the soil-borne bacterial community can also have a significant impact on root iron acquisition. On the other side of the spectrum, pathogens can compete for and restrict iron for plants, plant immunity responses affect bacterial iron homeostasis, and beneficial microbes can produce siderophores that pathogens cannot use, consequently suppressing pathogen growth. Overall, iron plays a complex role in regulating plant-microbe interactions.

### SUMMARY

Provided herein are methods for increasing resistance against a plant pathogen in a plant, plant part, or plant cell, comprising increasing expression and/or activity of an IRON MAN (IMA), and/or reducing expression and/or activity of a BRUTUS-Like E3 ligase (BTSL) in the plant, plant part, or plant cell, thereby increasing resistance against a plant pathogen in the plant, plant part, or plant cell.

In some aspects, the plant pathogen is a virus, bacterium, nematode, or fungus. In some aspects, the pathogen is a foliar bacterial pathogen. In some aspects, the pathogen is a pathogen that infects a shoot of the plant. In some aspects, the pathogen is Pseudomonas syringae, Ralstonia solanacearum, Xanthomonas oryzae, Xanthomonas campestris, Xanthomonas axonopodis, Erwinia amylovora, Xylella fastidiosa, Dickeya dadantii, or Pectobacterium carotovorum.

In some aspects, the increasing expression and/or activity of an IMA includes introducing one or more exogenous nucleic acid molecules that encode an IMA protein into the plant, plant part, or plant cell, thereby generating a gene-edited plant, gene-edited plant part, or gene-edited plant cell comprising the exogenous nucleic acid.

In some aspects, the IMA protein is a full-length IMA protein, or a C-terminal fragment of a full-length IMA protein. In some aspects, the C-terminal fragment of the IMA protein is 7 to 20 amino acids.

In some aspects, the C-terminal fragment of the IMA protein is 10 amino acids. In some aspects, the C-terminal fragment of the IMA protein includes G/N/D-D-D-D-x₍₁₋₆₎-D-x-A-P-A-A. In some aspects, the IMA protein is an IMA protein native to the plant, plant part, or plant cell, or an IMA1 protein ortholog. In some aspects, the full-length IMA protein includes at least 80%, 85%, 90%, 95%, 96%, 97%, 98%, 99%, or 100% sequence identity to any one of SEQ ID NOs: 1-134, or the C-terminal fragment comprises at least 10 of the C-terminal amino acids of any one of SEQ ID NOs 1-134.

In some aspects, the reducing expression and/or activity of a BTSL includes introducing one or more exogenous nucleic acid molecules that reduce expression of a BTSL gene and/or reduce activity of a protein encoded by the BTSL gene into a plant, a plant part, or a plant cell, thereby generating a gene-edited or transformed plant, gene-edited or transformed plant part, or gene-edited or transformed plant cell comprising the exogenous nucleic acid.

In some aspects, the introducing one or more exogenous nucleic acid molecules generates a deletion of or loss-of-function mutation in the BTSL gene. In some aspects, the one or more exogenous nucleic acid molecules include one or more guide nucleic acid molecules that can delete or mutate the BTSL gene.

In some aspects, the one or more exogenous nucleic acid molecules include one or more RNAi molecules, or generate one or more RNAi molecules, wherein the one or more RNAi molecules target mRNAs transcribed from the BTSL gene. In some aspects, the BTSL gene is a BTSL gene native to the plant, plant part, or plant cell, or a BTSL gene ortholog.

In some aspects, the method further includes introducing one or more Cas proteins or one or more nucleic acid molecules encoding a Cas protein into the plant, plant part, or plant cell.

In some aspects, the one or more exogenous nucleic acid molecules are operably linked to a promoter. In some aspects, the promoter is a non-native promoter. In some aspects, the promoter is constitutive promoter, tissue-specific promoter, or an inducible promoter. In some aspects, the tissue-specific promoter is active in a shoot cell, epidermal cell, or vascular cell of the plant. In some aspects, the promoter is a 35S promoter, or a plant ubiquitin (Ubi-1) promoter.

In some aspects, at least one of the one or more exogenous nucleic acid molecules that encode the IMA protein includes at least 80%, 85%, 90%, 95%, 96%, 97%, 98%, 99%, or 100% sequence identity to any one of SEQ ID NOs: 135-138.

In some aspects, the BTSL gene includes at least 80%, 85%, 90%, 95%, 96%, 97%, 98%, 99%, or 100% sequence identity to any one of SEQ ID NOs: 152-153; and/or the protein encoded by the BTSL gene comprises at least 80%, 85%, 90%, 95%, 96%, 97%, 98%, 99%, or 100% sequence identity to any one of SEQ ID NOs: 139-151.

In some aspects, the expression and/or activity of the IMA is increased as compared to a control plant, plant part, or plant cell; and the expression and/or activity of the BTSL is reduced as compared to a control plant, plant part, or plant cell. In some aspects, wherein the expression or activity of the IMA is increased by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 350%, 400%, 450%, 500%, 550%, 600%, 650%, or 700%; and/or wherein the expression or activity of the BTSL is reduced by at least 10%, 15%, 20%, 25%, 30%, 35%, 40%, 45%, 50%, 55%, 60%, 65%, 70%, 75%, 80%, 85%, 90%, 95%, or 100%.

In some aspects, the plant is a pennycress, soybean, canola, rice, tomato, Zea mays, or sorghum plant; the plant part is from a pennycress, soybean, canola, rice, tomato, Zea mays, or sorghum plant; and/or the plant cell is from a pennycress, soybean, canola, rice, tomato, Zea mays, or sorghum plant.

In some aspects, the resistance is increased by at least 10%, at least 20%, at least 30%, at least 40%, at least 50%, at least 75%, at least 90%, at least 100%, at least 200%, or at least 400% relative to a control plant, a control plant part, or a control plant cell.

In some aspects, the method further includes measuring mean resistance in the gene-edited plant, gene-edited plant part, or gene-edited plant cell as compared to the control plant, control plant part, or control plant cell.

Also disclosed herein are gene-edited plants, gene-edited plant parts, and gene-edited plant cells generated by any method disclosed herein.

In some aspects, the gene-edited plant, gene-edited plant part, or gene-edited plant cell includes a single locus conversion. In some aspects, the gene-edited plant, gene-edited plant part, or gene-edited plant cell further includes one or more additional exogenous nucleic acid(s) encoding a protein(s) that confers upon the gene-edited plant, gene-edited plant part, or gene-edited plant cell a desired trait, wherein the desired trait is one or more of herbicide tolerance, drought tolerance, heat tolerance, low or high soil pH level tolerance, salt tolerance, resistance to an insect, resistance to a bacterial disease, resistance to a viral disease, resistance to a fungal disease, resistance to a nematode, resistance to a pest, male sterility, site-specific recombination; abiotic stress tolerance, modified phosphorus characteristics, modified antioxidant characteristics; modified essential seed amino acid characteristics, decreased phytate, modified fatty acid metabolism, and modified carbohydrate metabolism.

Also provided herein are methods of producing a commodity plant product, which can include collecting or producing the commodity plant product from a gene-edited plant, gene-edited plant part, or gene-edited plant cell disclosed herein.

In some aspects, the commodity plant product comprises a non-native nucleic acid molecule or protein from the gene-edited plant, gene-edited plant part, or gene-edited plant cell. In some aspects, the commodity product comprises a protein concentrate, protein isolate, leaves, extract, oil, bean, and/or seed.

Also provided herein are methods of producing plant seed, comprising crossing a gene-edited plant disclosed herein with itself or a second plant.

Also provided herein are methods for breeding a plant with increased disease resistance, comprising crossing a gene-edited plant disclosed herein with a second plant; obtaining seed from the crossing; planting the seeds and growing the seeds to plants; and selecting from said plants those with increased disease resistance, optionally in comparison to a wild type plant, or in comparison to a control plant.

Also provided herein are recombinant nucleic acid molecules that include a nucleic acid sequence operably linked to a heterologous promoter, wherein the nucleic acid molecule encodes (a) an amino acid sequence with at least 80% sequence homology to any of SEQ ID NOs: 1-134, (b) an amino acid sequence that includes at least 10 contiguous C-terminal amino acids of any of SEQ ID NOs: 1-134, or (c) an amino acid sequence that includes G/N/D-D-D-D-x₍₁₋₆₎-D-x-A-P-A-A; and wherein expression of the recombinant nucleic acid molecule in a plant results in increased disease resistance in the plant when compared to a plant of the same species lacking the recombinant nucleic acid molecule.

Also provided are recombinant nucleic acid molecules disclosed herein, for use in any method disclosed herein.

In some aspects, in any of the method or plant part disclosed herein, the plant part is a protoplast, leaf, stem, root, root tips, anther, pistil, stamen, seed, embryo, pollen, ovule, microspore, protoplast, sporophyte, gametophyte, cotyledon, hypocotyl, flower, shoot, tissue, petiole, or meristematic cell.

### BRIEF DESCRIPTION OF THE DRAWINGS

The patent or application file contains at least drawing executed in color. Copies of this patent or patent application publication with color drawing(s) will be provided by the Office upon request and payment of the necessary fee.

FIGS. 1A-1G. flg22 represses iron uptake during iron deficiency.

(A-C) 15-day-old Arabidopsis seedling leaves in sufficient iron (50 μM) or low iron (10 μM) with or without low levels of flg22 (10 nM) treatment. (A) Shoots; Scale bar 1 cm, (B) total chlorophyll concentration of Col-0 shoots, (C) Iron concentration of Col-0 seedlings; 3 biological replicates. Error bars: s.e.m. Statistically analysis: two-sided t-test.

(D) Ferric chelate reductase activity in Col-0 roots grown for 7 days under +Fe conditions and transferred to +Fe, +Fe with flg22, −Fe and −Fe with flg22 liquid media for 2 days. 5 biological replicates. Error bars: s.e.m. Different letters indicate statistically significant differences between different conditions analyzed by one-way ANOVA and Tukey's test (p<0.05).

(E) Promoter activity of IRT1 in the root of pIRT1::NIS-2xYpet seedlings in response to +Fe, +Fe with flg22, −Fe and −Fe with flg22 treatment. Seedlings were grown on the +Fe medium and after 5 days transferred to the different liquid media for 24 hours treatment. MZ: Meristematic zone; EZ: elongation zone; Green: Nuclear localized Ypet; Red: propidium iodide (PI) cell wall stain. For each treatment, a representative single confocal section (single image, GFP/PI), Maximum Intensity Z-Projection (Z-max, GFP only), a single optical section of the transverse view, and the Z-projection of the transverse section are shown. Scale bar, 50 μm.

(F) Raw signal intensity quantification of pIRT1 reporter. Different letters indicate statistically significant differences between different conditions by one-way ANOVA and Tukey's test (p<0.05).

(G) Western blots showing IRT1 protein levels in Col-0 roots grown in +Fe, +Fe with flg22, −Fe and −Fe with flg22 treatment. Arrow indicates the IRT protein band. The asterisk indicates a non-specific band. Tubulin protein: internal control.

FIGS. 2A-2H. flg22 represses iron deficiency responses through IMA1.

(A) Venn diagram of differentially expressed genes from RNAseq experiment.

(B) Heat map of mean-centered Z-scores for well-known iron responsive genes (3 independent biological repeats): iron deficiency signaling components, iron uptake components and long-distance signaling components.

(C-E) Phenotypes in 15-day-old Col-0 and IMA1ox seedlings in response to sufficient iron (50 μM) or low iron (10 μM) with or without low level flg22 (10 nM) treatment. (C) Shoots; Scale bar, 1 cm. (D) Total chlorophyll concentrations of shoots. (E) Iron concentration of seedlings. (D, E) 3 biological replicates. Error bar: s.e.m. P-values from two-tailed Student t-test.

(F) Western blots showing IRT1 protein levels in Col-0 and IMA1ox roots in response to +Fe, +Fe with flg22, −Fe and −Fe with flg22 treatment. Internal control Tubulin.

(G) Quantitative analysis of ferric chelate reductase activities in Col-0 and IMA1ox roots grown for 7 days under +Fe conditions and transferred to +Fe, +Fe with flg22, −Fe and −Fe with flg22 liquid media for 2 days. 5 biological replicates. Error bars: s.e.m. Different letters indicate statistically significant differences between different conditions analyzed by one-way ANOVA and Tukey's test (p<0.05).

(H) Heat map of mean-centered Z-scores (normalized to Col-0+Fe) for well-known iron-responsive genes.

FIGS. 3A-3H. flg22 spatially represses IMA1 in the ground tissue of the root.

(A, B) IMA1 protein levels in pIMA1::EYFP-IMA1;ima8x roots. Internal control: Tubulin. (A) Response to +Fe, +Fe with flg22, −Fe and −Fe with flg22 treatment. (B) Time course: Seedlings were pre-treatment with −Fe for 36 hours, then treated with −Fe or −Fe+flg22 (1 μm flg22) for 0, 3 and 6 hours.

(C) IMA1 distribution in response to +Fe, +Fe with flg22, −Fe and −Fe with flg22 treatment in differentiation zone of the root. Five-day-old pIMA1::EYFP-IMA1;ima8x seedlings were grown on +Fe medium and then transferred to liquid treatment medium for 24 hours. Yellow: EYFP-IMA1 signals; Red: Propidium iodide (PI) cell wall stain. For each treatment, a representative single confocal section (single image, EYFP/PI), a maximal Z-projection of the Z-stack (Z-max, EYFP only), a single optical section of the transverse view, and the Z-projection of the transverse section is shown. Scale bar, 50 μm.

(D) Quantification of IMA1 fluorescence signal intensity in the differentiation zone of roots (n=15 biologically independent seedlings). Statistical analysis: Kruskal Wallis/two-tailed test followed by Multiple pairwise comparisons using the Steel-Dwass-Critchlow-Fligner procedure/two-tailed test (p<0.05).

(E) Schematic of quantification method for normalized signal diameter.

(F) Quantification of normalized IMA1 signal diameter in different treatment conditions in differentiation zones of the roots (n=15 biologically independent seedlings). Statistical analysis: Kruskal Wallis/two-tailed test followed by Multiple pairwise comparisons using the Steel-Dwass-Critchlow-Fligner procedure/two-tailed test (p<0.05).

(G, H) Western blots showing IRT1 protein levels in roots of transgenic plant with different tissue-specific promoter driving IMA1 expression. Internal control Tubulin. (G) All seedlings were grown in +Fe to avoid the endogenous IRT1 induction by low iron. Three independent lines of each transgenic plants are shown (refer to extended data FIG. 6E). (H) pPGP4::mCtirine-IMA1 roots in response to +Fe and +Fe with flg22 treatment.

FIGS. 4A-4I. flg22 represses iron deficiency responses via IMA1 degradation through BTSL1 and BTSL2.

(A-C) 15-day-old seedlings in +Fe (50 μM) or −Fe (10 μM) in without or low level flg22 (10 nM) treatment. (A) Shoots; scale bar, 1 cm. (B) Total chlorophyll concentrations of shoots. (C) Iron concentration of seedlings. (B, C) 3 biological replicates. Error bar: s.e.m. Two-tailed Student t-test.

(D) Ferric chelate reductase activities in Col-0 and btsl1,2 roots grown for 7 days under +Fe and transferred to +Fe, +Fe with flg22, −Fe and −Fe with flg22 liquid media for 2 days. 5 biological replicates. Error bar: s.e.m. One-way ANOVA and Tukey's test (p<0.05).

(E) Western blots showing IRT1 protein levels in Col-0 and btsl1,2 roots in +Fe, +Fe with flg22, −Fe and −Fe with flg22 treatment. Control: Tubulin.

(F) Transverse confocal microscopy sections of root differentiation zones of pBTSL1-GFP and pBSTL2-GFP in response to +Fe, +Fe with flg22, −Fe and −Fe with flg22 treatments. Green: GFP channel; scale bar, 50 μm.

(G) Confocal microscopy images of IMA1 in +Fe, +Fe with flg22, −Fe and −Fe with flg22 treatment in root differentiation zone. 5-day-old pIMA1::EYFP-IMA1;ima8x and pIMA1::EYFP-IMA1;btsl1,2 seedlings grown on +Fe medium, then transferred to liquid treatment media for 24 hours. Yellow: EYFP-IMA1; Red: Propidium iodide (PI) cell wall stain. Shown per treatment: representative single confocal section (single image, EYFP/PI), maximal Z-projection (Z-max, EYFP only), single optical section of the transverse view, Z-projection of the transverse section. Scale bar, 50 μm.

(H, I) IMA1 fluorescence signal intensity (H) and normalized IMA1 signal diameter (I) in +Fe, +Fe with flg22, −Fe and −Fe with flg22 treatment in root differentiation zone of pIMA1::EYFP-IMA1;ima8x and pIMA1::EYFP-IMA1;btsl1,2. Same dataset as shown in FIG. 3 e&f of pIMA1::EYFP-IMA1;ima8x as images were taken at the same time. Statistical analysis: Kruskal Wallis/two-tailed test followed by Steel-Dwass-Critchlow-Fligner procedure/two-tailed test (p<0.05).

FIGS. 5A-5H. IMA1 dependent rhizosphere acidification regulates root immune-responses and bacterial colonization.

(A, B) Rhizosphere acidification responses of Col-0, ima8x and UBQ10::mCitrine-IMA1 in +Fe and −Fe treatment. Bromocresol purple was used as pH indicator. (B) Standard media acidification by Col-0, ima8x and UBQ10::mCitrine-IMA1 in response to +Fe, +Fe with flg22, −Fe and −Fe with flg22 treatments. 6 biological replicates. Error bar: s.e.m. Statistical analysis: one-way ANOVA and Tukey's test (p<0.05). Two-tailed Student t-test shows statistically significant difference of media acidification of Col-0 and UBQ10::mCitrine-IMA1 under +Fe.

(C, D) Quantification of root length of Col-0, ima8x and UBQ10::mCitrine-IMA1 in +Fe and +Fe+flg22 conditions (C) without MES-KOH and (D) with 1 mM MES-KOH. P-values: two-tailed Student t-test.

(E) Quantification of flg22-mediated root growth responses (+flg22/−flg22, root length of +flg22 divided by mean of −flg22) of Col-0, ima8x and UBQ10::mCitrine-IMA1 with/without MES-KOH. Horizontal line: mean. Error bars s.e.m. P-values: two-tailed Student t-test.

(F) CYP71A12 transcript level in +Fe and +Fe with flg22 by quantitative RT-PCR. Roots were treated with 1 μM flg22 for 1 hour. Normalized to ACT2. 3 biological replicates. Error bars: s.e.m. Statistical analysis: one-way ANOVA and Tukey's test (p<0.05).

(G) Western blots showing MAPK phosphorylation by flg22 in Col-0, ima8x and UBQ10::mCitrine-IMA1 roots in response to flg22. The roots were treated with 1 μM flg22 for 0, 5 and 10 minutes. Internal control Tubulin.

(H) Colonization of 7-day-old Arabidopsis roots at 1 d post-inoculation by CHA0 under +Fe, −Fe and non-available iron (nAvFe) conditions. 3 biological replicates. Error bars: s.e.m. Statistical analysis: one-way ANOVA and Tukey's test (p<0.05).

FIGS. 6A-6H. IMA1 accumulation is distinctly modulated by surface dwelling or invading bacteria.

(A) pIRT1::NIS-2xYpet roots in +Fe, +Fe+CHA0, −Fe, −Fe+CHA0 and −Fe+flg22. Red: cell surface-localized CHA0-mcherry bacteria; green: nuclear localized Ypet. Representative single confocal section (single image, GFP/mCherry), a maximal Z-projection of the Z-stack (Z-max, GFP only) and a single optical section of the transverse view are shown. Scale bar: 50 μm.

(B) Normalized signal quantification in pIRT1::NIS-2xYpet roots in +Fe, +Fe+CHA0, −Fe, −Fe+CHA0 and −Fe+flg22. One-way ANOVA and Fisher's LSD test (two-sided, p<0.05).

(C) IMA1 distribution in +Fe, +Fe+CHA0, −Fe, −Fe+CHA0 and −Fe+flg22 in root differentiation zone of pIMA1::EYFP-IMA1;ima8x. Red: root surface-localized CHA0-mcherry bacteria; yellow: EYFP-IMA1 (yellow channel). Representative single confocal section (single image, EYFP/mCherry), maximal Z-projection of the Z-stack (Z-max, EYFP only), and single optical section of the transverse view are shown. Scale bar: 50 μm.

(D) Normalized IMA1 signal intensity in +Fe, +Fe+CHA0, −Fe, −Fe+CHA0 and −Fe+flg22. One-way ANOVA and Fisher's LSD test (two-sided, p<0.05).

(E) Normalized IMA1 signal intensity in epidermis-cortex cell layers in +Fe, +Fe+CHA0, −Fe, −Fe+CHA0 and −Fe+flg22. One-way ANOVA and Fisher's LSD test (two-sided, p<0.05).

(F) Normalized IMA1 signal diameter quantification in +Fe, +Fe+CHA0, −Fe, −Fe+CHA0 and −Fe+flg22. One-way ANOVA and Fisher's LSD test (two-sided, p<0.05).

(G) Representative images of EYFP-IMA1 in +Fe, +Fe+CHA0, −Fe, −Fe+CHA0 at emerging lateral root primordia. Red: Interior-localized CHA0-mcherry bacteria; yellow: EYFP-IMA1 signals. Asterisks: CHA0-mcherry entered the roots through lateral root primordia site. Arrow: Weak IMA1 signal in cortex. Scale bar: 50 μm. Single confocal section (single image, EYFP/mCherry), maximal Z-projection of the Z-stack (Z-max, EYFP only), and single optical section of the transverse view are shown. Scale bar: 50 μm.

(H) Normalized IMA1 signal intensity quantification in +Fe, +Fe+CHA0, −Fe, −Fe+CHA0 and −Fe+flg22 in lateral root primordia region. One-way ANOVA and Fisher's LSD test (two-sided, p<0.05).

FIGS. 7A-7M. Flg22 represses iron uptake through FLS2.

(A) Phenotype of 15-day-old Arabidopsis seedling leaves response to different iron concentration (shown in figures) with or without low levels of flg22 (10 nM) treatment. Scale bar: 1 cm.

(B, C) Phenotype of 15-day-old Arabidopsis seedling leaves response to sufficient iron (50 μM) or low iron (10 μM) with or without low levels of flg20 or flg22 (10 nM) treatment. (B) Shoots; scale bar: 1 cm. (C) Total chlorophyll concentration of Col-0 shoots. Bar chart centers show means of 3 biological replicates. Error bar: s.e.m. The numbers correspond to P-values were analyzed by two-tailed Student t-test.

(D) Quantitative analysis of ferric chelate reductase activities in Col-0 roots grown for 7 days under +Fe conditions and transferred to −Fe, −Fe with flg20 and −Fe with flg22 liquid medium for 2 days. The bar chart centers show means of 5 biological replicates. Error bar: s.e.m. Different letters indicate statistically significant differences between different conditions analyzed by one-way ANOVA and Tukey's test (p<0.05).

(E) Promoter activity of IRT1 in the root of pIRT1::NIS-2xYpet seedlings in response to −Fe, −Fe with flg20 and −Fe with flg22 treatment. Seedlings were grown on the +Fe medium and after 5 days transferred to the different liquid media for 24 hours treatment. Green: nuclear localized Ypet; red: propidium iodide (PI) cell wall stain. For each treatment, a representative single confocal section (single image, GFP/PI), Maximum Intensity Z-Projection (Z-max, GFP only), a single optical section of the transverse view, and the Z-projection of the transverse section are shown. Scale bar: 50 μm.

(F-H) Western blots showing IRT1 protein levels in Col-0 and irt1-1 roots grown in +Fe and −Fe (F) or Col-0 under +Fe, −Fe and −Fe with different concentrations of flg22 treatment (G) or Col-0 under +Fe, −Fe and −Fe with flg20 or flg22 treatment (H). Arrow indicates the IRT1 protein band. The asterisk indicates non-specific band. Tubulin protein was blotted as an internal control.

(I-K) Phenotype of 15-day-old Arabidopsis Col-0 and fls2 seedling leaves in response to sufficient iron (50 μM) or low iron (10 μM) with or without low levels of flg22 (10 nM) treatment. The numbers correspond to p-values that were analyzed by two-tailed Student t-test. (I) Shoots (scale bar: 1 cm); (j) total chlorophyll concentration of Col-0 shoots; (k) iron concentration of Col-0 seedlings; bar chart centers show means of 3 biological replicates. Error bar: s.e.m.

(L) Quantitative analysis of ferric chelate reductase activities in Col-0 and fls2 roots grown for 7 days under +Fe conditions and transferred to +Fe, +Fe with flg22, −Fe and −Fe with flg22 liquid medium for 2 days. The bar chart centers show means of 5 biological replicates. Error bar: s.e.m. Different letters indicate statistically significant differences between different conditions analyzed by one-way ANOVA and Tukey's test (p<0.05).

(M) Western blots showing IRT1 protein levels in Col-0 and fls2 roots in +Fe, +Fe with flg22, —Fe and −Fe with flg22 treatment. The tubulin protein was blotted as an internal control.

FIGS. 8A-8G. MAMPs regulate iron uptake through distinct mechanisms.

(A, B) Promoter activity of FRK1 in roots of pFRK1::NLS-3x mVenus seedlings in response to −Fe and −Fe with flg22 treatment (A) or promoter activity of MYB51 in roots of pMYB51::NLS-3x mVenus seedlings in response to −Fe and −Fe with elf18 treatment (B). 7-day-old seedlings are treated with −Fe and −Fe with 100 nM flg22 (A) or −Fe and −Fe with 100 nM elf18 (B) for 24 hours in liquid media. Green: nuclear localized mVenus signals; red: propidium iodide (PI) cell wall stain. In each treatment, a representative single confocal section (single image, GFP/PI) and Maximum Intensity Z-Projection (Z-max, GFP only) are shown. Scale bar: 50 μm.

(C) Promoter activity of CYP71A12 in roots of pCYP71A12::GUS seedlings in response to −Fe and −Fe with chitin treatment. 7-day-old seedlings are treated with −Fe and −Fe with 1 mg/mL chitin for 24 hours in liquid media. Scale bar: 0.5 cm.

(D) Quantitative analysis of ferric chelate reductase activities in Col-0 roots grown for 7 days under +Fe conditions and transferred to −Fe, −Fe only or −Fe with flg22, elf18 and chitin liquid medium for 2 days. The bar chart centers show means of 5 biological replicates. Error bars: s.e.m. Different letters indicate statistically significant differences between different conditions analyzed by one-way ANOVA and Tukey's test (p<0.05).

(E) Western blots showing IRT1 protein levels in Col-0 roots grown +Fe, −Fe only or −Fe with flg22, elf18 and chitin treatment. Tubulin protein was blotted as an internal control.

(F) Promoter activity of IRT1 in the root of pIRT1::NLS-2xYpet seedlings in response to −Fe only or −Fe with flg22, elf18 and chitin treatment. Green: Nuclear localized Ypet signals; red: propidium iodide (PI) cell wall stain. In each treatment, the Z-stack scan is processed to single confocal section (single image, GFP/PI), maximal Z-projection (Z-max, GFP only). Scale bar: 50 μm.

(G) Western blots showing IRT1 protein levels in 35S::3x HA-FIT roots in response to +Fe, +Fe with flg22, −Fe and −Fe with flg22 treatment. HA-tagged FIT protein was blotted using anti-HA antibody. Tubulin protein was blotted as an internal control.

FIGS. 9A-9F. Flg22 antagonistically regulates the iron deficiency transcriptional landscape through FLS2.

(A) Venn diagram of up/down-regulated genes of −Fe vs +Fe, +Fe+flg22 vs +Fe, and −Fe+flg22 vs −Fe, respectively. The statistical analysis of p-value was calculated by hypergeometric test (one-sided).

(B) Heat map of mean-centered Z-scores for 1290 differentially expressed genes identified across different treatments (+Fe, +Fe+flg22, −Fe and −Fe+flg22), arranged by k-means clustering. Box plot indicates the relative expression level based on median centered Z-score in different clusters. The GO terms analysis was performed using PANTHER17.0 (Fisher's Exact test, p-value<0.05) and indicated on the right side of the heatmap.

(C, D) Heat map of mean-centered Z-scores for differentially expressed genes (cluster 5 (C) and cluster 1 (D) in WT and fls2) identified across different treatments (+Fe, +Fe+flg22, −Fe and −Fe+flg22), arranged by k-means clustering.

(E) Heat map of mean-centered Z-scores (normalized by Col-0+Fe) for well-known iron responsive genes in response to +Fe, +Fe+flg22, −Fe and −Fe+flg22 in Col-0 and fls2 roots.

(F) Gene expression analysis in response to +Fe, +Fe with flg22, −Fe and −Fe with flg22 treatment in Col-0 and fls2 roots by quantitative RT-PCR. The gene expression level is normalized to ACT2 internal control. The bar chart centers show means of 3 biological replicates. Error bars: s.e.m. Different letters indicate statistically significant differences between different conditions analyzed by one-way ANOVA and Tukey's test (p<0.05).

FIGS. 10A-10E. Flg22 antagonistically regulates the iron deficiency transcriptional landscape through IMA1.

(A, B) Venn diagram of the iron deficiency up-regulated genes in Col-0 vs down-regulated genes of −Fe+flg22 vs −Fe in Col-0 vs down-regulated genes of −Fe+flg22 vs −Fe in UBQ10::mCitrine-IMA1 (A). Venn diagram of the iron deficiency up-regulated genes in Col-0 vs upregulated genes in UBQ10::mCitrine-IMA1 in +Fe vs Col-0+Fe (B). The statistical analysis of p-value was calculated by hypergeometric test.

(C, D) Heat map of mean-centered Z-scores for differentially expressed genes (cluster 5 and cluster 1 refer to extended data FIG. 3b in WT and UBQ10::mCitine-IMA1) identified across different treatments (+Fe, +Fe+flg22, −Fe and −Fe+flg22), arranged by k-means clustering.

(E) Heat map of mean-centered Z-scores (normalized by Col-0+Fe) for well-known PTI components in response to +Fe, +Fe+flg22, −Fe and −Fe+flg22, respectively.

FIGS. 11A-11H. Flg22 spatially regulates IMA1 protein level in the ground tissue of the root through FLS2.

(A) Western blots showing IRT1 protein levels in Col-0, ima8x and pIMA1::EYFP-IMA1;ima8x in different treatment conditions. Seedlings were treated with +Fe and −Fe (Col-0 and ima8x) and +Fe, +Fe with flg22, −Fe and −Fe with flg22 treatment (pIMA1::EYFP-IMA1;ima8x). Tubulin protein was blotted as an internal control.

(B) Quantitative analysis of ferric chelate reductase activities in pIMA1::EYFP-IMA1;ima8x roots grown for 7 days under +Fe conditions and transferred to +Fe, +Fe with flg22, −Fe and −Fe with flg22 liquid medium for 2 days. The bar chart centers show mean of 5 biological replicates. Error bars: s.e.m. Different letters indicate statistically significant differences between different conditions analyzed by one-way ANOVA and Tukey's test (p<0.05).

(C) IMA1 distribution in response to +Fe, +Fe with flg22, −Fe and −Fe with flg22 treatment in differentiation zone of the root. 5-day-old pIMA1::EYFP-IMA1;ima& and pIMA1::EYFP-IMA1;fls2 seedlings were grown on the +Fe medium and then transferred to different liquid media for 24 hours treatment. The cytosolic and nuclear localized EYFP-IMA1 signals (yellow channel) are visualized with propidium iodide (PI, cell wall staining, red channel). For each treatment, a representative single confocal section (single image, EYFP/PI), a maximal Z-projection of the Z-stack (Z-max, EYFP only), a single optical section of the transverse view, and the Z-projection of the transverse section are shown. Scale bar: 50 μm.

(D, E) Quantification of IMA1 fluorescence signal intensity (D) and normalized IMA1 signal diameter (E) in response to +Fe, +Fe with flg22, −Fe and −Fe with flg22 treatment in differentiation zone of the root in pIMA1::EYFP-IMA1;ima8x and pIMA1::EYFP-IMA1;fls2 (n=15 biologically independent seedlings from 3 biological repeats). The same dataset from FIGS. 3D and 3F of pIMA1::EYFP-IMA1;ima8x were used here as the images for quantification were taken at the same time. Different letters indicate statistically significant differences between different conditions analyzed by Kruskal Wallis/two tailed test followed by Multiple pairwise comparisons using the Steel-Dwass-Critchlow-Fligner procedure/two-tailed test (p<0.05).

(F) Western blots showing IMA1 protein levels in the roots of pIMA1::EYFP-IMA1;ima8x and pIMA1::EYFP-IMA1;fls2 in +Fe, +Fe with flg22, −Fe and −Fe with flg22 treatments. Tubulin protein was blotted as an internal control.

(G) Western blots showing IMA1 protein levels in pIMA1::EYFP-IMA1;ima8x and pIMA1::EYFP-IMA1;fls2 roots. The seedlings were pre-treated with −Fe for 36 hours, then treated with −Fe+flg22 (1 μm flg22) for 0, 3 and 6 hours. Tubulin protein was blotted as an internal control.

(H) Representative image of EYFP-IMA1 signal intensity profile in response to +Fe, +Fe with flg22, −Fe and −Fe with flg22 treatment in differentiation zone of root in pIMA1::EYFP-IMA1;ima8x (left panel) and pIMA1::EYFP-IMA1;fls2 (right panel). The white line in YFP channel indicates the line for signal quantification. The red dashed lines indicate the boundary between the ground tissue and the stele. Scale bar: 50 μm.

FIGS. 12A-12E. Flg22 does not fully repress IMA1 transcription in the ground tissue and flg22 dependent callose deposition is not required for IRT1 repression.

(A) Promoter activity of IMA1 in the root of pIMA1::mCitrine-NLS-mCitrine seedlings in response to +Fe, +Fe with flg22, −Fe and −Fe with flg22 treatment. 5-day-old seedlings were grown on the +Fe medium and then transferred to the different liquid media for 24 hours treatment. The nuclear localized mCitrine signals (Yellow channel) are visualized with propidium iodide (PI, red channel). For each treatment, a representative single confocal section (single image, GFP/PI), Maximum Intensity Z-Projection (Z-max, GFP only), a single optical section of the transverse view, and the Z-projection of the transverse section is shown. Scale bar: 50 μm.

(B, C) Normalized IMA1 promoter activity quantification in all cell layers (B) or in epidermis-cortex cell layers (C) in response to +Fe, +Fe+flg22, −Fe and −Fe+flg22 treatments (n=9, 3 repeats). Different letters indicate statistically significant differences between different conditions analyzed by one-way ANOVA and Fisher's LSD test (two-sided, p<0.05).

(D) Western blots showing IRT1 protein levels in Col-0 roots is in response to +Fe, +Fe with flg22, −Fe, −Fe with flg22 treatment and −Fe with flg22 and DDG treatment. Tubulin protein was blotted as an internal control.

(E) Representative images of cell-layer specific IMA1 expression transgenic plants. 3 individual lines are shown under +Fe condition. Scale bar: 100 μm.

FIGS. 13A-13D. IMA1 is not regulated through cell-to-cell movement but regulated through protein level in the epidermis-cortex under −Fe by flg22.

(A) Representative images of roots of plants with different tissue-specific promoters driving IMA1 expression. 5-day-old transgenic seedlings were grown on +Fe solid medium and then transferred to +Fe or +Fe+flg22 liquid medium for 24 hours treatment. For each treatment, the cytosolic and nuclear-localized mCitrine-IMA1 signals (yellow channel) are visualized with longitudinal section and transverse section. Scale bar: 50 μm.

(B) Representative images of roots of plants with pIMA1, pELTP or pPGP4 promoters driving IMA1 expression in response to +Fe, +Fe+flg22, −Fe and −Fe+flg22. For each treatment, mCitrine-IMA1 signals (yellow channel) and bright field are visualized with longitudinal section. Scale bar: 50 μm.

(C) Quantification of normalized IMA1 signal diameter in different treatment conditions in differentiation zones of the roots. Different letters indicate statistically significant differences between different conditions analyzed by one-way ANOVA and Fisher's LSD test (two-sided, p<0.05).

(D) Quantification of normalized IMA1 signal intensity of pPGP4::mCitrine-IMA1 in different treatment conditions. Different letters indicate statistically significant differences between different conditions analyzed by one-way ANOVA and Fisher's LSD test (two-sided, p<0.05).

FIGS. 14A-14K. Flg22 dependent IMA1 degradation in the ground tissue is regulated by BTSL1 and BTSL2 but not by BTS.

(A) Western blots showing IMA1 protein is degraded through ubiquitin-dependent proteasome mechanism under −Fe condition. The pIMA1::EYFP-IMA1;ima& was pre-treated with −Fe with DMSO only or −Fe with 10 μM MG132 for 36 hours, subsequently with 100 μM Cycloheximide (CHX) for the indicated time period. Tubulin protein was blotted as an internal control.

(B) Western blots showing IRT1 protein levels in Col-0 and bts-1 roots in +Fe, +Fe with flg22, —Fe and −Fe with flg22 treatment. Tubulin protein was blotted as an internal control.

(C) Quantitative analysis of Ferric Chelate Reductase activities in Col-0 and bts-1 roots grown for 7 days under +Fe conditions and transferred to +Fe, +Fe with flg22, −Fe and −Fe with flg22 liquid medium for 2 days. The bar chart centers show mean of 5 biological replicates. Error bars: s.e.m. Different letters indicate statistically significant differences between different conditions analyzed by one-way ANOVA and Tukey's test (p<0.05).

(D) IMA1 distribution in bts-1 in response to +Fe, +Fe with flg22, −Fe and −Fe with flg22 treatment in differentiation zone of the root. 5-day-old pIMA1::EYFP-IMA1;bts-1 seedlings were grown on the +Fe medium and then transferred to different liquid medium for 24 hours treatment. The cytosolic and nuclear localized EYFP-IMA1 signals (yellow channel) are visualized with propidium iodide (PI, red channel). For each treatment, a representative single confocal section (single image, EYFP/PI), a maximal Z-projection of the Z-stack (Z-max, EYFP only), a single optical section of the transverse view, and the Z-projection of the transverse section are shown. Scale bar: 50 μm.

(E) Western blots showing IMA1 protein levels in the roots of pIMA1::EYFP-IMA1;ima8x and pIMA1::EYFP-IMA1;btsl1,2 in +Fe, +Fe with flg22, −Fe and −Fe with flg22 treatments. Tubulin protein was blotted as an internal control.

(F) Western blots showing IMA1 protein levels in pIMA1::EYFP-IMA1;ima8x and pIMA1::EYFP-IMA1;btsl1,2 roots. The seedlings were pre-treated with −Fe for 36 hours, then treated with −Fe+flg22 (1 μm flg22) for 0, 3 and 6 hours. Tubulin protein was blotted as an internal control.

(G) Representative image of EYFP-IMA1 signal intensity profile in response to +Fe, +Fe with flg22, −Fe and −Fe with flg22 treatment in differentiation zone of roots in pIMA1::EYFP-IMA1;ima8x and pIMA1::EYFP-IMA1;btsl1,2. The white line in YFP channel indicates the line for signal quantification. The red dashed lines indicate the boundary between the ground tissue and the stele. Scale bar: 50 μm.

(H-J) Quantification of normalized total IMA1 signal intensity (H), normalized IMA1 signal intensity in epidermis and cortex (J) and IMA1 signal diameter (J) in differentiation zone of roots in pIMA1::EYFP-IMA1;ima8x and pIMA1::EYFP-IMA1;btsl1,2. The seedlings were pre-treated with +Fe/−Fe 36 hours with/without flg22 and treated with 100 μM CHX for 2 hours before imaging. Different letters indicate statistically significant differences between different conditions analyzed by one-way ANOVA and Fisher's LSD test (two-sided, p<0.05).

(K) Schematic of flg22-mediated IMA1 depletion in the outer cell layers (epidermis, cortex, and endodermis) and IRT1 repression in epidermis in response to +Fe, −Fe and −Fe with flg22 treatment respectively. By contrast, IMA1 is not fully degraded in btsl1,2 in the outer cell layers upon flg22 treatment, resulting in IRT1 level maintenance in epidermis.

FIGS. 15A-15E. IMA1 mediates defense responses to different pathogens in the shoot.

(A) Confocal images of pIMA1::EYFP-IMA1;ima8x responses to +Fe, +Fe with flg22, −Fe and −Fe with flg22 in the shoot. Three different zones were imaged: epidermal cells, mesophyll cells and vasculature. The cytosolic and nuclear localized EYFP-IMA1 signals (yellow channel) are visualized with bright field (bright field channel). For each treatment, a representative single confocal section is shown. Scale bar: 50 μm

(B) Gene ontology analysis of upregulated genes in IMA1ox compared with Col-0 in the shoot. GO term analysis is performed by using PANTHER17.0 (p-value<0.05). X axis: fold enrichment.

(C) Western blots showing MAPK phosphorylation by flg22 in Col-0, ima& and UBQ10::mCitrine-IMA1 shoot in response to flg22. The shoot parts were treated with 1 μM flg22 for 0, 5 and 10 minutes. Tubulin protein was blotted as an internal control.

(D) Gene expression analysis in response to +Fe and +Fe with flg22 by quantitative RT-PCR. The gene expression level is normalized to ACT2 internal control. The shoot parts were treated with 1 μM flg22 for 1 hour. The bar chart centers show means of 3 biological replicates and error bars show s.e.m. Different letters indicate statistically significant differences between different conditions analyzed by one-way ANOVA and Tukey's test (p<0.05).

(E) Growth of Pseudomonas syringae pv. tomato DC3000 in the leaves of Col-0 and UBQ10::mCitrine-IMA1. Bacteria were syringe-infiltrated at OD₆₀₀=0.001, and bacterial colony forming units (CFUs) were counted 0 and 48 hours after inoculation (hpi). n=22-24 biological replicates from three independent experiments. Different letters indicate statistically significant differences (adjusted P<0.01; two-tailed Student's t-test followed by Benjanini-Hochberg method). Results are shown as box plots with boxes displaying the 25th-75th percentiles, the center line indicating the median and whiskers extending to the minimum and maximum values no further than 1.5× interquartile range.

FIGS. 16A-16E. IRT1 and IMA1 accumulation is distinctly regulated by CHA0 or flg22 peptide.

(A) Western blots showing IRT1 protein levels in Col-0 roots in response to +Fe, +Fe+CHA0, −Fe, −Fe+CHA0 and −Fe+flg22 treatments. The inoculation of CHA0-gfp2 was detected by anti-GFP western blot. Tubulin protein was blotted as an internal control.

(B) Western blots showing IMA1 protein levels in pIMA1::EYFP-IMA1;ima8x roots in response to +Fe, +Fe+CHA0, −Fe, −Fe+CHA0 and −Fe+flg22 treatment. The EYFP-IMA1 and the inoculation of CHA0-gfp2 were detected by anti-GFP western blot. Tubulin protein was blotted as an internal control.

(C) The promoter activity of FRK1 in pFRK1::NLS-3xmVenus roots in responses to +Fe, +Fe+CHA0, −Fe, −Fe+CHA0 in differentiation zone or at lateral root primordia. Scale bar: 50 μm.

(D) Quantification of the total signal intensity of the promoter activity of FRK1 in response to +Fe, +Fe+CHA0, −Fe, −Fe+CHA0 in differentiation zone or at lateral root primordia. Different letters indicate statistically significant differences between different conditions analyzed by one-way ANOVA and Fisher's LSD test (p<0.05).

(E) Graphical summary of main findings. Red color gradient indicates IMA1 abundance. Left and middle columns: IMA1 and IRT expression in the root under +Fe (upper left), −Fe (lower left), +Fe+flg22 (upper middle), −Fe+flg22 (lower middle). In +Fe IMA1 and IRT1 are expressed at very low levels in the early differentiation zone of the root. In −Fe, IMA1 highly accumulates in the ground tissue of the root under; roots actively lower the rhizosphere pH and increase iron availability, but this decreases PTI responsiveness. In −Fe and flg22 treatment, IMA1 is degraded in a BTSL dependent manner. The rhizosphere pH is not decreased. Right column: IMA1 accumulation in relation to relation to root and shoot host-microbe interactions. Upper right: In +Fe conditions IMA1 levels are low and don't respond to commensal bacteria CHA0. Lower right: High levels IMA1 enhances a subset of PTI in the shoot. In −Fe IMA1 is accumulated in the root. In the early differentiation zone, surface dwelling CHA0 bacteria don't affect IMA1 levels; high IMA1 levels increase bacterial colonization on the surface. When CHA0 colonizes inner root tissues at lateral root primordia cracks, IMA1 decreases in cells adjacent to this colonization. IMA1 accumulation is distinctly regulated by CHA0 in early differentiation zone compared to emerging LR region. The differences in IMA1 signal intensity between the depictions in the panels relating to CHA0 are due to different experiment setting (half sucrose concentration and different laser power for real bacteria experiment; see methods section).

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

### I. Exemplary Protein and DNA Sequences of IMA

SEQ ID NOs: 1-8 are protein sequences from the IRON MAN (IMA) family found in Arabidopsis thaliana:

SEQ ID NO: 1 is an exemplary protein sequence of IMA1 (tr: Q3ECW0):
MMSFVANLAIKRFDHASTVYVEDVVDSSRVAYSENGGDDDDSGYDYAPAA
SEQ ID NO: 2 is an exemplary protein sequence of IMA2 (tr: Q8GUL3):
MMSYVANLVIKSFDRASVVYVEDVVDSSRATCVENGGDDDDSGYDYAPAA
SEQ ID NO: 3 is an exemplary protein sequence of IMA3 (tr: Q56Y35):
MAVVSHNNAEGRLYESTQTWPIAYLQIGGQENGGDDDDDDCDVAPAA
SEQ ID NO: 4 is an exemplary protein sequence of IMA4 (tr: Q1G327):
MISVSEFVLCIDDNVSGTCMRGKVVISDQAFVYAQSVYVEDGDNDDDDIYDYAPAA
SEQ ID NO: 5 is an exemplary protein sequence of IMA5 (gi: 682125022):
MFSIYKFVLCKWDQVGETFIRGDVTYNNGEFEYPQVAYVENGDDDDDIIXDYAPAA
SEQ ID NO: 6 is an exemplary protein sequence of IMA6 (gi: 682124983):
MVSVSELVLYVHENVYETCIGVNIANNDQVFEYAQTAFVENGDNDDDVIYDYAPAA
SEQ ID NO: 7 is an exemplary protein sequence of IMA7 (gi: 682125078):
MSSSLEFDFVLYYNVHYAFASFKNEGDDDDDVVYDYAPAA
SEQ ID NO: 8 is an exemplary protein sequence of IMA8:
MFSLSEFVFRIYDHISESCVGGDTTSYDKEIKYRQAAYAEIGDQNEDDIYDYAPAAGVHP
SYE

SEQ ID NOs: 9-12 are protein sequences from the IMA family found in Brassica napus (canola):

SEQ ID NO: 9 is an exemplary protein sequence of IMA1 (tr: A0A078GB29):
MSFAANLVIINFYCASAVCVEELLDNSLGSYTENGGDDDDSGYDYAPAA
SEQ ID NO: 10 is an exemplary protein sequence of IMA2 (tr: A0A078FTG9):
MISVREFVFCASNNNICEMCSGGVMANNDKRFEYAQAAYVENGDNDDDVIYDYAPAA
SEQ ID NO: 11 is an exemplary protein sequence of IMA3 (tr: A0A078F0R3):
MFSVSEFLFCTYDNVYGGDITNNDEAIQYAQAVFSENGDDDDDVIYDYAPAA
SEQ ID NO: 12 is an exemplary protein sequence of IMA4 (tr: A0A078EAK1):
MAVMSYNKAEGRLYESTQTRPVPYIQTVGQESGGDDDDDDSDVAPAA

SEQ ID NOs: 13-14 are protein sequences from the IMA family found in Thlaspi arvense (pennycress):

SEQ ID NO: 13 is an exemplary protein sequence of IMA1 (Thlar.0230s0029.1.p):
MSFVANLVIKSFLHASAEFEYVVDSSLGAYVENGGDDDDSGYDYAPAA
SEQ ID NO: 14 is an exemplary protein sequence of IMA2 (Thlar.0230s0033.1.p):
MSSIANFVIKSFNRASAELEDVVDSSLGAYVENGGDDDDSGYDYAPAA

SEQ ID NOs: 15-27 are protein sequences from the IMA family found in Glycine max (soybean):

SEQ ID NO: 15 is an exemplary protein sequence of IMA1 (gi: 734416477):
MVVFICKEEYGVPLSNDWAATHEFGHKFCISNEGDDDDDDNGVIDVAPAA
SEQ ID NO: 16 is an exemplary protein sequence of IMA2 (gi: 734309868):
MVVFVSCTKSGLPLFSKGNDWPATRFPIHQETDDDDDDDDDDGGIDIAPAA
SEQ ID NO: 17 is an exemplary protein sequence of IMA3 (gi: 734348816):
MVVFLCKEEYGVLLGNDWAATHEFGHNFCISNEGDDDDDDNGVIDVAPAA
SEQ ID NO: 18 is an exemplary protein sequence of IMA4 (gi: 734359117):
MSFTSKVIAPWCKKHGNDDVVDAPAATTFIGGNVCNWHGDFVSFVPIA YMEGDDDDD
GGYDYAPAA
SEQ ID NO: 19 is an exemplary protein sequence of IMA5 (gi: 255630230):
MSFTSKVIALWCKKHGNDDGVDVYDAPAATACIEGNVCNWHGDFVSFVPVALVEGDD
DDDDGGYDYAPAA
SEQ ID NO: 20 is an exemplary protein sequence of IMA6 (gi: 734316660):
MALTSKAINQECKKHACGNKDGDWYLYYAPTACTEGDDHKGNRDSCFGHIAYMKGD
DDGDIYDYAPAA
SEQ ID NO: 21 is an exemplary protein sequence of IMA7 (gi: 734354848):
MAFISMAINLIDCMTHACGNKDNDWYLYAPTACTEGDDPNGDVDSWFAYMEGDDDD
GYDYAPAA
SEQ ID NO: 22 is an exemplary protein sequence of IMA8 (gi: 255631054):
MTPEIKKHACDKKDGVLYHYDPTACAEGDDYNGNINYVFAYMEGDDDDDGGYDYAP
AA
SEQ ID NO: 23 is an exemplary protein sequence of IMA9 (g: 1734427312):
MASMSKAMTPEIKKHACDKKDGVSYHYDPTACAEGDDYNGNINYVFAYMEGDDDDD
GGYDYAPAA
SEQ ID NO: 24 is an exemplary protein sequence of IMA10 (gi: 734394461):
MVVFVSCTKSGLPLFSKGNDWQATRLSIHQEADDDDDDDDDGGIDIAPAA
SEQ ID NO: 25 is an exemplary protein sequence of IMA11 (tr: K7KB97):
MVVFICKEEYGVPLSNGWAATHEFGHKFCISNEGDDDDDDNGVIDVAPAA
SEQ ID NO: 26 is an exemplary protein sequence of IMA12 (tr: K7KWF9):
MALTSKAINQECKKHACGNKDGDWYLYYAPTACTEGDDHKGNGDSCFGHIAYMKGD
DDSDIYDYAPAA
SEQ ID NO: 27 is an exemplary protein sequence of IMA13 (tr: K7KWG1):
MASMSKAMTPEIKKHACDKKDGVLYHYDPTACAEGDDYNGNINYVFAYMEGDDDDD
GGYDYAPAA

SEQ ID NOs: 28-36 are protein sequences from the IMA family found in Solanum lycopersicum (tomato):

SEQ ID NO: 28 is an exemplary protein sequence of IMA1 (tr: M1AZY7):
MTEIYSFHLYNKIELTRPVISFCLNRELENDDDDDDDGKKVAPAA
SEQ ID NO: 29 is an exemplary protein sequence of IMA2 (tr: K4CEP8):
MVIVRGNTTPFLPGRIEARPVINFGLNREFEADDDDDDDDDDGKKVAPAA
SEQ ID NO: 30 is an exemplary protein sequence of IMA3 (tr: K4CEP9):
MVIVRGNTSRFHPYEIEARLVISFYLNRELENDVDDDDDDDDDGAKVAPAA
SEQ ID NO: 31 is an exemplary protein sequence of IMA4 (tr: K4DBK9):
MSQISTILMNSICNFNHDVGSHVYESRSMMDRHATGCIYVSATWFEDDGDDDDDDDDA
DYDYAPAA
SEQ ID NO: 32 is an exemplary protein sequence of IMA5 (tr: K4DBL0):
MSTIFMIIGFEKRRSCADGDYDYTSAASLEGDDDDGDYDYAPAASLEGNDDDGDYDYA
PAASLEGDDDDGDYDYAPAA
SEQ ID NO: 33 is an exemplary protein sequence of IMA6 (tr: K4DBL2):
MFGIFKIIGFEKIRRSCLDGDDDGDYDYAPAACLKRNGDDDGDYDYAPAAFLEGDDDD
RDYDCAPAATIDGDDDGDYDYAPAA
SEQ ID NO: 34 is an exemplary protein sequence of IMA7 (tr: K4DBL3):
MSGILKIIGFEKIRRSCLDGDDDSDYDYAPAACLERDGDDDGDYDYAPAASLEGDDDDR
DYDYVPAASLEGDDDGDYDYAPSGCMK
SEQ ID NO: 35 is an exemplary protein sequence of IMA8 (tr: K4DBL4):
MSGIFKIIGFEKIKRSCLDGDDDGDYDFAPAACLERDGDDDGDYDYAPAASLEGDDDDR
DYDYVPAASLEGDDDGDYDYAPAA
SEQ ID NO: 36 is an exemplary protein sequence of IMA9 (tr: K4DBL5):
MSSIFKIIGFQKRRSCSDGDDDGDYDYAPSACLEGGGDGDDGDYDYTPAASLEGDCND
QDYDYAPAVSFEGHDVDGDYDYAPAA

SEQ ID NOs: 37-38 are protein sequences from the IMA family found in Oryza sativa (rice):

SEQ ID NO: 37 is an exemplary protein sequence of IMA1 (tr: A6MZQ3):
MAPVSEASPLVHQDGGIIASFAVYAGAPCCSARGRMAETDGDDDDDDYDCAPAA
SEQ ID NO: 38 is an exemplary protein sequence of IMA2 (tr: Q8H4K5):
MAIAKSECERLAWALLLESNLLVGNRRSNGDDDDDDVDVAPAA

SEQ ID NO 39 is a protein sequence from the IMA family found in Zea mays:

SEQ ID NO: 39 is an exemplary protein sequence of IMA1 (tr: A0A096QUR9):
MAPVSSEAASYLVLIKGGSIAASSRAVYPWDGCSARGRMTETDSDDDDDDYDCAPAA

SEQ ID NO: 40 is a protein sequence from the IMA family found in Aegilops tauschii:

SEQ ID NO: 40 is an exemplary protein sequence of IMA1 (tr: M8AME8):
MAPASKVMSHIVQDGGIADYAVYAAAPCDAWCGGRHRKAESDGDDDDDDYDCAPAA

| | is an exemplary protein sequence |
|---|---|
| | of IMA1 (www.amborella.org: |
| | gn1:Amborella_b4_c13621): |
| SEQ ID NO: 41 | |
| | MLQRYDAPFVGQKWHQKRIGEGDDDDDDDYDCAPAA |
| | is an exemplary protein sequence |
| | of IMA2 (www.amborella.org: |
| | gn1:Amborella_b4_rep_c242199): |
| SEQ ID NO: 42 | |
| | MLQRYDAPFVGQKWHQKRIGEGDDDDDDDYDFAPAA |
| | is an exemplary protein sequence |
| | of IMA3 (www.amborella.org: |
| | gn1:Amborella_b4_c8363): |
| SEQ ID NO: 43 | |
| | MLDRHDAHLGCQKWHQKKILRTEGDDDDDDDDYDCAPAT |
| | is an exemplary protein sequence |
| | of IMA4 (www.amborella.org: |
| | gn1:Amborella_b4_rep_c242695): |
| SEQ ID NO: 44 | |
| | MLDRHDAHLCCLKWHQKKILRTEGDDDDDDDDYDCAPAA |
| | is an exemplary protein sequence |
| | of IMA5 (www.amborella.org: |
| | gn1:Amborella_b4_rep_c69998): |
| SEQ ID NO: 45 | |
| | MASEENPPNRRDDDDDDDDYDCAPAT |
| | is an exemplary protein sequence |
| | of IMA6 (www.amborella.org: |
| | gn1:Amborella_b4_rep_c101612): |
| SEQ ID NO: 46 | |
| | MASEENREGDDDDDDDYDCAPAA |

SEQ ID NOs: 47-53 are protein sequences from the IMA family found in Arabidopsis lyrata:

is an exemplary protein sequence of
IMA1 (tr:D7KAT2):
SEQ ID NO: 47
MMSFVANLAIKSLDRASAVYVEDVVDSSRVAYGENGGDDDDSGYDYAPAA
is an exemplary protein sequence of
IMA2 (tr:D7KAT0):
SEQ ID NO: 48
MMSFVANLVIKSFYRASAMYVEDMVDSSRATCLENGGDDDDSGYDYAPAA
is an exemplary protein sequence of
IMA3 (tr:D7KAT1):
SEQ ID NO: 49
MMYFFANLVSKSFDRASAVYVEDVVDCSRATCVENGGDDDDSGYDYAPAA
is an exemplary protein sequence of
IMA4 (tr:D7KAT4):
SEQ ID NO: 50
MISVTEFILCIDDNVGGTCIGGEVVISGQAFVYAQSVYVEDGDNDDDDIY
DYAPAA
is an exemplary protein sequence of
IMA5 (tr:D7KAT6):
SEQ ID NO: 51
MISVSEIVLYIHENVYETSIGVNIANNDKVFEYAQATFVENGDNDDDVIY
DYAPAA
is an exemplary protein sequence of
IMA6 (tr:D7KAT3):
SEQ ID NO: 52
MSSLSEFVLSIYDHVSESCVGSDTTSYDQEIKSRQAAYAENGDQDDDDIY
DYAPAA
is an exemplary protein sequence of
IMA7 (tr:D7KAT5):
SEQ ID NO: 53
MVSIYKFVLCKCDQVRETCIRGDVTYNNGEFEYHQVAFIENGDDDDDIIY
DYAPAA

SEQ ID NOs: 54-57 are protein sequences from the IMA family found in Arabis alpina:

is an exemplary protein sequence
of IMA1 (tr:A0A087H1Z8):
SEQ ID NO: 54
MISVTEFVLCIHENVYDKCNGDGIVNINGASDSATVENGDNDDDDIYDYA
PAA
is an exemplary protein sequence
of IMA2 (tr:A0A087H1Z7):
SEQ ID NO: 55
MISVTEFVLCIDDIVYEKCIAVSGAKSIQASEFTSVENGDNDDDVIYDYA
PAA
is an exemplary protein sequence
of IMA3 (tr:A0A087H1Z6):
SEQ ID NO: 56
MVFVFHYVLCKYDEVCETFIEGNAIKNCAELEYSQAGYVENGDDDDDNVY
DYAPAA
is an exemplary protein sequence
of IMA4 (tr:A0A087GN13):
SEQ ID NO: 57
MAVMSHNKAESRLHESTQACPSPYSVTRAHENGGDDDDDDCDVAPAA

SEQ ID NOs: 58-66 are protein sequences from the IMA family found in Brassica rapa:

is an exemplary protein sequence of IMA1
(tr:M4FIM8):
SEQ ID NO: 58
MFSVSEFLFCTYDNVYGGDITNNDEAVQYAQAVFSENGDDDDDVIYDYAP
AA
is an exemplary protein sequence of IMA2
(brassicadb.cn: A08: 3,778,606-3,778,776):
SEQ ID NO: 59
MINVCDFFPNNNISETCIIGGVMASNDQAFEYAQVQATYVENGDNDDDII
YDYAPAA
is an exemplary protein sequence of IMA3
(gi:685341480):
SEQ ID NO: 60
MYLVAHLVIKSFDGDYAVSAEDVVDTSRAAYIENGGDDDDGGYDYAPAA
is an exemplary protein sequence of IMA4
(brassicadb.cn: A08: 3,785,365-3,785,526):
SEQ ID NO: 61
MYAANLVIMNFDRSSAVCVEDMGDSSIGAFIENGGDDDDIGHGYACMIHL
THQM
is an exemplary protein sequence of IMA5
(brassicadb.cn: A05: 12,678,561-12,678,707):
SEQ ID NO: 62
MSFAANLVIINFYCASAVCVEELLDNSRGSYTENGGDDDDSGYDYAPAA
is an exemplary protein sequence of IMA6
(gi:685300759):
SEQ ID NO: 63
MAVMSHNKAEGRLYESTQTRLVPYIQTLGQESGGDDDDDDSDVAPAA
is an exemplary protein sequence of IMA7
(gi:685283687):
SEQ ID NO: 64
MAVMSHDKAEDRLYESAHTRPIPYNSQIVGQESGGDDDDDDSDVAPAA
is an exemplary protein sequence of IMA8
(brassicadb.cn: A03: 10,750,322-10,750,510):
SEQ ID NO: 65
MCYIKPSFIFFKASFQLSRVSKRMSAIEFDLVLHCNGHTQFARSFKNEGD
DDDDVVYDYAPAA
is an exemplary protein sequence of IMA9
(brassicadb.cn: A05: 2,168,745-2,168,867):
SEQ ID NO: 66
MYALVEFDLVLHYNGHTAFAISFKNEGDDDDDVVYDYAPAA

SEQ ID NOs: 67-78 are protein sequences from the IMA family found in Camelina sativa:

is an exemplary protein sequence of
IMA1 (gi:727437870):
SEQ ID NO: 67
MMTFVANLLSKSLDRASSVYVEDVVDSSRVAYGENGGDDDDSGYDYAPAA
is an exemplary protein sequence of
IMA2 (gi:727612111):
SEQ ID NO: 68
MMTFVANLLSKSLDRASSAYVEDVVDSSRVAYGENGGDDDDSGYDYAPAA
is an exemplary protein sequence of
IMA3 (gi:727577750):
SEQ ID NO: 69
MMTLLSKSLDRASSVYVEDVVDSSRVAYGENGGDDDDSGYDYAPAA
is an exemplary protein sequence of
IMA4 (gi:727437866):
SEQ ID NO: 70
MSFVANLVIKSFDRASTVCVEDVVDSFRAAYVENGGDDDDSGYDYAPAA
is an exemplary protein sequence of
IMA5 (gi:727612109):
SEQ ID NO: 71
MMSSVANLVIKSFDYASTVCVEDVVDSSRAAYVENGGDDDDSGYDYAPAA
is an exemplary protein sequence of
IMA6 (gi:727577744):
SEQ ID NO: 72
MSFVANLVIKSFDRASTVCVEDVVDSFRVAYVENGGDDDDSGYDYAPAA
is an exemplary protein sequence of
IMA7 (gi:727612098):
SEQ ID NO: 73
MSFVANLVIKSFDRASTVCVEDVVDSFRVAYVENGGDDDDSGYDYAPVA
is an exemplary protein sequence of
IMA8 (gi:727577748):
SEQ ID NO: 74
MMSSVANLVIKSFDYASTVCLEDVVDSSRAAYVENGGDDDDSGYDYAPAA
is an exemplary protein sequence of
IMA9 (gi:727577746):
SEQ ID NO: 75
MSSVADLVIKSFNHASTVCDEDVVDTFRAAYVESGGDDDDSGYDYAPAA
is an exemplary protein sequence of
IMA10 (gi:727612107):
SEQ ID NO: 76
MSSVADLVIKSFNHASTACDEDVVDSFRAAYVENGGDDDDSGYDYAPAA
is an exemplary protein sequence of
IMA11 (gi:727437868):
SEQ ID NO: 77
MMPSVANLVIKSFEYVSTVCLEDVKDSSRVAYVENGGDDDDSGYDYAPAA
is an exemplary protein sequence of
IMA12 (gi:727593545):
SEQ ID NO: 78
MAVLIVSRNNNGEGRLYESTRTQPIPYLQNGGQENGGDDDDDDCDVAPAA

SEQ ID NOs: 79 is a protein sequence from the IMA family found in Cicer arietinum:

is an exemplary protein sequence of
IMA1 (gi:502086844):
SEQ ID NO: 79
MASISMIIAPRCEKHAYGEGDRFCYISTACVELEDYHSGGGDFVSPQVTY
NEGDDDDDGGYDYAPAA

SEQ ID NOs: 80-82 are protein sequences from the IMA family found in Citrus clementina:

is an exemplary protein sequence of
IMA1 (gi:567918023):
SEQ ID NO: 80
MAPMSSSLEGITHGNVHHRDDDSIHVYGCPYYYRNEPFEGDGDDDDDDDD
GCDLAPAASMEGDGDDDDDDGGYDYAPAA
is an exemplary protein sequence of
IMA2 (gi:567918027):
SEQ ID NO: 81
MSLVSKSVMPSSSWTWCKKHGDGDDDDDDGYDYAPAACIEGYGDDDDDDG
DYDYAPAASMEGDDDGSYDYAPAA
is an exemplary protein sequence of
IMA3 (gi:567918024):
SEQ ID NO: 82
MSSSLEGITHGNVHHRDDDSIHVYGCPYYYRNEPFEGDGDDDDDDDDGCD
LAPAASMEGDGDDDDDDGGYDYAPAA

SEQ ID NO: 83 is an exemplary protein sequence from the IMA family found in Cucumis melo:

is an exemplary protein sequence of
IMA1 (gi:733563923):
SEQ ID NO: 83
MDVSIFEPVASTMIKNIIAYKDVKCGRQFSTNLTTTIIRREGDDDDDDGC
YDYAPAA

SEQ ID NO: 83 is an exemplary protein sequence of IMA1 (gi:733563923):

is an exemplary protein sequence of
IMA1 (tr:A0A0A0LF41):
SEQ ID NO: 84
MAPISRLPCVLGLKNLGGDGGHGYREGCDCGYTTLVSMAEGDSDDDDGGY
DFAPAA
is an exemplary protein sequence of
IMA2:
SEQ ID NO: 85
MVSTSKSVASMMIKNNVCEDVKCSRSFPIDLTKTIIRREGDDDDDDGCYD
YAPAA

SEQ ID NO: 86 is a protein sequence from the IMA family found in Erythranthe guttata:

is an exemplary protein sequence of IMA1
(tr:A0A022R5Q2):
SEQ ID NO: 86
PNHTHSNSWNLCSKRSELTSEEIRVSPNFGILITQLHDREGDDDDDDDDG
GTFVAPAA

SEQ ID NO: 87 is a protein sequence from the IMA family found in Gossypium hirsutum:

is an exemplary protein sequence of
IMA1 (gi:519750295):
SEQ ID NO: 87
MSPFSKVVASSCKKHVDGDYDDNDGFDYAPIACMEGNGDDDDDDDYDYAP
AASLDGDDDDDSYDYAPAA

SEQ ID NOs: 88-89 are protein sequences from the IMA family found in Jatropha curcas:

is an exemplary protein sequence of
IMA1 (tr:A0A067KJB1):
SEQ ID NO: 88
MSSVLLKAIASSWCNNQNLIIYDDGFDYASVVPSIDGDGGDDDDGDYDYA
PAASMEGDDDDDDDDGDCAPAA
is an exemplary protein sequence of
IMA2 (tr:A0A067KTA7):
SEQ ID NO: 89
MVIVDSKKLGFFRLVAGEGQAMACFCMSKQNDGDDDDDDDDGGADVAPAA

SEQ ID NO: 90 is a protein sequence from the IMA family found in Lotus japonicus:

is an exemplary protein sequence of
IMA1 (tr:13SAD1):

SEQ ID NO: 90

MVVLVCKESRLPKFFMAPPELQSFCIQNESDSDDDDDGDNDIDIAPAA

SEQ ID NOs: 91-105 are protein sequences from the IMA family found in Medicago truncatula:

is an exemplary protein sequence of
IMA1 (tr:A0A072VBU9):
SEQ ID NO: 91
MVFISMVIALNCKQHAYGEGEVDWFGCTSVSCIEEDYHNTDHDSYWEGDD
DDDGGYDYAPAA
is an exemplary protein sequence of
IMA2 (tr:A0A072VA36):
SEQ ID NO: 92
MASISMVIALNCKQHAYGEGNWFDYTSVSCIEEDYHNGDRDSYQEGDDDD
DGGYDYAPAA
is an exemplary protein sequence of
IMA3 (tr:G7IM93):
SEQ ID NO: 93
MASIFTVIAPLCKQNACGEGNGDWFGYTSVSCIVEDYRNGDQDSYKEGDD
DDDGGYDYAPAA
is an exemplary protein sequence of
IMA4 (tr:13S3V2):
SEQ ID NO: 94
MTFISTVIAPKCKQYAYNGEGDGDWFGYTCVSCIEEDYTNGDRNLYREGD
DDDDGGYDYAPAA
is an exemplary protein sequence of
IMA5 (tr:G7IM92):
SEQ ID NO: 95
MASISLAIIPKCEQHGYGEGNGDWISYTCVSCIEENYHNGDRDSCKEGDD
DDDGGYDYAPAA
is an exemplary protein sequence of
IMA6 (tr:G7IM94):
SEQ ID NO: 96
MASISLAITPKCKHHGYSEGNGDWFGYTSVSCIKEDNRNGDRDSCKEGDD
DDDGGYDYAPTA
is an exemplary protein sequence of
IMA7 (tr:A0A072VBB1):
SEQ ID NO: 97
MASISMAITPKCKEHGYDEGNGDWFGYTYVSCIEEDYRNGDRDSYMEGDD
DDDGGYDYAPAA
is an exemplary protein sequence of
IMA8 (tr:G7IM89):
SEQ ID NO: 98
MASISMVIAPKCKQHAFNGEGDCDWFGYTNISCIEEDYYNGEGDDDDDDG
GYDYAPAA
is an exemplary protein sequence of
IMA9 (tr:G7JFW8):
SEQ ID NO: 99
MASISIAIATKSIKNVYDGGEWFGYASVACIEDYHIGDVDSYKEGDDDDD
GGYDYAPAA
is an exemplary protein sequence of
IMA10 (tr:G7JFW4):
SEQ ID NO: 100
MASISIAIATRSIKHVYDEDEWFGYAASVACIEDYHTEDVDSSYKEGDDD
DDGGYDYAPAA
is an exemplary protein sequence of
IMA11 (tr:G7JFW9):
SEQ ID NO: 101
MASISIPTRSIKNACGEGEWFGFASVSCNDEDYHTGDVDSYREGDDDDDG
DYDYAPAA
is an exemplary protein sequence of
IMA12 (tr:G7JFW3):
SEQ ID NO: 102
MAFISISIATRSFQNACDEGEWFCYASVGCIEQDNHIGEEDSYREGDDDD
DGGYDYAPAA
is an exemplary protein sequence of
IMA13 (tr:G7IM91):
SEQ ID NO: 103
MTSISMVNISPKCNHAAYGECDGDWFGYASTICIKGNYYFRNEDSGSAHL
IAYIEGDDDDDGGYDYAPAA
is an exemplary protein sequence of
IMA14 (tr:A0A072VL77):
SEQ ID NO: 104
MASISIVNTAPKCNHAAYGECDVDSFGYASTVCIKGNYYIRNEDSGSADL
VTYMEGDDDDDGGYDYAPAA
is an exemplary protein sequence of
IMA15 (tr:G7L3N4):
SEQ ID NO: 105
MSSISNVVAPWCKKHGNDHDGCVVWYDYPPTVCDEGDDDDDGGYDYAPAA

SEQ ID NOs: 106-108 are protein sequences from the IMA family found in Morus notabilis:

is an exemplary protein sequence of
IMA1 (tr:W9R8S0):
SEQ ID NO: 106
MPHITFMNMVTARNGKDGDNGDHCYDHYFQYYNLSAPGEGDGDDDNDDDD
DSGYDYAPAA
is an exemplary protein sequence of
IMA2 (tr:W9R8W7):
SEQ ID NO: 107
MFPAVDKLIYSESLSKKREDGGNHEDGNDGISRRYAPTQVMENIYGASDR
DYNYLPNATMDGDDDDDDDDSSYDYAPAA
is an exemplary protein sequence of
IMA3 (tr:W9RJD7):
SEQ ID NO: 108
MSPVSEKNVAILAIMLCKKQNITYGDVTNGDFEYNLDPVTRIEGDDDDDD
DDDGGYDYAPAA

SEQ ID NO: 109 is a protein sequence from the IMA family found in Pisum sativum:

SEQ ID NO: 109 is an exemplary protein sequence
of IMA1:
MVIITSMESTLPMFLMDNHLSATEFICFCNQSKSEGDDDDGDIDIAPAA

SEQ ID NOs: 110-112 are protein sequences from the IMA family found in Phaseolus vulgaris:

SEQ ID NO: 110 is an exemplary protein sequence
of IMA1 (tr: V7C9W0):
MAFTSKLIAPCCNNHHALHQNHHAPPTFIEESVFNGHGDSVSFVPAASM
EGDDDDDDGSYDYAPAA
SEQ ID NO: 111 is an exemplary protein sequence
of IMA2 (tr: V7BK02):
MLLFISCTKSGLPLFIKGNDWPETIFPLHHEGDDDDDDDDGGIDVAPAA
SEQ ID NO: 112 is an exemplary protein sequence
of IMA3 (tr: V7B6K1):
MMFFVCKEYGLVLSNDWPATHDFHNFHEGDDDDDNGVIDVAPAA

SEQ ID NO: 113 is a protein sequence from the IMA family found in Populus trichocarpa:

SEQ ID NO: 113 is an exemplary protein sequence
of IMA1 (tr: B9H621):
MSPLSKTIIAFCTIHRHADGDDDGEYGYDYAPAACMEGDGDDDDSDYDY
APAAPMEGDDDDDGDYDYAPAA

SEQ ID NOs: 114-116 are protein sequences from the IMA family found in Ricinus communis:

SEQ ID NO: 114 is an exemplary protein sequence
of IMA1 (tr: B9STP4):
MVHMAFLTARSSGKCSSTNLADQNKDIDVLFGYNEFSMEAPLIEDDGDG
DDDDDDGGYDFAPAATLEGDGDDDDDGDYDYAPAA
SEQ ID NO: 115 is an exemplary protein sequence
of IMA2 (tr: B9STP2):
MSLFAISKAITISCCNKLADDCDNGDGCYFAPPPCIEGDGDDDDGDYDY
APAASSEGDDDDDDDGYDCAPAA
SEQ ID NO: 116 is an exemplary protein sequence
of IMA3 (tr: B9STP3):
MSPLSKVIASWCNKPVVEEEMGNDDDRVYEYAPATSTEGDDDDDDDDDK
DCAPAA

SEQ ID NOs: 117-129 are protein sequences from the IMA family found in Solanum tuberosum:

SEQ ID NO: 117 is an exemplary protein sequence
of IMA1 (tr: M0ZKJ9):
MAGKSGRKVVRGVSKSSKAVYFWKIRHGCGIIFGKSKKYKSCSYGSEDD
DDDEHDYAPATYLERDDDDDDGNYDYAPAALT
SEQ ID NO: 118 is an exemplary protein sequence
of IMA2 (tr: MIAZY7):
MVVIMNANNKKVSLGCPDKFMATEAKLGSTICIDKECEADDDYNDDDAS
KIAPAA
SEQ ID NO: 119 is an exemplary protein sequence
of IMA3 (tr: M1BUQ1):
MSGTFKIIGFQKRRSSSDGDDEGYNYAPVTCLEGDGDDNDAYYDYASAP
FLEGDDDDGDYDYAPATSLEGEDNDGDYDYAPAA
SEQ ID NO: 120 is an exemplary protein sequence
of IMA4 (tr: M1BUQ2):
MSTIFKIIGFQKRRSCSDGDDDSDDGYDYAPAACLEGDGDDNDGDYDYA
PAASLEGDDDDGDYDYAPAASLEGDDDDGDYDYAPAA
SEQ ID NO: 121 is an exemplary protein sequence
of IMA5 (tr: M1BUQ3):
MSTIFKIIGFEKRRSCPDGNYNYTLVASLEGDDDDGDYDYAPAASLEGD
DDDGDYDYAPAASLEGDDDDGNYDYAPAA
SEQ ID NO: 122 is an exemplary protein sequence
of IMA6 (tr: M1BUQ4):
MSSIFKIIGFQNKRSYSDGDDDGDYDFAPAAFLEGDDDDGDYDYAPAAS
LNGDDDDGDYDYVPAASLDGDDDDGDYDYAPAA
SEQ ID NO: 123 is an exemplary protein sequence
of IMA7 (tr: M1BUQ5):
MSSIFKIIGFHNRRSYSDGDDDGDYDYAPAAYLEGDDDDEDYDYAPAAS
LNGDDDDGDYDYAPAASLEGDDDDGDYDYAPAA
SEQ ID NO: 124 is an exemplary protein sequence
of IMA8 (tr: M1BUQ6):
MSSIFKIIGFQNRRSYSDGDDDGDYDYAPTAYLEGDDDDGDYDYAPVAS
LNGDDDDGDYDYAPATSLEGDDDDGDYDYAPAA
SEQ ID NO: 125 is an exemplary protein sequence
of IMA9 (tr: M1BUQ8):
MSSIFKIIGFEKRRSCLDGDDDGDYDYAPAACLERDGDDDGDYDYAPAA
SLEGDDDDRDYDYAPAASLEGDDDGDYDYAPAA
SEQ ID NO: 126 is an exemplary protein sequence
of IMA10 (tr: M1BUQ9):
MSSIFKIIGFQKRRLCLDGDDDGDYDYAPAACLEGGGDRDDGDYYYALA
ASLEGDDDDRDYDYAPAASLGGDGDDGDYDYAPVA
SEQ ID NO: 127 is an exemplary protein sequence
of IMA11 (tr: M1BUS5):
MSQISTILMNSICNLTFFDYHVERGNHDIGSHVYESTSMMDRHVIGCIY
VSATWFEDDGDDDDDDDDDDADYDYAPAA
SEQ ID NO: 128 is an exemplary protein sequence
of IMA12 (tr: M1BUS6):
MSEIFTIIGFEKIRRSCLDGDDDGDYDYAPASCLERDGDDDGDYDYAPA
ASLEGDDDDRDYDYVPAASLEGDDDGDYDYAPAA
SEQ ID NO: 129 is an exemplary protein sequence
of IMA13 (tr: M1CZG2):
MSSGIFKILGFEKRRLCSYGDDDGDYVYAPAACLNRDGDDDGEYDYAPA
ASLEGDDDDRDYDYAPATNLEGDDDRDYDYASAA

SEQ ID NO 130 is a protein sequence from the IMA family found in Theobroma cacao:

SEQ ID NO: 130 is an exemplary protein sequence
of IMA1 (tr: A0A061GM26):
MSSSKCIMHDEDNIKKIGSSSKNIMNDDVHDDKGRRDGYVSNSKSLVQG
GNSYTHVPSASVDGDGDDDDDDDYDFAPAA

SEQ ID NOs: 131-133 are protein sequences from the IMA family found in Triticum urartu:

SEQ ID NO: 131 is an exemplary protein sequence
of IMA1 (tr: M8A3M8):
MAPASKVMSHVVQDGGIADYAVYAAAPCDAWCGGRHRKAESDGDDDDDD
YDCAPAA
SEQ ID NO: 132 is an exemplary protein sequence
of IMA2 (tr: M8A646):
MAPASKIMSHIVVQDGGIAAYAVYAAAPCDAWCGGRHRKAESDGDDDDD
DYDCAPAA
SEQ ID NO: 133 is an exemplary protein sequence
of IMA3 (tr: M8ACF3):
MAPASKAMSHIVQDGGIATYAVYAAPCDAWCGGRHRKAETDGDDDDDDY
DCAPAA

SEQ ID NO 134 is a protein sequence from the IMA family found in Vitis vinifera:

SEQ ID NO: 134 is an exemplary protein sequence
of IMA1 (gi: 349713816):
MSSISMAIDSQSMMHDGHVRGEHDKHGHVYCTNDNGGCYYALTAPREGD
GDDDDGDGGYDFAPAA

SEQ ID NOs: 135-138 are DNA sequences from the IMA family found in Arabidopsis thaliana:

SEQ ID NO: 135 is an exemplary DNA sequence of IMA1 (AT1G47400; NM_103634):
ATGATGTCTTTTGTCGCAAACTTGGCCATCAAGAGATTTGACCATGCTTCCACCGTGT
ATGTTGAAGATGTGGTAGATAGTTCTCGAGTGGCATATAGTGAGAATGGTGGTGATGACGA
TGACAGTGGCTATGATTATGCTCCTGCTGCGTGA
SEQ ID NO: 136 is an exemplary DNA sequence of IMA2 (AT1G47395; NM_179449):
ATGATGTCTTACGTTGCTAACTTGGTCATCAAGAGTTTTGACCGTGCTTCCGTGGTGT
ATGTTGAAGATGTGGTGGATAGCTCTCGAGCGACATGTGTTGAGAATGGTGGTGATGACGA
TGACAGTGGCTATGATTATGCTCCTGCTGCGTGA
SEQ ID NO: 137 is an exemplary DNA sequence of IMA3 (AT2G30766; NM_001336300):
ATGGCAGTGGTGAGTCACAACAACGCAGAAGGCAGGCTATACGAATCAACTCAGAC
TTGGCCAATTGCTTACTTACAAATTGGTGGCCAAGAGAACGGAGGAGACGATGACGACGAT
GACTGTGACGTTGCACCGGCGGCTTGA
SEQ ID NO: 138 is an exemplary DNA sequence of IMA4 (DQ492201):
ATGATTTCTGTTTCTGAATTTGTTCTCTGCATAGATGACAATGTTAGTGGTACGTGCA
TGAGAGGTAAGGTCGTTATCAGTGATCAAGCATTTGTGTATGCTCAATCTGTTTATGTAGAA
GACGGTGACAACGATGATGACGATATCTATGATTATGCTCCAGCAGCATGA

### II. Protein and DNA Sequences of BTSL

SEQ ID NOs: 139-140 are protein sequences from the BRUTUS-LIKE (BTSL) family found in Arabidopsis thaliana:

SEQ ID NO: 139 is an exemplary protein sequence of BTSL1 (F4HVS0-1):
MGGGNLHSLPPENASVSASYAVTVGNTKLSDAPVLFFVYCHKAFRAQLVELRRFATDA
AEADSFSGDLAVELSRKFEFLKLVYKYHSAAEDEVIFLALDKRVKNIVSNYSLEHAGTDDLFTSIF
HWLHVLEEEIGSRSDVLREVILCIGTIQSSICQHMLKEERQVFPLLIEKFSFREQASLVWQFICSVP
VMVLEDFLPWMISHLSHEEKIEVENCIKDVAPNEDSLQQVISSWLLDDSQSSCGTPTEIMKGVQY
VNVSKSLKKSPESHPSSGCFQRFWEWSKKSLSIPNVGRSPIHGLRLFQNAIEKDLRDIQEGLCQAK
FQTLILDLDVLMARLNFLADVLVSYSNAFKKFFHPVLEEMTARRSSTAKQFNIDDCLENFQRLLY
KSADDKTKTDNFLLQLQEELESLIIQVTKQFAIQRTEVFPIISKNCNHEMQKQLLYTSIHVLPLGLL
KCVILWFSAHLSEEESQSILHFLSLEDSSPKKSFPRLLLQWLRFGYSGKTSVERFWKQLDVMFKV
RCSCQKEHTEEASGSFSNQTQLQLCKVSKDVYPRKKDKSSTCFMSMDLAVGDMYETPYSSRMN
QQMTFSGKLKPPLHLPDFFGEKNMDDPMIMDVKPIDLLFFFHKAMKMDLDYLVCGSTRLAADF
RFLAEFQQRFHMIKFLYQIHSDAEDEIAFPALEAKGQLKNISHSFSIDHELETKHFDKVSFILNEMS
ELNMLVSTINTTAADHDRKMKYERLCLSLREICKSMHKLLSEHIQHEETELWGLFRNCFSIEEQE
KIIGCMLGRISGEILQDMIPWLMESLTSDEQLAAMSLWRQATRKTMFVEWLTEWYNGHVLQEE
AGEANNDPFGDSDPLEIVWKYLFEASADGEKGSMRSSLLKLPKTNFTGIMNQPPPNYKVEVGKK
EEKDLERSESKKICRGSNQEGDKEQTDKMSQKVSQFGPSKKYEQLLTMSEEELVVVIKKISCDSS
LDPQKKDYIKQNLLMSRWNISQRTYNLEPSSLSSNMETVHGQHPSYRDPHSLIFGCNHYKRNCK
LLAPCCDKLFTCIRCHDEEADHSVDRKQITKMMCMKCLLIQPIGANCSNTSCKSSMGKYFCKICK
LYDDERKIYHCPYCNLCRVGKGLGIDYFHCMKCNACMSRTLVEHVCREKCLEDNCPICHEYIFT
SSSPVKALPCGHLMHSTCFQEYTCSHYTCPVCSKSLGDMQVYFKMLDALLAEEKMPDEYSNKT
QVILCNDCGRKGNAPYHWLYHKCTTCGSYNSRLL
SEQ ID NO: 140 is an exemplary protein sequence of BTSL2 (A0A654EB76):
MGVGDPLPLPPEKNRREVNKPPDIASTSSSSASAVNNARLSDAPILLFVYFHKAFRAQLA
ELQFLAGDTVRSGSDLA VELRSKFEFLKLVYKYHSAAEDEVIFSALDTRVKNIVFNYSLEHDATD
DLFTSVFHWLNVLEEEQGNRADVLREVVLCIGTIQSSICQHMLKEERQVFPLMIENFSFEEQASLV
WQFICSVPVMVLEEIFPWMTSLLSPKEKSEVETCFKEVVPNELSLQLVINSWLIDDSQSSLTALTKI
MKGVQSVEVSENMTNSQTNSSSSGVFQRFWQWSKKMSFSSPNTGHILVHGIHLWHNAIRKDLV
DIQKGLCQLTFPSLSLDLNVLVVRLNFLADVLIFYSNAFKTFFYPVFEDMVDQQHSSSSKQFTIDG
HVENFKKSLDLETRAGSDNFVITLQEKLESLILTVAKQFSIEETEVFPIISKNCNIEMQRQLLYRSIH
FLPLGLLKCVIMWFSAQLPEDECQSIIHYLSSEDSFPNKPFAHLLLQWFRFGYSGKTPVESFWNEL
SFMFKPRCSFEEELTEEASGSFFQQSPQKLFKVSDPYSMDPPAGYMNETPYSSAMNQQILIPGKLR
PLLHLPDLFGDKTIGEHLTMDLKPIDLIFYFHKAMKKDLDYLVRGSARLATDYSFLGEFQQRFHL
IKFLYQIHSDAEDEIAFPALEAKGKLQNISQSYSIDHELEVEHLNKVSFLLNELAELNMLVLDHKN
VKYEKLCMSLQDICKSIHKLLSEHLHREETELWCLFRDCFTIEEQEKIIACMLGRISGEILQDMIPW
LMESLIPDEQHAVMSLWRQATRKTMFGEWLTEWYNSHAVEEETEEANKDPSENSDPLDVVWS
YLFEGAADEYKGSICSKPLEETELKGIMNKPLGKAAPNNKVEFGNKEENHLEISGSKKVCTGADE
TKYKEQTDSNAQAFQMSHNTSQSGQDSRYECLLSMSQEDVEATIRRISRDSSLDPQKKSYIIQNL
LMSRWIATQRIYNLEPSILSSNREAVPGQNPSYRDPHKLIFGCKHYKRSCKLLAPCCNKLYTCIRC
HDEEVDHLLDRKQITKMMCMKCMIIQPVGASCSNISCSSSMGKYYCKICKLFDDDREIYHCPYC
NLCRLGKGLSIDYFHCMKCNACMSRLIVEHVCREKCLEDNCPICHEYIFTSNSPVKALPCGHVMH
STCFQEYTCSHYTCPICSKSLGDMQVYFRMLDALLAEQKMPDEYLNQTQVILCNDCGRKGNAP
YHWLYHKCSSCASYNTRLF
SEQ ID NOs: 141-142 are exemplary protein sequences from the BTSL family found in
Thlaspi arvense (pennycress):
SEQ ID NO: 141 (T. arvense v1.1|Thlar.0031s0280.1.p):
MGDGKLHRLPPENASTSGSAVSVENTKLADAPVLFFVYFHKAFLAQLVELRRFATDAAA
KAGSFSGDLAGDLRRKFEFLKLVYKYHSAAEDEVIFLALDERVKNIVSNYSLEHAGTDDLFTSVF
HWLQVLEEEVGSKSDVLRQVILCIGTIQSSISQHMLKEERQVFPLLIEKFSFKEQASLVWQFLCSV
PVMVLEDFLPWMMSYLSHEEKTEVESCIKDVVPAEDSLQQVISSWLLDDSQSTKILKEDVSKNM
KKSSHTHQSSGCFQRFWQWSKSPIFIPNVGHNPIHGLQLFQNAVEKDLRDIQKDLCQAKFPSLILD
IDVLMARLNFLADVLVSYSNAFKKFFHPVLEDIADGRSSTPKHFTIDVCLESFQRLLYKSADDQV
RTDNFILKLQEELESLIVQVTKQFSAQRREVFPIISKNCNHEMRRQLLYTSIHVLPLGLLKCVILWF
SAHLSEEESQSILQFLSLEYSSPKKSFPRLLLQWLRFGYSGKTSVESFWKQLSVMFKIRCFCQKEH
TEEASGSFSHHQAQLQLCQGSRVDDDSLVCPGKRNKSSTCFLAAGDVYETPYSSRMNQQMLFSG
KLRPPLHLPDFFGGNNMDDPFVMDVKPIDLLFFFHKAMKADLDYLVCGSARLAADFGFLGEFQ
QRFRLIKFLYQIHSDAEDEIAFPALEAKGKLQNISHSFSIDHELEIKHFDKVSFILNEMSELNMLVSS
IKFSASDHRKMKYERLCLSLQEICKSMHKILSQHFKHEETELWGLFRDCFAIEEQEKIIGCMLGRIS
GEILQDMIPWLMDSLTYDEQHVVMSLWRQATRKTMFVEWLTEWYNGHFIQEKTGDQANNNDP
FGDSDPLEIVWNYFFEGGSDGDRGSIGIKLVELSETDLADIMKKPLEKVAPYNNLEAGNKGGEYE
QLAERKKICREADKKEDKEQTDSNSQIMNLAPMSHKVSQFGQSGKYGHLLTLSQEELAMMIKK
LSCDASLDPQKKYYIRQNLLMSRWIISQRLYNLEPSTLPSNGKTVPGQHPSYRDPQGLIFGCMHY
KRNCKILAPCCDQLFTCIRCHDEEADHSVDRKQIKKMMCMKCLLIQPIGENCSNTSCKISMGKYF
CKICKLHDDERKIYHCPYCNLCRVGKGLGIDYFHCMKCNACMSRLLVEHVCREKCLEDNCPICN
EYIFTSSSPVKALPCGHLMHSTCFQEYTCSHYTCPVCSKSLGDMQVYFSMLDALLAEEKMPDEY
SNKTQVILCNDCGRKGNAPYHWLYHKCTSCGSYNSRVL
SEQ ID NO: 142 (T. arvense v1.1|Thlar.0062s0240.1.p):
MVVDGSRRSSSTFPGQEPPDIASTSSSSVVNARLSDSPILLFVYFHKAFRAQLAELQSLAG
DAARANSDLALELRRKLDFLKLVYKYHSAAEDEVIFSALDTRVKNIAFNYSLEHDDTEDLFTSVF
HWLHVLEDEERGNVADVLREVVLCIGTIQSSICQHMLKEERQVFPLIIENFSFQEQSSLVWQFISS
VPVMALEEIFPWMTSLLSPKEKSEVENCVKQVVPNEVSLQLVINSWLIEDSRSSSFPALTKIMKGV
QCVEVSENMSNSSHQADSSSGGLFQRFWQWSKTSFSSPNTGGGHTLAHGIQIWHTAIKRDLLEIQ
KGLYQLKFPSFSLELNVLVVRLNFLADVLIFYSNAFKKFFYPVFEEMVDQHSSTSKQFTIDVESFK
KSLNLETRTASDNFVITLQEKLESLILTVAKQFSVEETEVFPIISKNCNVEMQRQLLYRSLHVLPLG
LLKCVTMWFSAQLSEDESQSIIHFLSLEDSFPNKSFSRLLLQWFRFGYSGKTSIESFWNELSFMFKP
IFPFGDEHTEEASGSLCKGSDSHLLVRQGNKDKSSTCIESKDLHAGYMIETPYSSAMNQKILIPERI
KPLQQLPGFFSDKNIGNHLTMDLKPIDLLFLFHKAMKKDLDYLVCGSARLASDCSFLGEFHQRFH
LIKFLYQIHSDAEDEIAFPALEAKGKLQNISQSYSIDHELEVEHLNKVSFLLKEMAELNMLVSTTD
SNSLDHHRDVKYEKLCSSLQDVCKSIHKLLSEHLHREETELWWLFRDFTVEEQEKIIACMLGRM
SGEILQDVIPWLMESLIPEEQHAVMSLWSQATRKTMFAEWLTEWYNSHVIGEETKEANKNSYED
SDPLDIVWKYLFEGADHENGGSIGRKPLKLSEKDLKLIMSKPLGNDAPNNKAEIGNKKENHQEIS
ENKKVCVGAEEKRCIEQSDDINFQQTKPAHQVFQMVTKSNQCGEMSKYECLLSMSQEDVDATI
RRISCDAVLDSQKKSYIMQNLLTSRWIATQRIHNLEPSILLSSNKEAIPGQHPSYRDPHKLIFGCKH
YKRSCKLLASCCNQLFTCIRCHDEETDHSVDRKHITKMMCMKCLLIQPVGANCSNSSCSSSMGK
YYCKICKLFDDEREIYHCPYCNLCRGKVLRR

SEQ ID NOs: 143-148 are exemplary protein sequences from the BTSL family found in Glycine max (soybean):

SEQ ID NO: 143 (G. max Wm82.a2.v1|Glyma.17G096900.1.p):
MDDGDPSLSDKEEGENDEEDTPLLRVPLVDAPILLFVCFHKAFRSELDHLRRLAETASSL
EDEPRRCRQIVLQLQRRFQFLKLAHKYHCAAEDEVIFLALDTHVKNVICTYSLEHRSTNGLFGSV
FHFLDELMVPKENISKLFQELVYCIGILQTSIYQHMLKEEEQVFPLLIQKLSNKEQASLVWQFICSV
PIMLLEEVLPWMVSFLSANKQSEVTQCLNEIAPMEKAMQEVLVSWLRSSKQTCTETCFQSGEFQ
GVDGFLHIERSLELSYCNRNSEEISSPMKVNGKEIEDGANQVNVLHLWHNAIKKDLKDILEELHL
LRKSSCFQNLDSILIQLKFFADVLIFYSDAQKKFFHPVLNKHAYGWLSKSIEQFLGESNIEDIQQLL
FYNSESGILLSKFIEKLCQTLESFVSGVNKQFAFQENEVFPIFRKNCRNGMQERLLSLSLYMMPLG
LLRCVITWFSVRLSEKESSSILYCIKKGNNSVCKAFSSLLHEWFRIGYSGKTSIEKFRQELQHMFK
RRCSLLPEQIKEAHEFSFLNSEKQPHKVSGQNCLSYSSSSGSNNVNKYETPYSTGINLHIFFPSTVA
KLHQHPTLHAEERSSISFLDDPKPIDLIFFFHKAIKKDLEYLVLGSTQLEKNDKLLMDFHKRFHLIY
FLHQIHSDAEDEIVFPAMEARGKLKNISHAYTFDHKHEVDHFNKISHILDKMSGLHLSVSTIDPNV
KEKGILRYHHLCRKLQEMCKSMHKSLSDHINREEIEIWPIIRKFFSNHEQGRIIGCMLGRIRAEILQ
DMIPWLMASLTQEEQHVLMFLWSMATKNTMFDEWLGEWWDGYSLTKVTEGSNVAPLQPVEP
LEIISKYLSEEILDELQEESSANKSINFLQKDHNGDNVVLSNYNFDDKVKVHNAEQNNNQCSKLT
NQFHDHNKHACNEVTNIINPVNNEGKYSQLCDKSGRYDRLLKLSQDDLETVIRRVSRDSCLDPQ
KKSYIIQNLLMSRWIIRQQISSTEANIKNDELEFPGKHPSYRDPLKLIYGCKHYKRNCKLFAPCCN
QLHTCIHCHNEESDHSVDRKSITKMMCMKCLVIQPISATCSTISCNLSMAKYYCRICKLFDDEREI
YHCPYCNLCRVGKGLGVDYFHCMNCNACMSRSLMTHTCREKHLEDNCPICHEYIFTSCSPVKAL
PCGHVMHSTCFQEYTCFNYTCPICSKSLGDMQVYFRMLDALLAEERISDEISSQTQVLLCNDCEK
KGETPFHWLYHKCPSCGSYNTRVL
SEQ ID NO: 144 (G. max Wm82.a2.v1|Glyma.05G237500.1.p):
MASPLDGGGVAVLPNSVNKVDSSSVLNGGLKCSKPESPILIFLFFHKAIRNELDALHRLA
VAFATGNRSDIKPLSGRYHFLSSMYRHHCNAEDEVIFPALDIRVKNVAQTYSLEHKGESNLFDHL
FELLNSSINNVESFPKELASCTGALQTSVSQHMAKEEEQVFPLLIEKFSLEEQASLVWQFLCSIPVN
MMAEFLPWLSASISPDESQDLRNCLIKIVPEEKLLQKVVFTWMEGRSSINTVETCADHSQVQCSS
RALTHQLEKVNCACESTTTGKRKHSGSMIDVSDTTGTHPIDEILLWHSAIKKELSEIAVETRKIQH
SEDFTNLSAFNERFQFIAEVCIFHSIAEDKVIFPAVDGEFSFFQEHAEEESQFNDFRRLIESIQSEGAS
SNSDVEFYSKLCIHADHIMETIQRHFHNEEVQVLPLARKHFSFRRQCELLYQSLCMMPLKLIERV
LPWLVGSLTEDEAKTFQRNMQLAAPATDSALVTLFCGWACKARNEGLCLSSSASGCCPAQRLS
DIEENIVRPSCACASALSNRHCSVLAESGGNKRSVKRNILESHKNEDLPETSETENIQKQCCSARS
CCVPGLGVSSNNLGLSSLSTAKSLRSLSFCSSAPSLNSSLFIWETESSSCNVGSTQRPIDTIFKFHKA
IRKDLEYLDVESGKLSDGDETILRQFNGRFRLLWGLYRAHSNAEDDIVFPALESKEALHNVSHSY
MLDHKQEEQLFEDISCVLSEFSVLHEALQMTHMSDNLSESNFGTSDANTSDDIKKYNELATKLQ
GMCKSIRVTLDQHLFREECELWPLFGRHFTVEEQDKIVGRIIGTTGAEVLQSMLPWVTSALTQDE
QNKMMDIWKQATKNTMFNEWLSECWKESRVSTAQTETSDHSTSRRGAEYQESLDHNDQMFKP
GWKDIFRMNQNELESEIRKVYRDSTLDPRRKAYLVQNLLTSRWIAAQQKSPKALSEGSSNSVEIE
GLSPSFQDPEEHVFGCEHYKRNCKLRAACCGKLFTCRFCHDNVSDHSMDRKATSEIMCMRCLNI
QPIGPICMTPSCNGFSMAKYYCNICKFFDDERNVYHCPFCNLCRVGRGLGIDYFHCMKCNCCLGI
KSASHKCLEKGLEMNCPICCDDLFTSSATVRALPCGHYMHSACFQAYTCNHYTCPICSKSLGDM
AVYFGMLDALLAAEELPEEYKDRCQDILCHDCNRKGTSRFHWLYHKCGFCGSYNTRVIKCETS
NSSCS
SEQ ID NO: 145 (G. max Wm82.a2.v1|Glyma.07G093700.1.p):
MATPLTGLNGVGGGGGVAVLANPVSKVDSSANGGGGFGRSLSESPILIFSFFHKAIRNEL
DALHRLAMAFATGNCSDIQPLFQRYHFLTSMYRHHSNAEDEVIFPALDIRVKNVAQTYSLEHQG
ESDLFDHLFELLNSSIHNDESFPKELASCTGALQTSVSQHMAKEEEQVFPLLLEKFSLEEQASLVW
QFLCSIPVNMMTEFLPWLSTSISPDESQDLRKCLSKIVPEEKLLQKVVFTWMEGGSSANTVENCL
DHSQVRCSLNPLTHQNGKIKCACESTATGKRKYSGSIIDVSDTMRTHPIDEILLWHNAIKKELNEI
AAQTRKIQLSGDFTNLSAFNERLQFIAEVCIFHSIAEDKVIFPAVDGKFSFFQEHAEEESQFNEFRS
LIESIQSEGATSSSETEFYSTLCSHADHILETIQRHFHNEEVQVLPLARKHFSFKRQRELLYQSLCM
MPLKLIERVLPWLIRSLTEDEAQMFLKNMQSTAPAIDSALVTLFCGWACKARKDGLCLSSSVSG
CCPAQRFTDIEENTVHSSCTPASALSGRVCSVLAESDGTQQRSVKRNISEVHKNEDVSKTSESESF
QKQCCSAQSCCVPALGVNKNNLGLGSLSTTKSLRSLSFTASAPSLNSSLFIWETDNSSCEVGSTER
PIDTIFKFHKAIRKDLEYLDIESGKLCDGDETIIRQFSGRFRLLWGLYRAHSNAEDDIVFPALESKE
ALHNVSHSYTLDHKQEEKLFEDISCVLSELSVLHENLQRAHMSVDLSENDFGISDANDDDNIKKY
NELATKLQGMCKSIRVTLDQHIFREELELWPLFGKHFTVEEQDKIVGRIIGTTGAEVLQSMLPWV
TSALTQDEQNKMMDTWKQATKNTMFNEWLNECLKESPVSTSQTEASERSTSQRGGDYQESLNL
NEQMFKPGWKDIFRMNQNELESEIRKVYRDSTLDPRRKAYLVQNLMTSRWIASQQKLPKAPSGE
SSKQIEGCSPSFRDPEKQIFGCEHYKRNCKLRAACCGKLFTCRFCHDNASDHSMDRKATLEMMC
MQCLTIQPVGPICMSPSCNGLTMAKYYCNICKFEDDERNVYHCPFCNICRVGQGLGIDYFHCMK
CNCCLGIKSASHKCLEKGLEMNCPICCDDLFTSSATVRALPCGHYMHSSCFQAYTCSHYTCPICS
KSLGDMAVYFGMLDALLAAEELPEEYRDRYQDILCHDCDRKGTSRFHWLYHKCGSCGSYNTR
VIKSEAANSSCL
SEQ ID NO: 146 (G. max Wm82.a2.v1|Glyma.09G182600.1.p):
MATPLTGLNGGGGGVAVLTNPVNKVDSSANGGGGFGRSLSESPILIFSFFHKAIRNELDA
LHRLAMAFATGNCSDIQPLFQRYRFLRSMYSHHSNAEDEVIFPALDMRVKNVAQTYSLEHQGES
DLFDHLFELLNSSIHNDESFPKELASCTGALQTSVSQHMAKEEEQVFPLLLEKFSLEEQASLVWRF
LCSIPVNMMTEFLPWLSSSISPDESQDLQKCLSKIVPEEKLLQKVIFTWMEGRSSANTVENCLDHS
QVRCSPNPLTHQNGKIKCACESTATGKRKYSGSSIDVSDTMRTHPIDEILLWHNAIKKELNEIAAQ
SRKIQLSGDFTNLSAFNERLQFIAEVCIFHSIAEDKVIFPAVDGKFSFYQEHAEEESQFNEFRSLIESI
QSEEATSSSETEFYSTLCSHADHILEMIQRHFHNEEVQVLPLARKHFSFKRQRELLYQSLCMMPL
KLIERVLPWLIRSLTEDEAQMFLKNMQLAAPAIDSALVTLFCGWACKARKDGLCLSSSVSGCCP
AQRFTDIEENTVQSSCTSASALSGRVCSVLAESDGTQQRSVKRNISEVHKNEDVSKTSEIESIQKQ
CCSARSCCVPALGVNKNNLGLGSLSTTKSLRSLSFTASAPSLNSSLFIWETDNSSCDVGSTERPIDT
IFKFHKAIRKDLEYLDIESGKLCDGDETIIRQFSGRFRLLWGLYRAHSNAEDDIVFPALESKEALH
NVSHSYTLDHKQEEKLFEDISCVLSELSVLHENMQMTHMSVDLSENDFGISDANDNIKEYNELA
TKLQGMCKSIRVTLDQHIFREELELWPLFGKHFTVEEQDKIVGRIIGTTGAEVLQSMLPWVTSAL
TQDEQSKMMDTWKQATKNTMFNEWLNECLKETPVSTSQTEASERSTSQRGGDYQENLNLNEQ
MFKPGWKDIFRMNQNELESEIRKVYRDSTLDPRRKAYLVQNLMTSRWIAAQQKLPKALSGESSK
QIEGCSPSFRDPEKEIFGCEHYKRNCKLRAACCGKLFTCRFCHDNASDHSMDRKATLEMMCMQ
CLTIQPVGPICMSPSCNGLTMAKYYCNICKFEDDERNVYHCPFCNICRVGQGLGIDYIHCMKCNC
CLGIKSASHKCLEKGLEMNCPICCDDLFTSSATVRALPCGHYMHSSCFQAYTCSHYTCPICSKSL
GDMAVYFGMLDALLAAEELPEEYRDRYQDILCHDCDRKGTSRFHWLYHKCGSCGSYNTRVIKS
EATNSSCL
SEQ ID NO: 147 (G. max Wm82.a2.v1|Glyma.08G044700.1.p):
MATPLDGGGVAVLPNSVNKVDSSSALIGGLKCSKPESPILIFLFFHKAIRNELDALHRLAI
AFATGNRSDIKPLSERYHFLSSMYRHHCNAEDEVIFPALDIRVKNVAQTYSLEHKGESNLFDHLF
ELLNSSINNDESFPRELASCTGALQTSVSQHMAKEEEQVFPLLIEKFSLEEQASLVWQFLCSIPVN
MMAEFLPWLSTSISPDESQDMQNCLIKIVPQEKLLQKVVFSWMEGRSSINTIETCVNHSQVQCSS
RSLTHQVEKVNCACESTTTGKRKHSESMIDVSDTTGTHPIDEILLWHNAIKKELSEIAVEARNIQH
SGDFTNLSAFNERFQFIAEVCIFHSIAEDKVIFSAVDGEFSFFQEHAEEESQFKDFRHLIESIQSEGA
SSNSDVEFYSKLCTHADHIMETIQRHFHNEEVQVLPLARKHFSFRRQCELLYQSLCMMPLKLIER
VLPWLVGSLTQDEAKMFQRNMQLAAPATDSALVTLFCGWACKARNEGLCLSSGASGCCPAQR
LSDIEENIGWPSCACASALSNSHVLAESGGNNRPVKRNISELHKNEDLPETSEAEDIQKQCCSARP
CCVPGLGVSSNNLGLSSLSTAKSLRSLSFSSSAPSLNSSLFIWETESSSCNVGSTQRPIDTIFKFHKAI
RKDLEYLDVESGKLSDGDETILRQFNGRFRLLWGLYRAHSNAEDEIVFPALESKEALHNVSHSY
MLDHKQEEQLFEDISCVLSEFSVLHEALQMTHMSDNLTESNFGTSDANNSDDIKKYNELATKLQ
GMCKSIRVTLDQHLFREECELWPLFGRHFTVEEQDKIVGRIIGTTGAEVLQSMLPWVTSALTQDE
QNKMMDTWKQATKNTMFNEWLSECWKESPVSTAQTETSDHITSQRGAEYQESLDHNDQMFKP
GWKDIFRMNQNELESEIRKVYRDSTLDPRRKAYLVQNLMTSRWIAAQQKSPKALSEGSSNSVEI
EGLSPSFRDPGKHVFGCEHYKRNCKLRAACCGKLFTCRFCHDNVRDHSMDRKATSEMMCMRC
LNIQPIGPLCITPSCNGFSMAKYYCNICKFFDDERNVYHCPFCNLCRVGQGLGIDYFHCMKCNCC
LGIKSSSHKCLEKGLEMNCPICCDDLFTSSATVRALPCGHYMHSACFQAYTCSHYTCPICSKSLG
DMAVYFGMLDALLAAEELPEEYKDRCQDILCHDCDRKGTSRFHWLYHKCGFCGSYNTR VIKCE
TSNSSCS
SEQ ID NO: 148 (G. max Wm82.a2.v1|Glyma.09G115100.3.p):
MATAGGGGGGGCVAVMAGSCTVSTPSSSYGGGACSSNSKDTLIESPILIFCLFHKAISSEL
QSLHATAFDFVSNRRHSQPHSPLKIMSFSHRCHFLRTLYKHHCNAEDQVIFPALDKRVKNVAHT
YFLEHEGEGLLFDQLFKLPNSNLLNEESYGRELASCIGALRTSICQHMFKEKEQVFPLVIEKFSSEE
QGSLVWQFLCSIPVKMMAEFLPWLASYISSDEYQGLLSCLCTIIPKEKLLHQVIFGWMEGLKIKH
RKCTHDTKVQWKDVGMSNLLSHNEKVYSICGSSKTVKRKRVGLNEDPTNSNISCPLDELLLWH
KAIKQELSDLAETARKIQLSEEFSNLSSFSGRLQFITEVCISHSIAEDRVIFPAIKAELHFLQDHTDEE
LQFDKLRCLIDSIQSAGADSSSAEFYFKLSSHTEQITDTILKHFEDEEAQVLPLARKLFSPQRQREL
LYESLCSMPLKLIECVLPWLVGSLNQTEVRSFLQNMYMAAPATDHALVTLFSGWACNGYSRNS
CFYSSTTGLCPDRRLMEIPFCMCEPSFGLNEKSSAIQQEDENGCIGPVKHGKPESKQDNDVANLM
VTGKTLSSSLNQSCCVPELRVNASNLGLGSLALTKSLRELSDYPSAPSLNSSLFMWETNLVSADN
QCIARPIDNIFKFHKAIRKDLEYLDVESVKLNDCDEIFIQQFTGRFCLLWGLYRAHSNAEDDIVFP
ALESKDNLHNVSHSYTLDHQQEEKLFLDISSGLTQLTQLHELLYKKNWSDHITNCFSNYAGCYDI
DTVQNYNELSTKIQGMCKSIRVTLDQHILREELELWPLFDRHFSLEEQDKIVGHIIGTTGAEVLQS
MLPWVTSALAQDEQNKMIDTLKQATKNTMFCEWLNEWWKGPASSLNITTPGDYSLGTDAYED
SEHSFLAFRPGWKDIFRMNQNELESEIRKVSQDSTLDPRRKAYLIQNLMTSRWIASQQKSSQSLG
VESSKGDILETSLSFHDPEKKIFGCEHYKRNCKLRAACCGKLFTCQFCHDKVSDHLMDRKATTE
MMCMQCQKIQPAGPVCATPSCGSLLMAKYYCSICKLFDDERTVYHCPFCNLCRLGKGLGVDFF
HCMQCNCCMSKKLVDHICREKGLETNCPICCDELFTSSESVRALPCGHFMHSACFQAYTCSHYIC
PICSKSMGDMSVYFGMLDALLASEELPEEYRNQCQDILCNDCHEKGTAPFHWLYHKCGFCGSY
NTRVI

SEQ ID NOs: 149-151 are exemplary protein sequences from the BTSL family found in Solanum lycopersicum (tomato):

SEQ ID NO: 149 (S. lycopersicum ITAG4.0|Solyc03g113350.3.1):
MDDLGELEKDADMVDLTSSLAGVKLVDAPILFFVISHKAIILELADIHRVAVEASETGSQ
HVELVRDLSRRLEFLKIVYKYHCAAEDEVVFLALDAQVKNVVYTYSLEHKSIDDLFVSIFKCIDC
LQKETEEFAKLFNGLTCYIGTIETIISQHMLKEEEQIFPSLMKRFSSKEQARLVWQYLCSVPLLLLE
DFLPWVITSLSSVRKTDFLNFIHVVLPEETLIQEVFISWLDNNELSAQSCLEHGKGANSHYGIANM
KHMLKERTIVVHSSEMKLPTKKNPIDGFHLWHAAIRRDLKEILEELHHLRNSFCLSTLMSLVAQL
KFFAEVLNFYSIALDQTFYPLIDELNKSDASASHEQFIEGSQIEELQRLLYCKLQDVIQLNVLVEM
LCQEVESFVGRISKKLHFLETKVFLVIRENCSHELQLWLLYRSLQMLPLGLLKCMIIWFSAHLSED
ESKLILNNVMLGSPVVNRSFTSLLYEWVRTGYSGKISLEKFRKDLEEMFSSRSSLLEKSFNNSGSS
FLQSDMQSFDQSNNLLFQSASAVTSKNSVSYHPSPLGIIEKLDTSYSYGINTQIFSSDSQKSLSFFPG
TSSRSSNDLKVPIREFIPIDFVHFFHKALINDIQDVVSLSVKLAEDVGVLAEFHRRFHHLHFLLRIHS
NSEDEVAFPALESRVTLQNVSHSYSLDHNLEVENFNKISVILNKFISLQGDEVVDGKKFKYKRLC
LKLHNACISMQRTLTDHINHEEIELWPLFREHFSVEEQEKIVGNMLGRTKTEILQEMIPWLMASLT
PDEQRGMMMLWRKVTKHTKFFEWLGEWWEPVKREESLNAEMEPKIAPFLSIDPLEAVSTYLSR
NGVKQGIWNEKGSEFVNHDIYQHGSFTEDKAHNAKGKQNIDLSEDMARYSTVVDKNKYTETV
DSIAQKETTCQDIESCDKSRLQEHHLLTQEELVSVIRKISCDSSLDSEMRSHLMQSLLMSQWILTQ
KLSDSEVAAANDTEKFPGQCPSYRDEQEIIFGCSHYKRNCKLLAPCCKKLFPCIRCHDETTDHTL
DRKTITQMMCMKCLKIQPIGPSCSTPSCDGFSMGRYYCKICKLFDDERQIYHCPFCNLCRLGKGL
GVGYFHCMNCNACMSKSLSVHVCREKCLEENCPICHEYIFTSTNPVKALPCGHLMHSKCFQEYT
CSHYTCPICSKSLGDMQVYFEMLDALLSEQKIPEEYAGQTQAILCNDCEKRGTASFHWLYHKCS
SCGSYNTRLV
SEQ ID NO: 150 (S. lycopersicum ITAG4.0|Solyc06g071130.3.1):
MGGKEDDDDTLALPLPSCSDSVVVVDDEFVKQLVDSPILFFVLSHKAVEIELHQIRCVAV
EALDSGGEVVDELCKRLHFLKIVYKYHCVAEDEVLFQALDAQVKNVVFTYSLEHNSIDVLFSSIF
DCLDRLQEEKDEISVLFNELTCSIGTIQTTISQHMLKEEEQIFPLMMEKFSSEEQARLIWQYLCSVP
LMILEDFMRWLTASLSSHERAYFLKFIHIVLPEEKLIQEVFISWIDENKEASSRSCIEDGKGAKFHY
GKANMKYIFEMDVLMVQCKEMQHQKASEEHNPIDGFHIWHAAITQDLRVIMDELYQIRNTLCV
STLLSVITQLKFFADVFTFYSNALDQIYYPLVNQLNKDSPSPFYEQFIERSQIEELQKLLYYKLHEEI
QIKVFVDMLCQEVELFVGRMNKKLQFLETEVFVFIRKTCSYELQLWLLYISLHMLPLGLLKCMII
WFSAHLSENESKMMLNNIKLGSSVVSKSFSTLLYEWVRMGYSGKISVEKFRKDLEEMFSSGTYL
FEKWCKNSGSSSSHSEIHSPDRPYHPSTLDNIGKHDTPYSNGINLRIFFSDSLNGLFCHPETAVDG
MRLSRLDVKPIDFFHFFHKALKKDLQYALSLSVKLAEDVGLLAEFERHFHHVRFLYQLHSKSED
EIAFPALESKGQLRNVSHSYGIDHKLEVEQFDRISIVLNEITSLQGCVDMIDSNKLKYKKLCLNLH
DTCISMHKTLTDHIYREEVELWPLFKEHFSVEEQEKIIGDMLGRTKAEILREMIPWLMASLTPEEE
HGIMSIWRKVTKNTKFFEWLGEWWEGIKRDESVNAEKGSKLSLALAVDPLEVVSTYLPRDDFW
SSSVCHEKGENFLSTESADCDLDQSGSFAADKSQKAKGNKNVDRSTDITQHSTEVDKKICNDTID
IADKKEITCQDIKLYEQSRQKNHKEHHLILTQDKLVDAIRRVSRDFSLDSEKKSHLMQSLLMRQI
HFQLLFFDLFLRKNQWILTQEKFHSEVATAKDKEKITGQCPSFRDKTESVFGCKHYKRNCKLLAP
CCNELFPCIRCHDEISDHFLDRKSITQMMCMKCLKIQPICPSCLTLTCNNFSMAKYYCRICIVEDD
DRQIYHCPFCNLCRVGEGLGVGVFHCMTCNACMLSKSLSIHTCRENCLEDNCPICREDIFTSATP
VKQLPCGHLMHSTCFQDYTFTHYTCPICSKTIGDMKVLFELLDAFLSEEKIPEEYAGQIQVILCND
CQKRGTASFHWHYHKCPYCGSYNTRLI
SEQ ID NO: 151 (S. lycopersicum ITAG4.0|Solyc03g119300.4.1):
MATQGREGGGGVAVLCGGGVNAVDSSASSSNGVLEKETGGKQESPILFFLFFHKAIRLE
LDALHHSALAYATGQLEDIQPLLKRYRFLRSVYKHHCHAEDEVIFPALDIRVKNVAPTYSLEHKG
ENDLFDHLFEILNSEKQNCERFPRELASCTGALQTSVSQHMSKEEEQVFPLLTEKFSMDEQASLV
WQFLCSIPVNMMKKFLPWLSSSISPDEHKDMQKCLSMIIPKEKLLQQVIFSWMEGGKCVTAVGG
HDVDADPPGSVDFNSVTETYASGNEKCVCESSSPGKRKFRLKGDSFDTDSGNPIDEVLHWHNAI
KRELDEIAAEARRIELAGELSSLTAFYARLQFIAQVCIFHSIAEDKVIFPAVDGGLSFFQEHAEEEIQ
FNELRCLIESIQCTEVNSTSAAEFFSKLYSQADLIIETIKQHFHNEEVQVLPLARKHFTRDRQRKVL
YQSLCLMPLKLMEQVLPWLVGALSEDEARSFLKNLQLAAPEADTALVTLLSGWACKGRTDGVC
LSSSVTGCCAVKRFADIEEYYTGAPCPCFLSVHSDDSKRPFKRNLNSLCSKDDTLDLSKGVNACN
ISCNDQSCRVPGLGVSDNNLVLTTISTPKSLRSLTFSSAAPSLESSLFVWETDCTSSQPNHKVHPID
TIFKFHKAIQKDLEYLDVESGKLSDCPETFLRQFIGRFRLLWGLYRAHSNAEDEIVFPELESKEAL
HNVSHSYMLDHKQEEKLFEDISSALTNLSELHKGLKEAYQKESGSSILESTGLYDRDCKRKYNEL
ATKVQGMCKSIRVSLDQHIFREEHELWPLFGKHFSMEEQDIIVGRIIGSTGAEVLQSMLPWVTSA
LTQDEQNKMMDTLKQATRNTMFSEWLNDCWRRNPEVSSQSEALQNSYTNRGVDSHEGLDQSD
HMFKPGWKDIFRMNQTELESEIRKVHWDSTLDPRRKSYLIQNLMTSRWIASQQKSQASTEEISRS
EDVVGYSPSFRDKEKQIFGCEHYKRNCKLLAACCGKLFACRFCHDEVSDHSMDRKATLEMMC
MRCLKVQPIRPSCTTPSCNGFSMAKYYCSICKFFDDERPIYHCPSCNLCRVGHGLGIDFYHCMKC
NCCLGKGLVDHKCLEKALETNCPICCEFLFTSSATVRPLPCGHYMHSACFQEYASSNYICPICSKS
MGNMAVYFGMLDALLANEVLPEEYRNHWQDILCNDCEQKCRTPFHWLYHKCGFCGSYNTRVI
TLPTTASDCPT

SEQ ID NOs: 152-153 are DNA sequences from the BTSL family found in Arabidopsis thaliana:

SEQ ID NO: 152 is an exemplary DNA sequence of BTSL1 (NM_106135):
ATGGGAGGCGGAAATCTTCATTCCCTTCCGCCGGAAAACGCTTCCGTTTCTGCATCTT
ATGCGGTGACCGTTGGAAATACCAAGCTTTCTGATGCTCCGGTTCTTTTCTTCGTTTACTGCC
ACAAGGCTTTCCGTGCTCAGCTGGTGGAGCTCCGGCGTTTCGCTACTGACGCTGCTGAAGCC
GATTCCTTTAGTGGGGATCTGGCGGTGGAGCTCTCCCGGAAGTTTGAGTTCTTGAAGCTTGT
ATATAAGTATCATAGCGCAGCTGAAGATGAGGTTATATTTTTGGCGCTAGATAAACGAGTGA
AAAACATAGTTTCTAATTACTCACTTGAGCATGCTGGCACAGATGATCTCTTTACCTCAATTT
TCCACTGGCTACATGTTCTTGAAGAAGAAATAGGAAGTAGAAGCGATGTACTTCGTGAAGTT
ATTTTATGCATAGGCACCATCCAGTCATCTATATGCCAACATATGCTTAAAGAAGAGCGGCA
GGTGTTTCCTTTGTTAATAGAGAAGTTTTCTTTCCGAGAACAAGCATCACTTGTGTGGCAATT
CATTTGTAGTGTTCCAGTGATGGTGCTTGAAGACTTTTTGCCATGGATGATCTCTCATCTCTC
TCACGAGGAGAAAATTGAAGTTGAGAATTGTATAAAAGATGTTGCCCCAAATGAAGACTCA
TTGCAACAGGTCATAAGCTCTTGGCTGCTTGACGATAGTCAATCTTCTTGTGGGACTCCTACA
GAGATCATGAAAGGAGTCCAGTATGTTAATGTGTCTAAAAGTTTGAAGAAATCACCTGAGTC
ACATCCGTCAAGCGGATGTTTTCAACGCTTTTGGGAGTGGAGTAAAAAGTCCCTTTCAATTC
CAAATGTAGGACGGAGCCCAATTCATGGTCTTAGACTTTTTCAAAATGCAATTGAAAAAGAT
TTGAGAGATATTCAAGAAGGATTATGCCAAGCAAAATTCCAAACCCTTATTTTGGATCTTGA
TGTACTGATGGCTAGATTAAACTTCCTTGCTGATGTCCTTGTTTCTTATAGCAATGCATTTAA
GAAGTTCTTTCACCCAGTGTTAGAAGAAATGACAGCTCGCCGCTCTTCAACTGCCAAACAGT
TCAACATAGATGATTGTCTTGAGAATTTTCAGAGATTGTTATACAAAAGTGCTGATGATAAA
ACAAAGACGGACAATTTTCTATTGCAGCTTCAGGAGGAACTTGAGTCCCTTATAATCCAAGT
AACAAAGCAATTTGCCATACAGAGAACAGAGGTATTCCCAATCATCAGCAAGAATTGCAAC
CATGAAATGCAGAAGCAGCTCCTATACACAAGCATTCATGTCTTACCTCTTGGATTACTAAA
GTGTGTCATACTTTGGTTTTCTGCTCATTTATCAGAAGAAGAATCTCAATCCATTCTTCATTTC
TTAAGTTTGGAAGATTCTTCTCCCAAGAAATCATTTCCACGCCTCTTGCTACAGTGGCTTCGT
TTTGGCTATTCAGGCAAAACCTCTGTTGAAAGATTTTGGAAACAGTTGGATGTTATGTTCAA
AGTAAGATGTTCCTGCCAAAAGGAGCACACTGAAGAAGCATCAGGATCATTTTCCAACCAA
ACACAGCTGCAACTATGTAAAGTATCTAAAGATGTTTATCCTAGAAAGAAAGATAAGTCTTC
AACCTGCTTCATGTCAATGGATCTAGCTGTTGGAGACATGTATGAGACACCTTACTCAAGTC
GAATGAATCAACAAATGACTTTCTCAGGAAAGCTTAAGCCTCCTCTCCATCTCCCAGATTTTT
TTGGTGAGAAGAATATGGATGATCCTATGATTATGGATGTTAAACCAATTGACCTCCTTTTCT
TCTTCCACAAGGCAATGAAGATGGATTTGGACTACCTTGTTTGTGGATCAACAAGATTGGCA
GCCGACTTTCGTTTCCTCGCGGAGTTTCAGCAACGGTTCCATATGATAAAGTTTTTGTATCAG
ATACATTCTGATGCAGAAGATGAGATTGCATTTCCAGCATTGGAAGCCAAGGGACAGCTAA
AAAACATTAGTCACTCGTTTAGCATTGATCATGAGCTAGAAACTAAACACTTCGACAAAGTT
TCGTTCATTTTGAATGAGATGTCAGAGCTGAACATGTTGGTTTCTACCATCAATACCACTGCA
GCTGACCACGACCGAAAAATGAAGTATGAGCGGTTATGTCTCAGTCTCCGAGAGATTTGCAA
ATCAATGCACAAGCTATTGTCTGAGCATATCCAACATGAAGAAACTGAGCTTTGGGGTCTAT
TCAGGAACTGCTTCTCTATTGAAGAACAAGAGAAAATCATAGGATGCATGCTCGGGAGAAT
AAGTGGGGAAATATTGCAGGATATGATTCCTTGGTTAATGGAATCTTTAACCTCTGATGAAC
AGCTTGCCGCGATGTCCTTGTGGCGCCAAGCAACAAGGAAAACAATGTTTGTCGAATGGCTA
ACAGAATGGTATAACGGTCATGTTTTACAAGAGGAAGCAGGAGAAGCAAACAACGATCCAT
TTGGGGATTCAGATCCACTAGAGATTGTCTGGAAGTATCTCTTTGAAGCATCTGCTGATGGG
GAAAAAGGGAGCATGCGCAGCAGCCTTCTCAAACTCCCGAAGACGAATTTTACAGGTATTA
TGAATCAGCCTCCTCCTAATTACAAGGTTGAAGTTGGCAAAAAAGAGGAAAAAGATCTAGA
ACGGTCAGAAAGTAAAAAGATATGCAGAGGATCTAACCAAGAGGGGGACAAAGAACAAAC
TGACAAAATGTCTCAGAAGGTTAGCCAGTTTGGTCCATCCAAGAAATATGAACAACTGTTAA
CAATGAGTGAAGAAGAACTTGTGGTTGTGATTAAGAAAATATCATGCGACTCTTCCCTGGAT
CCTCAGAAGAAAGACTATATCAAGCAGAACTTGTTAATGAGTCGGTGGAATATCTCACAAC
GTACATATAATTTGGAACCATCCTCTCTCTCGAGCAACATGGAAACAGTTCATGGTCAACAT
CCATCATATCGAGATCCTCACAGTTTGATCTTTGGTTGCAATCACTACAAGAGAAATTGTAA
ACTTCTTGCTCCTTGTTGTGACAAGCTCTTCACATGTATACGATGCCATGATGAAGAAGCTGA
TCACTCGGTGGATAGGAAACAGATTACAAAGATGATGTGCATGAAATGCCTCTTGATTCAAC
CGATTGGTGCAAATTGCTCAAATACTTCGTGCAAGTCGTCAATGGGAAAATACTTCTGCAAA
ATATGCAAATTATATGACGATGAAAGGAAAATTTATCACTGTCCTTACTGCAACCTCTGTCG
AGTAGGGAAAGGATTGGGTATTGACTACTTCCACTGCATGAAATGCAATGCTTGTATGTCAC
GTACCTTAGTAGAGCATGTTTGCAGGGAAAAGTGCTTAGAAGATAATTGTCCGATTTGCCAT
GAGTATATCTTCACCTCCAGCTCCCCAGTGAAGGCTCTTCCTTGTGGTCACTTGATGCACTCC
ACATGCTTTCAGGAGTACACATGTTCACATTACACATGCCCTGTCTGCAGCAAGTCGCTTGG
AGATATGCAGGTCTACTTTAAGATGTTAGACGCATTGCTTGCAGAAGAGAAGATGCCTGATG
AATACTCCAACAAAACTCAGGTGATCTTGTGTAATGATTGTGGAAGAAAAGGAAATGCTCCT
TACCATTGGCTTTACCACAAATGCACAACCTGTGGCTCCTACAACTCAAGGCTCCTTTAG
SEQ ID NO: 153 is an exemplary DNA sequence of BTSL2 (NM_101748):
ATGGGAGTCGGAGATCCTCTTCCACTTCCGCCGGAAAAGAACCGTCGGGAGGTTAAT
AAACCGCCGGATATTGCGTCGACTTCCTCCTCCTCCGCTTCGGCGGTGAATAATGCTCGGTTG
TCCGATGCTCCGATTCTTTTGTTTGTTTATTTCCATAAGGCTTTCCGTGCGCAGCTAGCGGAG
CTCCAGTTTCTGGCCGGTGATACAGTCCGGTCCGGTTCTGATCTGGCCGTGGAGCTCCGCTCT
AAGTTTGAGTTTCTTAAGCTTGTATATAAGTATCATTCCGCAGCTGAAGATGAGGTTATATTT
TCAGCGTTGGACACTCGTGTGAAGAACATTGTTTTTAATTACTCGCTTGAACATGATGCCAC
AGATGATCTTTTCACTTCAGTTTTTCATTGGCTGAATGTTCTTGAAGAGGAACAAGGGAACA
GAGCCGATGTACTTCGTGAAGTTGTTTTATGCATTGGAACCATTCAGTCATCCATATGTCAAC
ATATGCTTAAAGAAGAACGACAGGTGTTTCCTTTGATGATTGAGAACTTCTCCTTTGAAGAA
CAGGCATCGTTAGTGTGGCAATTCATTTGTAGTGTTCCAGTGATGGTGCTGGAAGAGATTTT
CCCATGGATGACGTCTTTACTCTCTCCCAAGGAGAAATCTGAAGTTGAGACTTGCTTCAAAG
AAGTTGTCCCAAATGAACTCTCTTTGCAACTGGTCATAAACTCTTGGCTGATTGATGACAGTC
AATCTTCTTTGACGGCTCTTACAAAGATTATGAAAGGAGTTCAGTCTGTAGAAGTGTCTGAA
AATATGACCAACTCTCAGACAAATTCATCATCAAGTGGTGTGTTTCAACGTTTTTGGCAGTG
GAGTAAAAAAATGTCTTTTTCTAGTCCAAATACAGGACACATTTTGGTTCATGGTATCCATCT
TTGGCATAATGCAATTAGAAAAGATTTGGTAGACATTCAAAAAGGACTATGCCAATTGACAT
TTCCAAGCCTTTCATTGGACCTCAATGTTCTGGTGGTTCGGCTAAACTTCCTTGCTGATGTCC
TTATCTTTTATAGCAATGCATTTAAGACGTTCTTTTACCCAGTGTTTGAAGATATGGTGGATC
AACAACACTCTTCCTCTTCCAAACAATTCACCATAGATGGACATGTGGAAAACTTCAAGAAG
TCACTAGACCTTGAAACTAGAGCAGGGAGTGACAATTTTGTCATAACCCTTCAGGAGAAACT
GGAGTCTCTTATATTGACAGTAGCTAAGCAGTTCTCTATAGAGGAAACAGAGGTATTCCCGA
TCATCAGCAAGAACTGCAACATTGAAATGCAGAGGCAACTACTATATAGAAGCATCCATTTC
CTGCCTCTTGGATTGCTTAAGTGTGTCATAATGTGGTTCTCTGCTCAGTTACCAGAAGATGAA
TGTCAATCAATTATTCACTACTTAAGCTCTGAAGATTCCTTTCCCAACAAACCTTTTGCGCAC
CTCTTGTTGCAGTGGTTTCGCTTCGGTTATTCAGGCAAAACACCAGTTGAAAGTTTCTGGAAT
GAGCTGTCCTTTATGTTCAAACCAAGATGTTCTTTTGAAGAGGAACTCACTGAAGAAGCTTC
TGGATCTTTCTTTCAACAATCACCGCAGAAACTATTCAAAGTATCCGATCCCTATTCTATGGA
TCCACCTGCTGGTTACATGAATGAGACACCTTACTCAAGTGCGATGAATCAGCAAATACTTA
TCCCAGGAAAGCTCAGGCCACTTCTGCATCTTCCTGATCTTTTTGGTGACAAGACTATTGGTG
AACACTTAACTATGGACTTGAAACCAATCGATCTGATTTTCTACTTCCACAAGGCAATGAAG
AAAGATCTAGACTACCTTGTACGTGGTTCAGCTCGGCTCGCAACAGATTATAGCTTCCTTGG
AGAATTTCAACAGCGGTTTCATCTTATAAAGTTCTTGTATCAGATACATTCAGATGCAGAAG
ATGAGATTGCATTTCCAGCTTTGGAGGCAAAGGGTAAACTACAAAACATTAGTCAGTCATAC
AGTATCGATCACGAACTCGAAGTTGAGCACCTCAATAAAGTATCATTCCTTTTGAATGAGTT
GGCAGAATTGAACATGCTGGTACTAGACCACAAGAATGTGAAGTATGAGAAGTTGTGTATG
AGCCTCCAGGATATATGCAAATCCATTCACAAGCTATTGTCTGAGCATCTCCACCGAGAAGA
AACTGAGCTTTGGTGTTTATTCAGAGACTGCTTCACTATTGAAGAACAAGAAAAGATCATAG
CATGCATGCTTGGAAGAATAAGTGGAGAAATATTGCAAGATATGATTCCATGGCTAATGGA
ATCTTTGATCCCTGATGAACAACATGCTGTGATGTCCTTGTGGAGACAAGCAACAAGGAAAA
CTATGTTTGGTGAATGGCTAACAGAATGGTACAATTCTCATGCTGTCGAGGAGGAAACAGAG
GAAGCAAATAAGGATCCATCTGAGAATTCAGATCCATTAGACGTTGTCTGGAGTTATCTCTT
TGAAGGAGCTGCTGATGAGTACAAAGGGAGCATTTGCAGCAAACCTCTGGAAGAGACAGAG
TTAAAAGGTATCATGAATAAGCCTCTTGGAAAGGCTGCTCCTAATAACAAGGTAGAATTTGG
AAACAAAGAGGAGAATCATCTTGAGATCTCAGGAAGTAAAAAGGTGTGTACAGGAGCTGAT
GAAACGAAGTACAAAGAACAAACTGACAGCAACGCGCAAGCTTTTCAGATGTCGCATAATA
CTAGCCAATCTGGCCAAGACAGTAGATATGAGTGTCTGTTATCAATGAGTCAAGAGGATGTG
GAAGCTACAATTCGAAGAATATCTCGAGATTCTTCCCTGGATCCTCAGAAGAAATCGTATAT
CATTCAAAACTTGTTAATGAGCCGTTGGATTGCTACACAACGGATATATAATCTTGAACCAT
CCATTCTCTCAAGCAATAGGGAAGCAGTTCCGGGGCAGAATCCATCTTATCGAGATCCTCAC
AAACTGATCTTTGGTTGCAAACACTACAAGAGAAGTTGCAAACTTCTTGCTCCTTGTTGCAA
CAAGCTCTACACATGTATACGATGCCATGATGAAGAGGTTGATCACTTATTGGATAGAAAAC
AGATCACAAAGATGATGTGCATGAAATGCATGATAATTCAGCCAGTAGGTGCTAGTTGCTCA
AATATTTCATGTAGTTCATCAATGGGAAAATATTACTGTAAAATATGCAAGTTGTTTGACGA
TGATAGGGAAATCTATCATTGTCCATACTGCAATCTCTGCCGGCTAGGAAAAGGACTGAGCA
TAGACTACTTTCATTGCATGAAATGTAATGCTTGTATGTCGCGACTCATAGTAGAACATGTAT
GCAGAGAAAAGTGCTTAGAAGATAATTGTCCGATTTGTCATGAATACATATTTACCTCAAAT
TCTCCGGTGAAGGCTCTTCCATGCGGTCACGTGATGCATTCAACATGCTTTCAGGAGTACAC
ATGTTCGCATTACACATGCCCTATTTGCAGCAAGTCGCTCGGGGATATGCAGGTTTACTTTAG
AATGTTAGACGCACTGCTTGCAGAACAAAAGATGCCAGATGAATACTTAAACCAAACTCAG
GTAATACTGTGTAATGACTGTGGAAGAAAGGGGAATGCTCCTTACCATTGGCTCTATCACAA
GTGTTCTTCTTGTGCCTCCTACAACACCAGACTTTTCTAG

### III. DNA Sequences of Promoters

SEQ ID NO: 154 is an exemplary DNA sequence of a UBQ10 promoter from Arabidopsis thaliana:
ctagtctagctcaacagagcttttaacccaaattggtacaatagaatacaactttagatcataattctcaaaagaaagagattccttagctattctat
ctgccactccatttccttctcggcttgtatgcacaagcataaaatcctcaaacttgctaagtagatactttatgtcttggataattggattgagacttgacaagca
taactttcatgtaaccaaagacacaagttgctgagaatccacctcaaaaatgatcttcctataattgaatcgggataatgacagcacagcccatctaagagc
ctccacttctacttccagcacgcttcttacttttaccacagctcttgcacctaaccataacaccttccctgtatgatcgcgaagcacccaccctaagccacattt
taatccttctgttggccatgccccatcaaagttgcacttaacccaagattgtggtggagcttcccatgtttctcgtctgtcccgacggtgttgtggttggtgcttt
ccttacattctgagcctctttccttctaatccactcatctgcatcttcttgtgtccttactaatacctcattggttccaaattccctccctttaagcaccagctcgtttc
tgttcttccacagcctcccaagtatccaagggactaaagcctccacattcttcagatcaggatattcttgtttaagatgttgaactctatggaggtttgtatgaac
tgatgatctaggaccggataagttcccttcttcatagcgaacttattcaaagaatgttttgtgtatcattcttgttacattgttattaatgaaaaaatattattggtcat
tggactgaacacgagtgttaaatatggaccaggccccaaataagatccattgatatatgaattaaataacaagaataaatcgagtcaccaaaccacttgcct
tttttaacgagacttgttcaccaacttgatacaaaagtcattatcctatgcaaatcaataatcatacaaaaatatccaataacactaaaaaattaaaagaaatgg
ataatttcacaatatgttatacgataaagaagttacttttccaagaaattcactgattttataagcccacttgcattagataaatggcaaaaaaaaacaaaaagg
aaaagaaataaagcacgaagaattctagaaaatacgaaatacgcttcaatgcagtgggacccacggttcaattattgccaattttcagctccaccgtatattt
aaaaaataaaacgataatgctaaaaaaatataaatcgtaacgatcgttaaatctcaacggctggatcttatgacgaccgttagaaattgtggttgtcgacgag
tcagtaataaacggcgtcaaagtggttgcagccggcacacacgagtcgtgtttatcaactcaaagcacaaatacttttcctcaacctaaaaataaggcaatt
agccaaaaacaactttgcgtgtaaacaacgctcaatacacgtgtcattttattattagctattgcttcaccgccttagctttctcgtgacctagtcgtcctcgtctt
ttcttcttcttcttctataaaacaatacccaaagagctcttcttcttcacaattcagatttcaatttctcaaaatcttaaaaactttctctcaattctctctaccgtgatc
aaggtaaatttctgtgttccttattctctcaaaatcttcgattttgttttcgttcgatcccaatttcgtatatgttctttggtttagattctgttaatcttagatcgaagac
gattttctgggtttgatcgttagatatcatcttaattctcgattagggtttcatagatatcatccgatttgttcaaataatttgagttttgtcgaataattactcttcgatt
tgtgatttctatctagatctggtgttagtttctagtttgtgcgatcgaatttgtcgattaatctgagtttttctgattaacagg
SEQ ID NO: 155 is an exemplary DNA sequence of a Ubi1 promoter from maize:
ctgcagtgcagcgtgacccggtcgtgcccctctctagagataatgagcattgcatgtctaagttataaaaaattaccacatattttttttgtcacac
ttgtttgaagtgcagtttatctatctttatacatatatttaaactttactctacgaataatataatctatagtactacaataatatcagtgttttagagaatcatataaat
gaacagttagacatggtctaaaggacaattgagtattttgacaacaggactctacagttttatctttttagtgtgcatgtgttctcctttttttttgcaaatagcttca
cctatataatacttcatccattttattagtacatccatttagggtttagggttaatggtttttatagactaatttttttagtacatctattttattctattttagcctctaaatt
aagaaaactaaaactctattttagtttttttatttaataatttagatataaaatagaataaaataaagtgactaaaaattaaacaaataccctttaagaaattaaaaa
aactaaggaaacatttttcttgtttcgagtagataatgccagcctgttaaacgccgtcgacgagtctaacggacaccaaccagcgaaccagcagcgtcgc
gtcgggccaagcgaagcagacggcacggcatctctgtcgctgcctctggacccctctcgagagttccgctccaccgttggacttgctccgctgtcggcat
ccagaaattgcgtggcggagcggcagacgtgagccggcacggcaggcggcctcctcctcctctcacggcacggcagctacgggggattcctttccca
ccgctccttcgctttcccttcctcgcccgccgtaataaatagacaccccctccacaccctctttccccaacctcgtgttgttcggagcgcacacacacacaa
ccagatctcccccaaatccacccgtcggcacctccgcttcaaggtacgccgctcgtcctccccccccccccctctctaccttctctagatcggcgttccggt
ccatggttagggcccggtagttctacttctgttcatgtttgtgttagatccgtgtttgtgttagatccgtgctgctagcgttcgtacacggatgcgacctgtacgt
cagacacgttctgattgctaacttgccagtgtttctctttggggaatcctgggatggctctagccgttccgcagacgggatcgatttcatgattttttttgtttcgt
tgcatagggtttggtttgcccttttcctttatttcaatatatgccgtgcacttgtttgtcgggtcatcttttcatgcttttttttgtcttggttgtgatgatgtggtctggtt
gggcggtcgttctagatcggagtagaattctgtttcaaactacctggtggatttattaattttggatctgtatgtgtgtgccatacatattcatagttacgaattga
agatgatggatggaaatatcgatctaggataggtatacatgttgatgcgggttttactgatgcatatacagagatgctttttgttcgcttggttgtgatgatgtgg
tgtggttgggcggtcgttcattcgttctagatcggagtagaatactgtttcaaactacctggtgtatttattaattttggaactgtatgtgtgtgtcatacatcttcat
agttacgagtttaagatggatggaaatatcgatctaggataggtatacatgttgatgtgggttttactgatgcatatacatgatggcatatgcagcatctattcat
atgctctaaccttgagtacctatctattataataaacaagtatgttttataattattttgatcttgatatacttggatgatggcatatgcagcagctatatgtggatttt
tttagccctgccttcatacgctatttatttgcttggtactgtttcttttgtcgatgctcaccctgttgtttggtgttacttctgcag
SEQ ID NO: 156 is an exemplary DNA sequence of a 35S promoter:
tcgacgaattaattccaatcccacaaaaatctgagcttaacagcacagttgctcctctcagagcagaatcgggtattcaacaccctcatatcaac
tactacgttgtgtataacggtccacatgccggtatatacgatgactggggttgtacaaaggcggcaacaaacggcgttcccggagttgcacacaagaaatt
tgccactattacagaggcaagagcagcagctgacgcgtacacaacaagtcagcaaacagacaggttgaacttcatccccaaaggagaagctcaactca
agcccaagagctttgctaaggccctaacaagcccaccaaagcaaaaagcccactggctcacgctaggaaccaaaaggcccagcagtgatccagcccc
aaaagagatctcctttgccccggagattacaatggacgatttcctctatctttacgatctaggaaggaagttcgaaggtgaaggtgacgacactatgttcac
cactgataatgagaaggttagcctcttcaatttcagaaagaatgctgacccacagatggttagagaggcctacgcagcaggtctcatcaagacgatctacc
cgagtaacaatctccaggagatcaaataccttcccaagaaggttaaagatgcagtcaaaagattcaggactaattgcatcaagaacacagagaaagacat
atttctcaagatcagaagtactattccagtatggacgattcaaggcttgcttcataaaccaaggcaagtaatagagattggagtctctaaaaaggtagttccta
ctgaatctaaggccatgcatggagtctaagattcaaatcgaggatctaacagaactcgccgtgaagactggcgaacagttcatacagagtcttttacgact
caatgacaagaagaaaatcttcgtcaacatggtggagcacgacactctggtctactccaaaaatgtcaaagatacagtctcagaagaccaaagggctatt
gagacttttcaacaaaggataatttcgggaaacctcctcggattccattgcccagctatctgtcacttcatcgaaaggacagtagaaaaggaaggtggctc
ctacaaatgccatcattgcgataaaggaaaggctatcattcaagatctctctgccgacagtggtcccaaagatggacccccacccacgaggagcatcgtg
gaaaaagaagacgttccaaccacgtcttcaaagcaagtggattgatgtgacatctccactgacgtaagggatgacgcacaatcccactatccttcgcaag
acccttcctctatataaggaagttcatttcatttggagaggacacg

### DETAILED DESCRIPTION

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a cell” includes singular or plural cells and can be considered equivalent to the phrase “at least one cell.” As used herein, the term “comprises” means “includes.” For example, reference to “comprising a plant” includes one or a plurality of such plants. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−10% variation of the value it describes. Accordingly, in some embodiments, the numerical parameters set forth herein are approximations that can vary depending upon the desired properties sought to be obtained by a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various aspects, the following explanations of terms are provided:

Backcross: The mating of a hybrid to one of its parents. For example hybrid progeny, for example a first generation hybrid (F₁), can be crossed back one or more times to one of its parents. Backcrossing can be used to introduce one or more single locus conversions (such as one or more desirable traits) from one genetic background into another.

BTSL (BRUTUS-LIKE): BTSL as used herein includes both BTSL genes and BTSL proteins. BTSL genes as used herein include one or more native BTSL genes or fragments thereof, and/or one or more orthologous BTSL genes or fragments thereof. BTSL proteins as used herein includes one or more native BTSL proteins or fragments thereof, and/or one or more orthologous BTSL proteins or fragments thereof. Native BTSL genes as used herein refer to BTSL genes found in the genome of the present plant or other plants within the same species, without or before any artificial genetic modification to BTSL genes; native BTSL proteins refer to proteins encoded by these genes. Orthologous BTSL genes as used herein refer to non-native BTSL genes, which may be found in the genomes of plants not within the same species as the present plant, or may be artificial mutants or variants of naturally occurring BTSL genes; orthologous BTSL proteins refer to proteins encoded by these genes.

BTSL proteins are characterized by two predicted hemerythrin domains, one hemerythrin-like domain (which is a degenerate hemerythrin motif with a predicted alpha-helical bundle but lacking the Fe-binding histidine and glutamate residues), and an E3 ubiquitin ligase domain (which has a sequence of different zinc fingers, including CHY-type, CTCHY-type, and RING-type, and a zinc ribbon) (Rodriguez-Celma, Jorge et al. “Hemerythrin E3 Ubiquitin Ligases as Negative Regulators of Iron Homeostasis in Plants,” Frontiers in plant science vol. 10, 98 (2019); Rodriguez-Celma, Jorge et al. “Arabidopsis BRUTUS-LIKE E3 ligases negatively regulate iron uptake by targeting transcription factor FIT for recycling,” Proceedings of the National Academy of Sciences of the United States of America vol. 116, 35 (2019): 17584-17591). Different plants have different numbers of BTSL proteins, for example, ranging from one BTSL protein to six BTSL proteins. For example, two BTSL proteins are identified in Arabidopsis thaliana, namely, BTSL1 (SEQ ID NO: 139), and BTSL2 (SEQ ID NO: 140). BTSL proteins include any protein that comprises at least 50%, 55%, 60%, 65%, 70%, 75%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% sequence identity to a sequence set forth in any one of SEQ ID NOs: 139-151.

BTSL genes include any nucleic acid sequence that encodes any of the above BTSL proteins. In some aspects, BTSL genes include any gene that comprises at least 50%, 55%, 60%, 65%, 70%, 75%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% sequence identity to SEQ ID NO: 152 or 153.

Cas9: An RNA-guided RNA endonuclease enzyme that can cut DNA. Cas9 has two active cutting sites (HNH and RuvC), one for each strand of the double helix. Catalytically inactive (deactivated) Cas9 (dCas9) is also encompassed by this disclosure. In some examples, a dCas9 includes one or more of the following point mutations: D10A, H840A, and N863A.

Cas9 nucleic acid and protein sequences are publicly available. For example, GenBank® Accession Nos. nucleotides 796693 . . . 800799 of CP012045.1 and nucleotides 1100046 . . . 1104152 of CP014139.1 disclose Cas9 nucleic acids, and GenBank® Accession Nos. AMA70685.1 and AKP81606.1 disclose Cas9 proteins. In some examples, the Cas9 is a deactivated form of Cas9 (dCas9), such as one that is nuclease deficient (e.g., those shown in GenBank® Accession Nos. AKA60242.1 and KR011748.1). In certain examples, Cas9 has at least 80% sequence identity, for example at least 85%, at least 90%, at least 95%, at least 96%, at least 97%, at least 98%, or at least 99% sequence identity to such sequences, and retains the ability to cut DNA.

Cell: Cell as used herein includes a plant cell, whether isolated, in tissue culture or incorporated in a plant or plant part.

Codon optimization: When the codon usage of a DNA or RNA is adapted to that of a cell or organism of interest to improve the transcription rate of said recombinant nucleic acid in the cell or organism of interest. A target nucleic acid can be modified at one position due to the codon degeneracy, whereas this modification will still lead to the same amino acid sequence at that position after translation, which is achieved by codon optimization to take into consideration the species-specific codon usage of a target cell or organism.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

Control: Refers to a plant, plant part, or plant cell that has a similar (or the same) genetic makeup and/or phenotypic traits as a treated plant, plant part, or plant cell before receiving the treatment. The treatment, for example, includes gene editing that results in one or more modifications in one or more genes, or RNAi treatment that reduces targeted mRNA translation. In some aspects, the control plant, plant part, or plant cell is wild-type with respect to the gene(s) being modified by the treatment, or is wild-type.

CRISPRs (Clustered Regularly Interspaced Short Palindromic Repeats): The CRISPR RNA array is a defining feature of naturally occurring bacterial CRISPR systems. The term “CRISPR” refers to the architecture of the array which includes constant direct repeats (DRs) interspaced with the variable spacers. In some examples, a CRISPR array includes at least a DR-spacer-DR-spacer. CRISPRs are found in approximately 40% of sequenced bacteria genomes and 90% of sequenced archaea. CRISPRs are often associated with cas genes that code for proteins related to CRISPRs (such as Cas9 and Cas13d proteins). CRISPR/Cas systems can be used for DNA targeting, for example to detect a target DNA, modify a target DNA at any desired location, or cut the target DNA at any desired location.

The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to the mode of action of RNAi in eukaryotic organisms. CRISPR/Cas systems can be used for gene editing (adding, disrupting or changing the sequence of specific genes) and gene regulation. By delivering a Cas9 protein (to cut DNA) or Cas13d protein (to cut RNA) and appropriate guide RNAs into a cell, the organism's genome can be cut at any desired location (such as a plant BTSL gene).

CRISPR RNA (crRNA): The RNA strand responsible for hybridizing with target DNA sequences, and recruiting CRISPR endonucleases and/or CRISPR-associated effectors. crRNAs may be naturally occurring, or may be artificially synthesized.

Cross: Synonymous with hybridize or crossbreed; includes the mating of genetically different individual plants, such as the mating of two parent plants.

Cross-pollination: Fertilization by the union of two gametes from different plants.

Endogenous: With reference to a nucleic acid and/or protein refers to the nucleic acid and/or protein in question as found in a plant in its natural form (i.e., without any human intervention). “Endogenous” is synonymous with “native” as used herein. An endogenous gene can include alleles of naturally occurring genes that have been mutated according to any of the methods of the present disclosure, i.e. an endogenous gene could have been modified at some point by traditional plant breeding methods and/or next generation plant breeding methods.

Exogenous: As used herein with reference to a nucleic acid molecule, protein, vector, or cell, refers to any such molecule/cell that does not originate from that particular cell or plant as found in nature. Thus, a non-naturally-occurring nucleic acid or vector is exogenous to a cell once introduced into the cell. An “exogenous” nucleic acid can either not occur in a plant in its natural form, be different from the nucleic acid in question as found in a plant in its natural form, or can be identical to a nucleic acid found in a plant in its natural form, but integrated not within their natural genetic environment. For example, a promoter may be endogenous to plant A, but exogenous to plant B. In one example, a recombinant plant/plant part/cell provided herein includes an exogenous nucleic acid specific for a BTSL gene (e.g., RNAi molecule). In one example, a gene-edited or recombinant plant/plant part/cell provided herein includes an exogenous nucleic acid specific for an IMA gene (e.g., IMA1 coding sequence or fragment thereof), which in some examples is integrated at genetic loci of the plant.

F₁ hybrid: The first generation progeny of the cross of two stable parents that are nonisogenic or isogenic plants.

Fragment: The terms “at least a portion” or “fragment” of a nucleic acid or protein means a portion having the minimal size characteristics of such sequences, or any larger fragment of the full-length molecule, up to and including the full-length molecule. In one example, a fragment of an IMA protein is a C-terminal portion of the IMA protein, such as one that includes at least 20 contiguous amino acids of the C-terminus of the IMA, for example including the C-terminal amino acid. A fragment of a polynucleotide of the disclosure may encode a biologically active portion of a protein. For example, a fragment of an IMA nucleic acid encodes such a fragment of an IMA protein. In some examples, a fragment of an IMA protein includes at least 20, 19, 18, 17, 16, 15, 14, 13, 12, 11, 10, 9, 8, 7, 6, or 5 contiguous amino acids of the C-terminus of the IMA (such as any of SEQ ID NOs: 1-134), for example including the C-terminal amino acid. In some examples, a fragment of an IMA protein (such as any one of SEQ ID NOs: 1-134) is 20, 19, 18, 17, 16, 15, 14, 13, 12, 11, 10, 9, 8, 7, 6, or 5 contiguous amino acids of the C-terminus of the IMA, for example including the C-terminal amino acid.

Gene editing: A type of gene modification in which a nucleic acid molecule, such as DNA, is inserted, deleted or replaced in a native genome of a cell (such as a plant cell), for example using engineered nucleases, which create site-specific double-strand breaks (DSBs) at desired locations in the genome, and whose improper repair by endogenous natural mechanisms results in an altered/non-native genomic sequence. Thus, the resulting genome is one that does not occur in nature. In some examples, gene editing results in the introduction of an exogenous transgene (e.g., one that does not occur naturally in the cell into which it is introduced) into the genome. In other examples, a gene-edited plant/plant part/cell provided herein is edited using an exogenous nucleic acid (e.g., gRNA) specific for a BTSL gene, thereby altering the endogenous sequence of the BTSL gene, but the exogenous nucleic acid molecule is not inserted into the genetic material (e.g., it disrupts the DNA target). In either case, such edited plants, plant parts, and cells are referred to as gene-edited plants, gene-edited plant parts, and gene-edited plant cells, respectively. The induced double-strand breaks are repaired through nonhomologous end-joining (NHEJ) or homologous recombination (HR), resulting in targeted mutations or repairs. CRISPR/Cas methods can be used to edit the sequence of one or more target genes, such as one or more BTSL genes. For example, gene editing in a plant can be used to confer a desirable trait to the plant, such as drought resistance, flooding resistance, erosion resistance, resistance to pests, or increased carbon sequestration.

Genetic activation/up-regulation: When used in reference to the expression of a nucleic acid molecule, such as a gene (e.g., an IMA gene), refers to any process which results in an increase in production of a gene product and/or an increase in biological activity of a protein, such as an increase of at least 20%, at least 50%, at least 75%, at least 90%, at least 95%, at least 100%, at least 200%, at least 400% or at least 500%. A gene product can be RNA or protein. Gene up-regulation or activation includes processes that increase transcription of a gene or stability or translation of mRNA.

In some examples, upregulation of a target nucleic acid (e.g., an IMA gene) includes processes that increase translation of the target nucleic acid and thus can increase the presence of corresponding proteins (such as IMA proteins or a C-terminal fragment thereof). In one example CRISPR/Cas systems are used to upregulate a target nucleic acid, for example by expressing a target nucleic acid (e.g., IMA1 or an IMA1 ortholog, such as a C-terminal fragment of IMA1 or an IMA1 ortholog). For example, a genetic upregulation of a plant IMA, such as by expression of a C-terminal fragment of IMA, can produce a plant having increased disease resistance.

Upregulation or activation includes any detectable increase in the target nucleic acid or protein encoded thereby. In certain examples, detectable target nucleic acid (or protein) (e.g., plant IMA, such as a C-terminal fragment of IMA) in a cell or plant increases by at least 10%, at least 20%, at least 30%, at least 40%, at least 50%, at least 60%, at least 70%, at least 75%, at least 80%, at least 90%, at least 95%, at least 100%, at least 200%, at least 300%, at least 400%, or at least 500% (as compared to a control (such an amount of target nucleic acid or protein detected in a corresponding wild-type cell or plant). In one example, a control is a relative amount of expression in a wild-type plant cell or plant (e.g., one that has not been gene edited, such as one that is non-recombinant and does not include a Cas protein or guide sequence or other exogenous nucleic acid).

Genetic inactivation/gene silencing/down-regulation: When used in reference to the expression of a nucleic acid molecule, such as a gene (e.g., BTSL), refers to any process which results in a decrease in production of a gene product and/or a decrease in biological activity, such as a decrease of at least 20%, at least 50% or at least 75%. Gene silencing occurs when gene expression is significantly reduced (e.g., a reduction of at least 90%, at least 95%, or at least 99%) or even prevented. It can also be referred to as knocking out gene expression, when the gene is completely silenced. A gene product can be RNA or protein. Gene down-regulation or deactivation includes processes that decrease transcription of a gene or stability or translation of mRNA.

For example, a mutation, such as a substitution, partial or complete deletion, insertion, or other variation, can be made to a gene sequence that significantly reduces (and in some cases eliminates) production of the gene product or renders the gene product substantially or completely non-functional. For example, a genetic inactivation of BTSL can produce a plant having increased resistance to disease. Genetic inactivation is also referred to herein as “functional deletion”.

In some examples genetic inactivation/gene silencing/down-regulation is not complete elimination of the target nucleic acid product or target nucleic acid function. In one example, downregulation or knock down does not result in complete elimination of detectable target nucleic acid expression or target nucleic acid activity. In some examples, the target nucleic acid is a genomic or coding DNA. In some examples, downregulation or knock down of a target RNA includes processes that decrease translation of the target RNA and thus can decrease the presence of corresponding proteins. In one example RNAi or CRISPR/Cas systems are used to downregulate a target nucleic acid (e.g., BTSL).

Downregulation or knock down includes any detectable decrease in the target nucleic acid or protein encoded thereby. In certain examples, detectable target nucleic acid (or protein) (e.g., plant BTSL1) in a cell or plant decreases by at least 10%, at least 20%, at least 30%, at least 40%, at least 50%, at least 60%, at least 70%, at least 75%, at least 80%, at least 90%, at least 95%, at least 96%, at least 97%, at least 98%, at least 99%, or 100% (such as a decrease of 40% to 90%, 40% to 80% or 50% to 95%) as compared to a control (such an amount of target nucleic acid or protein detected in a corresponding wild-type cell or plant). In one example, a control is a relative amount of expression in a wild-type plant cell or plant (e.g., one that has not been gene edited, such as one that is non-recombinant and does not include a Cas protein or guide sequence or other exogenous nucleic acid).

Genotype: The genetic constitution of a cell, an organism, or an individual (i.e., the specific allele makeup of the individual) usually with reference to a specific character under consideration.

Growing or regeneration: Growing a whole, differentiated plant from a plant cell, a group of plant cells, a plant part (including seeds), or a plant piece (e.g., from a protoplast, callus, or tissue part).

Guide sequence: A polynucleotide sequence having sufficient complementarity with a target polynucleotide sequence to hybridize with the target sequence and direct sequence-specific binding of a Cas protein (such as Cas9) to the target sequence (such as a BTSL). In some examples, the guide sequence is RNA. In some examples, the guide sequence is DNA. The guide nucleic acid can include modified bases or chemical modifications (e.g., see Latorre et al., Angewandte Chemie 55:3548-50, 2016). In some aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about, or at least about, 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, a guide sequence is 15-25 nucleotides (such as 18-22 or 18 nucleotides).

The ability of a guide sequence to direct sequence-specific binding of a CRISPR complex to a target sequence may be assessed by a suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

Heterologous: A substance coming from some source or location other than its native source or location. A heterologous nucleic acid can refer to a nucleic acid sequence that is not naturally found in the particular organism. In one example, a “heterologous promoter” refers to a promoter that has been taken from one source organism and utilized in another organism, in which the promoter is not naturally found. In another example, a “heterologous promoter” refers to a promoter that is from within the same source organism, but has merely been moved to a novel location, in which said promoter is not normally located. Heterologous gene sequences can be introduced into a cell (such as a plant cell) by using an “expression vector,” which can be a eukaryotic expression vector, for example a plant expression vector. Methods used to construct vectors are well known to a person skilled in the art and described in various publications. In particular, techniques for constructing suitable vectors, including a description of the functional components such as promoters, enhancers, termination and polyadenylation signals, selection markers, origins of replication, and splicing signals, are known.

Homologous sequences/homologs: With reference to a gene or gene products, nucleic acids and proteins thought, believed, or known to be functionally related. A functional relationship may be indicated by, for example (a) degree of sequence identity and/or (b) the same or similar biological function. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA). Homologous genes/proteins become separated in evolution in two possible ways: separation of two populations with the ancestral gene into two species or gene duplication of the ancestral gene within a lineage. Genes/proteins separated by speciation are called orthologs. Genes/proteins separated by gene duplication events are called paralogs.

IMA (IRON MAN): IMA as used herein includes both IMA genes and IMA proteins. IMA genes as used herein include one or more native IMA genes or fragments thereof, or one or more orthologous IMA genes or fragments thereof. IMA proteins as used herein include one or more native IMA proteins or fragments thereof, or one or more orthologous IMA proteins or fragments thereof. Native IMA genes as used herein refer to IMA genes found in the genome of the present plant or other plants within the same species, without or before any artificial genetic modification to IMA genes; native IMA proteins refer to proteins encoded by these genes. Orthologous IMA genes as used herein refer to non-native IMA genes, which may be found in the genome of plants not within the same species as the present plant, or may be artificial mutants or variants of naturally occurring IMA genes; orthologous IMA proteins refer to proteins encoded by these genes.

IMA is a ubiquitous family of genes/proteins that control iron transport in plants (Grillet, L., Lan, P., Li, W. et al; IRON MAN is a ubiquitous family of peptides that control iron transport in plants; Nature Plants 4, 953-963 (2018); https://doi.org/10.1038/s41477-018-0266-y). Different plants have different numbers of IMA proteins, for example, ranging from one IMA protein to thirteen IMA proteins. For example, eight IMA proteins are identified in Arabidopsis thaliana, namely, IMA1 (SEQ ID NO: 1), IMA2 (SEQ ID NO: 2), IMA3 (SEQ ID NO: 3), IMA4 (SEQ ID NO: 4), IMA5 (SEQ ID NO: 5), IMA6 (SEQ ID NO: 6), IMA7 (SEQ ID NO: 7), and IMA8 (SEQ ID NO: 8).

IMA proteins include any protein that comprises at least 50%, 55%, 60%, 65%, 70%, 75%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% sequence identity to any sequence set forth in SEQ ID NOs: 1-134, and retain an IMA function. In some aspects, IMA proteins have the aforementioned overall identity to any of the sequence set forth in SEQ ID NOs: 1-134, and have at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% C-terminal identity to any of SEQ ID NOs: 1-134, wherein C-terminal is the 30, 29, 28, 27, 26, 25, 24, 23, 22, 21, 20, 19, 18, 17, 16, 15, 14, 13, 12, 11 or 10 amino acids at the C-terminus. Fragments of IMA proteins are fragments of any length shorter than the IMA proteins and of contiguous sequence of the IMA proteins. In some aspects, fragments of IMA proteins are no more than 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, or 5 amino acids in length, and/or no less than 5, 6, 7, 8, 9, 10, 11, 12, 13, 14, 15, 16, 17, 18, 19, or 20 amino acids in length. In some aspects, fragments of IMA proteins are C-terminal fragments. In some aspects, fragments of IMA proteins are functional fragments that retain an IMA function. In some aspects, fragments of IMA proteins comprise G/E/D-G/N/D-D-D-D-x₍₁₋₆₎-D-x-A-P-A/S-A/G, or G/N/D-D-D-D-x₍₁₋₆₎-D-x-A-P-A-A, for example, at the C-termini, wherein x is any naturally occurring amino acid, and the number subscript refers to the number of x present, which can be any integer from 1 to 6.

IMA genes include any nucleic acid sequence that encodes any of the above IMA proteins. In some aspects, IMA genes include any gene that comprises at least 50%, 55%, 60%, 65%, 70%, 75%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% sequence identity to any sequence set forth in SEQ ID NOs: 135-138. In some aspects, IMA genes have the aforementioned overall identity to any sequence set forth in SEQ ID NOs: 135-138, and have at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% C-terminal identity to any of SEQ ID NOs: 135-138, wherein C-terminal is the part of the gene that encodes the 30, 29, 28, 27, 26, 25, 24, 23, 22, 21, 20, 19, 18, 17, 16, 15, 14, 13, 12, 11 or 10 amino acids at the C-terminus. Fragments of IMA genes include any nucleic acid sequence that encodes any of the above fragments of IMA proteins. In some aspects, fragments of IMA genes are no more than 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, or 20 nucleic acids in length, and/or no less than 10, 15, 20, 25, 30, 35, 40, 45, 50, 55, 60, 65, 70, 75, or 80 nucleic acids in length.

Increase or Decrease: A statistically significant positive or negative change, respectively, in quantity from a control value. An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95% or no more than 99%. In some examples, the control value is a value or range of values expected for the same plant that is not gene-edited, e.g., a wild-type plant (e.g., if the test plant is a gene-edited canola plant, the control can be a native or wild-type canola plant of the same variety).

Isolated: An “isolated” biological component (such as a protein, nucleic acid, guide sequence, or cell) has been substantially separated, produced apart from, or purified away from other biological components in the cell or tissue of a plant in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated inhibitory nucleic acid molecules (such as guide nucleic acids or vector comprising such), or cells containing such, in some examples are at least 50% pure, such as at least 75%, at least 80%, at least 90%, at least 95%, at least 98%, or at least 100% pure.

Next generation plant breeding: Refers to a host of plant breeding tools and methodologies that are available to a plant breeder. One distinguishing feature of next generation plant breeding is that the breeder is no longer confined to relying upon observed phenotypic variation, in order to infer underlying genetic causes for a given trait. Rather, next generation plant breeding can include the utilization of molecular markers and marker assisted selection (MAS), such that the breeder can directly observe movement of alleles and genetic elements of interest from one plant in the breeding population to another, and is not confined to merely observing phenotype. Further, next generation plant breeding methods are not confined to utilizing natural genetic variation found within a plant population. Rather, the breeder utilizing next generation plant breeding methodology can access a host of modern genetic engineering tools that directly alter/change/edit the plant's underlying genetic architecture in a targeted manner, in order to bring about a phenotypic trait of interest. In aspects, the plants bred with a next generation plant breeding methodology are indistinguishable from a plant that was bred in a traditional manner, as the resulting end product plant could theoretically be developed by either method. In particular aspects, a next generation plant breeding methodology may result in a plant that comprises: a genetic modification that is a deletion or insertion of any size; a genetic modification that is one or more base pair substitution; a genetic modification that is an introduction of nucleic acid sequences from within the plant's natural gene pool (e.g., any plant that could be crossed or bred with a plant of interest) or from editing of nucleic acid sequences in a plant to correspond to a sequence known to occur in the plant's natural gene pool; and offspring of said plants.

Naturally occurring: As applied to a nucleic acid, a polypeptide, a cell, or an organism, refers to a nucleic acid, polypeptide, cell, or organism that is found in nature. The term “naturally occurring” may refer to a gene or sequence derived from a naturally occurring source.

Non-naturally occurring or engineered: Terms used herein as interchangeably and indicate the involvement of the hand of man. The terms, when referring to nucleic acid molecules or polypeptides indicate that the nucleic acid molecule or the polypeptide is at least substantially free from at least one other component with which they are naturally associated in nature and as found in nature. In addition, the terms can indicate that the nucleic acid molecules or polypeptides have a sequence not found in nature. Thus, for the purposes of this disclosure, a “non-naturally occurring” sequence is a sequence that has been synthesized, mutated, engineered, edited, or otherwise modified to have a different sequence from known natural sequences. In some embodiments, the modification may be at the protein level (e.g., amino acid substitutions). In other embodiments, the modification may be at the DNA level (e.g., nucleotide substitutions).

Nucleotide change/nucleotide modification: Refers to, e.g., nucleotide substitution, deletion, and/or insertion, as is well understood in the art. For example, such nucleotide changes/modifications include mutations containing alterations that produce silent substitutions, additions, or deletions, but do not alter the properties or activities of the encoded protein or how the proteins are made. As another example, such nucleotide changes/modifications include mutations containing alterations that produce replacement substitutions, additions, or deletions, that alter the properties or activities of the encoded protein or how the proteins are made.

Offspring: Refers to any plant resulting as progeny from a vegetative or sexual reproduction from one or more parent plants or descendants thereof. For instance, an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

Operably linked: A first nucleic acid sequence is operably linked with a second nucleic acid sequence when the first nucleic acid sequence is placed in a functional relationship with the second nucleic acid sequence. For instance, a promoter is operably linked to a nucleic acid sequence (such as a guide nucleic acid sequence or a coding sequence) if the promoter affects the transcription or expression of the nucleic acid sequence. Generally, operably linked DNA sequences are contiguous and, where necessary to join two protein-coding regions, in the same reading frame. Coding sequences can be operably linked to regulatory sequences in a sense or antisense orientation. In one example, complementary RNA regions can be operably linked, either directly or indirectly, 5′ to the target mRNA, or 3′ to the target mRNA, or within the target mRNA, or a first complementary region is 5′ and its complement is 3′ to the target mRNA.

Protein/peptide/polypeptide: These terms are used interchangeably herein, and refer to a polymeric form of amino acids of any length, which can include coded and non-coded amino acids, chemically or biochemically modified or derivatized amino acids, and polypeptides having modified peptide backbones.

Ortholog: Refers to genes in different species that evolved from a common ancestral gene by speciation. Normally, orthologs retain the same function in the course of evolution. Identification of orthologs is useful for reliable prediction of gene function in newly sequenced genomes.

Plant: Includes reference to an immature or mature whole plant, including a plant from which seed, roots, or leaves have been removed. Seed or embryo that will produce the plant is also considered to be the plant. The present disclosure also includes seeds produced by the plants provided herein. In one example, the seeds can include one or more exogenous nucleic acid molecules encoding a repressor that reduces expression of one or more BTSL genes, or the seeds can include one or more loss-of-function mutations in one or more BTSL genes. In another example, the seeds can include one or more exogenous nucleic acid molecules encoding an activator that increases expression of one or more IMA genes, or the seeds can include one or more exogenous nucleic acid molecules encoding one or more IMA genes. In one embodiment, the seeds can develop into plants with increased disease resistance, as compared to a control plant, such as a wild-type plant.

Any commercially or scientifically valuable plant can be used in accordance with this disclosure. Exemplary plants include plants belonging to the super family Viridiplantae, such as monocotyledonous and dicotyledonous plants including a fodder or forage legume, ornamental plant, food crop, tree, or shrub, such as Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lo tonus bainesli, Lotus spp., Macro tyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canadensis, Phormium cookianum, Photinia spp., Picea glauca, Pinus spp., Pisum sativum, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinmfera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, switchgrass, Miscanthus, Setaria, fescue, eucalyptus, a tree, an ornamental plant, a perennial grass and a forage crop. In a specific example the plant is a pennycress plant, such as Thlaspi arvense. In a specific example the plant is a soybean plant, such as Glycine max. In another example the plant is a canola plant, such as Brassica napus. In another example the plant is a rice plant, such as a plant of the genus Oryza, or such as Oryza sativa. In another example, the plant is a sorghum or great millet plant, such as Sorghum bicolor.

Plant cell: Includes a single plant cell or a plurality of plant cells; includes protoplasts, gamete producing cells, cells that can regenerate into a whole plant, embryos, and callus tissue.

Plant part: Includes protoplasts, leaves, stems, roots, root tips, anthers, pistils, seed, embryo, pollen, stamen, ovule, microspore, protoplast, sporophyte, gametophyte, cotyledon, hypocotyl, flower, shoots, fruit, seeds, tissue, pollen, petiole, cells, meristematic cells and the like. Includes plant cells of a tissue culture from which plants can be regenerated. In one example a plant part is a plant cell (e.g., single cells, protoplasts, embryos, and callus tissue). Includes differentiated and undifferentiated tissues.

Plant cells include, without limitation, cells from seeds, suspension cultures, embryos, meristematic regions, callus tissue, leaves, roots, shoots, gametophytes, sporophytes, pollen and microspores. The plant tissue may be in a plant or in a plant organ, tissue or cell culture.

Plant pathogen: Any biological agent, such as a virus, bacterium, nematode, or fungus, that causes diseases in plants, including Pseudomonas syringae. Ralstonia solanacearum, Xanthomonas oryzae, Xanthomonas campestris, Xanthomonas axonopodis, Erwinia amylovora, Xylella fastidiosa, Dickeya dadantii, Pectobacterium carotovorum, Agrobacterium tumefaciens, Clavibacter michiganensis, Xylella fastidiosa, Ralstonia solanacearum, Pseudomonas syringae. Erwinia amylovora, Xanthomonas oryzae, Xanthomonas campestris, Xanthomonas axonopodis, Dickeya dadantii, Pectobacterium carotovorum, Phytophthora infestans, Fusarium oxysporum, Botrytis cinerea, Ustilago maydis. Magnaporthe oryzae. Rhizoctonia solani, Tobacco mosaic virus (TMV), Potato virus Y (PVY), Cucumber mosaic virus (CMV), Tomato yellow leaf curl virus (TYLCV), and Citrus tristeza virus (CTV).

Polynucleotide/nucleic acid molecule/nucleotide sequence: These terms are used interchangeably herein and refer to a polymeric form of nucleotides of any length, either ribonucleotides or deoxyribonucleotides, or analogs thereof. Refers to the primary structure of the molecule, and thus includes double- and single-stranded DNA, as well as double- and single-stranded RNA. Includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. It also includes modified nucleic acids such as methylated and/or capped nucleic acids, nucleic acids containing modified bases, backbone modifications, and the like. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms are understood to include, as applicable to the embodiments being described, single-stranded (such as sense or antisense) and double-stranded polynucleotides.

Progeny: Offspring; descendants.

Promoter: An array of nucleic acid control sequences which direct transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In some examples, a promoter used for recombinant expression of a nucleic acid molecule is not naturally occurring in the cell into which it is introduced, is not native to the nucleic acid molecule to which it is attached, or both. In one example, a promoter used is not endogenous (i.e., is exogenous) to the plant in which it is introduced. Exemplary promoters that can be used include the CaMV 35S promoter and the ubiquitin promoter.

Protein modification: Refers to, e.g., amino acid substitution, amino acid modification, deletion, and/or insertion, as is well understood in the art.

Recombinant construct: An artificial combination of nucleic acid fragments, e.g., regulatory and coding sequences that are not found together in nature. For example, a chimeric construct may include regulatory sequences and coding sequences that are derived from different sources, or regulatory sequences and coding sequences derived from the same source, but arranged in a manner different than that found in nature. Such construct may be used by itself or may be used in conjunction with a vector. Different independent transformation events can result in different levels and patterns of expression (Jones et al., (1985) EMBO J. 4:2411-2418; De Almeida et al., (1989) Mol. Gen. Genetics 218:78-86), and thus that multiple events can be screened in order to obtain lines displaying the desired expression level and pattern. Such screening may be accomplished, for example, by Southern analysis of DNA, Northern analysis of mRNA expression, immunoblotting analysis of protein expression, or phenotypic analysis, among others. As used herein, the term “expression” refers to the production of a functional end-product e.g., an mRNA or a protein (precursor or mature).

Recombinant or host cell: A cell that has been genetically altered, or is capable of being genetically altered by introduction of an exogenous polynucleotide, such as a recombinant plasmid or vector, such as one that expresses one or more exogenous nucleic acid molecules that reduce expression of a BTSL gene or increase expression of an IMA gene. Typically, a host cell is a cell in which a vector can be propagated and its nucleic acid expressed. In specific examples, such cells are plant cells, such as from a monocot or dicot. The term also includes any progeny of the subject host cell. It is understood that all progeny may not be identical to the parental cell since there may be mutations that occur during replication. However, such progeny are included when the term “host cell” is used. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived.

Regeneration: The development of a plant from tissue culture. The cells may, or may, not have been genetically modified. Plant tissue culture relies on the fact that all plant cells have the ability to generate a whole plant (totipotency). Single cells (protoplasts), pieces of leaves, or roots can often be used to generate a new plant on culture media given the required nutrients and plant hormones.

Ribonucleoprotein (RNP): A complex of ribonucleic acid and DNA-binding protein. In some examples, the RNP includes one or more, such as 2, 3, 4, or 5 different ribonucleic acids, such as gRNAs specific for different targets. In some examples, the DNA-binding protein is a Cas protein, such as a Cas9 native or mutant protein.

Self-pollination: The transfer of pollen from the anther to the stigma of the same plant.

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of protein sequences known and disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. These sequence identity ranges are provided for guidance only; it is entirely possible that strongly significant homologs could be obtained that fall outside of the ranges provided.

Variants of the disclosed nucleic acid sequences are typically characterized by possession of at least about 80%, at least 90%, at least 95%, at least 96%, at least 97%, at least 98% or at least 99% sequence identity counted over the full length alignment with the nucleic acid sequence using the NCBI Blast 2.0, gapped blastn set to default parameters. One of skill in the art will appreciate that these sequence identity ranges are provided for guidance only; it is possible that sequences coding for the disclosed proteins (e.g., IMA1, BTSL1, BTSL2, or ortholog thereof) could be obtained that fall outside of the ranges provided.

Tissue culture: A composition that includes isolated cells of the same or a different type or a collection of such cells organized into parts of a plant. In some example, the tissue culture includes a homogenous population of plant cells. In some example, the tissue culture includes a callus tissue. In some example, the tissue culture includes an anther culture or apical stem tip meristem culture. In another example, the tissue culture includes a hairy root culture.

Tolerance and resistance: Tolerance to a disease or condition refers to plants that can tolerate or withstand a certain amount of disease or condition, but do not display significant symptoms or yield losses. Resistance to a disease refers to the ability of a plant to resist or overcome attempted infection by a plant pathogen. A “tolerant plant” or “tolerant plant variety” need not possess absolute or complete tolerance. Instead, a “tolerant plant,” “tolerant plant variety,” or a plant or plant variety with “improved tolerance” will have a level of resistance or tolerance which is higher than that of a comparable susceptible plant or variety.

Traditional plant breeding: Refers to the utilization of natural variation found within a plant population as a source for alleles and genetic variants that impart a trait of interest to a given plant. Traditional breeding methods make use of crossing procedures that rely largely upon observed phenotypic variation to infer causative allele association. That is, traditional plant breeding relies upon observations of expressed phenotype of a given plant to infer underlying genetic cause. These observations are utilized to inform the breeding procedure in order to move allelic variation into germplasm of interest. Further, traditional plant breeding has also been characterized as comprising random mutagenesis techniques, which can be used to introduce genetic variation into a given germplasm. These random mutagenesis techniques may include chemical and/or radiation-based mutagenesis procedures. Consequently, one feature of traditional plant breeding, is that the breeder does not utilize a genetic engineering tool that directly alters/changes/edits the plant's underlying genetic architecture in a targeted manner, in order to introduce genetic diversity and bring about a phenotypic trait of interest.

Transformation: The introduction of exogenous genetic material (e.g., guide nucleic acids and vectors containing such) into cells, for example a plant cell. Exemplary mechanisms that are to transfer DNA into plant cells include (but not limited to) electroporation, microprojectile bombardment, Agrobacterium-mediated transformation and direct DNA uptake by protoplasts.

Transgene: An exogenous gene or other genetic material (e.g., guide nucleic acids and vectors containing such) that has been transferred into the genome of a plant or plant cell, for example by genetic engineering methods. Exemplary transgenes include cDNA (complementary DNA) segment, which is a copy of mRNA (messenger RNA), and the gene itself residing in its original region of genomic DNA. In one example, describes a segment of DNA containing a gene sequence that is introduced into the genome of a plant or plant cell. This non-native segment of DNA may retain the ability to produce RNA or protein in the transgenic plant, or it may alter the normal function of the transgenic plant's genetic code. In some examples, the transferred nucleic acid is incorporated into the plant's germ line. Transgene can also describe any DNA sequence, regardless of whether it contains a gene coding sequence or it has been artificially constructed, which has been introduced into a plant or vector construct in which it was previously not found.

Under conditions sufficient for: A phrase that is used to describe any environment that permits a desired activity. In one example the desired activity is expression of a nucleic acid that can specifically reduce or inhibit expression of a BTSL, in combination with other necessary elements, for example to decrease expression of the BTSL in a plant to increase its disease tolerance. In one example the desired activity is expression of a nucleic acid that can specifically increase expression of an IMA gene (such as a C-terminal fragment of IMA), in combination with other necessary elements, for example to increase expression of the IMA (such as a C-terminal fragment of IMA) in a plant to increase its disease tolerance.

Vector: A nucleic acid molecule into which a foreign nucleic acid molecule can be introduced without disrupting the ability of the vector to replicate and/or integrate in a host cell. In one example, a vector is not native to the cell into which it is introduced. Vectors include, but are not limited to, nucleic acid molecules that are single-stranded, double-stranded, or partially double-stranded; nucleic acid molecules that comprise one or more free ends, no free ends (e.g., circular); nucleic acid molecules that include DNA, RNA, or both; and other varieties of polynucleotides known in the art. In one example a vector includes a guide nucleic acid specific for a BTSL operably linked to a non-native promoter (e.g., promoter that does not occur naturally in the plant into which it is introduced, or a promoter that is not a BTSL promoter). In one example a vector includes a nucleic acid that encodes an IMA (such as encodes a C-terminal fragment of IMA) operably linked to a non-native promoter (e.g., promoter that does not occur naturally in the plant into which it is introduced, or a promoter that is not a IMA).

A vector can include nucleic acid sequences that permit it to replicate in a host cell, such as an origin of replication. A vector can also include one or more selectable marker genes and other genetic elements known in the art. An integrating vector is capable of integrating itself into a host nucleic acid. An expression vector is a vector that contains the necessary regulatory sequences to allow transcription and translation of encoded gene or genes, without vector integration into a host nucleic acid.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, herpes simplex viruses, baculoviruses, replication defective adenoviruses, and adeno-associated viruses). Viral vectors also include recombinant plant viruses, such as TMV-mediated (transient) transfection into tobacco (Tuipe, T-H et al (1993), J. Virology Meth, 42: 227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al (2008, “Geminivirus strain demarcation and nomenclature”. Archives of Virology 153:783-821, incorporated herein by reference in its entirety), and Khan et al. (Plant viruses as molecular pathogens; Publisher Routledge, 2002, ISBN 1560228954, 9781560228950). Examples of non-viral vectors include, but are not limited to, liposomes, polyamine derivatives of DNA, and the like. Vectors also include bphagemids, cosmids, artificial/mini-chromosomes (e.g., ACE), bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

The eukaryotic expression vectors in some examples also contain prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known and some are commercially available from companies such as Stratagene, La Jolla, Calif.; Invitrogen, Carlsbad, Calif.; Promega, Madison, Wis. or BD Biosciences Clontech, Palo Alto, Calif. In one embodiment the expression vector comprises at least one nucleic acid sequence which is a regulatory sequence necessary for transcription and translation of nucleotide sequences that encode for a peptide/polypeptide/protein of interest (such as IMA1).

### II. IMA and BTSL Nucleic Acid Molecules and Proteins

In accordance with the present disclosure, an IMA gene (such as the IMA1 gene of Arabidopsis thaliana and its orthologs) (including C-terminal fragments thereof, such as those that encode no more than 20, 19, 18, 17, 16, 15, 14, 13, 12, 11, 10, 9, 8, 7, 6, or 5 contiguous amino acids of the C-terminus of IMA, for example including the C-terminal amino acid), when under the control of appropriate promoters are useful for generating plants that have increased disease resistance when comparted to appropriate control plants. Thus, the disclosed IMA gene sequences can be used in breeding programs. In accordance with the present disclosure, decreasing expression of a BTSL gene (such as BTSL1 and/or BTSL2 of Arabidopsis thaliana and their orthologs) or activity of proteins encoded by these genes, is useful for generating plants that have increased disease resistance when comparted to appropriate control plants. Thus, the disclosed BTSL gene sequences can be used in breeding programs, for example by designing appropriate inhibitory RNA molecules, guide nucleic acid molecules, or other nucleic acid molecules that mutate a BTSL gene. See, for example, Gentzbittel et al. (1998, Theor. Appl. Genet. 96:519-523). IMA and BTSL sequences can thus be used to modulate plant disease resistance. See, generally, Sambrook et al. (1989) Molecular Cloning: A Laboratory Manual (2nd ed., Cold Spring Harbor Laboratory Press, Plainview, N.Y.). The sequences of the present disclosure can also be used to generate variants (e.g., by ‘domain swapping’) for the generation of new plant types with increased plant disease resistance.

The disclosure encompasses isolated or substantially purified nucleic acid or protein compositions, such as those that comprise an IMA gene (including C-terminal fragments thereof, such as those that encode no more than 20, 19, 18, 17, 16, 15, 14, 13, 12, 11, 10, 9, 8, 7, 6, or 5 contiguous amino acids of the C-terminus of IMA, for example including the C-terminal amino acid), as well as purified nucleic acid compositions that decrease a BTSL gene or protein activity, such as RNAi molecules, guide nucleic acid molecules, or other nucleic acid molecules that mutate a BTSL gene.

An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. In one example, an “isolated” polynucleotide is free of sequences (especially protein encoding sequences) that naturally flank the polynucleotide (i.e., sequences located at the 5′ and 3′ ends of the polynucleotide) in the genomic DNA of the organism from which the polynucleotide was derived. In some embodiments, the isolated polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide was derived. A polypeptide that is substantially free of cellular material includes preparations of protein having less than about 30%, 20%, 10%, 5%, (by dry weight) of contaminating protein. When the protein of the disclosure or biologically active portion thereof is recombinantly produced, culture medium suitably represents less than about 30%, 20%, 10%, or 5% (by dry weight) of chemical precursors or non-protein-of-interest chemicals.

Exemplary full length IMA coding sequences are provided in SEQ ID NOs: 135-138, and exemplary full length IMA protein sequences are provided in SEQ ID NOs: 1-134. Exemplary full length BTSL coding sequences are provided in SEQ ID NOs: 152-153, and exemplary full length BTSL protein sequences are provided in SEQ ID NOs: 139-151.

A portion of an IMA nucleotide sequence that encodes a biologically active portion of a IMA in some examples encodes no more than about 5, 6, 7, 8, 9, 10, 11, 12, 13, 14, 15, 16, 17, 18, 19, 20, 21, 22, 23, 24, 25, 30, 40, 50, 60, 70, 80, 90, 100 contiguous amino acid residues (for example at the C-terminus), or almost up to the total number of amino acids present in a full-length IMA polypeptide of the disclosure. Portions of an IMA or BTSL nucleotide sequence and/or upstream and downstream of the IMA or BTSL gene that are useful as hybridization probes or PCR primers generally need not encode a biologically active portion of a IMA or BTSL polypeptide.

A portion of an IMA or BTSL nucleotide sequence may encode a biologically active portion of an IMA or BTSL polypeptide, or it may be a fragment that can be used as a hybridization probe or PCR primer. A biologically active portion of an IMA polypeptide can be prepared by isolating a portion of an IMA nucleotide sequence of the disclosure, expressing the encoded portion of the IMA polypeptide (e.g., by recombinant expression in vitro), and assessing the activity of the encoded portion of the IMA polypeptide. Nucleic acid molecules that are portions of an IMA or BTSL nucleotide sequence comprise no more than about 10, 11, 12, 13, 14, 15, 16, 17, 18, 19, 20, 25, 30, 50, 75, 100, 150, 200, 250, 300, 350, 400, 450, 500, 550, 600, 650, 700, 750, 800, 850, 900, 950, 1000, 1500, 2000, 2500, 3000, or 3500 nucleotides, or almost up to the number of nucleotides present in a full-length IMA or BTSL nucleotide sequence disclosed herein (for example, about from 30 to 100 nucleotides for SEQ ID NOs: 135-138, or about from 100 to 2000 nucleotides for SEQ ID NOs: 152-153).

The disclosure also contemplates using variants of the disclosed nucleotide sequences. Nucleic acid variants can be naturally occurring, such as allelic variants (same locus), paralogous (different locus), and orthologues (different organism) or can be non-naturally occurring. Naturally occurring variants such as these can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of an IMA or BTSL polypeptides of the disclosure. Variant nucleotide sequences also include synthetically derived nucleotide sequences, such as those generated, for example, by using site-directed mutagenesis but which still encode an IMA or BTSL polypeptide of the disclosure. Generally, variants of a particular nucleotide sequence of the disclosure have at least about 30%, at least 40%, at least 50%, at least 55%, at least 60%, at least 65%, at least 70%, at least 75%, at least 80%, at least 85%, at least 90%, at least 95%, at least 98%, at least 99%, or 100% sequence identity to that particular nucleotide sequence as determined by sequence alignment programs described elsewhere herein using default parameters.

Variant nucleotide sequences also encompass sequences derived from mutagenic or recombinant procedures such as ‘DNA shuffling’ which can be used for swapping domains in a polypeptide of interest with domains of other polypeptides. With DNA shuffling, one or more different IMA coding sequences can be manipulated to create a new IMA sequence possessing desired properties. In this procedure, libraries of recombinant polynucleotides are generated from a population of related polynucleotides comprising sequence regions that have substantial sequence identity and can be homologously recombined in vitro or in vivo. For example, using this approach, sequence motifs encoding a domain of interest may be shuffled between an IMA gene of the disclosure and other IMA genes to obtain a new gene coding for a protein with an improved property of interest, such increasing plant disease resistance. Strategies for DNA shuffling are known e.g., Stemmer (1994, Proc. Natl. Acad. Sci. USA 91:10747-10751; 1994, Nature 370:389-391); Crameri et al. (1997, Nature Biotech. 15:436-438); Moore et al. (1997, J. Mol. Biol. 272:336-347); Zlang et al. (1997 Proc. Natl. Acad. Sci. USA 94:450-44509); Crameri et al. (1998, Nature 391:288-291); and U.S. Pat. Nos. 5,605,793 and 5,837,458.

The present disclosure provides nucleotide sequences for IMA and BTSL, homologs of IMA and BTSL, orthologs of IMA and BTSL, paralogs of IMA and BTSL, and fragments and variations thereof.

In some embodiments, the present disclosure provides a nucleotide sequence encoding IMA and/or functional fragments and variations thereof comprising a nucleotide sequence that shares at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, or about 99.9% sequence identity to any of SEQ ID NOs: 135-138. In some embodiments, a nucleotide sequence encoding IMA has the nucleic acid sequence of any of SEQ ID NOs: 135-138.

In some embodiments, the present disclosure provides nucleotide sequences for IMA1, homologs of IMA1, orthologs of IMA1, paralogs of IMA1, and fragments and variations thereof comprising nucleotide sequences that share at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, or about 99.9% sequence identity to SEQ ID NO: 135.

In some embodiments, the present disclosure provides a nucleotide sequence encoding a BTSL, and/or functional fragments and variations thereof comprising a nucleotide sequence that shares at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, or about 99.9% sequence identity to any of SEQ ID NOs: 152-153. In some embodiments, a nucleotide sequence encoding BTSL has the nucleic acid sequence of any of SEQ ID NOs: 152-153.

In some embodiments, the present disclosure provides nucleotide sequences for BTSL1, homologs of BTSL1, orthologs of BTSL1, paralogs of BTSL1, and fragments and variations thereof comprising nucleotide sequences that share at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, about 99.9%, or 100% sequence identity to SEQ ID NO: 152.

In some embodiments, the present disclosure provides nucleotide sequences for BTSL2, homologs of BTSL2, orthologs of BTSL2, paralogs of BTSL2, and fragments and variations thereof comprising nucleotide sequences that share at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, or about 99.9%, or 100% sequence identity to SEQ ID NO: 153.

In some embodiments, nucleotide sequences for IMA or BTSL, homologs of IMA or BTSL, orthologs of IMA or BTSL, paralogs of IMA or BTSL, and fragments and variations thereof can be expressed in plants or can be mutated to decrease expression/activity. In some embodiments, said nucleotide sequences can be used to be incorporated into an expression cassette, which is capable of directing expression (or reducing expression) of a nucleotide sequence for IMA or BTSL, homologs of IMA or BTSL, orthologs of IMA1 or BTSL, paralogs of IMA or BTSL, and fragments and variations thereof in a plant cell, plant tissue, plant part or whole plant. This expression cassette comprises a promoter operably linked to the nucleotide sequence of interest (including an IMA coding sequence or sequence to mutate a BTSL gene) which can be operably linked to termination signals. It can also include sequences required for proper translation of the nucleotide sequence. A coding region usually codes for a protein of interest, (e.g., IMA). In some embodiments, the expression cassette is chimeric so that at least one of its components is heterologous with respect to at least one of its other components.

In some embodiments, the expression cassette is naturally occurring but has been obtained in a recombinant form useful for heterologous expression. The expression of the nucleotide sequence in the expression cassette can be under the control of a constitutive promoter or of an inducible promoter which initiates transcription only when the host cell is exposed to some particular external stimulus. Also, the expression of the nucleotide sequence in the expression cassette can be under the control of a tissue-specific promoter, such as specific root tissues, including, but not limited to, the phellogen, pericycle or procambium. In the case of a multicellular organism, the promoter can also be specific to a particular tissue or organ or stage of development in animal and/or plant.

The present disclosure provides polypeptides and amino acid sequences comprising at least a portion of the proteins encoded by nucleotide sequences for IMA or BTSL, homologs of IMA or BTSL, orthologs of IMA or BTSL, homeologs of IMA or BTSL, paralogs of IMA or BTSL, and fragments and variations thereof.

The present disclosure also provides an amino acid sequence encoded by the nucleic acid sequences of IMA or BTSL, homologs of IMA or BTSL, orthologs of IMA or BTSL, paralogs of IMA or BTSL, and/or fragments and variations thereof. In some embodiments, the present disclosure provides an isolated polypeptide comprising an amino acid sequence that shares at least about 70%, about 75%, about 80%, about 85%, at least about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence encoded by the nucleic acid sequences of IMA or BTSL, homologs of IMA or BTSL, orthologs of IMA or BTSL, paralogs of IMA or BTSL, and/or fragments and variations thereof. In one embodiment, the present disclosure provides an isolated polypeptide comprising an amino acid sequence that shares at least about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence encoded by the nucleic acid sequences of IMA or BTSL, homologs of IMA or BTSL, orthologs of IMA or BTSL, paralogs of IMA or BTSL, and/or fragments and variations thereof.

The disclosure also encompasses variants and fragments of proteins of an amino acid sequence encoded by the nucleic acid sequences of IMA or BTSL, homologs of IMA or BTSL, orthologs of IMA or BTSL, and paralogs of IMA or BTSL. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both.

Functional fragments and variants of a polypeptide include those fragments and variants that maintain one or more functions of the parent polypeptide. It is recognized that the gene or cDNA encoding a polypeptide can be mutated without materially altering one or more of the polypeptide's functions. First, the genetic code is degenerate, and thus different codons encode the same amino acids. Second, even where an amino acid substitution is introduced, the mutation can be conservative and have no material impact on the essential function(s) of a protein. See, e.g., Stryer Biochemistry 3rd Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. J. Immunol. 159(5): 2502-12, 1997). Other modifications that can be made without materially impairing one or more functions of a polypeptide can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications, as will be readily appreciated by those well skilled in the art. A variety of methods for labelling polypeptides, and labels useful for such purposes, are well known in the art, and include radioactive isotopes such as 32P, ligands which bind to or are bound by labelled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues. In a specific example, a fragment of an IMA protein includes at least 5 amino acids from the C-terminal end of IMA, such as at least 5, at least 6, at least 7, at least 8, at least 9, at least 10, such as 5, 6, 7, 8, 9, 10, 11, 12, 13, 14, or 15 amino acids of the C-terminal end of IMA, which may include the C-terminal amino acid. Thus, in one example, a fragment of an IMA protein includes 5-20 amino acids from the C-terminal end (e.g., the last 5-20 amino acids of IMA, such as the last 5, 6, 7, 8, 9, 10, 11, 12, 13, 14, 15, 16, 17, 18, 19, or 20 C-terminal amino acids of IMA), which in some examples, has 1, 2, 3, 4, or 5 (such as 1-5 or 1-3) substitutions, such as conservative amino acid substitutions, as compared to a native IMA sequence. Mutations can be natural or purposely changed. In some embodiments, mutations containing alterations that produce silent substitutions, additions, or deletions, but do not alter the properties or activities of the proteins or how the proteins are made are an embodiment of the disclosure.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751 757, 1987), O'Regan et al. (Gene, 77:237 251, 1989), Sahin Toth et al. (Protein Sci., 3:240 247, 1994), Hochuli et al. (Bio/Technology, 6:1321 1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90%/identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. Table 1 shows exemplary conservative amino acid substitutions.

TABLE 1
Exemplary conservative amino acid substitutions

| | | Highly Conserved | Conserved |
|---|---|---|---|
| | Very Highly - | Substitutions (from | Substitutions (from |
| Original | Conserved | the Blosum90 | the Blosum65 |
| Residue | Substitutions | Matrix) | Matrix) |
| Ala | Ser | Gly, Ser, Thr | Cys, Gly, Ser, Thr, |
| | | | Val |
| Arg | Lys | Gln, His, Lys | Asn, Gln, Glu, His, |
| | | | Lys |
| Asn | Gln; His | Asp, Gln, His, Lys, | Arg, Asp, Gln, Glu, |
| | | Ser, Thr | His, Lys, Ser, Thr |
| Asp | Glu | Asn, Glu | Asn, Gln, Glu, Ser |
| Cys | Ser | None | Ala |
| Gln | Asn | Arg, Asn, Glu, His, | Arg, Asn, Asp, Glu, |
| | | Lys, Met | His, Lys, Met, Ser |
| Glu | Asp | Asp, Gln, Lys | Arg, Asn, Asp, Gln, |
| | | | His, Lys, Ser |
| Gly | Pro | Ala | Ala, Ser |
| His | Asn; Gln | Arg, Asn, Gln, Tyr | Arg, Asn, Gln, Glu, |
| | | | Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, |
| | | | Ser, |
| Met | Leu; Ile | Gln, Ile, Leu, Val | Gln, Ile, Leu, Phe, |
| | | | Val |
| Phe | Met; Leu; Tyr | Leu, Trp, Tyr | Ile, Leu, Met, Trp, |
| | | | Tyr |
| Ser | Thr | Ala, Asn, Thr | Ala, Asn, Asp, Gln, |
| | | | Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, |
| | | | Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionally similar to the disclosed an amino acid sequences encoded by the nucleic acid sequences of IMA or BTSL, homologs of IMA or BTSL, orthologs of IMA or BTSL, paralogs of IMA or BTSL, and/or fragments and variations thereof.

In some embodiments, variants may differ from the disclosed sequences by alteration of the coding region to fit the codon usage bias of the particular organism into which the molecule is to be introduced. In other embodiments, the coding region may be altered by taking advantage of the degeneracy of the genetic code to alter the coding sequence such that, while the nucleotide sequence is substantially altered, it nevertheless encodes a protein having an amino acid sequence substantially similar to the disclosed an amino acid sequences encoded by the nucleic acid sequences of IMA or BTSL, homologs of IMA or BTSL, orthologs of IMA or BTSL and/or paralogs of IMA or BTSL, and/or fragments and variations thereof.

In some embodiments, functional fragments derived from the IMA of the present disclosure are provided. The functional fragments can still confer the ability to increase disease resistance when expressed in a plant. In some embodiments, the functional fragments contain one or more conserved region shared by two or more IMA (including homologs, paralogs, and orthologs), for example, shared by two or more orthologs in the same plant genus, shared by two or more dicot IMA orthologs, and/or shared by two or more monocot IMA orthologs. The conserved regions can be determined by any suitable computer program, such as NCBI protein BLAST program and NCBI Alignment program, or equivalent programs. In some embodiments, the functional fragments are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids shorter compared to full-length IMA of the present disclosure. In some embodiments, the functional fragments are made by deleting one or more amino acids of full-length IMA of the present disclosure. In some embodiments, the functional fragments share at least 80%, 85%, 90%, 95%, 96%, 97%, 98%, 99%, or more identity to full-length IMA of the present disclosure.

In some embodiments, functional chimeric or synthetic polypeptides derived from the IMA (including homologs, paralogs, and orthologs) of the present disclosure are provided. The functional chimeric or synthetic polypeptides can still confer the ability to increase plant disease resistance when expressed. In some embodiments, the functional chimeric or synthetic polypeptides contain at least the conserved region of a wild type IMA, or functional variants thereof. In some embodiments, the functional chimeric or synthetic polypeptides contain one or more conserved region shared by two or more IMA, for example, shared by two or more IMA orthologs in the same plant genus, shared by two or more monocot IMA orthologs, and/or shared by two or more dicot IMA orthologs. The conserved regions can be determined by any suitable computer program, such as NCBI protein BLAST program and NCBI Alignment program, or equivalent programs. In some embodiments, the functional chimeric or synthetic polypeptides share at least 80%, 85%, 90%, 95%, 96%, 97%, 98%, 99%, or more identity to the IMA of the present disclosure. In some embodiments, the synthetic polypeptide comprises, G/E/D-G/N/D-D-D-D-x₍₁₋₆₎-D-x-A-P-A/S-A/G, and has a total of 12, 13, 14, 15, 16, 17, 18, 19, or 20 amino acids. In some embodiments, the synthetic polypeptide comprises G/N/D-D-D-D-x₍₁₋₆₎-D-x-A-P-A-A, and has a total of 11, 12, 13, 14, 15, 16, 17, 18, 19, or 20 amino acids.

In some embodiments, IMA proteins share at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or more identity to any of the sequences set forth in SEQ ID NOs: 1-134.

In some embodiments, BTSL proteins share at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or more identity to any of the sequences set forth in SEQ ID NOs: 139-141.

Sequences of conserved regions can also be used to knock-down the level of one or more IMA and BTSL (including homologs, paralogs and orthologs). In some embodiments, sequences of conserved regions can be used to make gene silencing molecules to target one or more IMA or BTSL genes or gene products (e.g., mRNA). In some embodiments, the gene silencing molecules are selected from the group consisting of double-stranded polynucleotides, single-stranded polynucleotides or Mixed Duplex Oligonucleotides. In some embodiments, the gene silencing molecules comprises a DNA/RNA fragment of about 10 bp, 15 bp, 19 bp, 20 bp, 21 bp, 25 bp, 30 bp, 40 bp, 50 bp, 60 bp, 70 bp, 80 bp, 90 bp, 100 bp, 150 bp, 200 bp, 250 bp, 300 bp, 350 bp, 400 bp, 500 bp, 600 bp, 700 bp, 800 bp, 900 bp, 1000 bp, or more polynucleotides, wherein the DNA/RNA fragment share at least 90%, 95%, 99%, or more identity to a conserved region of the IMA or BTSL sequences of the present disclosure, or complementary sequences thereof.

### III. Increasing Disease Resistance in Plants with Gene Editing

A gene editing system can be used to regulate expression/activity of genes in plants. For example, IMA expression/activity (or a fragment thereof) can be increased, for example by expressing IMA or a fragment thereof under control of a promoter. In another or additional example, BTSL expression/activity is decreased or even eliminated, for example by mutagenesis methods. In some examples a gene editing system is used that includes one or more nucleic acid (e.g., DNA or RNA)-binding domains or components and one or more nucleic acid (e.g., DNA or RNA)-modifying domains or components, or isolated nucleic acids, e.g., one or more vectors, encoding said nucleic acid (e.g., DNA or RNA)-binding and nucleic acid (e.g., DNA or RNA)-modifying domains or components. Gene editing systems can be used for modifying the nucleic acid of a target gene and/or for modulating the expression of a target gene. In some examples, the one or more nucleic acid (e.g., DNA or RNA)-binding domains or components are associated with the one or more nucleic acid (e.g., DNA or RNA)-modifying domains or components, such that the one or more nucleic acid (e.g., DNA or RNA)-binding domains target the one or more nucleic acid (e.g., DNA or RNA)-modifying domains or components to a specific nucleic acid site. Methods and compositions for enhancing gene editing is known. See example, U.S. Patent Application Publication No. 2018/0245065.

Exemplary gene editing systems include but are not limited to, zinc finger nucleases, transcription activator-like effector nucleases (TALENs); clustered regularly interspaced short palindromic repeats (CRISPR)/Cas systems, meganuclease systems, and viral vector-mediated gene editing.

In some embodiments, the methods for gene editing/cloning utilizing DNA nucleases. CRISPR complexes, transcription activator-like effector nucleases (TALENs), zinc finger nucleases (ZFNs), or FokI restriction enzymes. These enzymes target their nuclease activities to desired target loci through interactions with guide regions engineered to recognize target sequences. In some embodiments, CRISPR-based gene editing methods are sed to genetically modify the genome of plant species of the present disclosure in order to increase disease resistance of plant cells, plant tissues, plant parts or whole plants.

### A. CRISPR Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001, 18:688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in Class 2 systems all functions of the effector complex are carried out by a single protein (e.g., Cas9 or Cpf1). In some embodiments, the present disclosure provides using type II and/or type V single-subunit effector systems.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340. For example, the Cse (Cas subtype, E. coli) proteins (e.g., CasA) form a functional complex, Cascade, which processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

### B. CRISPR/Cas9

Provided are methods of gene editing using a Type II CRISPR system. Type II systems rely on a i) single endonuclease protein, ii) a transactiving crRNA (tracrRNA), and iii) a crRNA where a ˜20-nucleotide (nt) portion of the 5′ end of crRNA is complementary to a target nucleic acid. The region of a CRISPR crRNA strand that is complementary to its target DNA protospacer is hereby referred to as “guide sequence.”

In some embodiments, the tracrRNA and crRNA components of a Type II system can be replaced by a single guide RNA (sgRNA), also known as a guide RNA (gRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks, and are recruited to target DNA by a combination of a crRNA and a tracrRNA oligos, which tether the endonuclease via complementary hybridization of the RNA CRISPR complex.

In some embodiments, DNA recognition by the crRNA/endonuclease complex uses additional complementary base-pairing with a protospacer adjacent motif (PAM) (e.g., 5′-NGG-3′) located in a 3′ portion of the target DNA, downstream from the target protospacer. (Jinek, M., et. al., Science. 2012, 337:816-821). In some embodiments, the PAM motif recognized by a Cas9 varies for different Cas9 proteins.

In some embodiments the Cas9 disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27,156(5):935-49; Jinek M. et al. Science. 2012 337:816-21; and Jinek M. et al. Science. 2014 Mar. 14, 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641. Thus, in some embodiments, the systems and methods disclosed herein can be used with the wild type Cas9 protein having double-stranded nuclease activity, Cas9 mutants that act as single stranded nickases, or other mutants with modified nuclease activity.

Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, provided are tools for genome editing techniques in plants such as crops and methods of gene editing using CRISPR-associated (cas) endonucleases including SpyCas9, SaCas9, St1Cas9. See example, Song et al. (2016), The Crop Journal 4:75-82, Mali et al. (2013) Science 339: 823-826; Ran et al. (2015) Nature 520: 186-191; Esvelt et al. (2013) Nature methods 10(11): 1116-1121.

### C. CRISPR/Cpf1

In some embodiments, the present disclosure provides methods of gene editing using a Type V CRISPR system. In some embodiments, the present disclosure provides methods of gene editing using CRISPR from Prevotella, Francisella, Acidaminococcus, Lachnospiraceae, and Moraxella (Cpf1).

The Cpf1 CRISPR systems of the present disclosure can include i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 are at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, certain Cpf1 systems prefer a “TTN” PAM motif that is located 5′ upstream of its target. This is in contrast to the “NGG” PAM motifs located on the 3′ of the target DNA for common Cas9 systems such as Streptococcus pyogenes Cas9. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on Zetsche B. et al. 2015. (“Cpf1 Is a Single RNA-Guided Endonuclease of a Class 2 CRISPR-Cas System” Cell 163, 759-771).

### D. Guide Nucleic Acids

In some embodiments, a guide nucleic acid (e.g., RNA or DNA) of the present disclosure includes two coding regions, encoding for crRNA and tracrRNA, respectively. In other embodiments, the guide RNA is a single guide RNA (sgRNA) synthetic crRNA/tracrRNA hybrid. In other embodiments, the guide RNA is a crRNA for a Cpf1 endonuclease.

Unless otherwise noted, all references to a single guide nucleic acid (e.g., sgRNA or sgDNA) in the present disclosure can be read as referring to a guide nucleic acid (e.g., gRNA or gDNA). Therefore, embodiments described in the present disclosure which refer to a single guide nucleic acid (e.g., sgRNA or sgDNA) will also be understood to refer to a guide nucleic acid (e.g., gRNA orsgDNA).

The guide is designed to recruit the CRISPR endonuclease to a target nucleic acid region. Such methods are known in the art. Software programs can be used to identify candidate CRISPR target sequences on both strands of an input DNA sequence based on desired guide sequence length and a CRISPR motif sequence (PAM, protospacer adjacent motif) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR, with PAM sequence NNAGAAW, may be identified by searching for 5′-Nx-NNAGAAW-3′ both on the input sequence and on the reverse-complement of the input. The PAM sequence for Cas9 of S. pyogenes is 5′-NGG-3′.

Since multiple occurrences in the genome of the DNA or RNA target site may lead to nonspecific genome editing, after identifying all potential sites, sequences may be filtered out based on the number of times they appear in the relevant reference genome or modular CRISPR construct. For those CRISPR enzymes for which sequence specificity is determined by a ‘seed’ sequence (such as the first 5 bp of the guide sequence for Cpf1-mediated cleavage) the filtering step may also account for any seed sequence limitations.

In some embodiments, algorithmic tools identify potential off target sites for a particular guide sequence. For example, in some embodiments Cas-Offinder can be used to identify potential off target sites for Cpf1 (see Kim et al., 2016. Nature Biotechnology 34, 863-868). Any other publicly available CRISPR design/identification tool may also be used, including for example the Zhang lab crispr.mit.edu tool (see Hsu, et al. 2013 “DNA targeting specificity of RNA guided Cas9 nucleases” Nature Biotech 31, 827-832).

In some embodiments, the user can choose the length of the seed sequence. The user can specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

In the guide RNA or DNA, the “spacer/guide sequence” sequence is complementary to the “proto spacer” sequence in the nucleic acid target. The gRNA scaffold for a single stranded gRNA structure (or gDNA scaffold for a single stranded gDNA structure) is recognized by the Cas protein.

In some embodiments, the transgenic plant, plant part, plant cell, or plant tissue culture taught herein includes a recombinant construct, which includes at least one nucleic acid sequence encoding a guide RNA or guide DNA. In some embodiments, the nucleic acid is operably linked to a promoter. In other embodiments, a recombinant construct further comprises a nucleic acid sequence encoding a Clustered regularly interspaced short palindromic repeats (CRISPR) endonuclease. In other embodiments, the guide RNA or DNA is capable of forming a complex with said CRISPR endonuclease, and said complex is capable of binding to and creating a double strand break in a target nucleic acid sequence of said plant genome. In some embodiments, the CRISPR endonuclease is Cas9. In some embodiments, the CRISPR endonuclease is Cas13d.

In further embodiments, the target sequence is a nucleic acid for IMAor BTSL, homologs of IMA or BTSL, orthologs of IMA or BTSL and/or paralogs of IMA or BTSL, and/or fragments and variations thereof. In some embodiments, the present disclosure teaches the gene editing of IMA or BTSL in plants using genetic engineering techniques.

In some embodiments, the modified plant cells include one or more modifications (e.g., insertions, deletions, or mutations of one or more nucleic acids) in the genomic DNA sequence of an endogenous target gene (or RNA of a target sequence) resulting in the altered function the endogenous gene or gene product, thereby increasing disease resistance in plant cells, plant tissues, plant parts and whole plants. In some embodiments, the modified plant cells include a modified endogenous target gene, such as a mutation, or include additional nucleic acid sequences in an endogenous genome, for example to increase IMA activity. In some embodiments, the modifications in the genomic DNA sequence cause mutation, thereby altering the function of a BTSL protein. In some embodiments, the modifications in the genomic DNA sequence results in amino acid substitutions, thereby altering the normal function of the encoded protein. In some embodiments, the modifications in the genomic DNA sequence encode a modified endogenous protein with modulated, altered, stimulated, enhanced, or reduced function compared to the unmodified version of the endogenous protein. In some embodiments, the modifications in the genomic DNA sequence results in additional protein expression, for example of IMA or a fragment thereof.

In some embodiments, modified plant cells include one or more modified endogenous target genes, wherein the one or more modifications result in an altered function of a gene product (i.e., a protein) encoded by the endogenous target gene compared to an unmodified plant cell. For example, in some embodiments, a modified plant cell demonstrates expression of a protein or an upregulated expression of said protein. In some embodiments, the expression of the gene product (such as genetically-engineered IMA or a fragment thereof) in a modified plant cell is enhanced by at least 0.5%, 1%, 2%, 3%, 4%, 5% or higher compared to the expression of the gene product (such as IMA or a fragment thereof) in an unmodified plant cell. In other embodiments, the expression of the gene product (such as genetically-engineered IMA) in a modified plant cell is enhanced by at least 10%, 20%, 30%, 40%, 50%, 60%, 70%, 80%, 90%, or more compared to the expression of the gene product (such as IMA or a fragment thereof) in an unmodified plant cell. In some embodiments, the modified plant cells described herein demonstrate enhanced expression and/or function of gene products encoded by a plurality (e.g., two or more) of endogenous target genes compared to the expression of the gene products in an unmodified plant cell. For example, in some embodiments, a modified plant cell demonstrates enhanced expression and/or function of gene products from 2, 3, 4, 5, 6, 7, 8, 9, 10, or more endogenous target genes compared to the expression of the gene products in an unmodified plant cell.

In some embodiments, the modified plant cells described herein comprise one or more modified endogenous target genes, wherein the one or more modifications to the target DNA sequence results in expression of a protein with reduced or altered function (e.g., a “modified endogenous protein”) compared to the function of the corresponding protein expressed in an unmodified plant cell (e.g., a “unmodified endogenous protein”). In some embodiments, the modified plant cells described herein comprise 2, 3, 4, 5, 6, 7, 8, 9, 10, or more modified endogenous target genes encoding 2, 3, 4, 5, 6, 7, 8, 9, 10, or more modified endogenous proteins, such as those from the BTSL family.

In some embodiments, the modified endogenous protein demonstrates enhanced or altered binding affinity for another protein expressed by the modified plant cell or expressed by another cell; enhanced or altered signaling capacity; enhanced or altered enzymatic activity; enhanced or altered DNA-binding activity; or reduced or altered ability to function as a scaffolding protein.

The present disclosure also provides a transformed host cell comprising the modified gene as described above. In one embodiment, said host cell is a bacteria, yeast, filamentous fungi, algae, animal cell, or plant cell.

### E. Exemplary Methods of BTSL Gene Downregulation

In some examples, gene edited plants are generated using CRISPR/Cas technologies, for example using a guide nucleic acid molecule specific for a BTSL gene, that can mutate the target, resulting it its decreased expression and/or activity. In some examples, a CRISPR/Cas system is used.

Unlinked transgenic sequences (comprising the gRNA, the Cas9 cassette and the Kan^{R} cassette) will naturally segregate away from any gene-edited site in ¼ of the T1 generation. Thus, it is possible for plants to segregate away from the gRNA/Cas9 transgenes in subsequent generations, thus producing transgene-free, gene-mutated plants. In one example, the use of recombinant DNA in the construction of gene-edited plants is avoided, and instead plant leaf tissues are transformed using pre-assembled gRNAs and Cas9 ribonucleotprotein (RNP) complexes (e.g., wherein the gRNA(s) targets WEEP or a WEEP gene ortholog). In one example, polyethylene glycol (PEG)-transformation of protoplasts (e.g., see Woo et al., Nat. Biotechnol., 2015. 33(11): 11624) or gene gun bombardment of immature embryos with RNPs (e.g., see Zhang et al., Nat Commun, 2016. 7:12617; Liang et al., Nat Commun, 2017. 8:14261) is used.

gRNAs can be produced using commercial kits, such as the Invitrogen GeneArt™ Precision gRNA Synthesis Kit. To produce more gRNAs, a DNA template can be assembled by PCR with forward and reverse overlapping oligonucleotides that contain the target DNA sequence, together with the T7 promoter and universal reverse primers supplied with the kit. In vitro transcripts can be produced by T7 RNA polymerase and purified by phenol/chloroform extraction and ethanol precipitation. The RNP complexes of 1-5 μg gRNA and 1 μg GeneArt™ Platinum™ Cas9 nuclease with nuclear-targeting signal (Invitrogen) can be assembled and incubated for 10 min at room temperature. The RNP complexes can be mixed with 1 mg 0.6 μm gold particles sterilized by 70% ethanol for gene gun bombardment. Seeds can be sterilized by 10% bleach for 15 min and rinsed three times with sterile water. Seeds can then be germinated. Leaf bases from the first true leaves of 3-week old young plants or callus generated from embryos can be used as explants for bombardment. The bombarded plant tissues can be cultured on MS medium supplemented with Gamborg vitamins, 3% sucrose and 16.8 μM thidiazuron (TDZ) until shoot formation. Regenerated shoots can be transferred onto MS medium without TDZ but containing 1 μg/l indole-3-butyric acid (IBA) to induce root formation. Fully regenerated plantlets can be transferred to soil and allowed to produce seeds under isolated conditions.

Since CRISPR-mediated gene editing occurs in T₀ plants, the integration of the gRNA/Cas cassettes into the plant genome can be examined by PCR on T₀ plants. Cas9 expression can be validated using a 3×FLAG antibody to detect the epitope-tagged Cas9 protein in Western blot analysis.

In one example, the gene editing method is free of recombinant technology and does not involve T-DNA, Ti-plasmids (or other plasmids), Agrobacterium or other pathogenic microbes. Once the gRNA/Cas9 RNP complex is delivered into leaf tissue, it can be rapidly degraded and lost from cells. Gene edited plants without any transgene can be produced immediately from edited plant cells.

Using such methods allows for editing of more than one gene at a time (e.g., multiple homologs of BTSL, or a BTSL gene and an unrelated gene simultaneously) by bombarding leaf tissues with two gRNA/Cas9 RNP complexes. Since there is no selectable marker delivered into leaf tissues, regenerated plantlets can be screened for gene-editing individually.

In some examples, gene edited plants are generated using Agrobacterium-mediated transformation, which stably integrates a single copy of an exogenous nucleic acid into plant genomes (e.g., see Deschamps and Simon, Plant Cell Rep., 2002. 21:359-364; Phippen and Simon, Cell. Dev. Biol., 2000. 36: 2504) to produce gene-edited plants. Seeds can be germinated and the leaf tissues taken as explant for Agrobacterium inoculation for 30 min. The EHA105 strain of Agrobacterium can be transformed with a CRISPR-editing vector. The infected plant tissues can be cultured on MS medium supplemented with Gamborg vitamins, 3% sucrose and 16.8 μM thidiazuron (TDZ) for 3 days, after which plant tissues can be transferred to the same medium containing 300 μg/ml cefotaxime to inhibit the further growth of Agrobacterium and 50 μg/ml kanamycin to select transformed tissues and regenerate transgenic shoots. Regenerated transgenic shoots can be transferred onto MS medium without TDZ but containing 25 μg/ml kanamycin and 1 μg/I indole-3-butyric acid (IBA) to induce root formation. Fully regenerated transgenic plantlets can be transferred to soil and allowed to produce seeds. To transgenic plants can be examined for the integration of the transgenes by PCR analysis and the mutation of genes, such as BTSL1 and/or BTSL2 genes or a BTSL1 and/or BTSL2 gene ortholog.

In one example, antisense or inhibitory RNA (RNAi) technology is used to reduce or eliminate the activity of one or more genes from the BTSL family. For example, a plant or plant cell can be engineered to contain a cDNA that encodes an antisense molecule that reduces or prevents one or more genes from the BTSL family from being translated. The term “antisense molecule” encompasses any nucleic acid molecule or nucleic acid analog (e.g., peptide nucleic acids) that contains a sequence that corresponds to the coding strand of an endogenous BTSL gene. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axehead structures, provided the molecule cleaves RNA.

### F. Exemplary Methods of Screening Gene Edited Plants

Gene-edited plants generated using the provided methods, such as those generated to contain a non-native BTSL gene sequence, can be screened to identify or confirm the presence of a mutation introduced. Similarly, gene-edited plants generated using the provided methods, such as those generated to contain additional or non-native IMA (or fragment thereof) sequences or additional or non-native IMA (or fragment thereof) ortholog sequences, can be screened to identify or confirm the presence of a nucleic acid molecule introduced.

PCR primers can be used to amplify all or a portion of an IMA and/or BTSL gene sequence (or fragment thereof), such as gDNA fragments spanning the selected gene target sites. Restriction enzyme digestion can be carried out on the PCR products. Since restriction enzyme sites can be included at the sites of editing, loss of the ability to digest a PCR product in some examples indicates a gene-edited plant. The undigested PCR fragments can be sequenced to confirm the presence and nature of any mutations or added sequences. RFLP methods can be used to screening large numbers of candidate mutant plants.

A T7E1 assay can be used to screen regenerated mutant plants. This assay allows mutated, edited sites to be detected based on their incomplete hybridization to the WT sequence (due to a mismatch between the WT and edited hybridized DNA strands at the edited site). PCR fragments spanning the mutation sites can be denatured at 95° C. and cooled down to 22° C. slowly using a thermal cycler. Annealed PCR products can be incubated with T7 endonuclease 1 (NEB) at 37° C. for 20 min and analyzed by electrophoresis in 1-2% agarose gel.

A TaqMan probe-based qPCR analysis can be used. TaqMan probes can be designed for each of the WT target sites and synthesized with fluorescence labeling on the 5′ end and minor groove binder-nonfluorescent quencher (e.g., MGB-NFQ) on the 3′ end. In qPCR analysis, the biallelic mutant will not produce any fluorescent signal, while the WT plant will produce double the signal compared to the monoallelic mutant (e.g., see Li et al., Plant Physiol., 2015. 169(2): 960-70). This TaqMan-qPCR method in the 96-well format used by the StepOnePlus qPCR System (Applied Biosystems) can be used to screen a large number of regenerated plants, produced by the gene gun bombardment with RNP complexes. This method generates gene edited plants that do not carry selectable marker genes.

Mutations from biallelic T₀ mutants are expected to be inherited in the next generations. For transgenic mutant plants produced by Agrobacterium-mediated transformation, gene-specific PCR assays can be used to screen for T₁ plants that have segregated out the Cas9 and Kan^{R} genes. The monoallelic T₀ mutants are expected to segregate according to the Mendelian law with a 1:2:1 ratio.

### IV. Exemplary Promoters

Nucleic acid molecules (such as coding sequences or guide sequences) included in expression vectors are typically driven by a nucleotide sequence comprising a regulatory element, for example, a promoter. Thus, promoter sequences can be operably linked to an IMA (or fragment thereof) coding sequence or guide nucleic acid, or to a nucleic acid molecule that reduces BTSL activity (such as an RNAi or guide nucleic acid). Examples include a plant promoter, such as one from Arabidopsis or other plant (e.g., constitutive promoter from the Arabidopsis serine carboxypeptidase-like gene AtSCPL30, PD1 from Arabidopsis, HVA22E, PLDdelta, AtS1, and AtS3). In some examples, the promoter is heterologous to the plant into which it is introduced. In some examples, the promoter is heterologous to the sequence to which it is operably linked. Exemplary promoters that can be used with the disclosure include those shown in SEQ ID NOS: 154-156, as well as sequences with at least 90%, at least 95%, at least 96%, at least 97%, at least 98% or at least 99% sequence identity to SEQ ID NO: 154, 155, or 156.

Promoter includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter that is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions, and cell types.

### A. Inducible Promoters

In some examples, an inducible promoter is operably linked to a gene or coding sequence (or other sequence, such as a guide or RNAi) for expression in a plant. In another example, an inducible promoter is operably linked to a nucleotide sequence encoding a signal sequence which is operably linked to a gene or coding sequence (or other sequence, such as a guide or RNAi) for expression in a plant. With an inducible promoter the rate of transcription increases in response to an inducing agent.

Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). One exemplary inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. USA 88:0421 (1991).

### B. Constitutive Promoters

In some examples, a constitutive promoter is operably linked to a gene or coding sequence (or other sequence, such as a guide or RNAi) for expression in a plant. In another example, a constitutive promoter is operably linked to a nucleotide sequence encoding a signal sequence which is operably linked to a gene or coding sequence (or other sequence, such as a guide or RNAi) for expression in a plant.

Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO 96/30530.

In a specific example the constitutive promoter is CaMV-35S (an exemplary sequence provided in SEQ ID NO: 156), CaMV-35Somega, UBQ10 from Arabidopsis (an exemplary sequence provided in SEQ ID NO: 154), Ubi1 from maize/rice (an exemplary sequence provided in SEQ ID NO: 155), or barley leaf thionin BTH6 promoter).

### C. Tissue-Specific or Tissue-Preferred Promoters

In some examples, a tissue-specific promoter is operably linked to a gene or coding sequence (or other sequence, such as a guide or RNAi) for expression in a plant. In another example, a tissue-specific promoter is operably linked to a nucleotide sequence encoding a signal sequence which is operably linked to a gene or coding sequence (or other sequence, such as a guide or RNAi) for expression in a plant. A tissue-specific promoter can be operably linked to a gene for expression in a plant. Optionally, the tissue-specific promoter is operably linked to a nucleotide sequence encoding a signal sequence which is operably linked to a gene for expression in a plant. Plants transformed with a gene or coding sequence (or other sequence, such as a guide or RNAi) operably linked to a tissue-specific promoter produce the product of the gene or coding sequence (or other sequence, such as a guide or RNAi) exclusively, or preferentially, in a specific tissue.

Any tissue-specific or tissue-preferred promoter can be utilized in the instant disclosure. Exemplary tissue-specific or tissue-preferred promoters include, but are not limited to, a root-preferred promoter—such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

In some examples, a tissue-specific or tissue-preferred promoter is a native promoter of FACT gene, HORST gene, ASFT gene, GPAT5 gene, RALPH gene, and/or MYB84 gene.

### V. Constructs

The present disclosure provides constructs comprising expression cassettes comprising promoters operably-linked to a nucleic acid molecule, such as one that increases IMA (or fragment thereof) activity/expression, or reduces BTSL activity/expression, or combinations thereof. When these constructs are transformed into plants they enables those transgenic plants to increase disease resistance without negatively impacting plant health.

### VI. Exemplary Methods for Plant Transformation

The disclosed polynucleotides for increasing IMA (or fragment thereof) activity/expression or reducing BTSL activity/expression, or combinations thereof, of the present disclosure can be transformed into plant cells, plant tissues, plant parts and whole plants.

Methods of producing transgenic plants are known. Transgenic plants can now be produced by a variety of transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)).

Exemplary methods include Agrobacterium-mediated nucleic acid transfer (e.g., see U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. Nos. 5,731,179, 7,250,554, EP068730, WO9516031, U.S. Pat. Nos. 5,693,512, 6,051,757 and EP904362A1), microprojectile bombardment, injection into plant cells or tissues, direct incubation of an exogenous nucleic acid molecule with germinating pollen, and electroporation.

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

For efficient plant transformation, a selection method is used such that whole plants are regenerated from a single transformed cell and every cell of the transformed plant carries the nucleic acid of interest. These methods can employ positive selection, whereby a foreign nucleic acid is supplied to a plant cell that allows it to utilize a substrate present in the medium that it otherwise could not use, such as mannose or xylose (for example, refer U.S. Pat. Nos. 5,767,378; 5,994,629). Negative selection can be used, utilizing selective agents such as herbicides or antibiotics that either kill or inhibit the growth of non-transformed plant cells and reducing the possibility of chimeras. Resistance genes that are effective against negative selective agents are provided on the introduced foreign nucleic acid used for the plant transformation. For example, kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)) can be used. However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2(7): 1099-1104 (1983).

The expression control elements used to regulate the expression of a given nucleic acid can either be the expression control element that is normally found associated with the coding sequence (homologous expression element) or can be a heterologous expression control element. A variety of homologous and heterologous expression control elements are known and can readily be used to make expression units for use in the present disclosure. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Plant promoters such as prolifera promoter, fruit specific promoters, Ap3 promoter, heat shock promoters, seed specific promoters, etc. can also be used.

A gamete-specific promoter, a constitutive promoter (such as the CaMV or Nos promoter), an organ specific promoter (such as the E8 promoter from tomato), or an inducible promoter can be ligated to the nucleic acid to be expressed. The expression unit may be further optimized by employing supplemental elements such as transcription terminators and/or enhancer elements.

Thus, for expression in plants, the expression units typically contain, in addition to the nucleic acid to be expressed, a plant promoter region, a transcription initiation site and a transcription termination sequence. Unique restriction enzyme sites at the 5′ and 3′ ends of the expression unit are typically included to allow for easy insertion into a pre-existing vector.

In some examples, the promoter is positioned about the same distance from the heterologous transcription start site as it is from the transcription start site in its natural setting. However, some variation in this distance can be accommodated without loss of promoter function.

In addition to a promoter sequence, the expression cassette can also contain a transcription termination region downstream of the nucleic acid to be expressed to provide for efficient termination. The termination region may be obtained from the same gene as the promoter sequence or may be obtained from different genes. If the mRNA encoded by the nucleic acid to be expressed is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835 846 (1984)) or the nopaline synthase signal (Depicker et al., Mol. and Appl. Genet. 1:561 573 (1982)). The resulting expression unit is ligated into or otherwise constructed to be included in a vector that is appropriate for higher plant transformation. One or more expression units may be included in the same vector. The vector typically contains a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, can be included to allow the vector to be cloned in a bacterial or phage host; in one example a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector. For instance, in the case of Agrobacterium transformations, T DNA sequences can be included for subsequent transfer to plant chromosomes.

To introduce a nucleic acid to be expressed by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques circumvent these limitations by enabling introduction of specific genes for desirable traits, such as improved fatty acid composition, and to introduce these genes into already useful varieties of plants. Once the foreign genes have been introduced into a plant, that plant can then be used in imp plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

An expression construct which includes nucleotide sequences that increase expression/activity of IMA1 (or a fragment thereof), decrease expression/activity of TSL1 and/or BTSL2, or combinations thereof, as well as homologs, orthologs, and/or paralogs. The expression construct(s) can be introduced into embryogenic callus of any plant genus or species and the resulting transformed cells can be regenerated into plants. The transgenic plants are expected to have expression of the exogenous nucleic acid molecule.

The phrase “embryogenic callus cell” used herein refers to an embryogenic cell contained in a cell mass produced in vitro.

Several approaches can be utilized to transform and co-express these polynucleotides in plant cells.

Each nucleic acid molecule to be expressed (e.g., those that increase expression/activity of IMA (or a fragment thereof), those that decrease expression/activity of BTSL, or combinations thereof) can be separately introduced into a plant cell by using separate nucleic-acid constructs. In some embodiments, two or more nucleic acid molecules to be expressed sequences can be co-introduced and co-expressed in the plant cell using a single nucleic acid construct. Such a construct can be designed with a single promoter sequence co-which can transcribe a polycistronic message including the nucleic acid molecules to be expressed. To enable co-translation of multiple nucleic-acid constructs, the polynucleotide sequences can be inter-linked via an internal ribosome entry site (IRES) sequence which facilitates translation of polynucleotide sequences positioned downstream of the IRES sequence. In this case, a transcribed polycistronic RNA molecule encoding the individual nucleic-acid constructs can be translated from both the capped 5′ end and the two internal IRES sequences of the polycistronic RNA molecule to thereby express each nucleic acid molecule to be expressed.

In some examples, the two or more nucleic acid molecules to be expressed are translationally fused via a protease recognition site cleavable by a protease expressed by the cell to be transformed with the nucleic acid construct. In this case, a chimeric polypeptide translated will be cleaved by a cell-expressed protease to thereby generate the plurality of polypeptides.

In other embodiments, a nucleic acid construct includes multiple promoter sequences each capable of directing transcription of a specific polynucleotide sequence.

Suitable promoters which can be used include constitutive, inducible, or tissue-specific promoters.

Exemplary constitutive promoters include, for example, CaMV 35S promoter (Odell et al., Nature 313:810-812, 1985); maize Ubi 1 (Christensen et al., Plant Sol. Biol. 18:675-689, 1992); rice actin (McElroy et al., Plant Cell 2:163-171, 1990); pEMU (Last et al., Theor. Appl. Genet. 81:581-588, 1991); and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable inducible promoters can be pathogen-inducible promoters such as, for example, the alfalfa PR10 promoter (Coutos-Thevenot et al., Journal of Experimental Botany 52: 901-910, 2001 and the promoters described by Marineau et al., Plant Mol. Biol. 9:335-342, 1987; Matton et al. Molecular Plant-Microbe Interactions 2:325-331, 1989; Somsisch et al., Proc. Natl. Acad. Sci. USA 83:2427-2430, 1986: Somsisch et al., Mol. Gen. Genet. 2:93-98, 1988; and Yang, Proc. Natl. Acad. Sci. USA 93:14972-14977, 1996.

Suitable tissue-specific promoters include, but not limited to, leaf-specific promoters such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993.

A nucleic acid construct can also include at least one selectable marker such as nptII. In one example, the nucleic acid construct is a shuttle vector, which can propagate both in E. coli (wherein the construct comprises an appropriate selectable marker and origin of replication) and be compatible for propagation in cells. In some examples, a construct can be, for example, a plasmid, a bacmid, a phagemid, a cosmid, a phage, a virus or an artificial chromosome.

Following transformation, the transformed cells can be micropropagated to provide a rapid, consistent reproduction of the transformed material. Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that utilizes alteration of culture medium or growth conditions between stages. The micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Integration of an exogenous nucleic acid molecule in the genome of the transformed plants can be determined using standard molecular biology techniques, such as PCR and Southern blot hybridization.

In some examples the transformation is stable. n some examples the transformation is transient.

In one example, transformation is by viral infection. Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman et al. (Communications in Molecular Biology: Viral Vectors, Cold Spring Harbor Laboratory, New York, pp. 172-189, 1988). Pseudovirus particles for use in expressing an exogenous nucleic acid molecule in many hosts, including plants, is described in WO 87/06261.

Suitable modifications can be made to a DNA virus. Alternatively, the virus can first be cloned into a bacterial plasmid for ease of constructing the desired viral vector with the exogenous nucleic acid molecule. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA.

If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

In one embodiment, a plant viral nucleic acid is provided in which the native coat protein coding sequence has been deleted from a viral nucleic acid, a non-native plant viral coat protein coding sequence and a non-native promoter, such as the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the exogenous nucleic acid molecule within it, such that a product is produced. The recombinant plant viral nucleic acid may contain one or more additional non-native subgenomic promoters. Each non-native subgenomic promoter can transcribe or express adjacent genes or nucleic acid sequences in the plant host and incapable of recombination with each other and with native subgenomic promoters. Exogenous nucleic acid molecules can be inserted adjacent the native plant viral subgenomic promoter or the native and a non-native plant viral subgenomic promoters if more than one nucleic acid sequence is included. The exogenous nucleic acid sequences are transcribed or expressed in the host plant under control of the subgenomic promoter to produce the desired products.

In some examples, the native coat protein coding sequence is placed adjacent one of the non-native coat protein subgenomic promoters instead of a non-native coat protein coding sequence.

In some examples, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Exogenous nucleic acid molecules can be inserted adjacent the non-native subgenomic plant viral promoters such that the sequences are transcribed or expressed in the host plant under control of the subgenomic promoters to produce the desired product.

In some examples, a recombinant plant viral nucleic acid is provided in which the native coat protein coding sequence is replaced by a non-native coat protein coding sequence.

The viral vectors can be encapsidated by the coat proteins encoded by the recombinant plant viral nucleic acid to produce a recombinant plant virus. The recombinant plant viral nucleic acid or recombinant plant virus can be used to infect appropriate host plants. The recombinant plant viral nucleic acid can be capable of replication in the host, systemic spread in the host, and transcription or expression of foreign gene(s) (isolated nucleic acid) in the host to produce the desired product.

In some examples, the exogenous nucleic acid sequences can also be introduced into a chloroplast genome thereby enabling chloroplast expression.

### VII. Exemplary Breeding Methods

In one example, open-pollinated methods are used for crops such as rye, many maizes and sugar beets, herbage grasses, legumes such as alfalfa and clover, and tropical tree crops such as cacao, coconuts, oil palm and some rubber.

Population improvement methods fall into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

In one example, a population is changed en masse using a selection procedure. The outcome is an improved population that is indefinitely propagable by random-mating within itself in isolation. Second, the synthetic variety attains the same end result as population improvement but is not itself propagable as such; it has to be reconstructed from parental lines or clones These plant breeding procedures for improving open-pollinated populations are known and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988). For population improvement methods specific for soybean see, e.g., J. R. Wilcox, editor (1987) SOYBEANS: Improvement, Production, and Uses, Second Edition, American Society of Agronomy, Inc., Crop Science Society of America, Inc., and Soil Science Society of America, Inc., publishers, 888 pages.

In one example, mass selection methods are used. In mass selection, desirable individual plants are chosen, harvested, and the seed composited without progeny testing to produce the following generation. Since selection is based on the maternal parent only, and there is no control over pollination, mass selection amounts to a form of random mating with selection. The purpose of mass selection is to increase the proportion of superior genotypes in the population.

In one example, a synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance ensure that some inbreeding occurs. Clonal parents will, of course, remain unchanged and highly heterozygous.

Whether a synthetic can go straight from the parental seed production plot to the farmer or first undergoes one or two cycles of multiplication depends on seed production and the scale of demand for seed. Generally, grasses and clovers are generally multiplied once or twice and are thus considerably removed from the original synthetic.

In some examples, progeny testing is used for polycrosses, because of their operational simplicity and relevance to the objective, namely exploitation of general combining ability in a synthetic.

The number of parental lines or clones that enters a synthetic can vary. In some examples, numbers of parental lines range from 10 to several hundred, with 100-200 being the average. Broad based synthetics formed from 100 or more clones can be more stable during seed multiplication than narrow based synthetics.

In some examples, hybrids are generated. A hybrid is an individual plant resulting from a cross between parents of differing genotypes. Commercial hybrids are used in many crops, including corn (maize), sorghum, sugar beet, sunflower and broccoli. Hybrids can be formed, for example by crossing two parents directly (single cross hybrids), by crossing a single cross hybrid with another parent (three-way or triple cross hybrids), or by crossing two different hybrids (four-way or double cross hybrids).

Most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids can include the isolated production of both the parental lines and the hybrids which result from crossing those lines. For a detailed discussion of the hybrid production process, see, e.g., Wright. Commercial Hybrid Seed Production 8:161-176, In Hybridization of Crop Plants.

In some examples, bulk segregation analysis (BSA) is used. BSA, a.k.a. bulked segregation analysis, or bulk segregant analysis, is described by Michelmore et al. (Michelmore et al., 1991, Proceedings of the National Academy of Sciences, USA, 99:9828-9832) and Quarrie et al. (Quarrie et al., 1999, Journal of Experimental Botany, 50(337):1299-1306). For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., resistant to pathogen), and the other from the individuals having reversed phenotype (e.g., susceptible to pathogen), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

In some examples, gene pyramiding is used to combine into a single genotype a series of target genes identified in different parents. The first part of a gene pyramiding breeding is called a pedigree and is aimed at cumulating one copy of all target genes in a single genotype (called root genotype). The second part is called the fixation steps and is aimed at fixing the target genes into a homozygous state, that is, to derive the ideal genotype (ideotype) from the root genotype. Gene pyramiding can be combined with marker assisted selection (MAS) or marker based recurrent selection (MBRS).

VIII. Exemplary Plants for Use with the Disclosed Methods

The present disclosure teaches plants transformed with a plant transformation construct or vector. The methods for targeted gene-editing as described herein can be used to confer desired traits on essentially any plant. A wide variety of plants and plant cell systems may be engineered for the desired physiological and agronomic characteristics described herein using the nucleic acid constructs of the present disclosure and the various transformation methods. In some embodiments, the plant for the transformation is a monocotyledonous plant (monocot) or a dicotyledonous plant (dicot).

Monocots are flowering plants having embryos with one cotyledon or seed leaf, parallel leaf veins, and flower parts in multiples of three. Examples of monocots that can be used for transformation, genetic engineering or gene-editing include, but are not limited to turfgrass, corn/maize, rice, oat, annual ryegrass, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (Kentucky bluegrass), Lolium spp. (ryegrass species including annual ryegrass and perennial ryegrass), Festuca arundinacea (tall fescue) Festuca rubra commutata (Chewings fescue), Cynodon dactylon (bermudagrass, Pennisetum clandestinum (kikuyu grass), Stenotaphrum secundatum (St. Augustine grass), Zoysia japonica (zoysia grass), and Dichondra micrantha.

Other exemplary plants that can be used for transformation, genetic engineering or gene-editing include, but are not limited to angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

In some embodiments, plants and plant cells for transformation, genetic engineering or gene-editing include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some embodiments, fruit crops such as tomato, apple, peach, pear, plum, raspberry, black raspberry, blackberry, cane berry, cherry, avocado, strawberry, wild strawberry, grape and orange.

In some embodiments, plants and plant cells for transformation, genetic engineering or gene-editing include, but are not limited to, Canola (Brassica napus), Soybean (Glycine max), Cotton (Gossypium hirsutum), Rice (Oryza sativa), Lotus (Lotus japonicus), Radish (Raphanus sativus), Setaria (Setaria italica), Sorghum (Sorghum bicolor), Pennycress (Thlaspi arvense), Southern cattail (Typha domingensis), Wheat (Triticum aestivum), and Maize (Zea mays).

In some embodiments, the plant for transformation, genetic engineering or gene-editing is a dicot. In some embodiments, the plant, plant part, or plant cell is a species selected from Abrabidopsis genus, Brassica genus, Glycine genus, Gossypium genus, Oryza genus, Raphanus genus, Setaria genus, Sorghum genus, Thlaspi genus, Typha genus, Triticum genus, and Zea genus.

In some embodiments, the plant, plant part, or plant cell is from Abrabidopsis thaliana.

In some embodiments, the plant, plant part, or plant cell is from the Brassica genus, such as Brassica balearica (Mallorca cabbage), Brassica carinata (Abyssinian mustard or Abyssinian cabbage), Brassica elongata (elongated mustard), Brassica fruticulosa (Mediterranean cabbage), Brassica hilarionis (St. Hilarion cabbage), Brassica juncea (Indian mustard, brown and leaf mustards, Sarepta mustard), Brassica napus (rapeseed, canola, rutabaga, Siberian kale), Brassica narinosa (broadbeaked mustard), Brassica nigra (black mustard), Brassica oleracea (kale, cabbage, collard greens, broccoli, cauliflower, kai-lan, brussels sprouts, kohlrabi), Brassica perviridis (tender green, mustard spinach), Brassica rapa (Chinese cabbage, turnip, rapini, komatsuna), Brassica rupestris, Brassica spinescens, or Brassica tournefortii (Asian mustard).

In some embodiments, the plant, plant part, or plant cell is from the Thlaspi genus and is Thlaspi alliaceum (roadside penny-cress), Thlaspi arcticum (arctic penny-cress), Thlaspi arvense (field penny-cress), Thlaspi caerulescens (alpine penny-cress), Thlaspi californicum (Kneeland Prairie penny-cress), Thlaspi cyprium (Cyprus penny-cress), Thlaspi fendleri (Fendler's penny-cress), Thlaspi idahoense (Idaho penny-cress), Thlaspi jankae (Slovak penny-cress), Thlaspi montanum (alpine penny-cress), Thlaspi parviflorum (meadow penny-cress), Thlaspi perfoliatum (Cotswold penny-cress), Thlaspi praecox (early penny-cress), or Thlaspi rotundifolium (round-leaved penny-cress).

In some embodiments, the plant, plant part, or plant cell is from the Glycine genus and is Glycine albicans, Glycine aphyonota, Glycine arenaria, Glycine argyria, Glycine canescens, Glycine clandestine, Glycine curvata, Glycine cyrtoloba, Glycine falcata, Glycine gracei, Glycine hirticaulis, Glycine hirticaulis subsp., Glycine lactovirens, Glycine latifolia, Glycine latrobeana, Glycine microphylla, Glycine montis-douglas, Glycine peratosa, Glycine pescadrensis, Glycine pindanica, Glycine pullenii, Glycine remota, Glycine rubiginosa, Glycine stenophita, Glycine syndetika, Glycine tabacina, Glycine tomentella, Glycine soja, or Glycine max.

The present disclosure provides a plant, plant part, or plant cell disclosed above, which is transformed with plant transformation vectors comprising: a nucleic acid encoding an IMA1 gene sequence (or fragment thereof) and/or a nucleic acid that reduces expression of BTSL1 and/or BTSL2; and ii) one or more promoter operably linked to the nucleic acid(s). In some embodiments, the promoter is an inducible promoter, a constitutive promoter, or a cell-type specific promoter.

In some embodiments, the plant, plant part, or plant cell thereof has increased plant disease resistance as compared to an untransformed control plant. In some embodiments, a transformed plant tissue is produced from the transformed plant cell. In some embodiments, a transformed plantlet is produced from the transformed plant tissue and wherein the transformed plantlet has increased plant disease resistance as compared to an untransformed control plantlet. In some embodiments, a progeny of the transformed plantlet is produced and wherein the progeny has increased plant disease resistance as compared to an untransformed control plantlet. In some embodiments, the transformed plantlet or the progeny of the transformed plantlet is grown into a mature transformed plant, and wherein the mature transformed plant has increased plant disease resistance as compared to a mature untransformed control plant. In some embodiments, the mature transformed plant or clone of the mature transformed plant is used in a breeding method taught herein.

In some embodiments, the plant, plant part, or plant cell of the present disclosure has at least about 5% more, about 10% more, about 15% more, about 20% more, about 25% more, about 30% more, about 35% more, about 40% more, about 45% more, about 50% more, about 60% more, about 70% more, about 80% more, about 90% more, about 100% more, about 200% more, plant disease resistance as compared to an untransformed control plant. In some embodiments, the plant, plant part, or plant cell is a genetically-modified, genetically-engineered, or gene-edited one.

In some embodiments, the disclosure teaches a method of producing a plant having increased plant disease resistance, comprising crossing the genetically-modified or gene-edited plant of the disclosure with itself or another plant; and selecting a progeny plant having increased suberin production.

In other embodiments, the method further comprises using the selected progeny in a breeding method taught herein.

In some embodiments, the disclosure teaches a method for increasing plant disease resistance comprising growing the gene-edited plant of the disclosure, or a progeny thereof, for example in soil or other growth media.

The present disclosure provides a solution for increasing plant disease resistance. The present disclosure provides methods of identifying genetic materials that can increase plant disease resistance. Also, the present disclosure provides methods of transferring genetic materials to plants in order to give rise to traits that significantly increase plant disease resistance. Furthermore, the present disclosure teaches newly identified genetic components and methods of generating genetically modified plants, plant cells, tissues, and seeds with altered root system architecture for increasing plant disease resistance.

### IX. Exemplary Additional Genes

### Herbicide Resistance

One or more herbicide resistance genes can be used with the methods and plants provided herein. In particular examples, a herbicide resistance gene confers tolerance to an herbicide comprising glyphosate, sulfonylurea, imidazalinone, dicamba, glufosinate, phenoxy proprionic acid, cyclohexone, triazine, benzonitrile, broxynil, L-phosphinothricin, cyclohexanedione, chlorophenoxy acetic acid, or combinations thereof.

In one example the herbicide resistance gene is a gene that confers resistance to an herbicide that inhibits the growing point or meristem, such as an imidazalinone or a sulfonylurea. Exemplary genes in this category code for mutant ALS and AHAS enzyme as described, for example, by Lee et al. (1988. Embryo J. 7:1241-8) and Miki et al. (1990. Theoret. Appl. Genet. 80:449-458).

Resistance genes for glyphosate (resistance conferred by mutant 5-enolpyruvl-3 phosphikimate synthase (EPSP) and aroA genes, respectively) and other phosphono compounds such as glufosinate (phosphinothricin acetyl transferase (PAT) and Streptomyces hygroscopicus phosphinothricin-acetyl transferase (bar) genes) can be used (e.g., see U.S. Pat. No. 4,940,835). Examples of specific EPSPS transformation events conferring glyphosate resistance are described, for example, in U.S. Pat. No. 6,040,497.

DNA molecules encoding a mutant aroA gene are known (e.g., ATCC accession number 39256 and U.S. Pat. No. 4,769,061), as are sequences for glutamine synthetase genes, which confer resistance to herbicides such as L-phosphinothricin (e.g., U.S. Pat. No. 4,975,374), phosphinothricin-acetyltransferase (e.g., U.S. Pat. No. 5,879,903). DeGreef et al. (1989. Bio/Technology 61-64) describe the production of gene-edited plants that express chimeric bar genes coding for phosphinothricin acetyl transferase activity. Exemplary genes conferring resistance to phenoxy propionic acids and cyclohexones, such as sethoxydim and haloxyfop are the Acct-S1, Accl-S2 and Acct-S3 genes described by Marshall et al. (1992. Theor Appl Genet. 83:435-442).

Exemplary genes conferring resistance to an herbicide that inhibits photosynthesis include triazine (psbA and gs+genes) and benzonitrile (nitrilase gene) (see Przibilla et al., 1991. Plant Cell. 3:169-174). Nucleotide sequences for nitrilase genes are disclosed in U.S. Pat. No. 4,810,648, and DNA molecules containing these genes are available under ATCC Accession Nos. 53435, 67441, and 67442. Cloning and expression of DNA coding for a glutathione S-transferase is described by Hayes et al. (1992. Biochem. J. 285:173).

U.S. Patent Publication No: 20030135879 describes dicamba monooxygenase (DMO) from Pseuodmonas maltophilia, which is involved in the conversion of a herbicidal form of the herbicide dicamba to a non-toxic 3,6-dichlorosalicylic acid and thus can be used for producing plants tolerant to this herbicide.

The metabolism of chlorophenoxyacetic acids, such as, for example 2,4-D herbicide, is well known. Genes or plasmids that contribute to the metabolism of such compounds are described, for example, by Muller et al. (2006. Appl. Environ. Microbiol. 72(7):4853-4861), Don and Pemberton (1981. J Bacteriol 145(2):681-686), Don et al. (1985. J Bacteriol 161(1):85-90) and Evans et al. (1971. Biochem J 122(4):543-551).

### EXAMPLES

It is disclosed herein that microbial patterns such as flagellin lead to suppression of root iron acquisition via a localized degradation of the systemic iron deficiency signaling peptide Iron Man 1 (IMA1). This response is also elicited when bacteria enter root tissues, but not when they dwell on the outer root surface. IMA1 itself has a role in modulating immunity in root and shoot, affecting the levels of root colonization and the resistance to a bacterial foliar pathogen. The present disclosure uncovers an adaptive molecular mechanism of nutritional immunity that affects iron bioavailability and uptake, as well as immune responses.

The present disclosure reveals that the extended presence of the flg22 MAMP can abolish major components of the iron deficiency response in Arabidopsis thaliana. These responses are only mounted when bioavailable iron is scarce in the environment (otherwise the plant wouldn't be iron deficient). The present disclosure shows that the antagonistic function between the IMA1-mediated iron deficiency response and the flg22-elicited defense response might be critical to avoid making iron bioavailable for potential pathogens and to avoid impairing plant defense responses. The present disclosure also shows that the modulation of iron deficiency responses is not a constitutive response that is triggered merely by the presence of bacteria but one that is triggered according to the presence of cues indicative of threats (e.g. sustained presence of high levels of flg22 or tissue damage). This finely tuned modulation may maintain a healthy rhizosphere during iron limiting conditions, as acidic (reducing) conditions and coumarins generally promote iron solubility in the rhizosphere. Shutting iron acquisition down might constitute a way to avoid enabling harmful bacteria the easy access to iron, but at the same time limits available iron for the plant itself as well as beneficial bacteria. Limiting iron availability in the rhizosphere generally might contain the risk for promoting the virulence of bacteria found in the rhizosphere, if it resembles the situation in the mammalian gut, where metabolic cooperativity and iron levels have been shown to suppress virulence.

A close linkage of nutrient stress response and the plant immune system has been observed for plant responses to phosphate and points towards a general and complex intertwinement of nutrient acquisition and plant immune responses. For the conduit between iron and the immune system. IMAs are found herein to be a component mediating a set of complex and multifaceted functions. On one hand IMA local degradation allows for shutting down root acidification, thereby enabling root responses to flg22, such as growth arrest and full phosphorylation of MAPKs. Consistent with these data, lack of IMA led to less colonization of the root with a surface-dwelling commensal bacteria strain. On the other hand, IMA1 overexpressing plants were more resistance to foliar Pseudomonas syringae pv. tomato DC3000.

### Example 1

### Materials and Method

### Plant Material and Plant Growth Conditions

For all experiments, Arabidopsis thaliana ecotype Columbia (Col-0) was used as wild-type control. Plant seeds were sterilized in 70% ethanol for 8 minutes and washed with sterilized MilliQ water. Seeds were sowed on agar medium and stratified at 4° C. for 3 days in the dark. Seedlings were grown in Percival growth chamber (GENEVA SCIENTIFIC) at 22° C. and 16 hours light, 8 hours dark cycle. Iron sufficient (+Fe) medium was prepared according to the recipe of standard media as previously described. The medium contains: 750 M of MgSO₄-7H₂O, 625 μM of KH₂PO₄, 1000 μM of NH₄NO₃, 9400 μM of KNO₃, 1500 μM of CaCl₂-2H₂O, 0.055 M of CoCl₂-6H₂O, 0.053 M of CUCl₂-2H₂O, 50 μM of H₃BO₃, 2.5 μM of KI, 50 μM of MnCl₂-4H₂O, 0.52 μM of Na₂MoO₄-2H₂O, 15 μM of ZnCl₂, 75 μM of Na—Fe-EDTA (+Fe) or 10 μM of Na—Fe-EDTA (phenotype analysis), 1000 μM of MES adjusted to pH 5.5 with KOH, 0.5% Sucrose, and 1% Difco Agar (BD, Cat #214530, only for seedling growth on solid medium).

### Generation of Transgenic Lines

To generate the different promoter driving IMA1 transgenic plants, the upstream promoter region based on the previous study of UBQ10 (AT4G05320, 1986 bp), PGP4 (AT2G47000, 2174 bp), ELTP (At2g48140, 464 bp) and LBD16 (AT2G42430, 2564 bp) was cloned into p5′ (pDONR P4-Plr), the mCitrine CDS without stop codon was cloned into p221 (pDONR 221) and the IMA1 CDS with stop codon was cloned into p3′ (pDONR P2r-P3) through BP reactions. The destination construct was combined using pB7m34GW, p221-mCitrine, p3′-IMA1 and one of the p5′ vector using the multiple gateway LR reaction. The destination constructs were transformed into Col-0 and selected via Basta resistance to obtain homozygous T3 transgenic lines. To generate pIMA1::mCitrine-NLS-mCitrine, the same promoter region of pIMA1::EYFP-IMA1;ima& used in previous study was cloned into p5′ (pDONR P4-Plr) through BP reactions. The destination construct was combined using pB7m34GW, p221-mCitrine, p3′-NLS-mCitrine and p5′-IMA1pro using the multiple gateway LR reaction. The destination constructs were transformed into Col-0 and selected via Basta resistance to obtain homozygous T3 transgenic lines. To generate pIMA1::EYFP-IMA1 in bts-1,fls2 and btsl1,2 mutant background, pIMA1::EYFP-IMA1;ima8x was crossed to bts-1,fls2 and btsl1,2 mutants, respectively, and the F3 homozygous transgenic plants were obtained for experimental analysis.

### Elicitor Preparation and Treatment

Flg22 oligopeptide (QRLSTGSRINSAKDDAAGLQIA) and flg20 oligopeptide (QRLSTGSRINSAKDDAAGLQ) were synthesized by the Salk Peptide Synthesis Core. The elf18 oligopeptide (Ac-SKEKFERTKPHVNVGTIG) was obtained from EZbiolab. The peptides were dissolved in deionized water. Chitin (Frontier Scientific, Cat. JK399372) was dissolved in deionized water in 4° C. with overnight rotating.

For elicitor treatment, flg22 and flg20 was added to the liquid medium to obtain the respective final concentration (depending on the time scale of the assay 2 μM, 1 μM, 100 nM or 10 nM as described in the figure legends). Chitin was diluted to 1 mg/mL as the final concentration. Seedlings were treated in +Fe (75 μM FeEDTA) or no Fe (0 μM FeEDTA, 50 μM FerroZine (ACROS Organics, Cat. 410570050)) liquid medium containing the elicitors for 24 hours, unless otherwise specified.

### Protein Extraction and Western Blot.

7-day-old seedlings grown on +Fe plates were transferred to liquid medium with +Fe (75 μM Fe), +Fe+flg22 (75 μM Fe+100 nM flg22), −Fe (50 μM FerroZine), −Fe+flg22 (50 μM FerroZine+100 nM flg22) and treated for 24 hours. For the protein extraction and western blot procedure, previously published method was used with minor modifications. For IRT1 and EYFP-IMA1 detection, 15 roots cut from the pretreated seedlings were harvested immediately in liquid nitrogen. The samples were ground with liquid nitrogen and lysed directly in 80 μL total protein extraction buffer (50 mM Tris-Cl pH 7.4, 150 mM NaCl, 5 mM EDTA, 1% SDS, and 1% TritonX-100 supplemented with 1× NuPAGE™ LDS Sample Buffer (Invitrogen™, Cat. NP0008) and 1× NuPAGE™ Sample Reducing Agent (Invitrogen™, Cat. NP0009) for 15 minutes on ice. The protein samples were denatured by heating for 10 minutes at 90° C. and centrifuge at 13000 rpm for 10 minutes. The supernatant protein samples were separated by NUPAGE 10% Bis-Tris Plus Gel (Invitrogen™, Cat.NW00105BOX) and transferred onto Nitrocellulose membrane by iBlot 2 Dry Blotting system (Invitrogen™, Cat. IB23001). IRT1 was detected by western blot with corresponding antibody (primary antibody, anti-IRT1 (Agrisera, Cat. No. AS111780) 1:2000 diluted in 5% non-fat milk; secondary antibody: Goat Anti-Rabbit IgG (H+L)-HRP Conjugate (Bio-Rad, Cat. No. 170-6515) 1:5000 in 5% non-fat milk). EYFP-IMA1, mCitrine-IMA1, and free GFP that expressed in CHA0 bacteria were detected by anti-GFP HRP conjugate antibody (Miltenyi Biotec, Cat. No. 130-091-833, 1:2000 diluted in 5% non-fat milk). FIT-3xHA was detected by anti-HA HRP conjugate antibody (Roche, Cat. No. 12013819001, 1:2000 diluted in 5% non-fat milk). The same membrane was stripped by the following steps (i) wash the membrane with 1 M NaOH for 5 minutes, (ii) wash the membrane with 1×TBST, 5 minutes for 3 times. Then the membrane was re-blotted with Tubulin antibody as the internal control (Invitrogen™, Cat. 32-2500, 1:5000 in 5% non-fat milk; Goat Anti-Mouse IgG (H+L)-HRP Conjugate (Bio-Rad, Cat. No. 170-6516), 1:5000 in 5% non-fat milk).

### Western Blot for MAPK Activity Analysis

20 seedlings of 7-day-old light-grown Col-0 and UBQ10::mCtritine-IMA1 on the +Fe plates without MES were treated in Fe sufficient liquid medium without MES with 1 μM flg22 peptide for 5 and 10 minutes. The root and shoot parts of the seedlings were harvested separately then the samples were ground with liquid nitrogen and lysed directly in 80 μL (for root samples) or 150 μL (for shoot samples) total protein extraction buffer (Same as described above). The phosphorylation status of MPK3,6 was detected by western blot with corresponding antibody (phospho-P44/42 MAPK antibodies (Cell Signaling, Cat. No. #4370) 1:2000 diluted in 1% BSA, Merck/Calbiochim, Cat. No. 12657; Goat Anti-Rabbit IgG (H+L)-HRP Conjugate (Bio-Rad, Cat. No. 170-6515) 1:5000 in 5% non-fat milk). The same membrane was re-blotted with Tubulin antibody (root samples) (Invitrogen™, Cat. 32-2500, 1:5000 in 5% non-fat milk; Goat Anti-Mouse IgG (H+L)-HRP Conjugate (Bio-Rad, Cat. No. 170-6516), 1:5000 in 5% non-fat milk as an internal control.

### Ferric Chelate Reductase Activity Measurement

7-day-old seedlings grown on +Fe plates were transferred to liquid medium with +Fe (75 μM Fe), +Fe+flg22 (75 μM Fe+100 nM flg22), −Fe (50 μM FerroZine), or −Fe+flg22 (50 μM FerroZine+100 nM flg22) and treated for 2 days. The seedlings were washed with deionized water for 5 times to remove any residual chemicals and 8-10 seedlings were pooled as one sample. The seedlings were incubated in the assay solution containing 0.1 mM Fe (III)-EDTA and 0.3 mM FerroZine in the dark for 1 hour. The ferric chelate reductase activity was measured by spectrophotometry (Beckman Coulter, DU 730) with the absorbance (562 nm) of the Fe (II)-Ferrozine complex. The results were calculated on a root fresh weight basis by the formula:

$$FCR activity ( nmol / ( g . hour ) ) = OD ( 5 \; 62 ) / 29800 * V ( ml ) / Fw ( g ) / T ( hour ) * {10}^{6}$$

### Total Chlorophyll Concentration Measurement

The total chlorophyll content measurement is based on spectrophotometric analysis. Briefly, four shoots of the plants were harvested and pooled as one sample. Any liquid was carefully removed from the sample before the fresh weight was measured. Chlorophyll was extracted with 80% acetone in TissueLyser until the pellet became white. The leaf extracts were measured by spectrophotometry (Beckman Coulter, DU 730, 1 cm width cuvettes) with the absorbance (663 nm and 647 nm), respectively. The results were calculated on a root fresh weight basis by the formula:

$$Total chlorophyll ( a + b ) ( μg / mL ) = ( 7.15 * A 663 ) + ( 18.71 * A 647 )$$

$$Total chlorophyll concentration = ( Total chlorophyll * extract volume ( mL ) / Fw ( g )$$

### Iron Concentration Measurement

The total iron content was measured by a spectrophotometric method that was described previously. The seedlings were harvested and rinsed with de-ionized water for 5 times and dried in an oven at 65° C. for two days. After measuring the dry weight, the tissues were digested with 65% (v/v) HNO₃ at 95° C. for 6 hours, followed by adding 30% (v/v) H₂O₂ at 56° C. for 2 hours. The iron content was analyzed in the assay solution (1 mM bathophenanthroline disulfonate (BPDS), 0.6 M sodium acetate, and 0.48 M hydroxylamine hydrochloride). The FeCl₃ solution was used to prepare the standard curve. The resulting Fe²⁺-BPDS₃ complex was measured with absorbance (535 nm) using a microplate reader. The iron content in each sample was calculated by the absorbance values against the standard curve and normalized by the dry weight (DW).

Confocal microscopy for pIRT1::NLS-2xYPet and pIMA1::EYFP-IMA1 with PI staining

Seedlings were precultured on +Fe (75 μM Fe) solid media for 4 days. For the treatment, around 8-10 seedlings were transferred to 5 mL of +Fe (75 μM Fe), +Fe+flg22 (75 μM Fe+100 nM flg22), −Fe (50 μM FerroZine), and −Fe+flg22 (50 μM FerroZine+100 nM flg22) liquid medium in a 6-well plate and treated for 24 hours. For PI staining, pre-treated seedlings were stained with PI solution (2 μg/mL, dissolved in MilliQ water) for 5 minutes and rinsed in water. Imaging experiment was performed on a Zeiss LSM710 confocal microscope with C-Apochromat 40×/1.20 W Korr M27 water objective lens. Ypet was excited with a 488 nm laser and fluorescence emission was filtered by a 505/550 nm filter. The PI signal is excited with either 488 or 514 nm laser and fluorescence emission was filtered by a 600/650 nm filter. For the quantification of the raw intensity of the Ypet signal, the process has been automatized using the Fiji® software Macro, MACRO_Min_pIRT1_LSM710_PI-staining.

For EYFP-IMA1 imaging in the leaf, 4-day-old pIMA1::EYFP-IMA1;ima8x seedlings were incubated in 5 mL of +Fe (75 μM Fe), +Fe+flg22 (75 μM Fe+100 nM flg22), −Fe (50 μM FerroZine), and −Fe+flg22 (50 μM FerroZine+100 nM flg22) liquid medium in a 6-well plate for 24 hours. Before imaging, cotyledons were excised and mounted in water, abaxial side of epidermis, mesophyll and vasculature regions were imaged using C-Apochromat 40×/1.20 W Korr M27 water objective lens. The bright field was imaged at the same time as a control. YFP was excited with a 514 nm laser and fluorescence emission was filtered by a 520/570 nm filter.

### Microscopy Setup for EYFP-IMA1 Quantification

Imaging experiments except when indicated below, were performed with the following Zeiss LSM710 confocal microscope set up: inverted Zeiss microscope using a Plan-Apochromat 20×/0.8 M27 objective lens. YFP was excited with a 514 nm laser (60 mW) and fluorescence emission was filtered by a 519/580 nm filter.

### Calculation of the EYFP-IMA1 Mean Gray Values

Eight Z-stack images were acquired in bright field and YFP signal representing a root section between 35 and 45 μm. The brightfield images were processed with a Z-projection using the standard deviation method. From these images, the root area was detected using the plugin “Wavelet a trou” (http://www.ens-lyon.fr/RDP/SiCE/METHODS.html). The YFP images were processed with a Z-projection using the sum method. On those images the root area previous determined was reported and the mean gray value measured in this area to obtain the Mean gray value of the YFP channel. The process has been automatized using the Fiji® software Macro, MACRO_Intensity_IMA1.

### Calculation of the EYFP-IMA1 Diameter

Eight Z-stack images were acquired in bright field and YFP signal representing a section between-35 and 45 μm. The brightfield images were processed with a Z-projection using standard deviation method. The YFP images were processed with a Z-projection using the sum method. On Fiji® software, the root width was calculated using the straight line and on the same zone the width of the YFP signal was determined. The ratio of the YFP width was divided by the root width and multiplied by 100 to obtain the percentage of YFP-IMA1 tissue lateral diffusion.

### Rhizosphere Acidification Assay and pH Quantification

Col-0, ima8x and UBQ10::mCitrine-IMA1 seedlings were precultured on +Fe (75 μM Fe) solid medium for 7 days. The seedlings were transferred to +Fe solid medium or no Fe (0 μM Fe, 50 μM FerroZine) solid medium for 3 days before the pH assay was carried out. The seedlings were then placed on a 1% agar plate containing 0.05% (w/v) bromocresol purple (pH 6.5 adjusted with NaOH) for 24 hours before being photographed.

Quantification of rhizosphere pH was conducted with a spectrophotometric assay that was published with some modification. 7-day-old seedlings grown on +Fe plates without MES were transferred to liquid medium with +Fe (75 μM Fe), +Fe+flg22 (75 μM Fe+100 nM flg22), −Fe (50 μM FerroZine), or −Fe+flg22 (50 μM FerroZine+100 nM flg22) without MES and treated for 2 days. Then the seedlings were transferred to the same liquid media supplemented with pH indicator bromocresol purple (0.005%) for 1 day in 48-well plates. Proton extrusion capacity was analyzed by reading the absorption at 590 nm (A590) with an automated microplate reader.

### CHA0 Bacteria Strain Growth Condition and Inoculation

The GFP or mCherry-labeled Pseudomonas protegens strain, CHA0-gfp2 (CHA0::attTn7-gfp2; Gm^{r}) and CHA0-mCherry (CHA0::attTn7-mCherry; Gm) were used for the bacteria inoculation assay. The CHA0-gfp2 or CHA0-mCherry strain was cultured in liquid LB medium (Miller's LB Broth, Research products international, Cat. L24040) supplemented with 25 μg/ml gentamycin at 28° C. overnight. Bacteria cells were harvested by centrifugation (1 minute, 5000 rpm) and resuspended in sterile MilliQ water for 5 times to prevent potential element contamination from the LB medium.

For the bacteria inoculation experiment, 4-day-old seedlings (imaging for IMA1 in DZ) or 7-day-old seedlings (imaging for IMA1 in LRP) were precultured on +Fe (75 μM Fe) solid medium were treated with liquid medium. Around 8-10 seedlings were transferred to 5 mL of (Gruber medium with 0.25% sucrose)+Fe (75 μM Fe), +Fe+CHA0 (75 μM Fe+CHA0-gfp2 or CHA0-mCherry), −Fe (100 μM FerroZine), −Fe+CHA0 (100 μM FerroZine+CHA0-gfp2 or CHA0-mCherry), and −Fe+flg22 (100 μM FerroZine+100 nM flg22) liquid medium in a 6-well plate and treated for 24 hours. For the liquid treatment with CHA0, the bacterial suspension was added in the well to a final OD₆₀₀ of 0.05.

The effect of iron on CHA0 colonization of Arabidopsis roots was performed according to the previous studies with some modification. Briefly, 7 days old seedlings of Col-0, ima8x and UBQ10::mCitrine-IMA1 were transferred to 5 mL of (Gruber medium without MES and sucrose)+Fe (50 μM FeEDTA pH 5.5), −Fe (100 μM FerroZine), or non-available iron (nAvFe 50 μM FeCh with pH 7.0) liquid medium in a 6-well plate and treated for 24 hours with prewashed CHA0-mcherry at the final OD 0.02. After the treatment, the roots were gently washed with sterilized de-ionized water to remove the non-attached bacteria and the root length was measured. The roots were harvested in 1 mL extraction buffer (10 mM MgCl₂, 0.01% Silwet L-77) and homogenized using TissueLyser with stainless steel beads. The samples were diluted serially from 10¹ to 10⁶, and then spread on LB agar plates supplemented with 30 μg/mL gentamycin. The CFUs were counted after 30 hours incubation at 28° C. The calculated CFUs were normalized by the root length.

Pseudomonas syringae pv. Tomato DC3000 Infection Assay

Arabidopsis thaliana Col-0 and UBQ10::mCitrine-IMA1 were grown in a chamber at 22° C. with a 12-h light period and 60-70% relative humidity for 30-31 days in the soil. Pseudomonas syringae pv. tomato DC3000 was cultured in the King's B (KB) liquid medium with antibiotics (Rifampicin and Tetracyclin) at 28° C. Bacteria were harvested by centrifugation and resuspended in sterile water to an OD₆₀₀ of 0.001 (approximately 5×10⁶ colony forming unit (CFU) ml⁻¹). A. thaliana leaves (2-3 fully-expanded leaves per plant) were infiltrated with bacterial suspensions using a needleless syringe. Two days after bacterial infiltration, two leaf discs (0.13 cm²) per leaf were homogenized in 200 μl of MgSO₄, and a dilution series was streaked on KB plates. The plates were incubated at 28° C. for approximately two days before CFUs were counted.

### Quantitative RT-PCR

7-day-old seedlings grown on +Fe solid medium were transferred to 5 mL+Fe, +Fe+flg22, −Fe, or −Fe+flg22 liquid medium in a 6-well plate and treated for 24 hours unless otherwise specified. The root total RNA was isolated from around 15 roots (as a pool for one biological replicate) by using Spectrum™ Plant Total RNA Kit (Sigma-Aldrich, Cat. STRN250). 500 ng of total RNA was reverse transcribed to cDNA using Maxima™ H Minus cDNA Synthesis Master Mix with dsDNase (Thermo Fisher, Cat. M1682). qRT-PCR was performed with Bio-Rad CFX384 Real-Time System and Luna qPCR mix (New England Biolabs, M3003L) according to the manufacturer's instructions. All the primers used for qRT-PCR analysis are listed in Table 2.

TABLE 2

| Cloning | | SEQ ID NO: |
|---|---|---|
| ELTPpro GF | GGGGACAACTTTGTATAGAAAAGTTGTAaatttgagttattattgttca | 157 |
| ELTPpro GR | GGGGACTGCTTTTTTGTACAAACTTGAtttaattggcctgcttaatct | 158 |
| LBD16pro GF | GGGGACAACTTTGTATAGAAAAGTTGTAgaaattcgaaagcgacg | 159 |
| | atgca | |
| LBD16pro GR | GGGGACTGCTTTTTTGTACAAACTTGAcggcgaaacgaacaaaaaa | 160 |
| | gt | |
| PGP4pro GF | GGGGACAACTTTGTATAGAAAAGTTGTAtaaaggatttgggtctattc | 161 |
| | ga | |
| PGP4pro GR | GGGGACTGCTTTTTTGTACAAACTTGAagatacctcacgatcctaagc | 162 |
| | a | |
| p3′ IMA 1cds GF | GGGGACAGCTTTCTTGTACAAAGTGGTAATGATGTCTTTT | 163 |
| | GTCGCAAACT | |
| p3′ IMA 1cds GR | GGGGACAACTTTGTATAATAAAGTTGATCACGCAGCAGG | 164 |
| | AGCATAA | |
| p5′-IMA 1pro-GF | GGGGACAACTTTGTATAGAAAAGTTGTAGTGTCTTAGTC | 165 |
| | CTTAACCACAATCAC | |
| p5′-IMA1pro-GR | GGGGACTGCTTTTTTGTACAAACTTGATGATATTTTTTGT | 166 |
| | TGGATATGAATGAAAG | |
| Genotyping | | |
| fls2 sail LP | ACATGTCCGGTACTATCGCAG | 167 |
| fls2 sail RP | TCCATCAAGACAGCTAATGAGC | 168 |
| bts-1 LP | CCAAATGCGTTCGTAGGTAAG | 169 |
| bts-1 RP | TCAGATTTACACAAATTTGCAGC | 170 |
| opt3-2 LP | CAAAATCCATTCGGACATGTC | 171 |
| opt3-2 RP | GAGGCTAAAACTCCACCCAAG | 172 |
| bts11-1 LP | TGCTTGGCATAATCCTTCTTG | 173 |
| bts11-1 RP | GAACTCTCTTGCTTCCTGAAGC | 174 |
| bts12-2 LP | TCGGTTATTCAGGCAAAACAC | 175 |
| bts12-2 RP | CCCTTTGTACTCATCAGCAGC | 176 |
| qPCR primers | | |
| qbHLH38-F | ACGGTGCCGGAGATAACCTA | 177 |
| qbHLH38-R | GTCGGTCACGTTCACTAGCA | 178 |
| qbHLH39-F | CCGTTCATGTCTTCCTGCCT | 179 |
| qbHLH39-R | GCCTTTGGTGGCTGCTTAAC | 180 |
| qRT_FRO2_F | GCCACATCTGCGTATCAAGTT | 181 |
| qRT_FRO2_R | TCCCAAACAAGCTACGACCA | 182 |
| qRT_IRT1_F | GAATGTGGAAGCGAGTCAGCGA | 183 |
| qRT_IRT1_R | GATCCCGGAGGCGAAACACTTA | 184 |
| qRT_CYP71A12_F | ATTGGTAACCTCCACCAGCTT | 185 |
| qRT_CYP71A12_R | CATAGGGACCAAACACCACAT | 186 |
| PR1 qF | TTCTTCCCTCGAAAGCTCAA | 187 |
| PR1 qR | AAGGCCCACCAGAGTGTATG | 188 |
| PR4 qF | GTACCACCGCGGACTACTGT | 189 |
| PR4 qR | CGTGGAGCAATAAGCACTCA | 190 |
| WRKY46 qF | CATCCTTAAGCGAAGCCTTG | 191 |
| WRKY46 qR | TCGATGCGTGCATCTGTAAT | 192 |
| FRK1 qF | CAGGATGTTGATGCCATGAG | 193 |
| FRK1 qR | AAGGTTGGAGAAGGCTGGAT | 194 |
| PER5 qF | CTCGAGGGAACATCAACGAT | 195 |
| PER5 qR | ACTTTCACTGCCCATTTTGG | 196 |
| qACT2-F | TGTGCCAATCTACGAGGGTTT | 197 |
| qACT2-R | TTTCCCGCTCTGCTGTTGT | 198 |
| ERF1 qF | GGCGGAGAGAGTTCAAGAGT | 199 |
| ERF1 qR | AGACCCCAAAAGCTCCTCAA | 200 |
| JAZ1 qF | GCCGGGCAAGTGATTGTATT | 201 |
| JAZI qR | TCCAAGAACCGGTGAAGTGA | 202 |
| ACS4 qF | AATGTCAAGTTTCGGCCTTG | 203 |
| ACS4 qR | AGGTCTCATGTCGACCCAAC | 204 |
| ERF4 qF | CGACGTTAGTTGTGCCTTCA | 205 |
| ERF4 qR | TCTAAACGCCGATGTCACAG | 206 |
| JAR1 qF | CACCGAAAGAGACCTTCAGC | 207 |
| JAR1 qR | AACTAACGTAACCCGCATCG | 208 |

### RNAseq and Data Analysis

For transcriptomic analysis, 7-day-old seedlings grown on +Fe medium plates were transferred to 5 mL+Fe, +Fe+flg22, −Fe, or −Fe+flg22 liquid medium in a 6-well plate and treated for 24 hours. 20 roots were pooled harvested as one biological replicate, with a total of three independent replicates per condition in each genotype (total sample numbers=36). The root samples were ground in liquid nitrogen and the RNA was extracted with the Spectrum™ Plant Total RNA Kit (Sigma-Aldrich, Cat. STRN250). The RNA quality and quantity were determined using a 2100 Bioanalyzer tape station (Agilent Technologies) and Qubit Fluorometer (Invitrogen). The sequencing libraries were generated by the Salk Next Generation Sequencing Core according to Illumina manufacturer's instructions. Sequencing was performed using the Illumina Nextseq2000 platform.

### Read Alignment and Generation of Counts

RNA-seq short reads were mapped to the TAIR 10 reference genome which were obtained from the Arabidopsis Information resource web site (http://www.arabidopsis.org) using the Splice Transcripts Alignments to Reference (STAR) version 2.7.0a. A STAR index was built using the following parameters before mapping:
$ STAR--runThreadN 4
--runMode genomeGenerate\
--genomeDir ara_star_index\
--genomeFastaFiles<TAIR10.fa>\
--sjdbGTFfile<TAIR10.gtf>\
--sjdbOverhang 99

Then, RNA-seq reads in the FASTQ files were aligned to TAIR 10 and raw count files were generated using the following STAR command:
$ STAR--genomeDir ara_star_index\
--runThreadN 8 \
--sjdbOverhang 99 \
--sjdbGTFfile<TAIR10.gtf>\
--outSAMtype BAM SortedByCoordinate\
--outFileNamePrefix/output/small_\
--outReadsUnmapped Fastx\
--quantMode GeneCounts\
--readFilesIn<fastq>

A custom R script (min_R_codes_for_manuscript) was used to combine counts per gene from count data produced from STAR cross all samples.

### Differential Expression Analysis

Normalization of the read counts and differential gene expression analysis were performed using the R package, edgeR (version 3.36.0). The CPM (counts per million) function from edgeR was used to normalize the counts and differentially expressed genes (DEGs) by comparing the +Fe and −Fe treatment without or with flg22 were identified using gbmLRT function from edgeR. A false discovery rate (FDR<0.05) and log FC (>0 or <0) were used as the criteria values for identification of up-regulated and down-regulated DEGs.

The DEG analysis was conducted by comparing different combinations of iron and flg22 treatments in wild type using ANOVA with Benjamini-Hochberg-corrected (FDR<0.05) plus maximal absolute value of log 2-converted fold change between pairs of treatment conditions larger than 1. Differentially expressed genes were visualized and k-means clustering method was used to classify DEGs via the ComplexHeatmap package in R. The cluster number (k=5) was determined by total within-cluster sum of squared error and Bayesian information criterion (BIC). The boxplots were used to display scaled expression of genes from each cluster.

Gene ontology enrichment analysis was conducted using online tools of GENEONTOLOGY website: http://geneontology.org/

### Statistical Analysis

Statistical significance of overlap between DEGs from was assessed by hypergeometric distribution test. Hypergeometric test function in R was used to calculate statistical significance:
$ phyper(q-1, m, n-m, k, lower.tail=FALSE, log.p=FALSE)
q=the number of genes in common between two sets
m=the number of genes in Set 1;
n=the total number of genes in RNA-Seq counts table (33,602)
k=the number of genes in Set 2
   Statistical analysis was performed by Graphpad Prism 9 and XLSTAT 2020.4.1.1032

### Box & Whiskers Plots

For all the Box & whiskers plots (unless specified in the figure legend), the upper and lower boundary show min to max. The horizontal line in the box represents the median value. The upper quartile represents 75th percentile and the lower quartile represents 25th percentile.

### Statistics and Reproducibility

Each experiment was repeated independently for at least 3 times with consistent results except for the western blot of FIG. 3g were repeated for two times and showing consistent results.

### Example 2

### Flg22 Represses Root Responses to −Fe

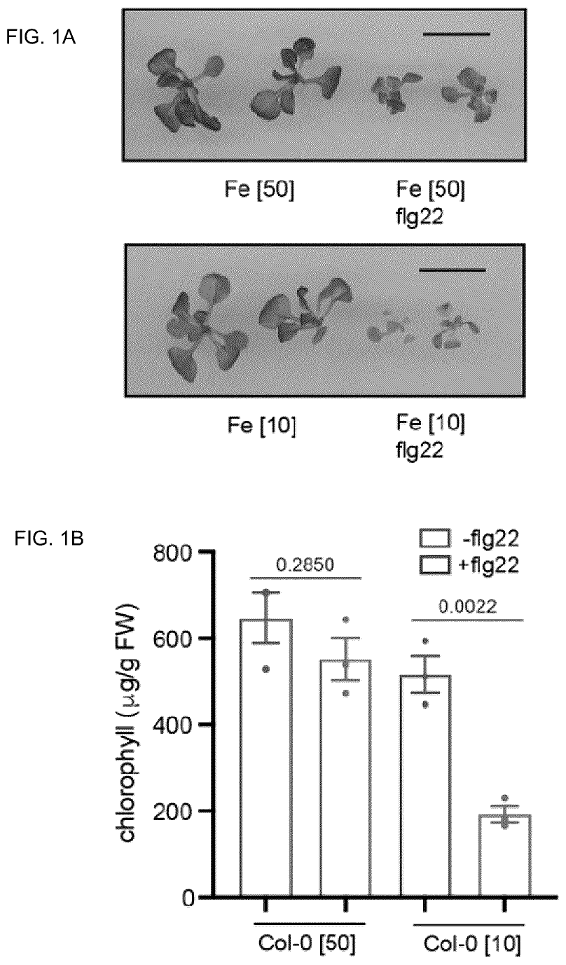

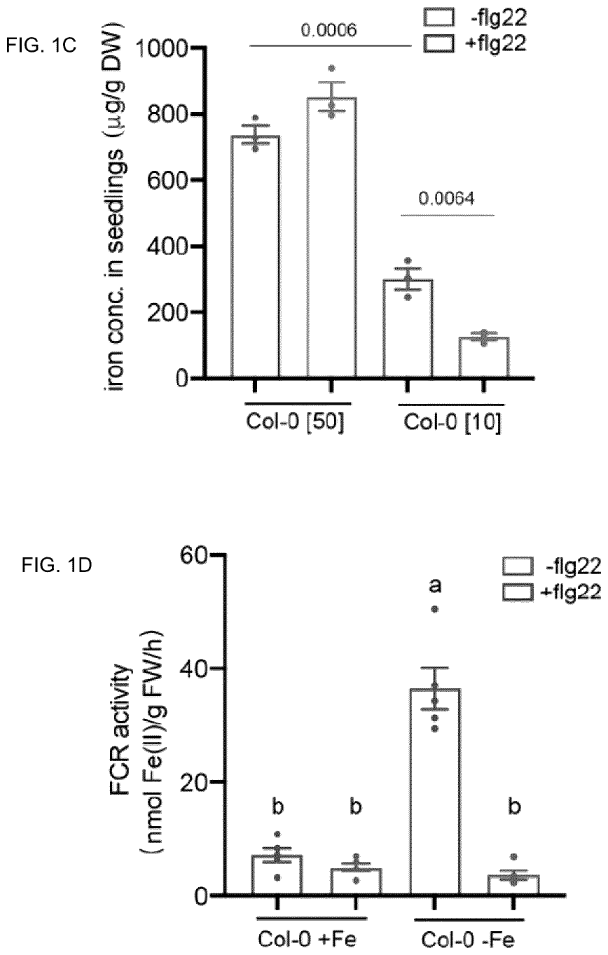

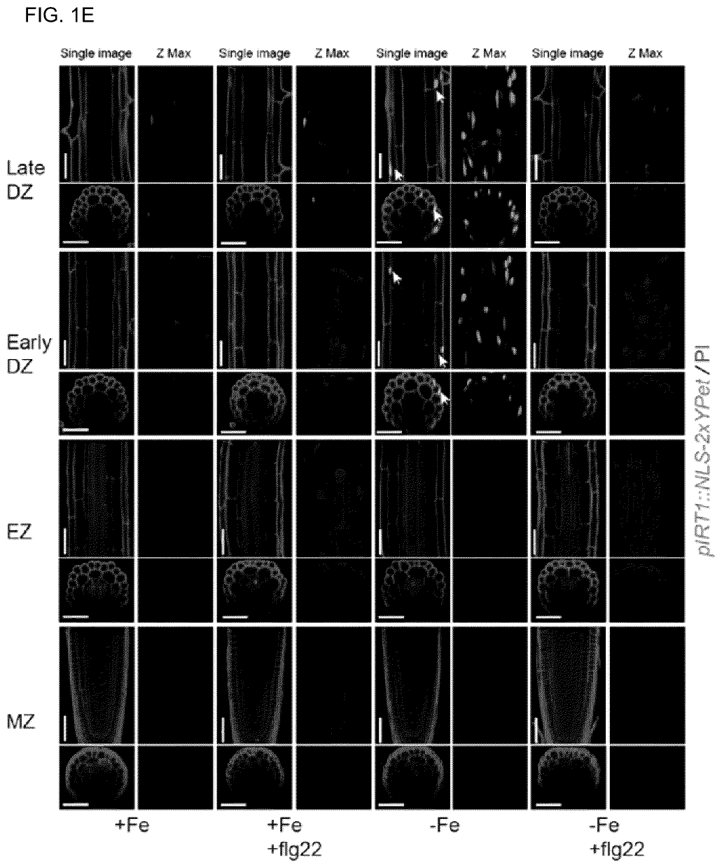

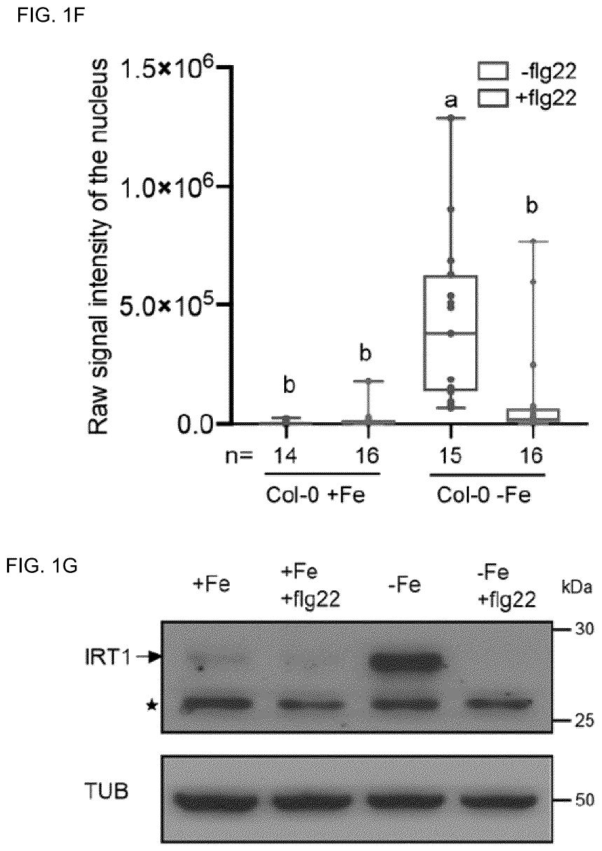

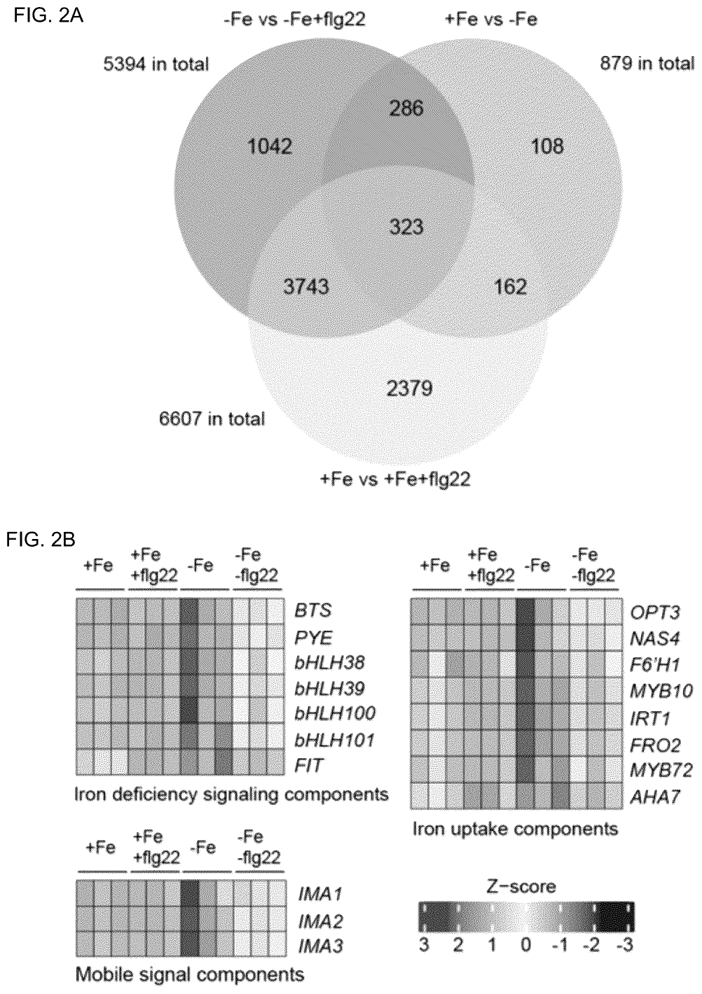

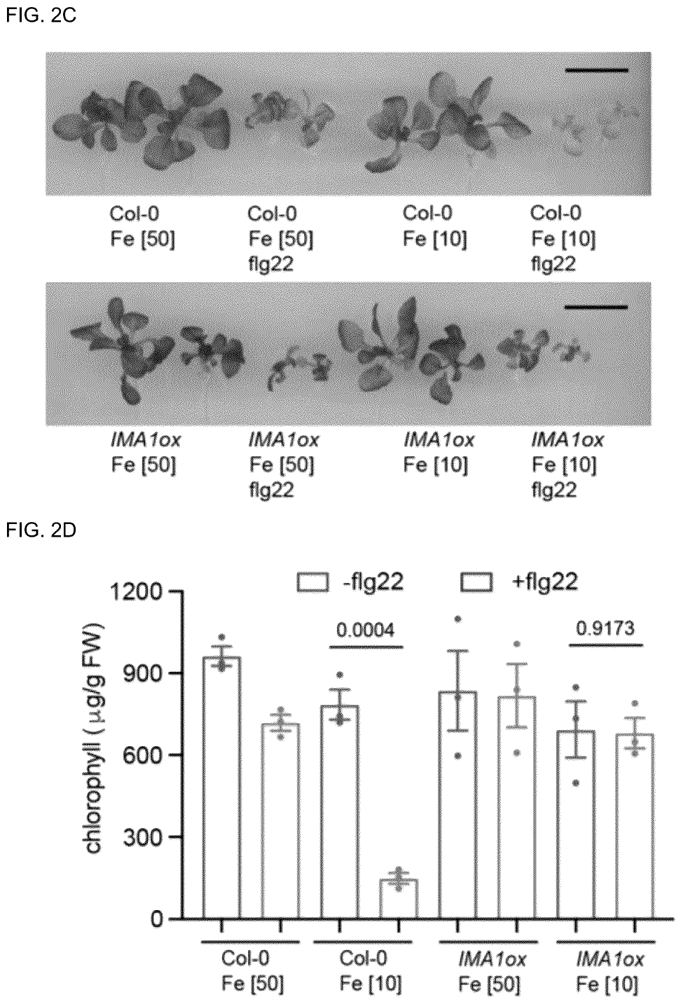

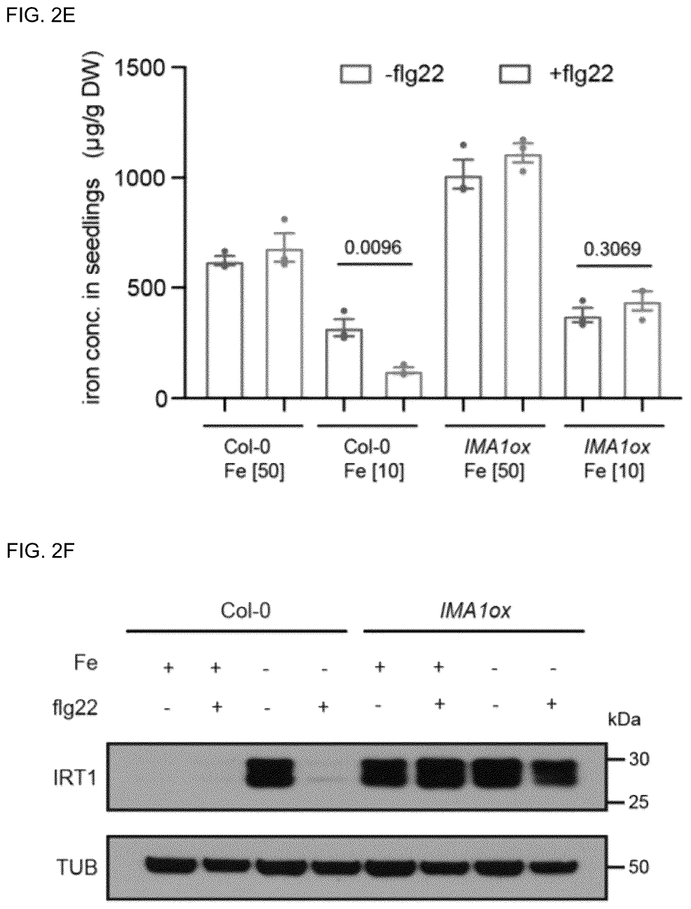

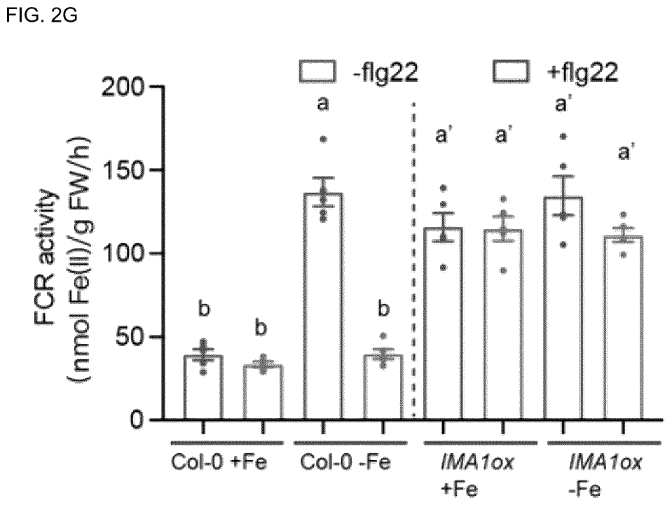

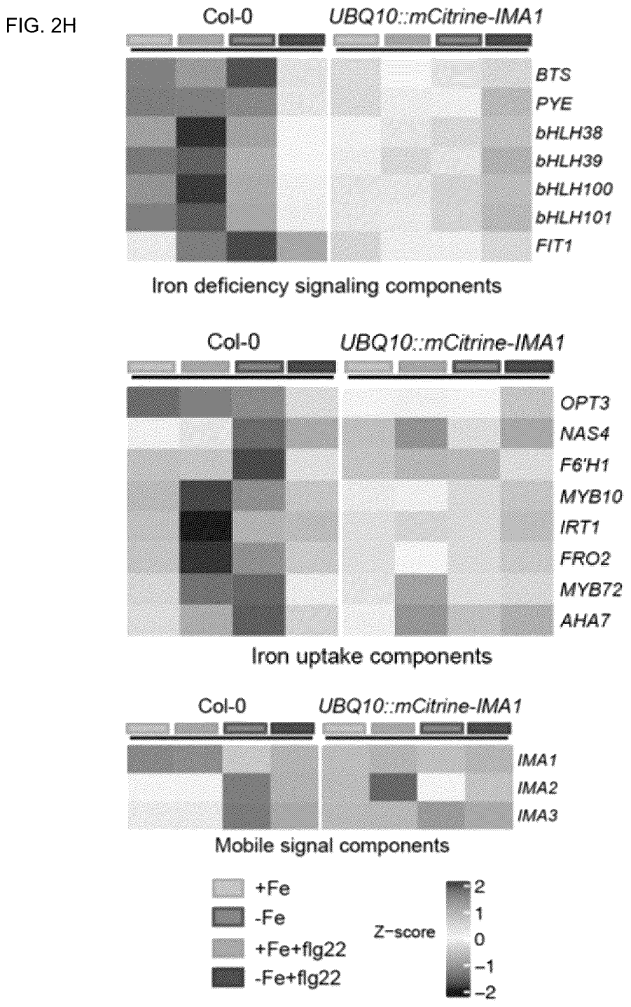

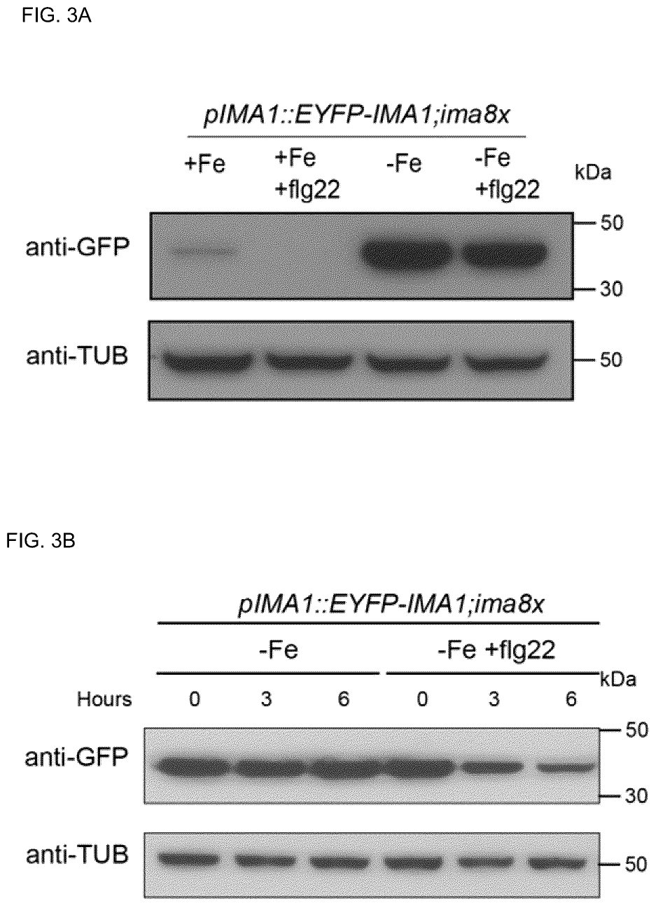

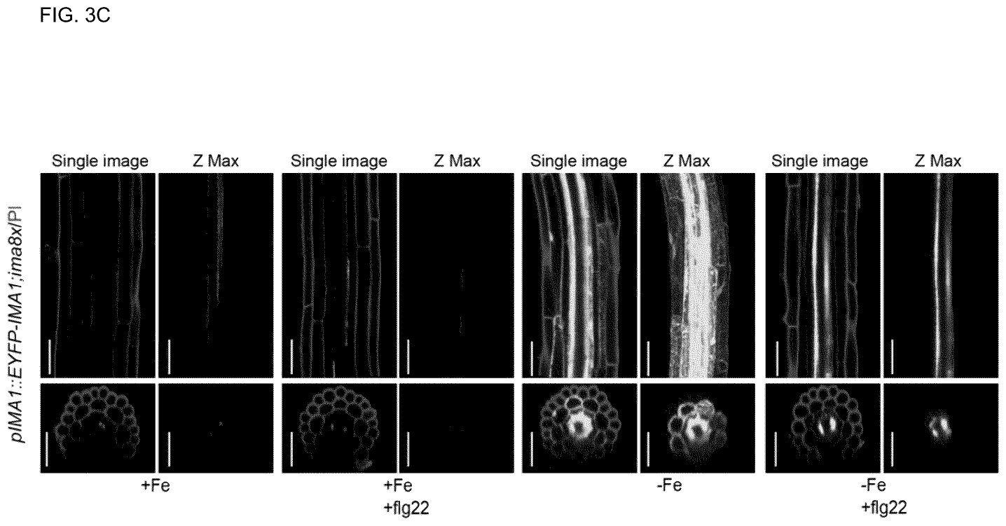

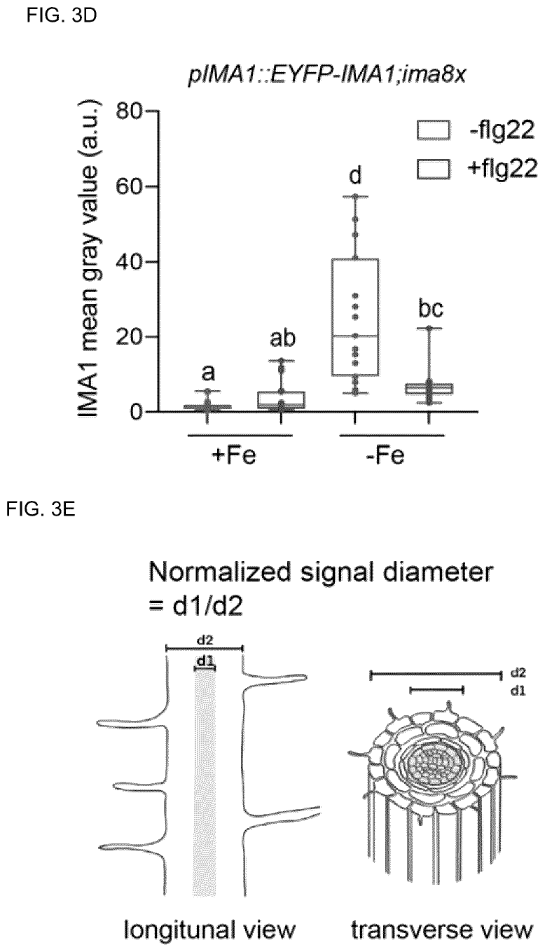

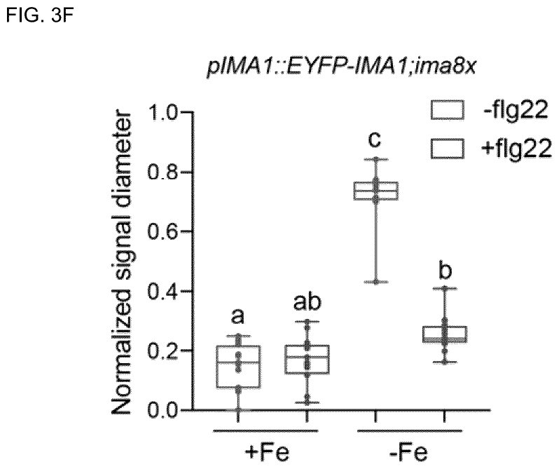

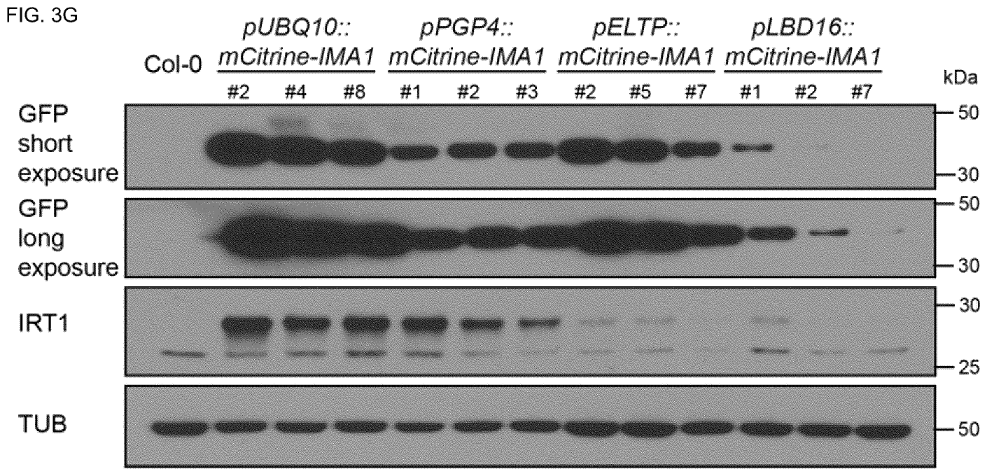

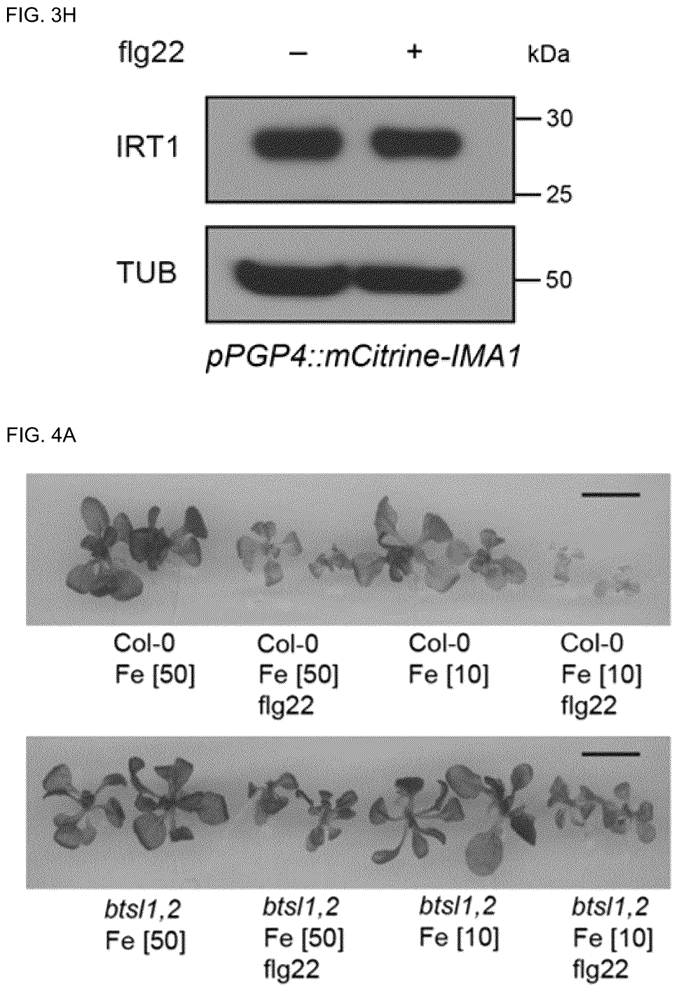

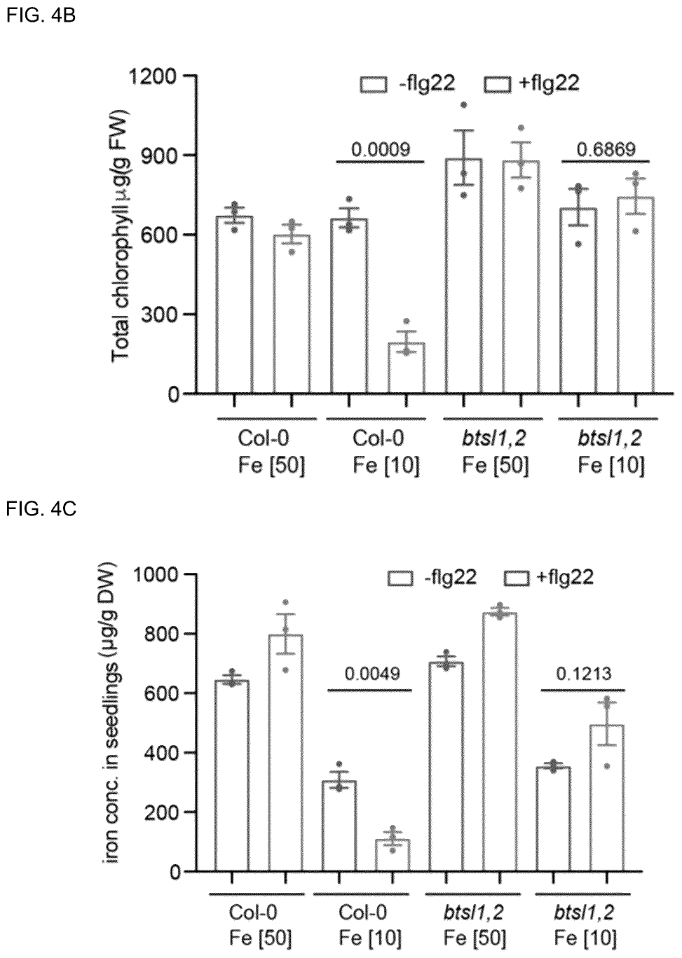

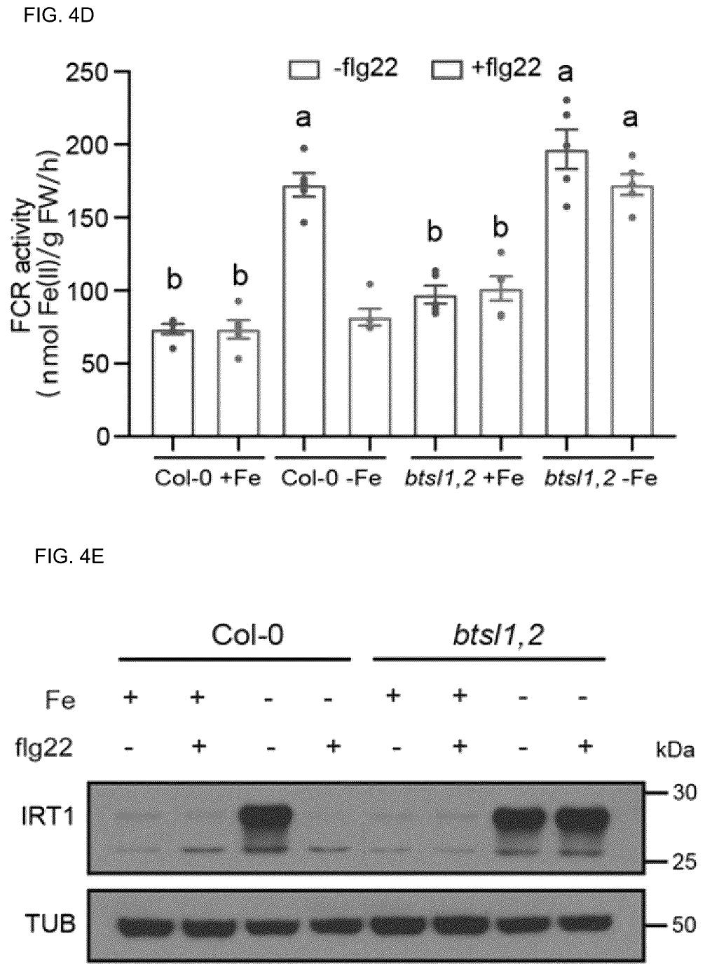

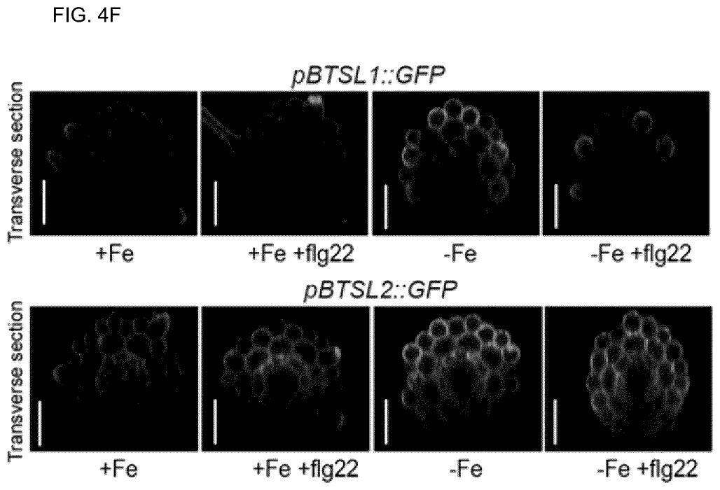

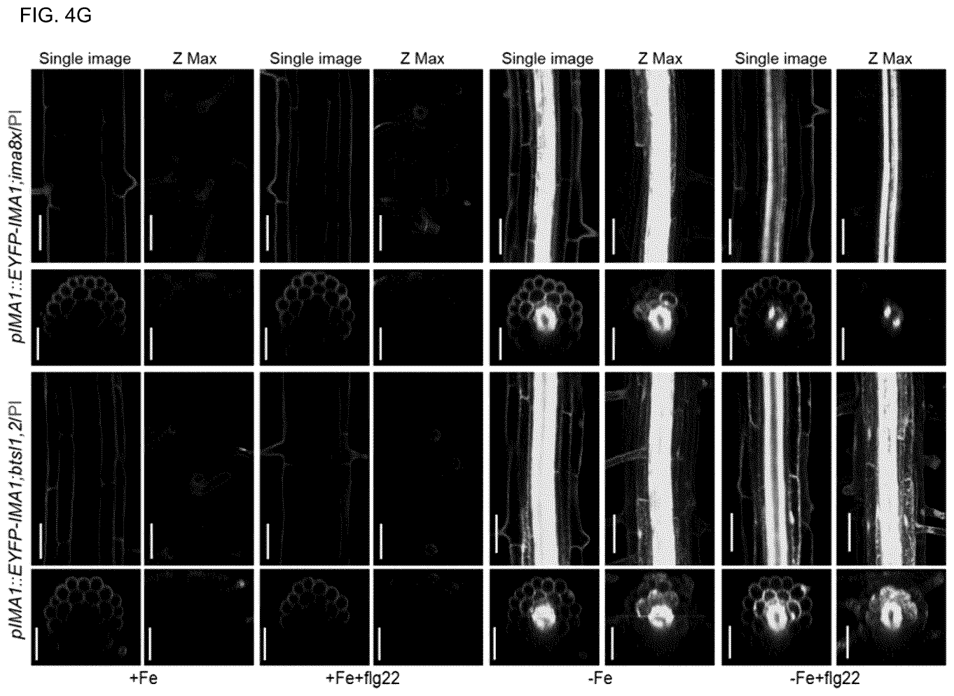

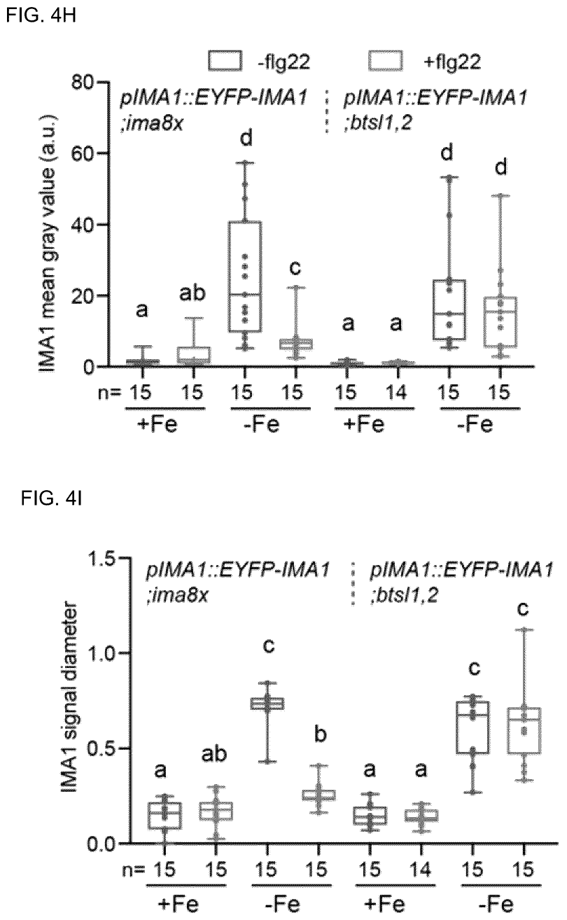

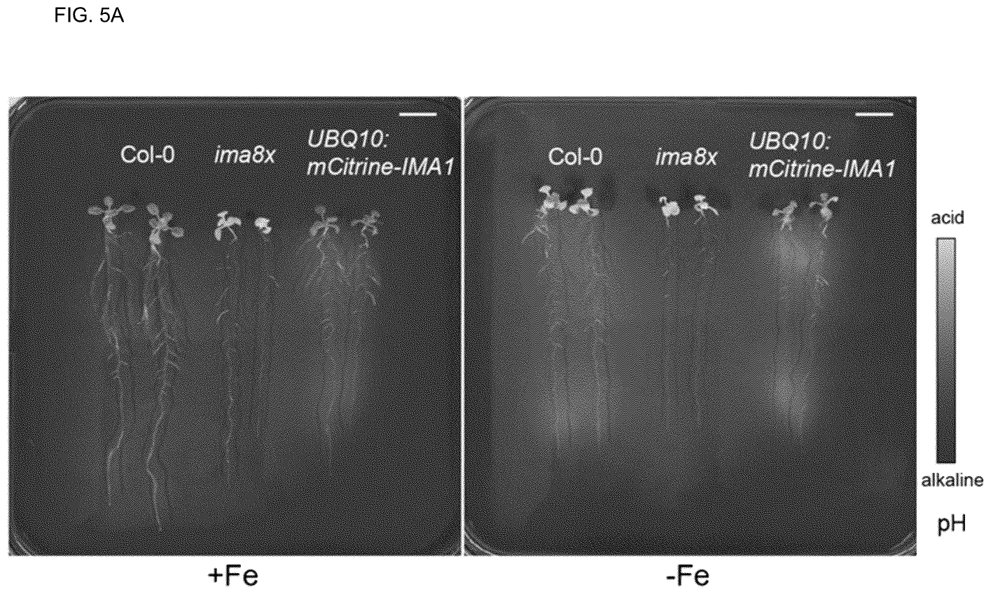

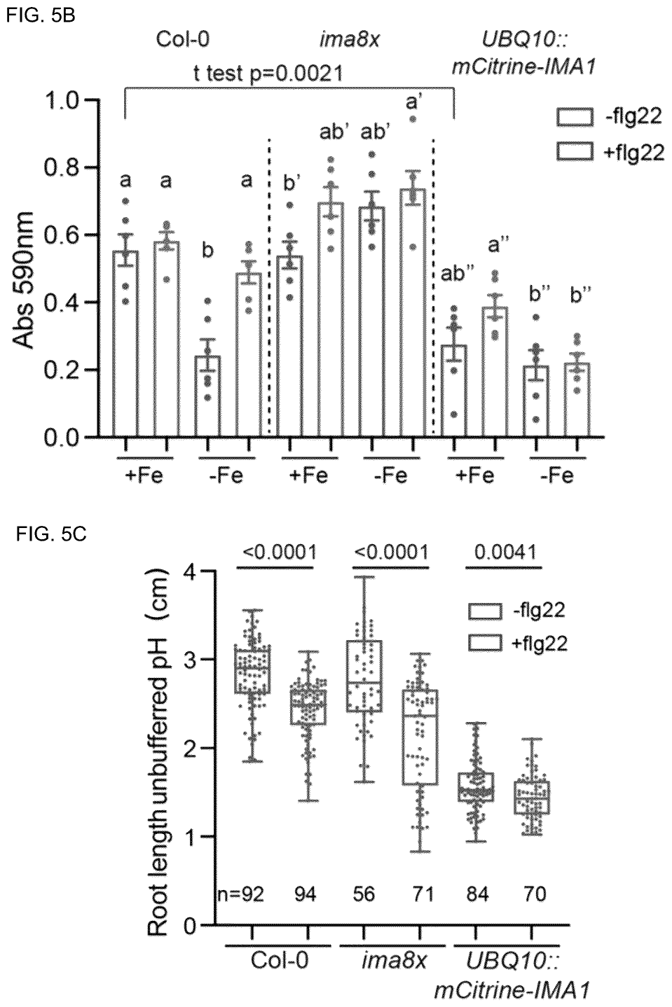

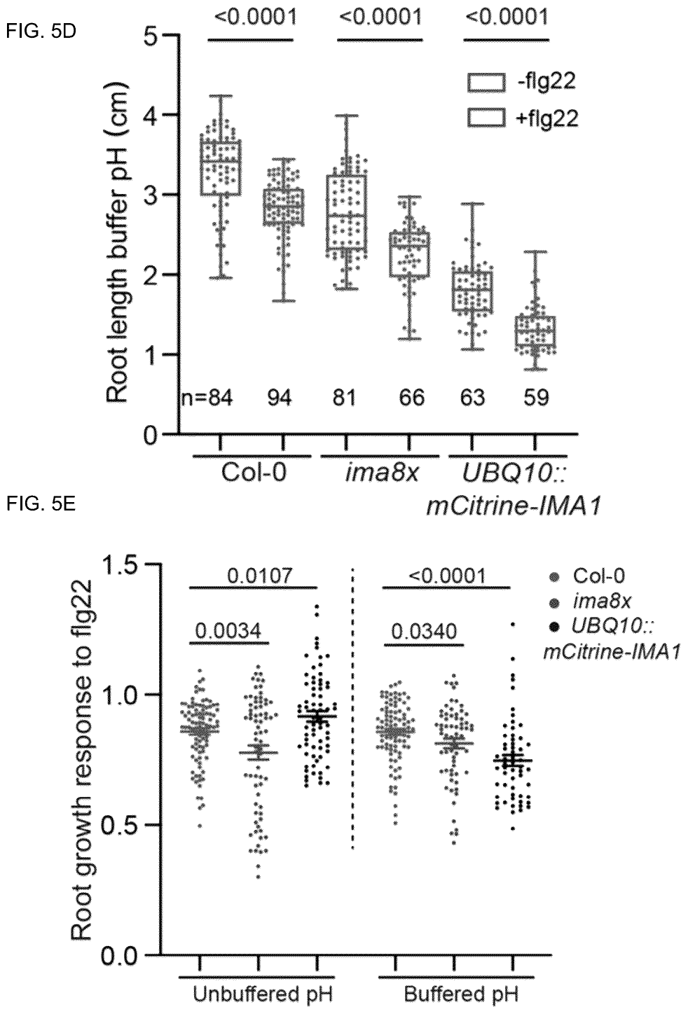

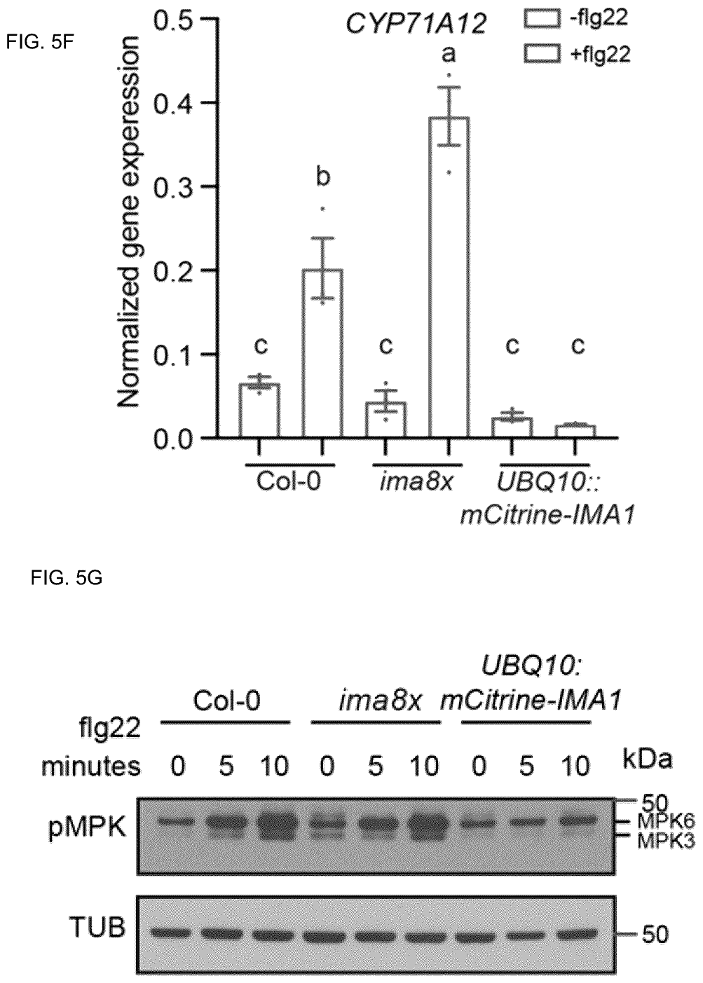

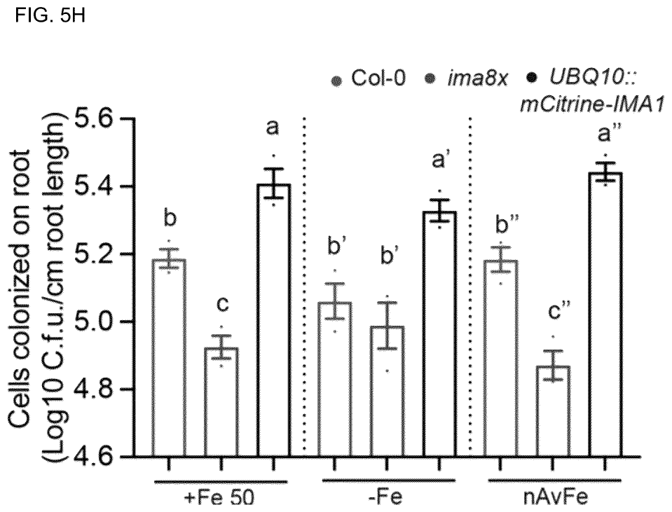

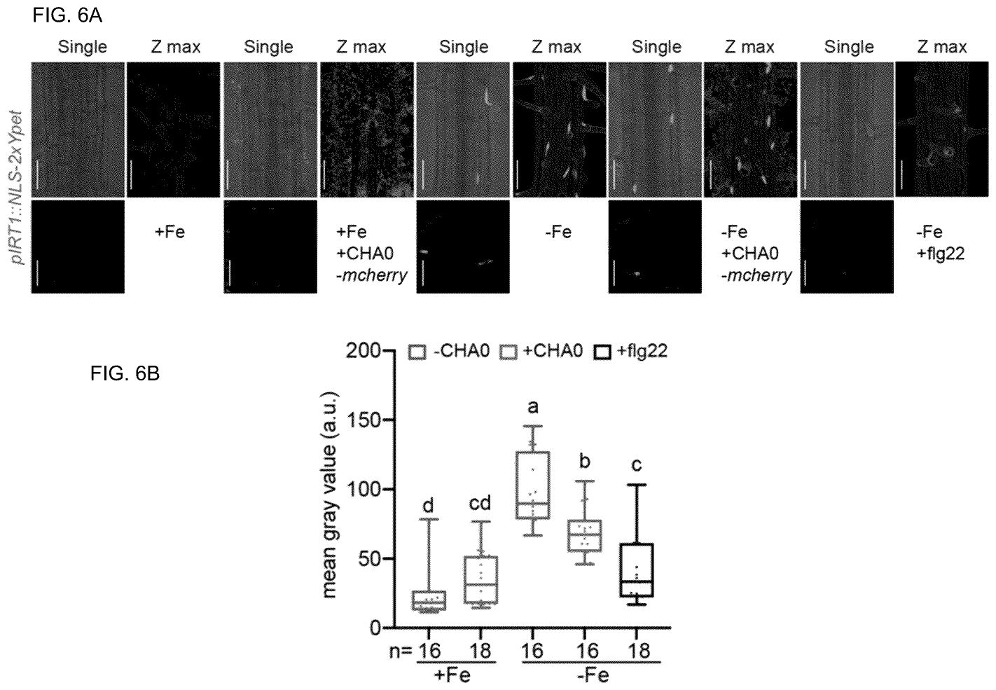

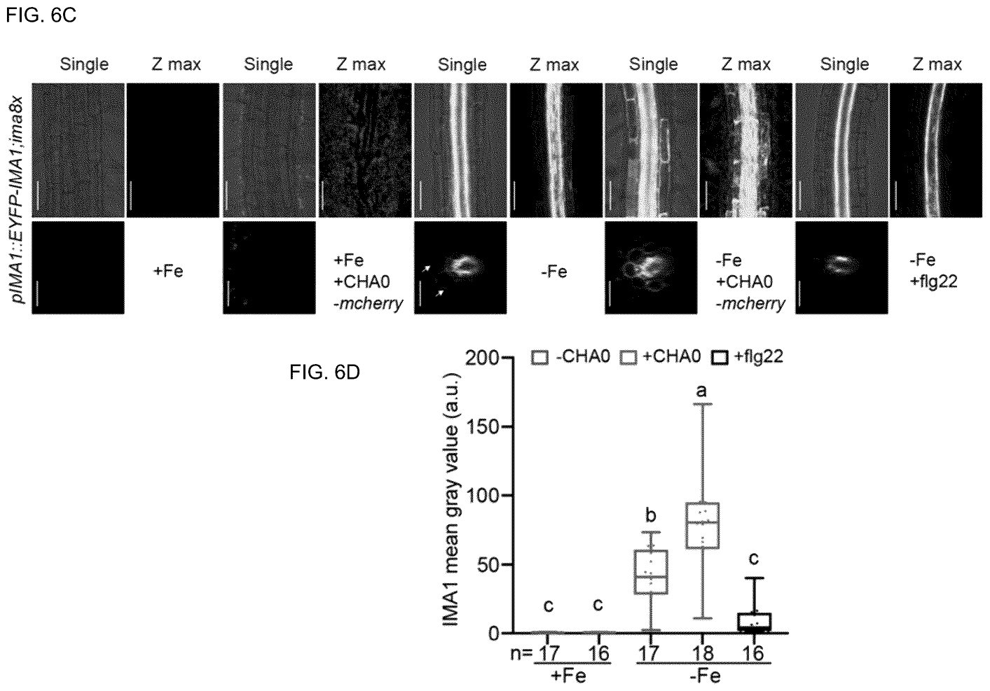

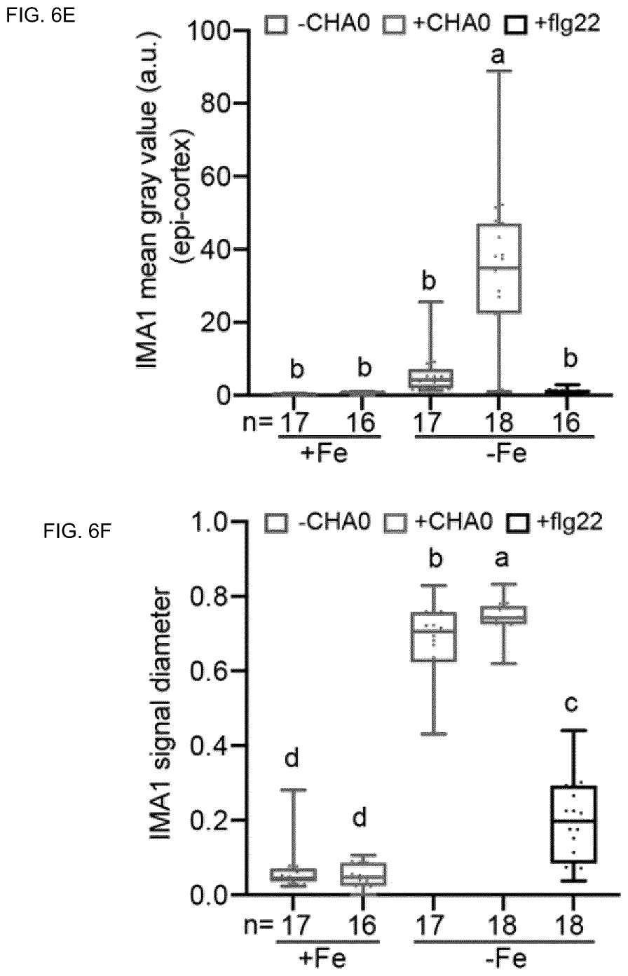

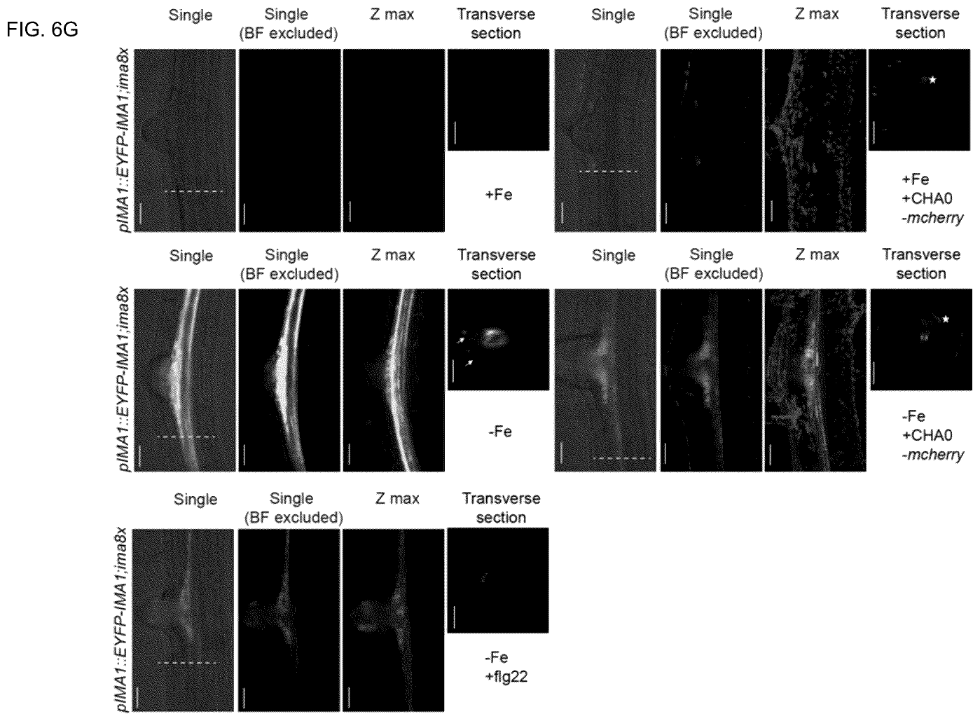

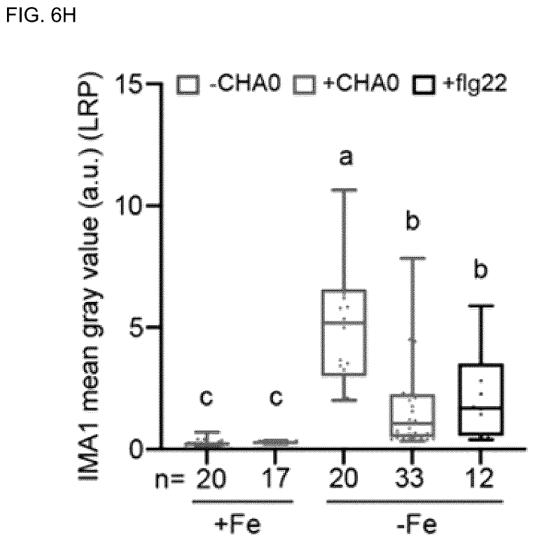

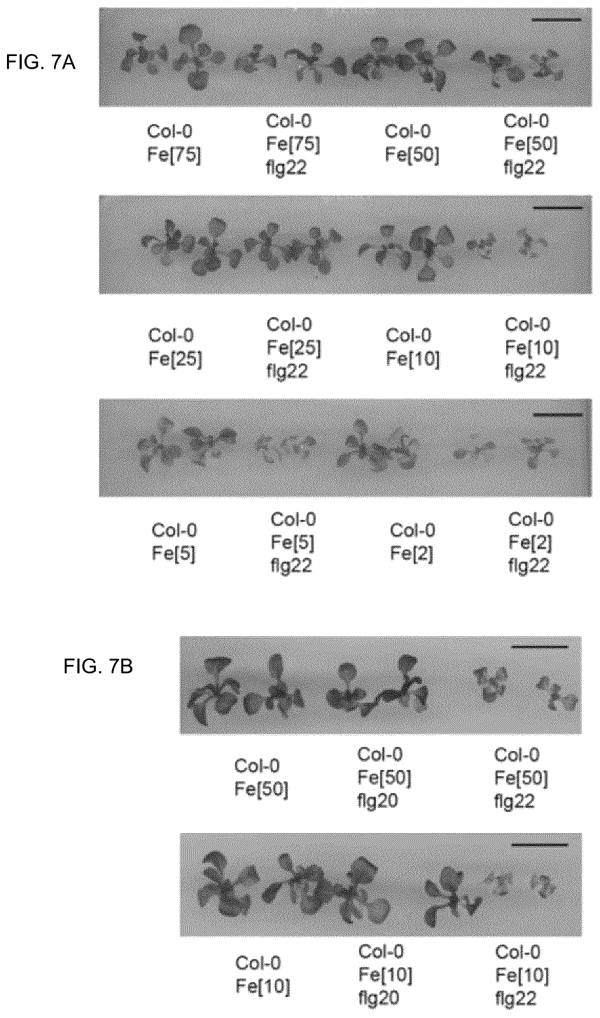

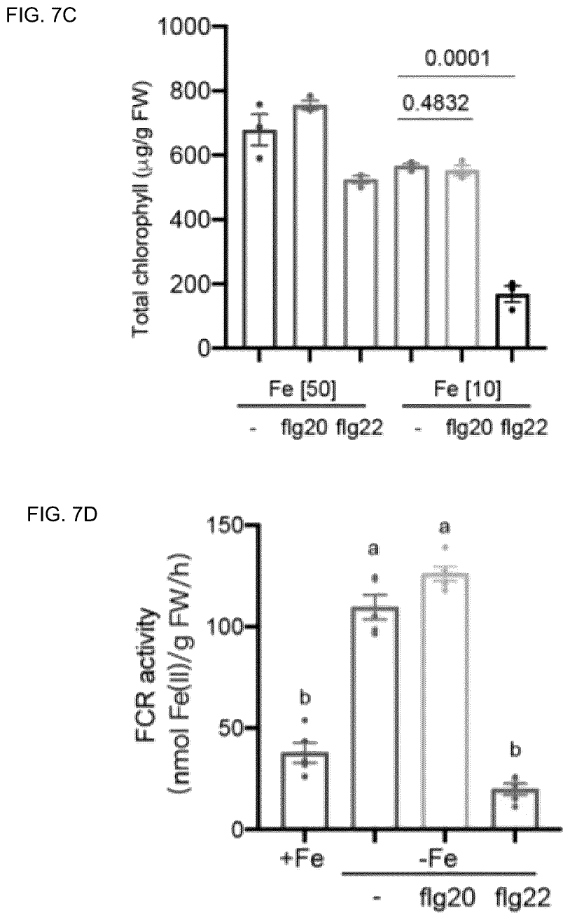

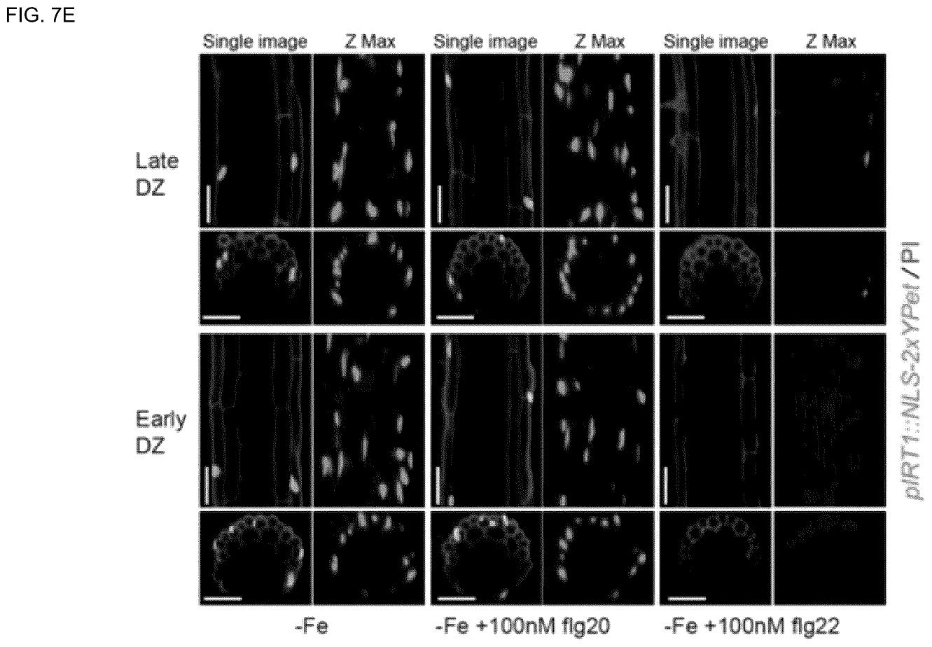

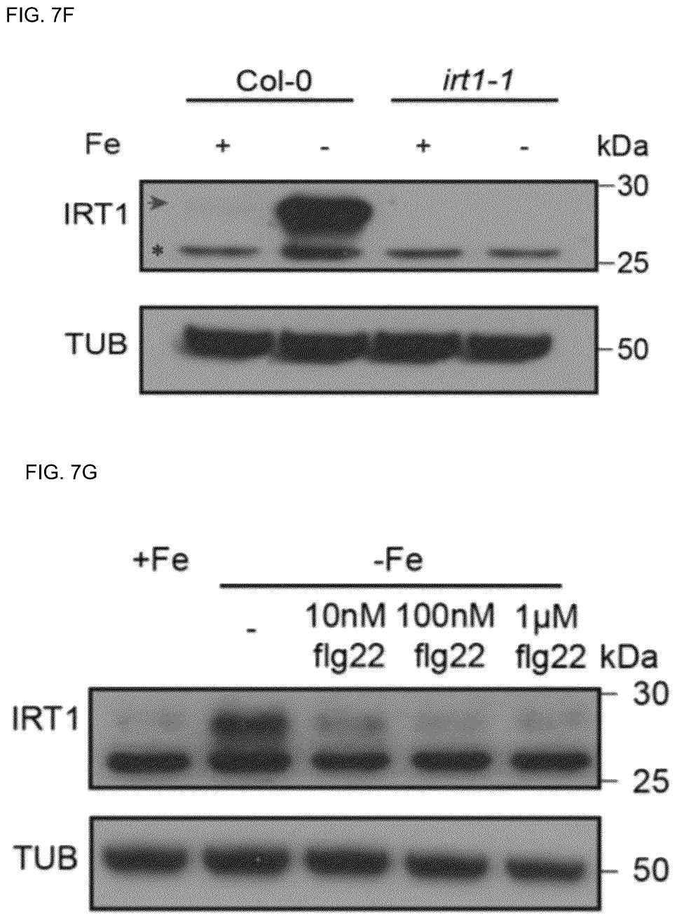

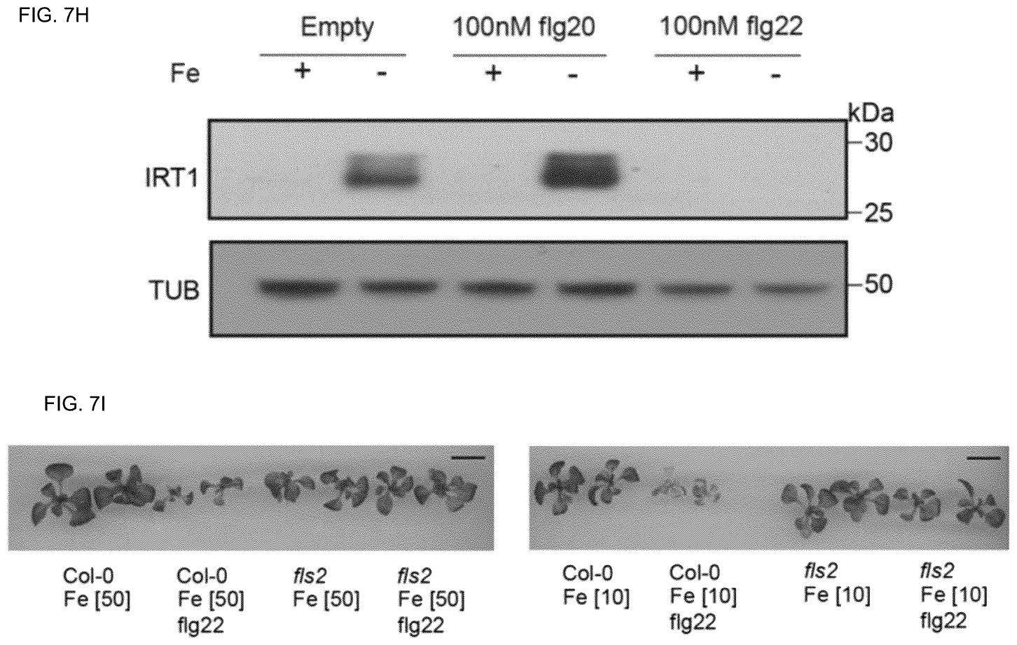

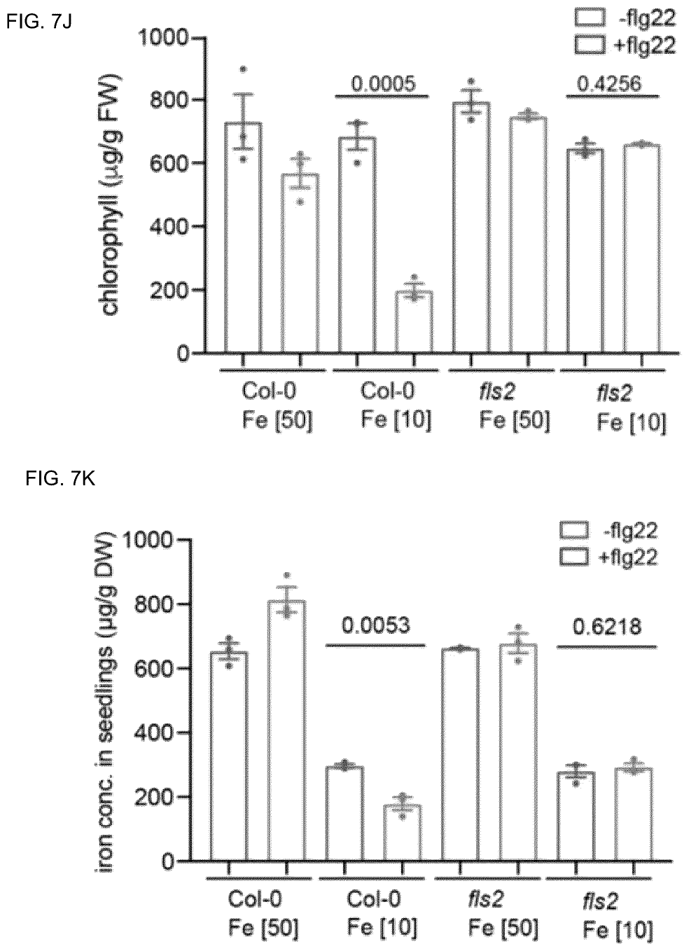

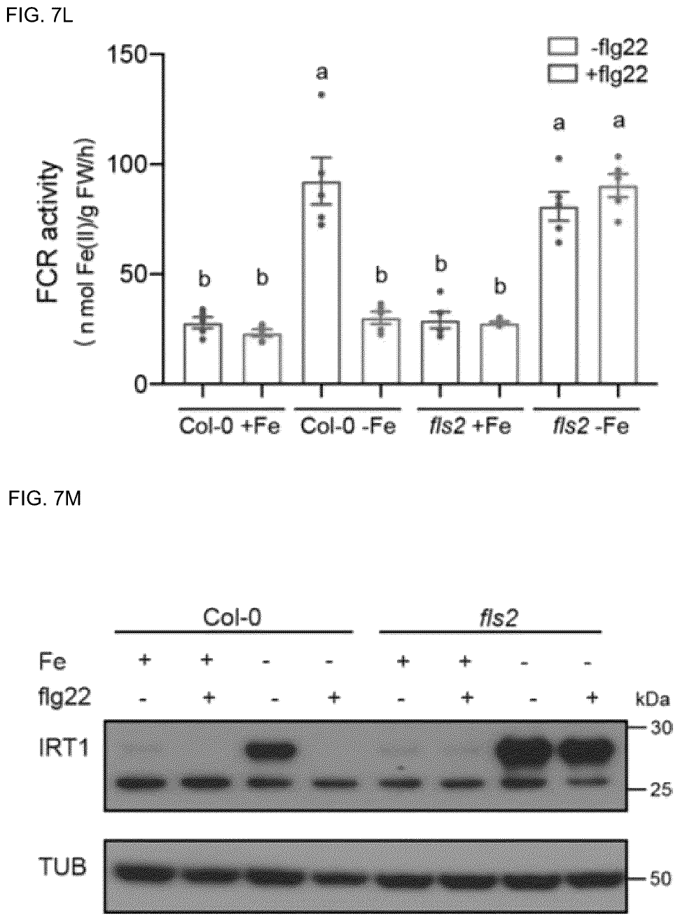

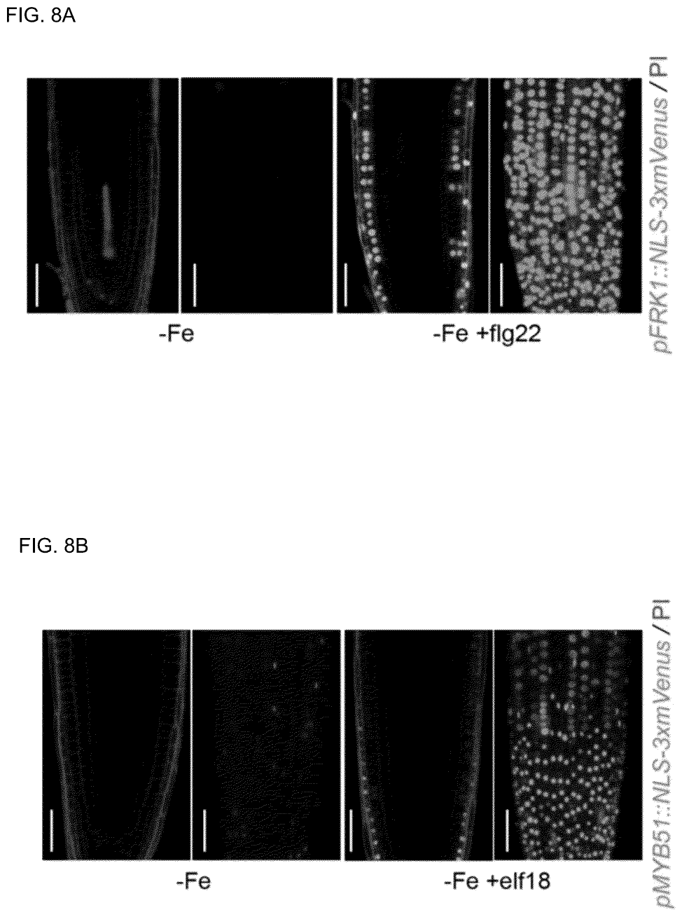

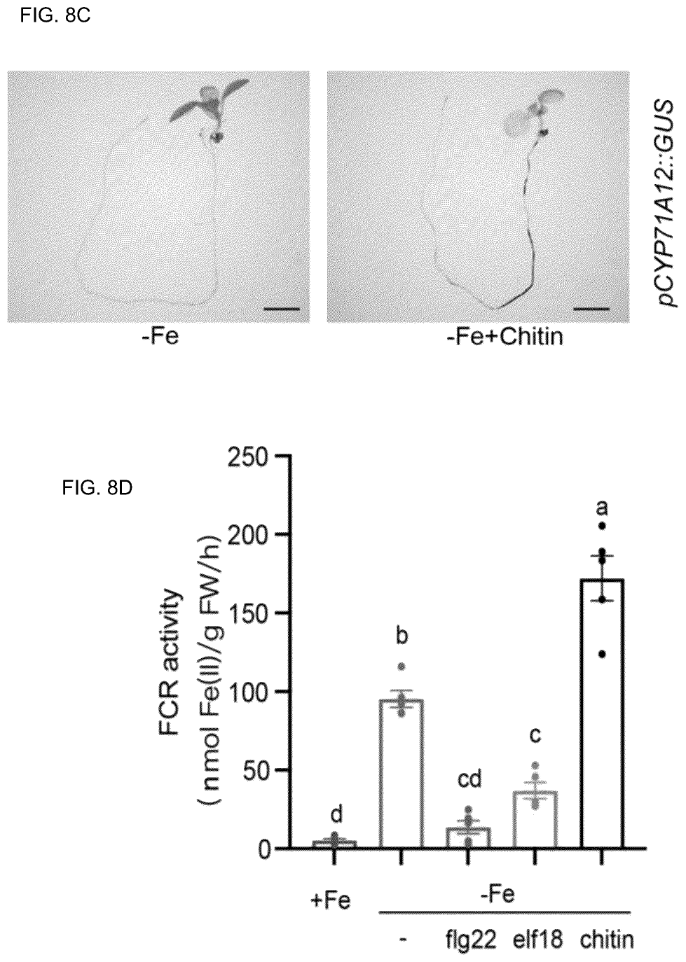

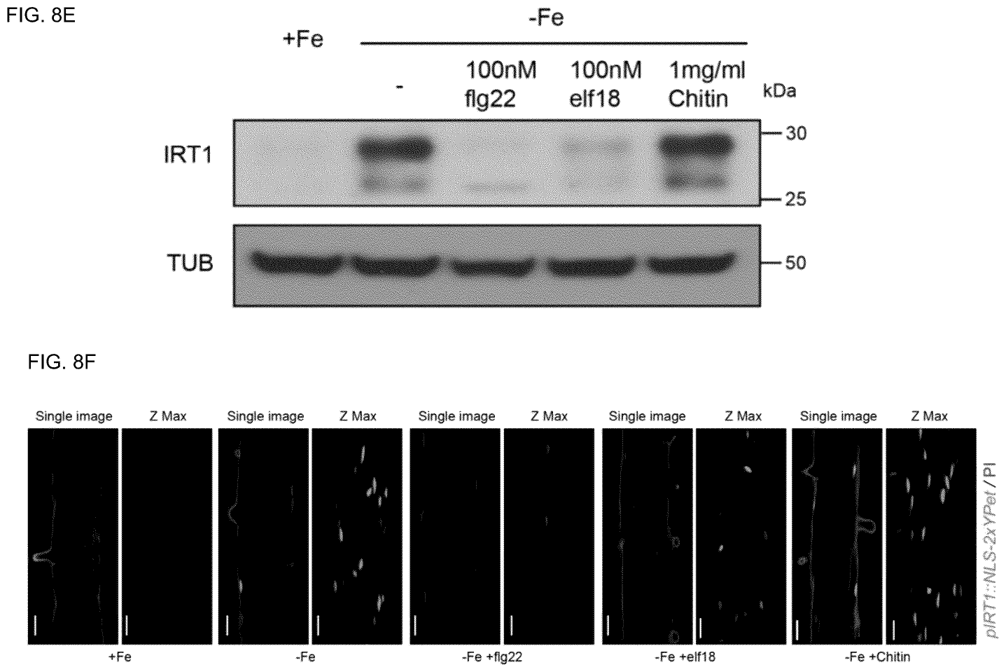

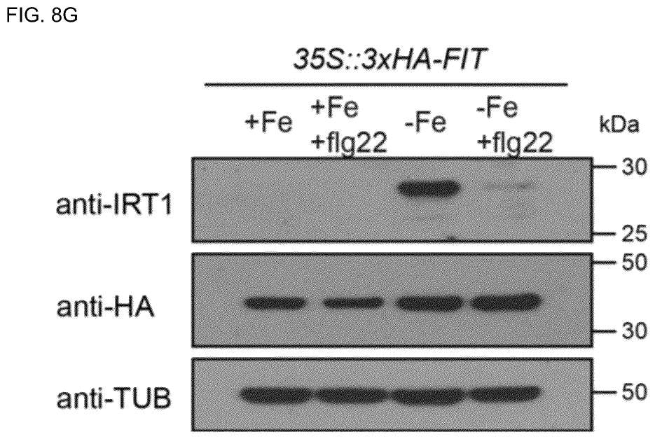

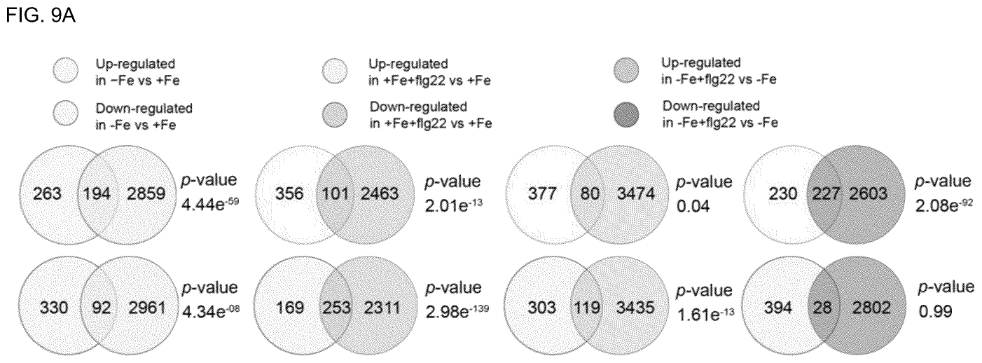

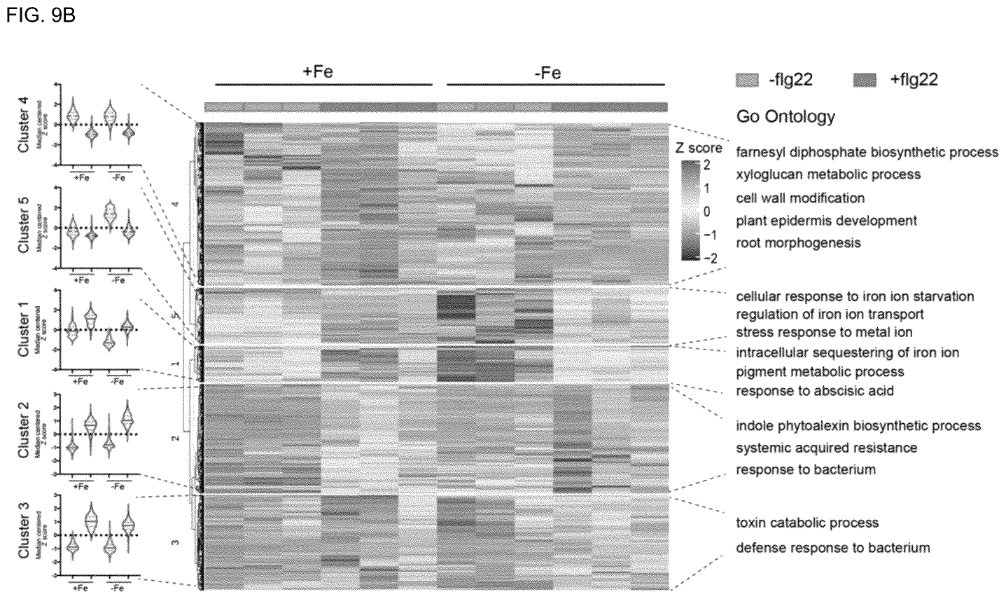

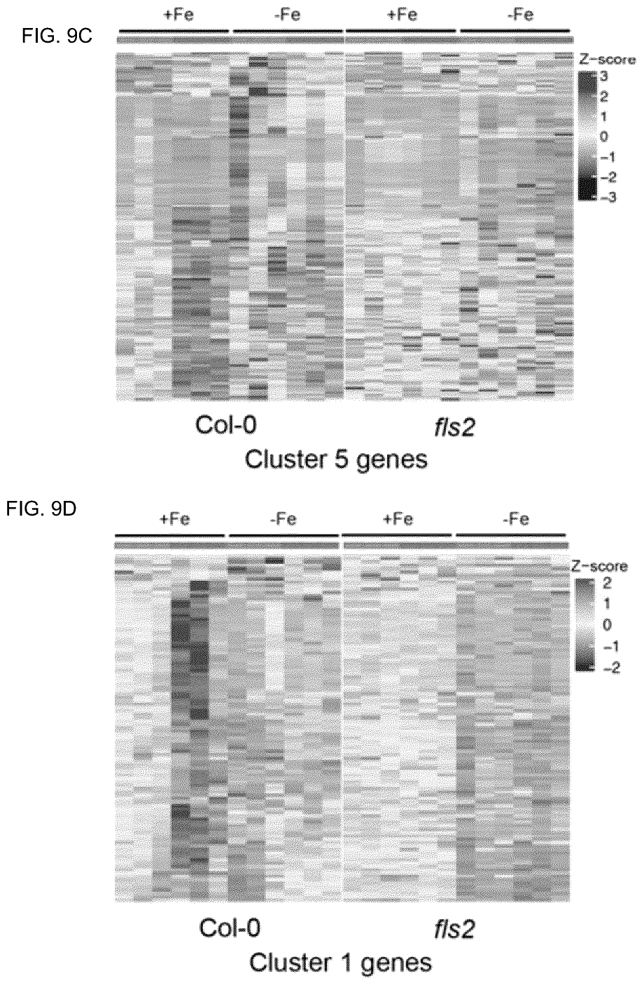

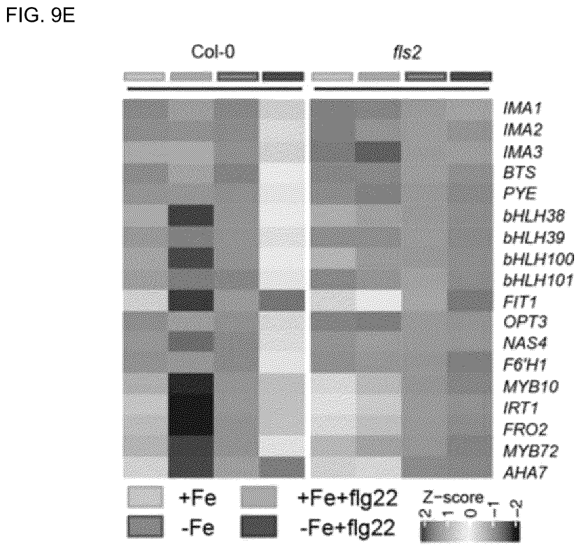

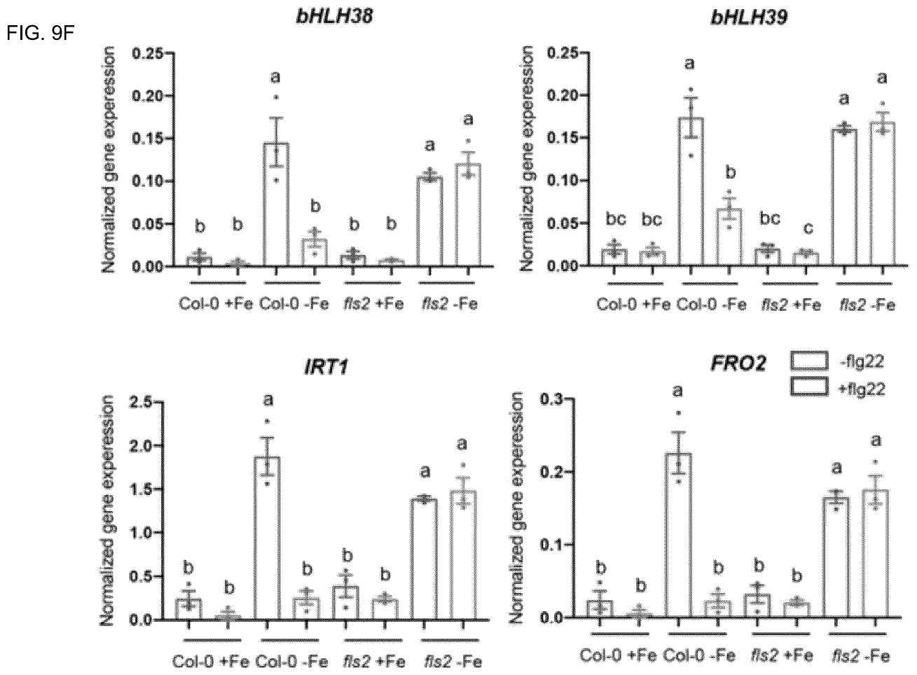

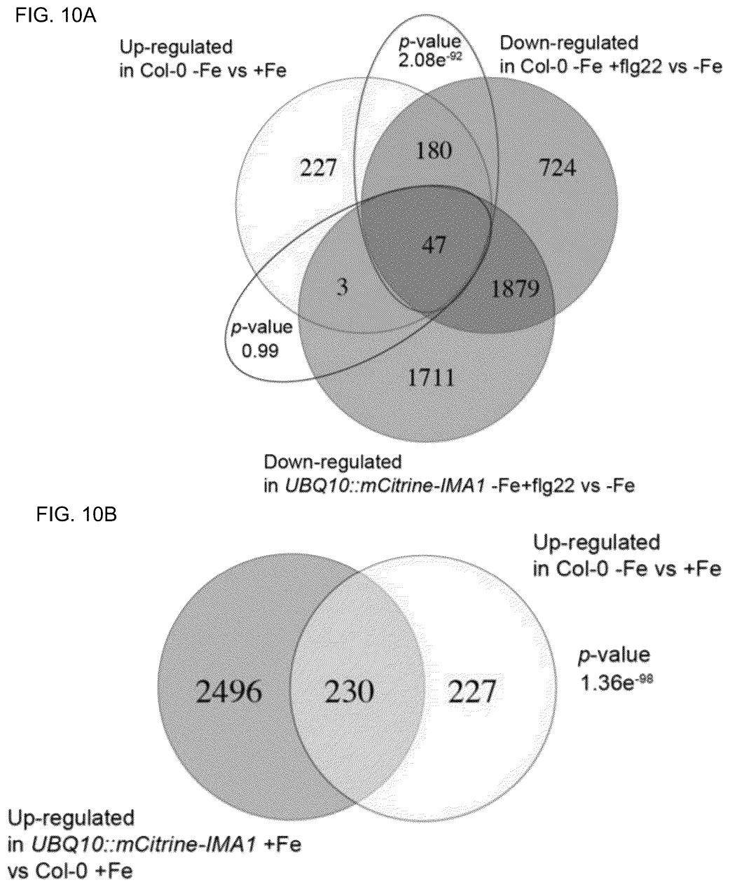

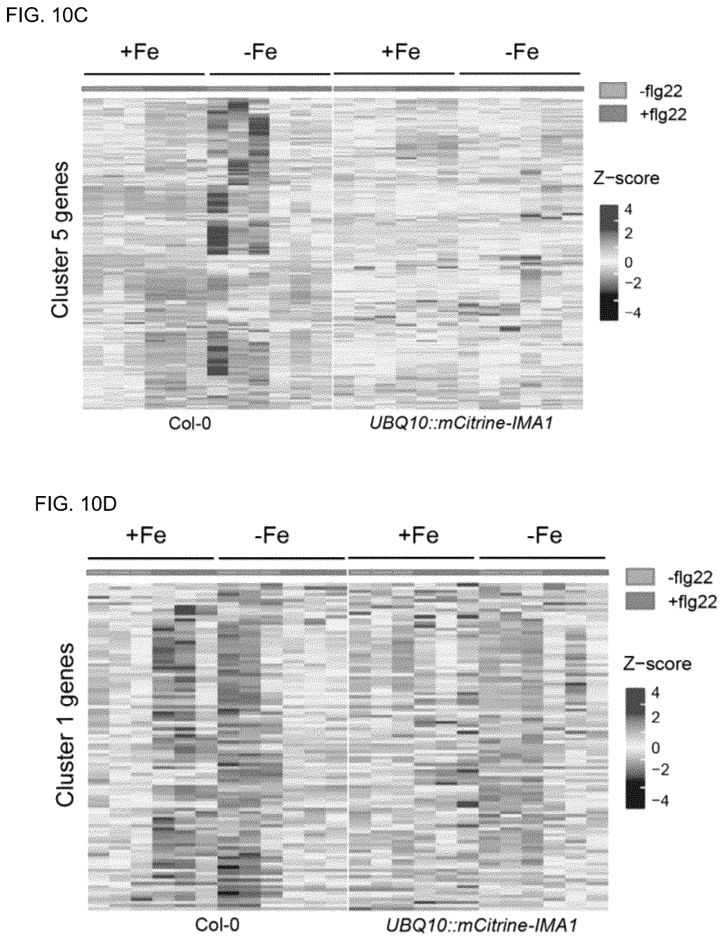

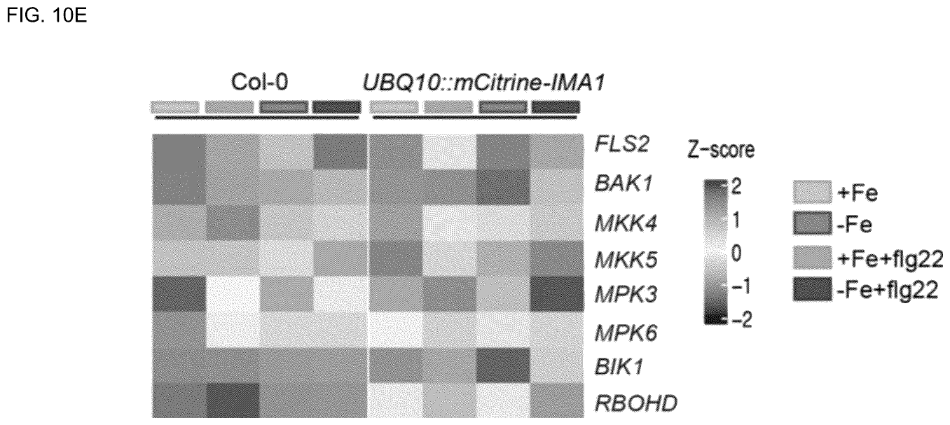

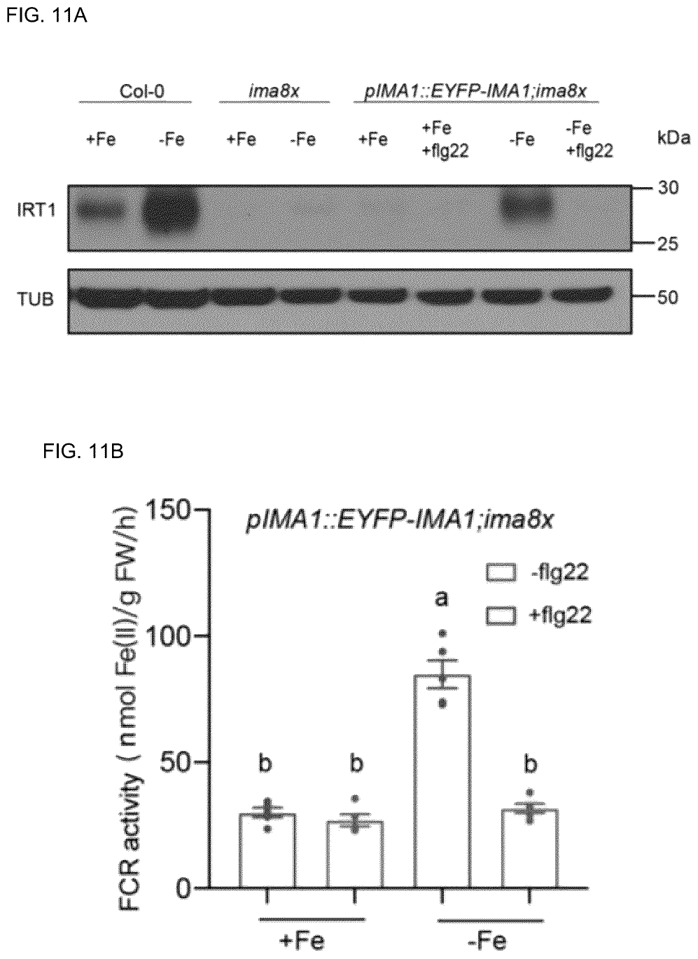

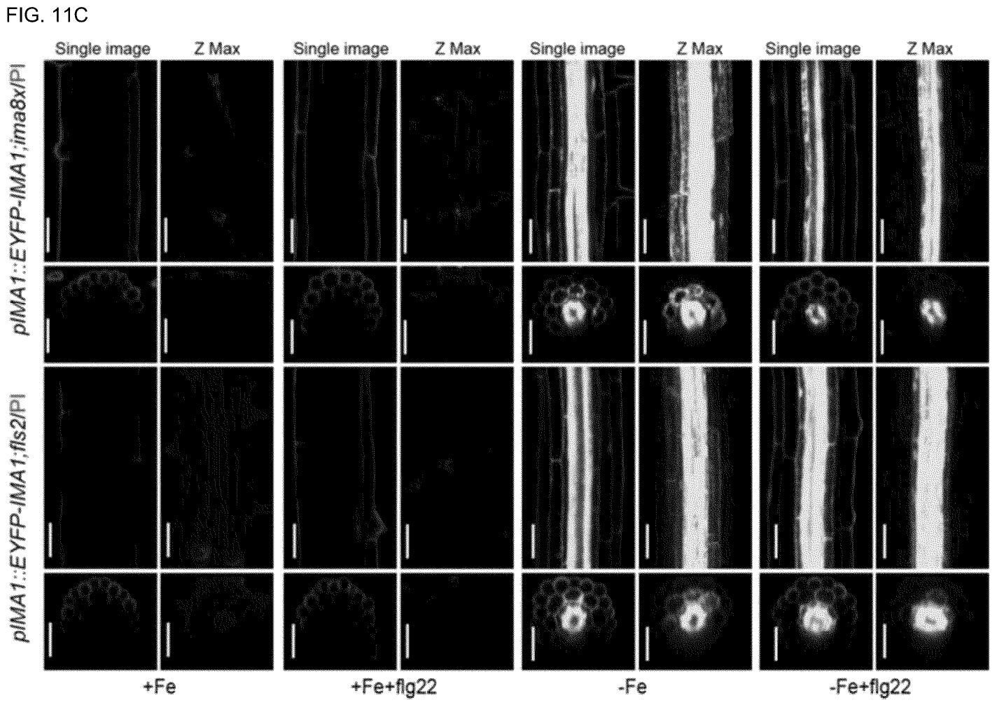

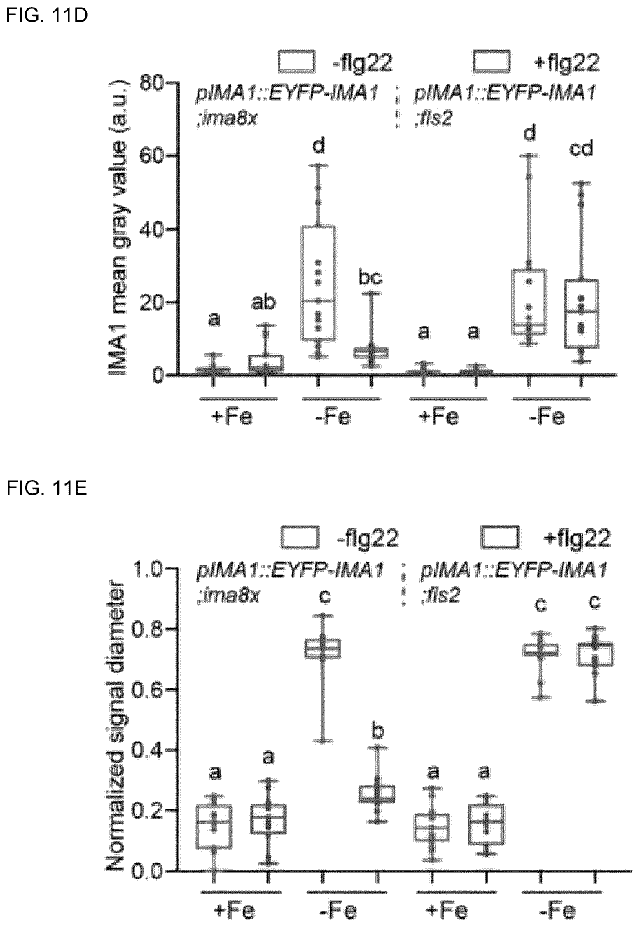

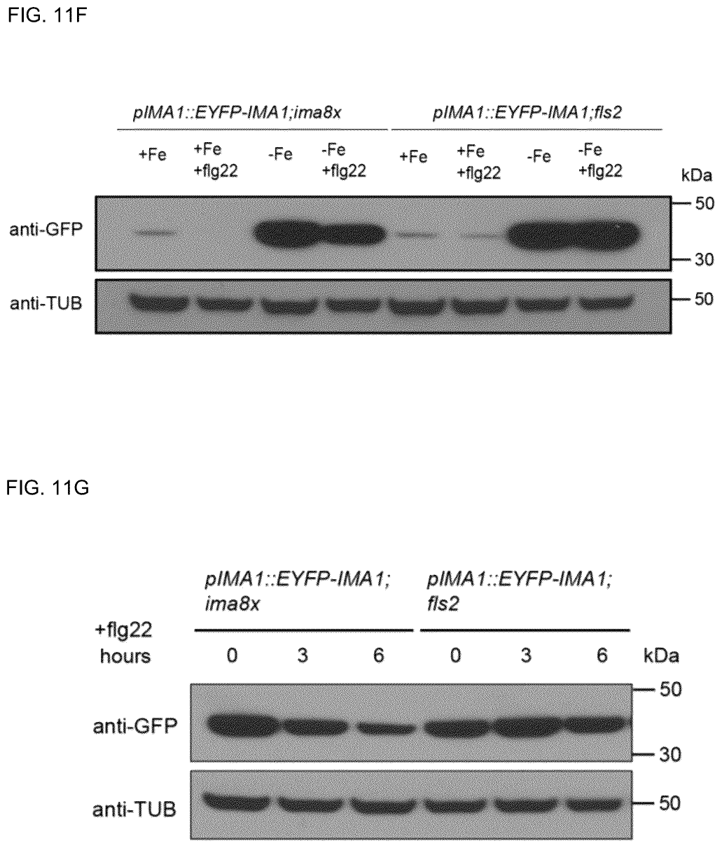

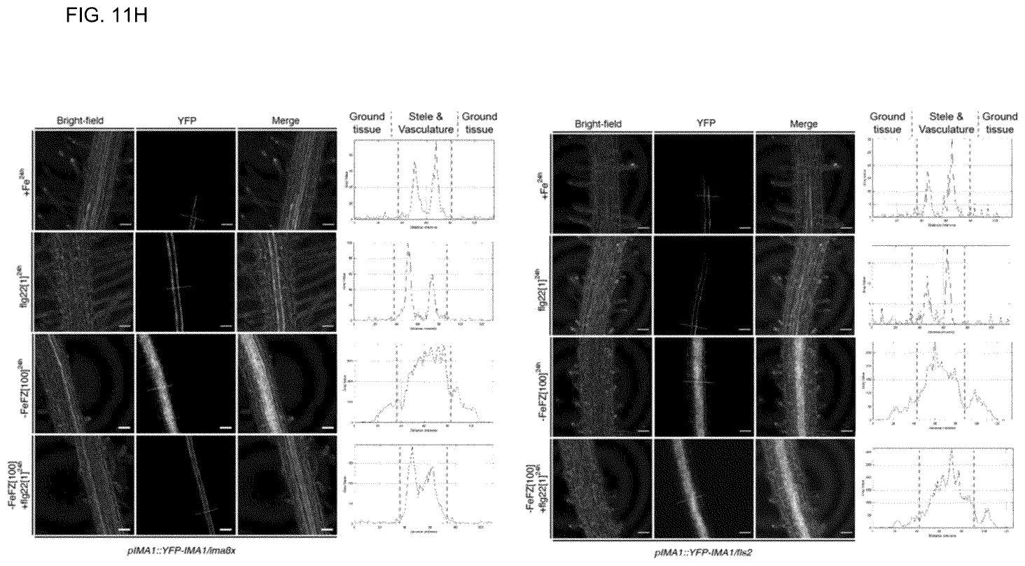

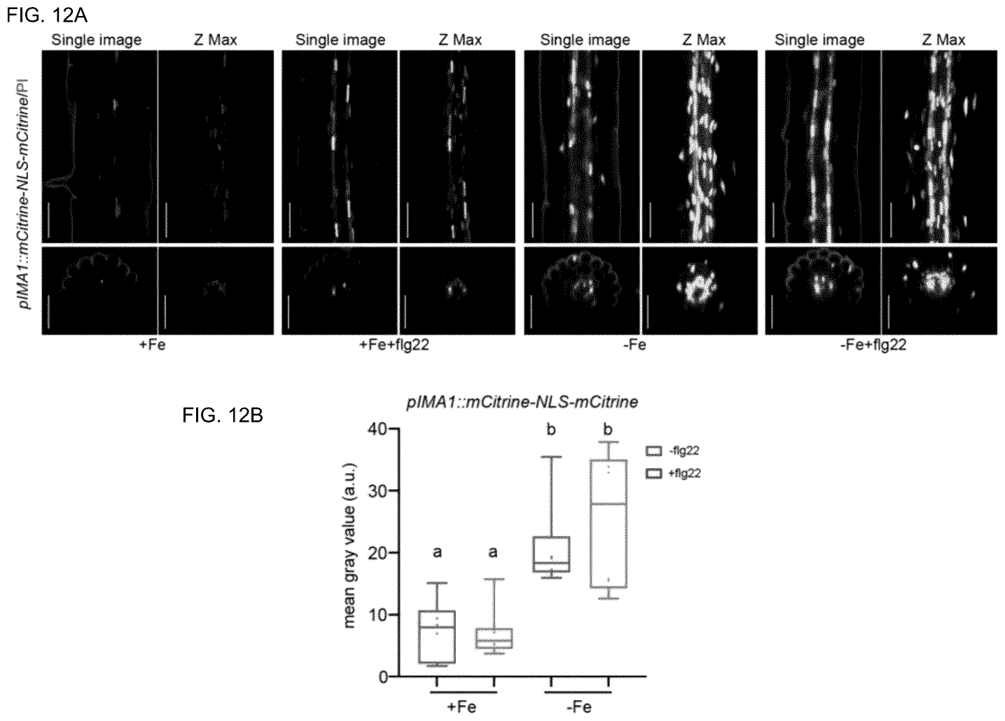

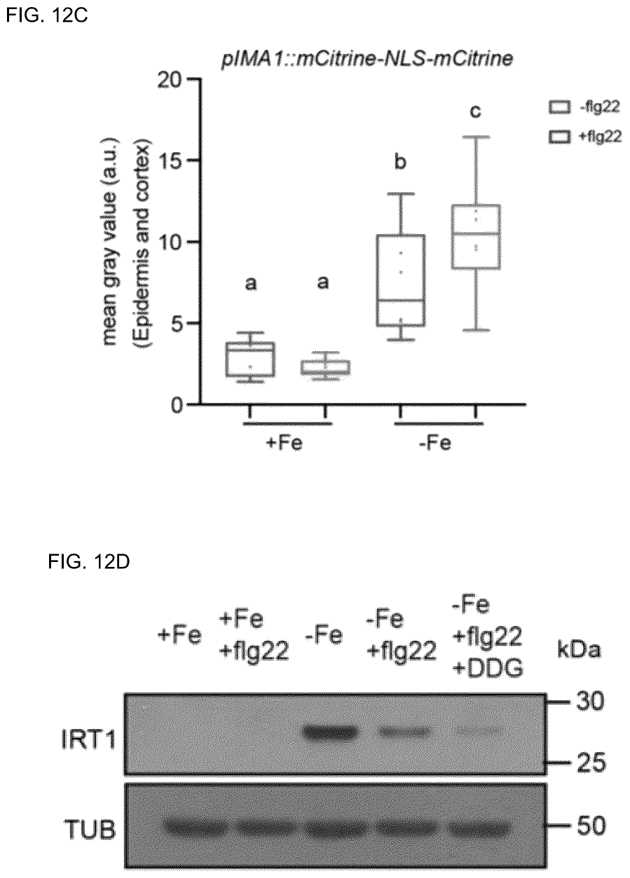

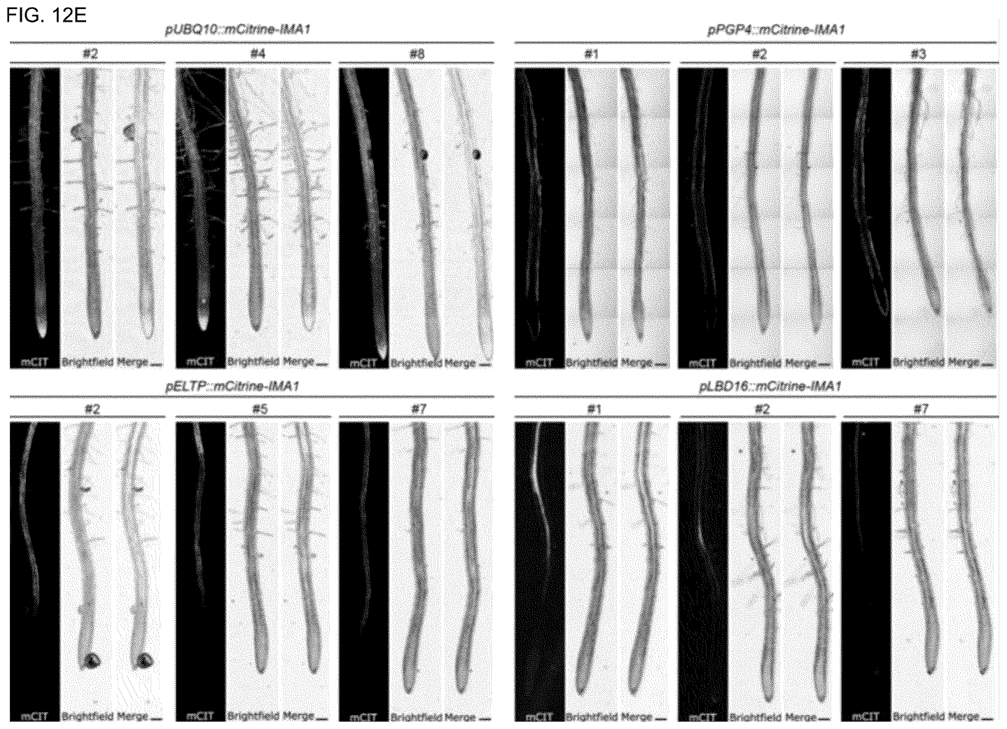

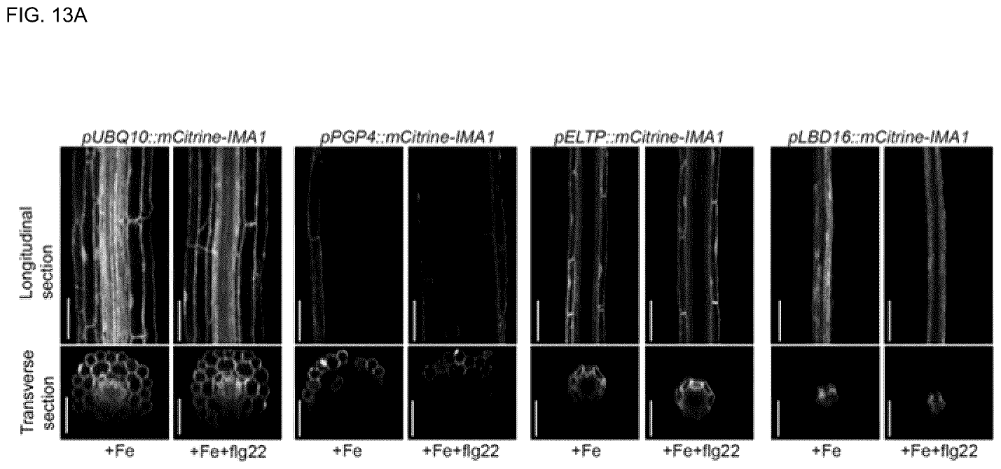

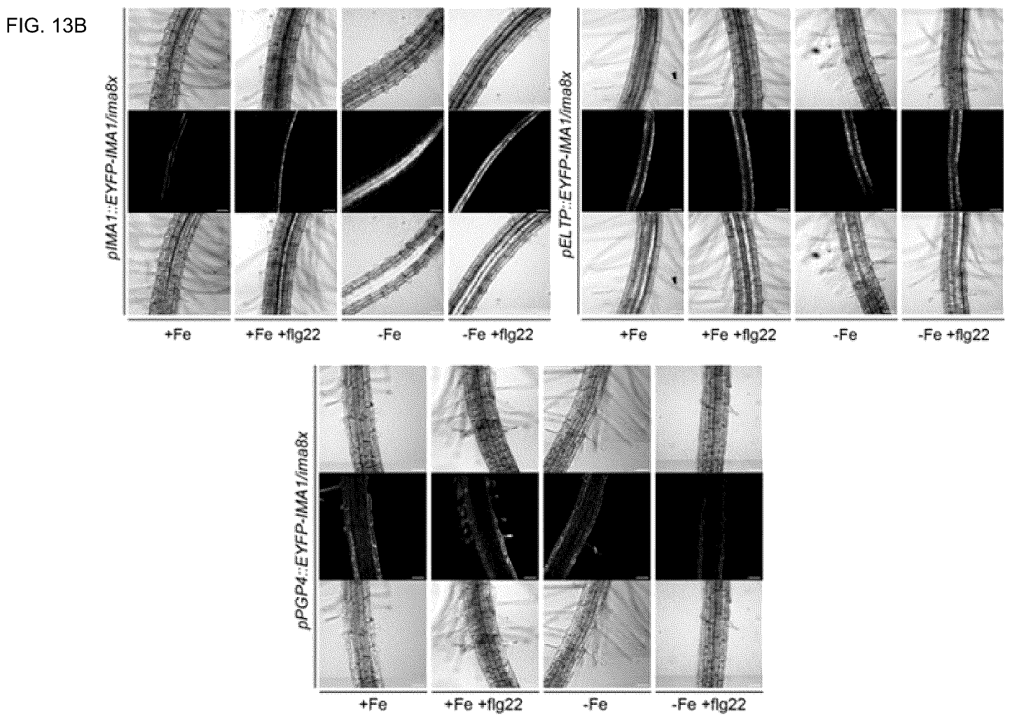

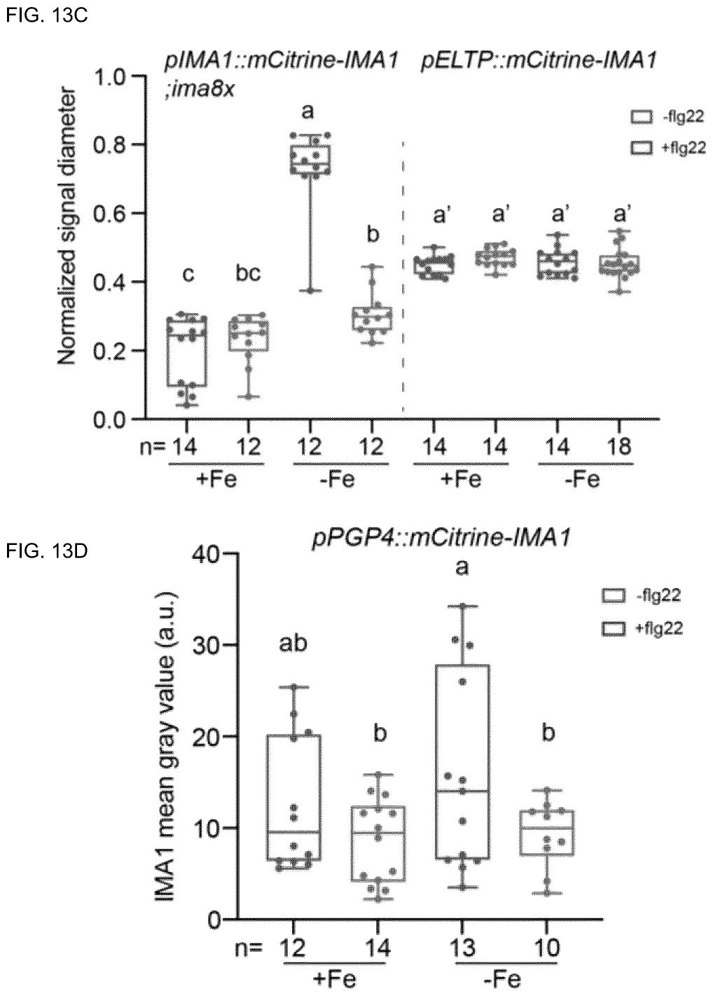

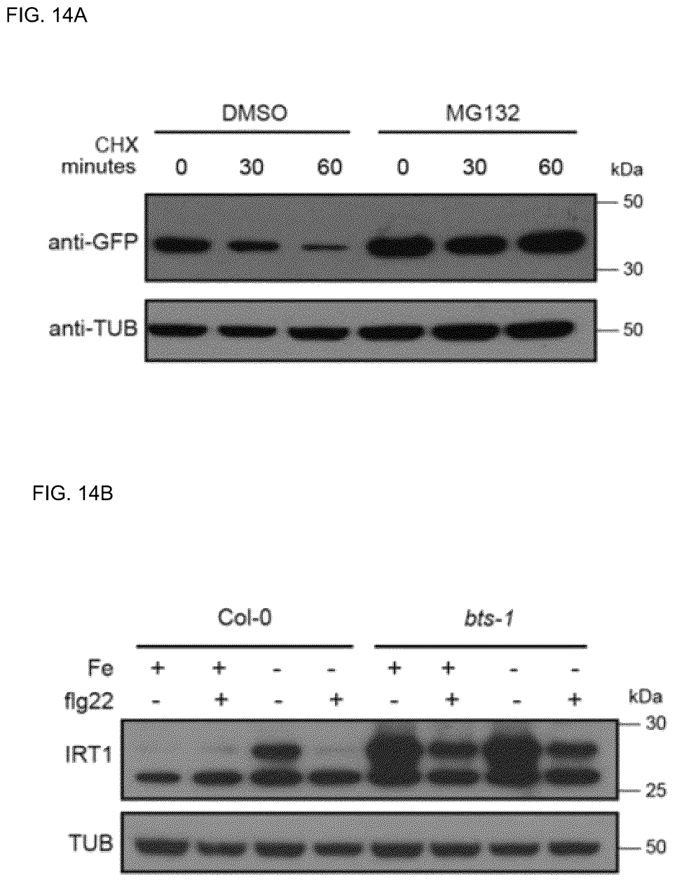

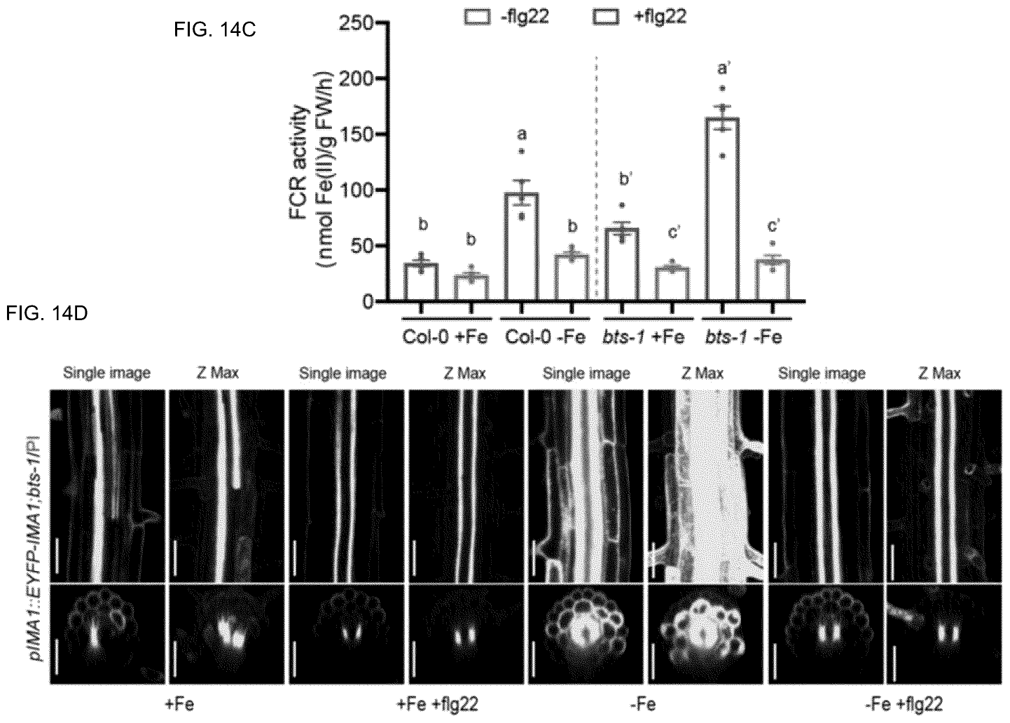

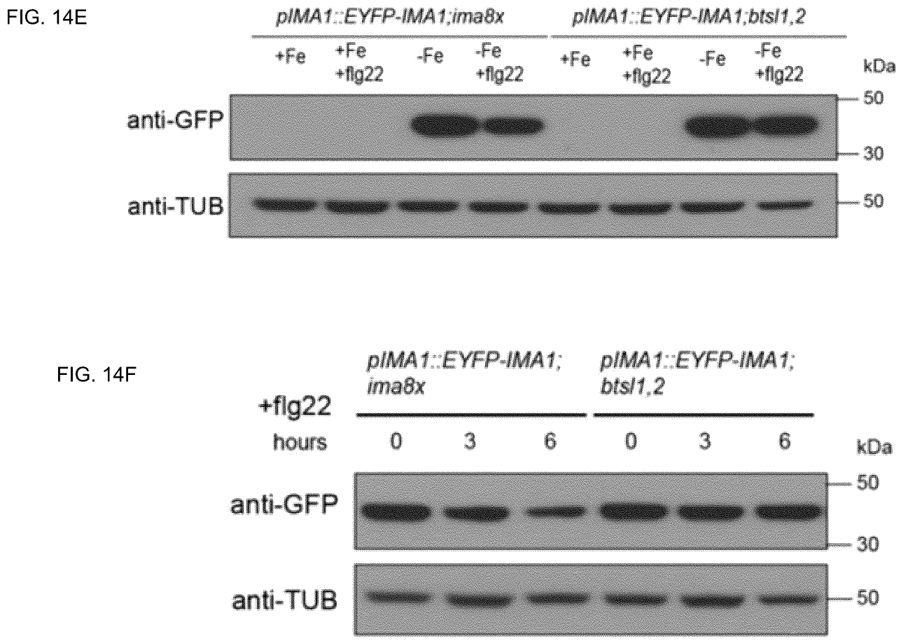

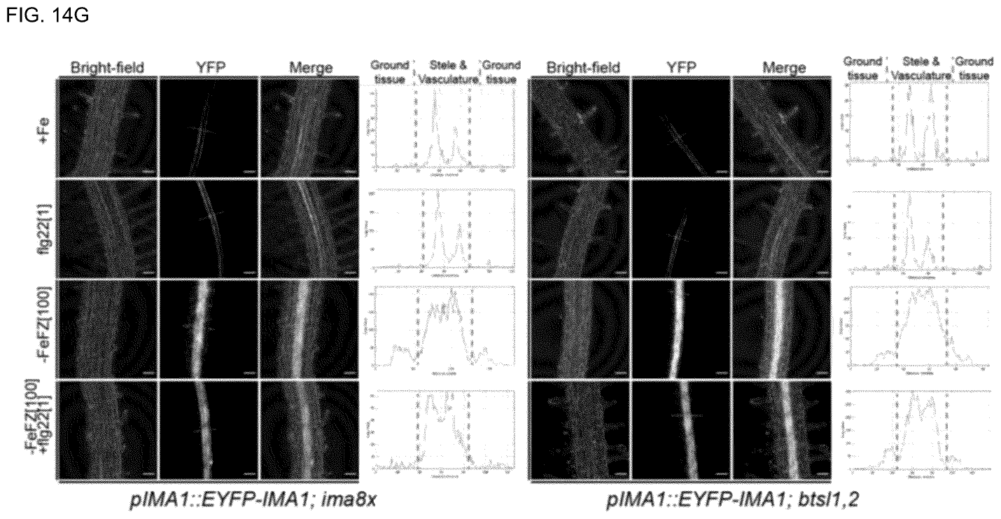

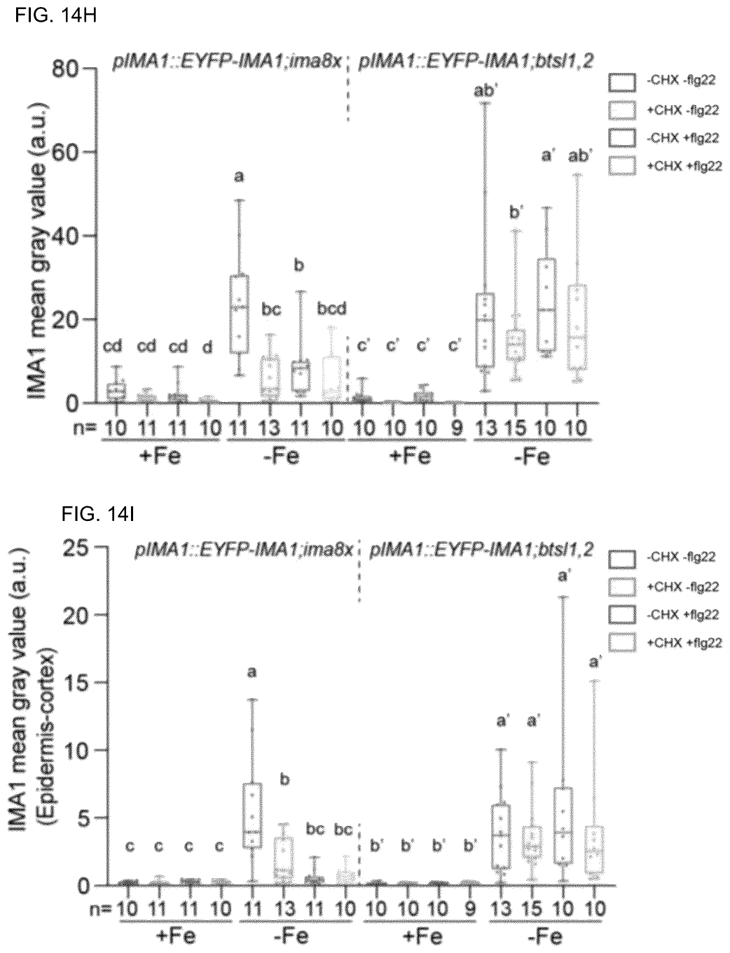

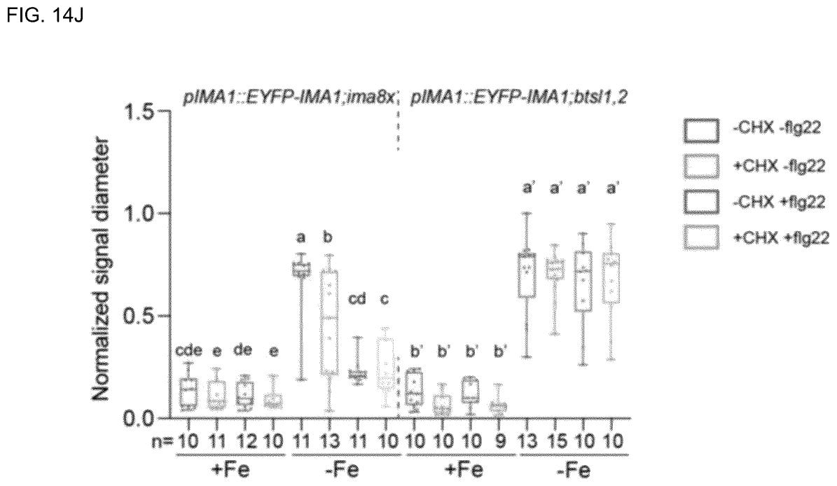

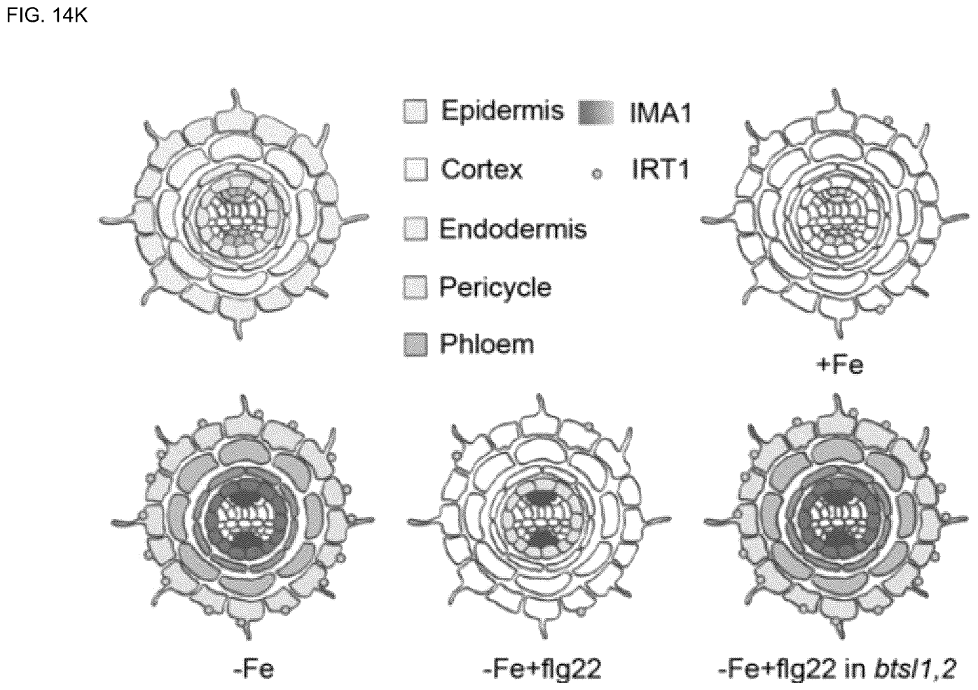

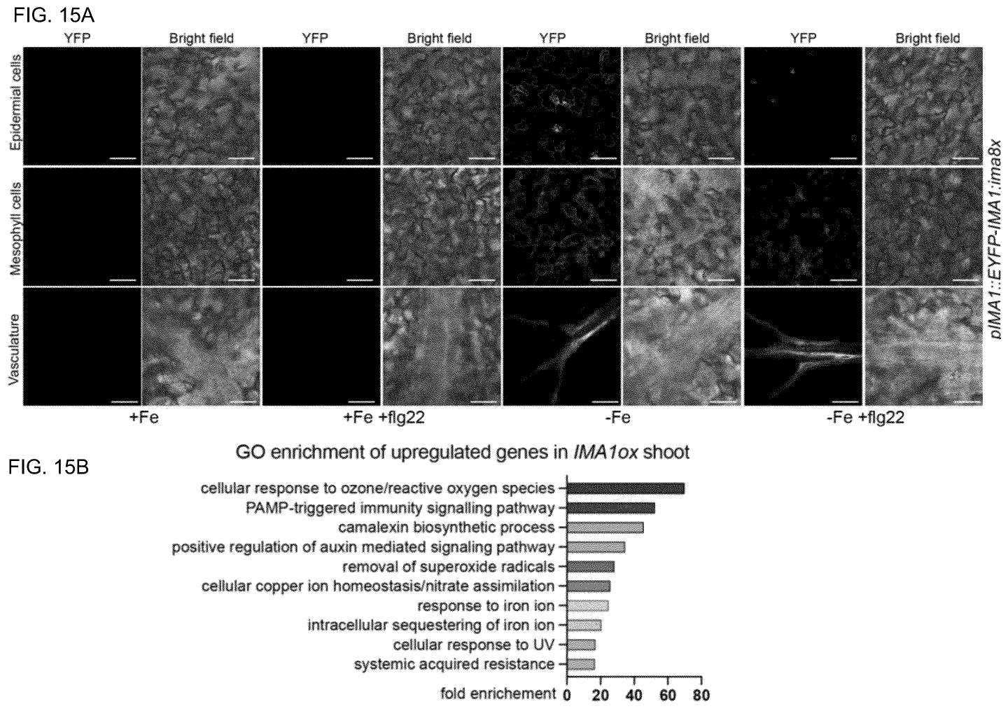

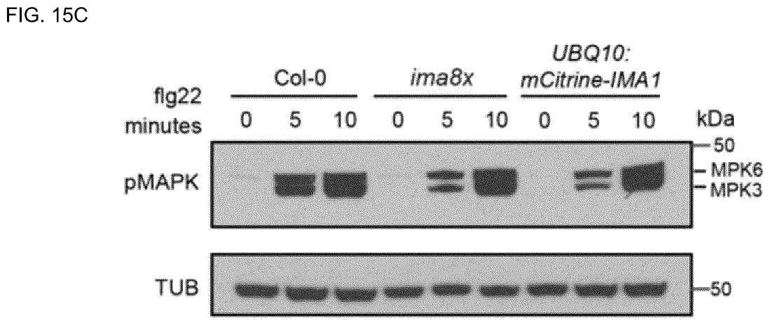

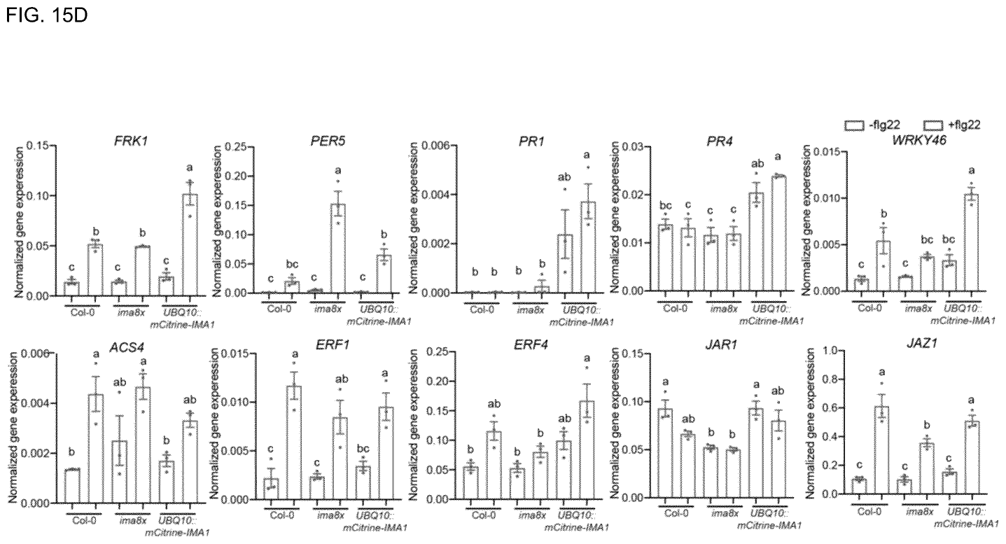

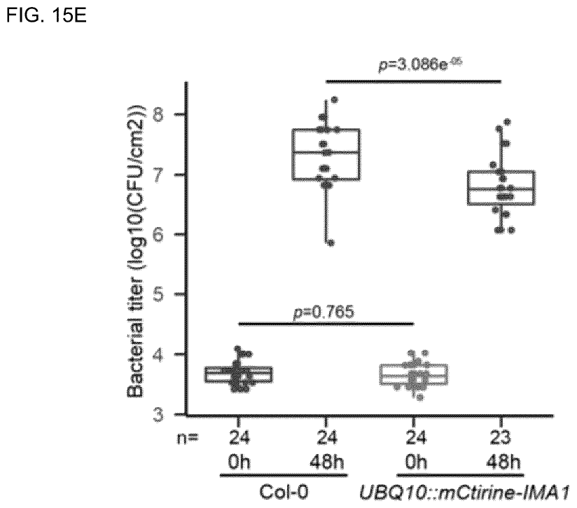

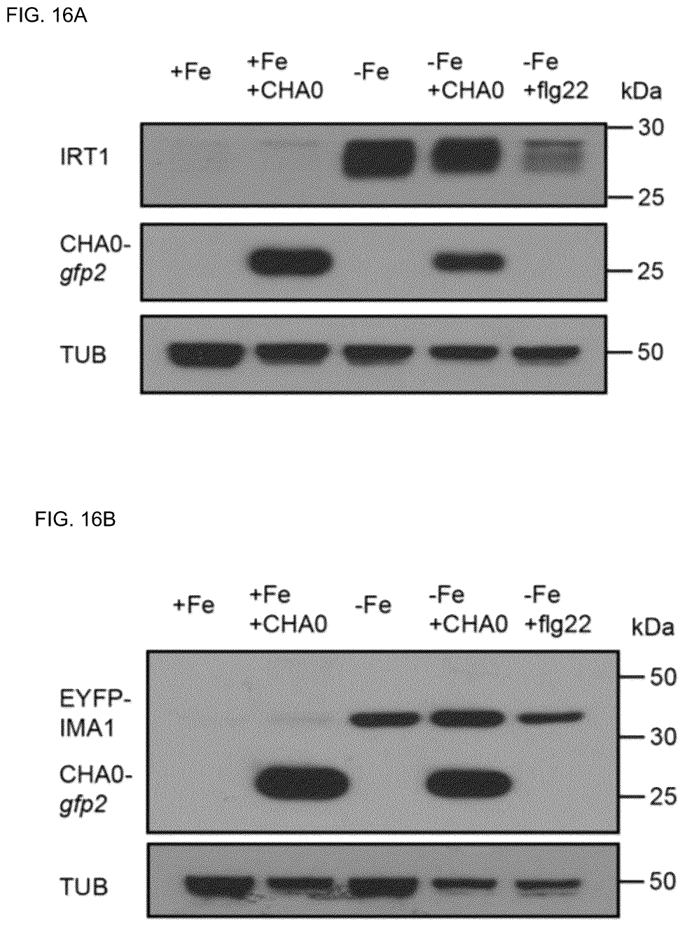

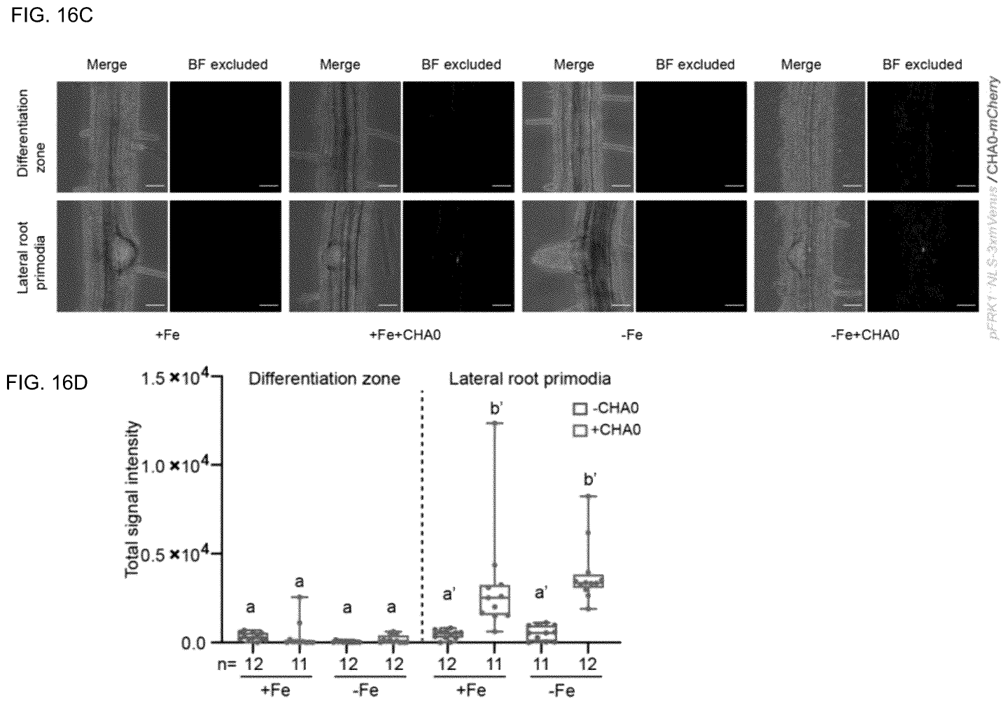

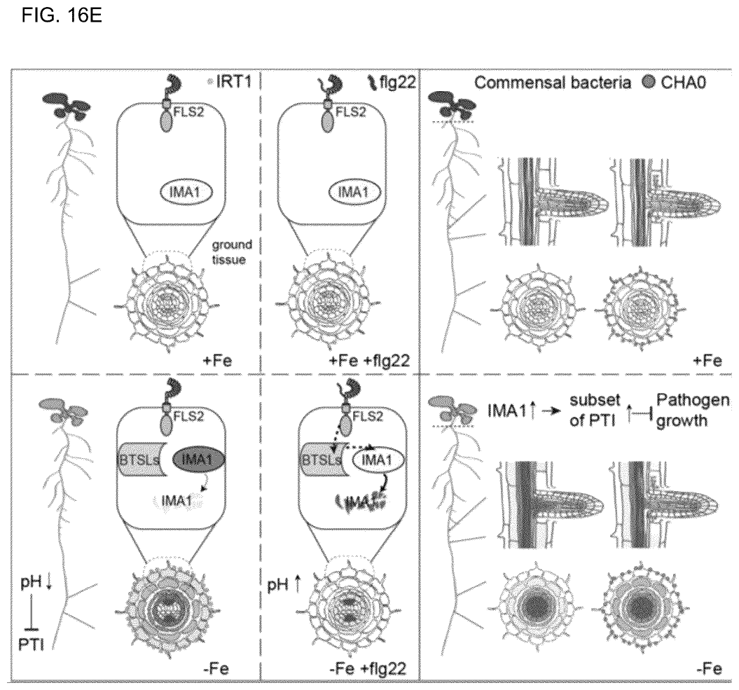

A pronounced interplay of responses to low iron and immunity within the first few hours of roots exposed to an iron depletion environment was previously reported. To investigate this interplay at a longer timescale, Arabidopsis seedlings were grown in iron sufficient and low iron media with and without the elicitor flg22, a peptide fragment of bacterial flagellin (FIG. 1A). Compared to seedlings grown under iron sufficient conditions (+Fe), seedlings grown on low iron conditions (−Fe) were slightly more chlorotic and contained less iron. This was drastically exacerbated when co-treated with flg22 (FIGS. 1A-IC; FIG. 7A), but not when treated with a non-immunogenic form of flagellin, Flg20 (FIGS. 7B-7C). It is therefore hypothesized that flg22 abolishes root iron uptake. To test this hypothesis, ferric chelate reductase (FCR) activity and expression of the IRON REGULATED TRANSPORTER1 (IRT1) were measured, which are part of the canonical iron deficiency response in Arabidopsis and facilitate iron uptake. While FCR activity in roots was induced under −Fe without flg22 treatment, FCR activity was not induced in −Fe in the presence of flg22 (FIG. 1D; FIG. 7D). Likewise, lack of iron triggered the induction of the pIRT1::NLS-2xYpet marker in the epidermal cells of the differentiation zone, and treatment with flg22, but not flg20, abolished these responses (FIGS. 1E-1F; FIG. 7E). IRT1 protein levels reflected this response (FIG. 1G; FIGS. 1F-1H). This was dependent on flg22 reception by the FLS2 immune receptor as flg22-induced chlorosis, and flg22-triggered FCR activity and IRT1 repression in −Fe conditions were abolished in fls2 mutants (FIGS. 7I-7M). Overall, these data demonstrated that flg22-elicited immune responses can repress the iron deficiency program. Other microbe-associated molecular patterns (MAMPs) were then tested. The bacteria derived MAMPs, flg22 and elf18 triggered immune responses in the root tip and chitin, a fungal-derived MAMP, triggered immune responses in the differentiation zone as indicated by the upregulation of the reporters for several defense genes such as FRK1, MYB51 and CYP71A12 (FIGS. 8A-8C). Like flg22, elf18 also repressed FCR activity and IRT1 activation upon −Fe in differentiated epidermal cells (FIGS. 8D-8F). However, chitin treatment neither repressed FCR activity nor IRT1 activation in −Fe (FIGS. 8D-8F). Taken together, the results suggest that different MAMPs modulate iron deficiency responses in the root in a specific manner.

### Example 3

### IMA1 Facilitates Flg22 −Fe Crosstalk

The induction of FCR and IRT1 upon iron deficiency is controlled by the transcription factor FIT. However, flg22 was still able to strongly repress IRT1 protein accumulation in plants that constitutively overexpress FIT (FIG. 8G), suggesting that this response to flg22 is not dependent on a regulation of FIT. To elucidate the program responsible for the flg22 triggered repression of IRT1, an RNAseq of roots grown under +/−Fe and/or +/−flg22 was conducted. During the response to flg22, the expression of a large portion of iron responsive genes were modulated (FIG. 2A). The most significantly overlapping gene sets were constituted by genes that were downregulated in response to −Fe and upregulated in response to −Fe/+flg22 (hypergeometric test; P<2.98*10⁻¹³⁹), and vice versa (hypergeometric test; P<2.08*10⁻⁹²) (FIG. 9A). K-means clustering of all differentially expressed genes (DEGs) that responded to either flg22, −Fe, or both (−Fe with flg22 double treatment) led to 5 clusters whose genes showed distinct expression patterns (FIG. 9B). Cluster 5 was of most interest to us as it contained genes that were induced by −Fe, but strongly repressed by additional flg22 treatment. A gene ontology (GO) enrichment analysis for the genes contained in cluster 5 revealed that this gene list was highly enriched for the GO term “response to iron starvation”. Among genes in cluster 5 were many of the canonical iron deficiency responsive genes, including those related to iron deficiency signaling (BTS, PYE, bHLH38, bHLH39, bHLH100, bHLH101), iron uptake-related components (OPT3, NAS4, F6′H1, MYB10, IRT1, FRO2, MYB72, AHA7) and mobile iron deficiency signaling (IMA1, IMA2, IMA3) (FIG. 2B). As expected, these responses were not observable in fls2 mutant plants that don't perceive flg22 (FIGS. 9C-9F). The extensive and distinct interplay of gene expression changes induced by flg22 and −Fe suggests that flg22 represses iron deficiency signaling by regulating key components of the iron deficiency signaling pathway.

Notably, flg22 repressed the upregulation of three IMAs in the root upon −Fe treatment (FIG. 2B). As IMAs act upstream of most known iron deficiency responses including FIT, IMAs might be downstream of the flg22 signaling relay. The overexpression line IMA1ox in which the iron deficiency pathway is constitutively activated was therefore tested in several conditions. Flg22-triggered leaf chlorosis and reduced iron concentration in the −Fe treated seedlings were largely restored in IMA1ox seedlings (FIGS. 2C-2E). Moreover, iron deficiency-induced IRT1 accumulation and FCR activation were insensitive to flg22 treatment in −Fe conditions in the IMA1ox line (FIGS. 2F-2G). An RNAseq experiment using UBQ10::mCitrine-IMA1 and WT showed that unlike in Col-0 where a large proportion of genes that were upregulated under −Fe conditions were downregulated under −Fe/+flg22 (227/457, 49.7%; p-value: 2.08*10⁻⁹², hypergeometric test), there was no significant overlap of such genes in UBQ10::mCitrine-IMA1 (FIG. 10A, lower panel, 50/457, 10.9%; p-value: 0.99, hypergeometric test). The −Fe induced genes in Col-0 were upregulated in UBQ10::mCitrine-IMA1 under +Fe condition, and shows less sensitivity to flg22 treatment (FIGS. 10B-10D). Consistent with previous findings, expression levels of IMA2 and IMA3 were decreased in UBQ10::mCitrine-IMA1, compared to Col-0 throughout all treatment conditions, suggesting that there might be a gene dosage compensation mechanism within the IMA gene family (FIG. 2H). The expression levels of key flg22-dependent PTI (Pattern triggered immunity) components were also examined, and it was found that some of these were also affected by IMA1 overexpression (FIG. 10E). Taken together, the data show that the repression of iron deficiency responses by flg22 is abolished when IMA1 is continuously expressed, that the flg22 modulation of iron deficiency responses involve a downregulation of IMAs, and that IMA1 signaling can perturb expression of PTI response genes.

### Example 4

### Flg22 Causes IMA1 Depletion

IMA1 is a phloem mobile signal that is triggered upon iron deficiency. To investigate whether and how flg22 abolishes IMA function, the IMA octuple mutant line ima8x, and the rescue line, pIMA1::EYFP-IMA;ima8x were obtained. As expected, it was observed that IRT1 was not induced in iron deficiency condition in the ima8x mutant line and that the IMA1 transgene successfully rescued IRT1 induction in ima8x (FIG. 11A). As in wildtype, flg22 was able to repress IRT1 accumulation and FCR induction in pIMA1::EYFP-IMA;ima8x (FIGS. 11A-11B). Surprisingly, the highly increased EYFP-IMA1 protein levels that were observed upon −Fe treatment in whole root tissue, were only slightly reduced upon flg22 treatment (FIG. 3A). To investigate this partial depletion of IMA1 protein in higher detail, pIMA1::EYFP-IMA;ima8x lines were pretreated with −Fe to strongly elevate IMA1 protein levels, and only subsequently treated with flg22. Consistent with the previous experiment, flg22 treatment for 6 hours only partially depleted the −Fe treatment-induced IMA1 in the root (FIG. 3B). As iron deficiency responses can be highly localized, confocal microscopy of EYFP-IMA1 in the early differentiation zone of the root was conducted, which is the region of IRT1 induction under iron deficient conditions. As expected, −Fe highly induced IMA1 protein in stele, as well as in the pericycle, endodermis and cortex tissues in the roots of these seedlings (FIGS. 3C-3D). Concomitant treatment with −Fe and flg22 strongly reduced IMA1 protein accumulation in the endodermis and cortex and partially in the pericycle, but it did not strongly reduce IMA1 level in the vasculature (FIGS. 3C-3D). To quantify this pattern, the IMA1 signal diameter, and IMA1 signal intensity profile compared to the width of the whole root (FIG. 3E; FIG. 11H) were measured. Under −Fe, the IMA signal spread throughout the whole width of the root. In +Fe or when treated with flg22 in −Fe, it was restricted around the vasculature (FIG. 3F; FIG. 10H). As expected, these responses were abolished in the fls2 receptor mutant that does not perceive flg22 (FIG. 11C-11H). The mechanism for this cell-type specific depletion of IMA1 upon flg22 treatment was then investigated. To study a potential transcriptional regulation, pIMA1::mCitrine-NLS-mCitrine plants were obtained. The IMA1 transcriptional activity was low under +Fe, and −Fe strongly induced IMA1 transcription in all cell layers (FIGS. 12A-12B). Strikingly, flg22 led to a further induction of IMA1 transcriptional activity in the ground tissue under −Fe (FIGS. 12A and 12C). This suggested that the depletion of IMA1 in the ground tissue was more likely to be regulated at the post-transcriptional level.

IMA1 had been shown to be a mobile signal, and intercellular molecular movement can be regulated in plants by flg22-dependent callose deposition at plasmodesmata. To test whether this could explain the flg22-mediated repression of −Fe responses, 2-deoxy-d-glucose (DDG), a well-characterized callose synthase inhibitor, was applied. To assess this, IRT1 expression was used as a read-out as the repression of IMA1 in the ground tissue coincided with the flg22 elicited repression of IRT1 and FCR. A rescue of the IRT1 protein level was not observed (FIG. 12D). This suggests that flg22 mediated callose deposition may not play a role in regulating the iron deficiency responses.

mCitrine-IMA1 was then ectopically expressed in a cell type specific manner (FIG. 12E) and the IRT1 induction under iron sufficient condition was measured, as this allowed exclusion of the effect of endogenous IMA, which is only induced by iron deficiency. IRT1 was strongly induced in pUBQ10::mCitrine-IMA1 (expressing in all cell layers), and pPGP4::mCitrine-IMA1 lines (preferentially expressing in the epidermis and partially in the cortex) (FIG. 3G). This suggests that the presence of IMA1 in the epidermis/cortex is required and sufficient to induce IRT1 in the root. IRT1 was not strongly induced in the pLBD16::mCitrine-IMA1 (pericycle) or in pELTP::mCitrine-IMA1 lines (endodermis) compared to Col-0 wildtype plants (FIG. 3G). Interestingly and unlike pIMA1::EYFP-IMA1;ima8x, the signal of pELTP::mCitrine-IMA1 did not extend to the outer cell layers under both +Fe and −Fe conditions, suggesting that IMA1 needs to be locally expressed in the cortex and epidermis to induce IRT1 (FIGS. 13A-13C).

Whether constitutive presence of IMA1 in the epidermis and cortex was sufficient to abolish the repression of iron deficiency responses by flg22 was then tested. pPGP4::mCitrine-IMA1 roots driving IMA1 continuously in the epidermis and cortex root tissues were insensitive to the flg22-mediated IRT1 repression under +Fe conditions (FIG. 3H). However, the IMA1 levels were slightly decreased upon flg22 treatment in −Fe in the pPGP4::mCitrine-IMA1 lines, suggesting that IMA1 protein was degraded in this condition (FIG. 13D). Taken together, these data strongly suggest that flg22 treatment leads to the repression of IRT1 and FCR activation under iron deficiency through downregulation of IMA1 in the outer tissue layers (ground tissue).

### Example 5

### BTSLs Deplete IMA1 in the Ground-Tissue

Whether the partial degradation of IMA1 in −Fe flg22 conditions acted through the ubiquitin dependent protein degradation pathway was then investigated. MG132, an inhibitor of 26S proteasome, strongly reduced the protein degradation of IMA1 under −Fe (FIG. 14A). As a previous study had shown that BRUTUS (BTS), a regulator of iron homeostasis, ubiquitinates IMA1 to mediate IMA1 degradation to regulate iron homeostasis, it was hypothesized that BTS is required to mediate flg22-triggered IMA1 degradation. However, even though bts-1 roots showed induced IRT1 expression and FCR activity compared to Col-0 under +Fe and −Fe conditions, IRT1 expression and FCR activity, as well as IMA1 in the ground tissue were still repressed upon flg22 treatment in bts-1 mutant plants (FIGS. 14B-14D). This suggests that BTS is not required for the flg22 mediated IMA1 degradation and iron deficiency response repression. There are two BTS homologs in Arabidopsis (BTSL1 and BTSL2) that are expressed in the root and function redundantly to regulate iron homeostasis. To test whether BTSL1 and BTSL2 are involved in regulating flg22-mediated iron responses, the btsl1,2 double mutant was phenotyped in response to −Fe and flg22. Compared to Col-0, the btsl1,2 mutant plants developed less leaf chlorosis in response to flg22 under low iron conditions, suggesting that btsl1,2 plants are less sensitive to the flg22-triggered repression of iron deficiency responses (FIGS. 4A-4C). Consistent with the chlorosis phenotype, FCR activity and IRT1 protein accumulation were significantly less responsive to flg22 treatment in −Fe conditions in btsl1,2 mutant plants compared Col-0 (FIGS. 4D-4E). These data suggest that BTSL1 and BTSL2 are required for regulating flg22-mediated repression of iron deficiency responses.

Next, the expression pattern of BTSL1 and BTSL2 was analyzed using plant carrying pBTSL1-GFP and pBTSL2-GFP transcriptional reporters. It was found that the expression levels of BTSL1 and BTSL2 were induced by −Fe (FIG. 4F). BTSL1 is mainly expressed in the epidermis in response to iron deficiency. flg22 strongly repressed BTSL1 expression under −Fe. This is in contrast to BTSL2, which is expressed in the entire ground tissue in iron deficiency conditions. flg22 treatment didn't repress BTSL2 expression (FIG. 4F). Since the spatial expression patterns of BTSL1 and BTSL2 coincided with the area in which IMA1 was repressed upon flg22 treatment, it was hypothesized that BTSL1 and BTSL2 are involved in the flg22-regulated IMA1 depletion in the ground tissue. IMA1 protein level was reduced upon flg22 treatment in pIMA1::EYFP-IMA1;ima8x, but not in pIMA1::EYFP-IMA1;btsl1,2 (FIGS. 14E-14F). Consistent with the hypothesis, the strong increase of IMA1 in the ground tissue upon iron deficiency was not abolished by flg22 treatment in btsl1,2 (FIGS. 4G-4I; FIG. 14G). Cycloheximide (CHX) treatment decreased IMA1 level under −Fe, particularly in the epidermis and cortex, similarly to the effect of flg22 indicating that IMA1 is degraded in low iron conditions. This reduction of IMA1 by either CHX or flg22 was restored in btsl1,2 (FIGS. 14H-14J) indicating that BTSL1&2 are required for degrading IMA1 protein under these conditions.

Taken together, these data suggest that the flg22-induced suppression of the iron deficiency response is mediated predominantly through a BTSL1/2-dependent IMA1 degradation in the ground tissue of the root in the differentiation zone (FIG. 14K).

### Example 6

### IMA1 has Root and Shoot Immune Functions

The iron deficiency response and an effective immune response trigger antagonistic responses in the root. For instance, the iron deficiency response includes the acidification of the rhizosphere for promoting iron solubility. However, the defense response leads to an alkalization of the root meristem, which further promotes immunity. The above data suggest that IMA1 might be a central player at the interface of both pathways. While the role of IMA1 in iron deficiency signaling has been characterized, a role of IMA1 in the regulation of immunity had not been described yet. Whether IMA1 played a role in regulating the rhizosphere acidification capacity was therefore first investigated. It was found that under +Fe conditions, Col-0 and ima8x exhibited no acidified roots whereas UBQ10::mCitrine-IMA1 displayed constitutive root acidification (FIGS. 5A-5B-). Iron deficiency triggered root acidification around the root tip in Col-0, but not in ima8x. flg22 treatment repressed root acidification in Col-0, but not in UBQ10::mCitrine-IMA1 (FIGS. 5A-5B). This suggests that IMA depletion is required for abolishing the root acidification that is triggered by flg22.

Although UBQ10::mCitrine-IMA1 plants showed slightly shorter roots when they were grown on iron sufficient medium compared to Col-0 and ima8x, they exhibited less sensitivity to flg22-mediated root growth inhibition, whereas ima8x exhibited enhanced flg22-mediated root growth inhibition (FIGS. 5C and 5E). Consistent with the data that the activity of the flg22 receptor FLS2 is dependent on environmental conditions, such as root apoplastic pH, the less sensitive response of UBQ10::mCitrine-IMA1 to flg22 was restored by adding the pH buffering reagent MES (FIGS. 5D-5E). Expression of CYP71A12 was then studied, which is a flg22 response gene whose promoter activation is dampened by low pH. CYP71A12 induction by flg22 was impaired in UBQ10::mCitrine-IMA1 plants, but was stronger in ima8x compared to Col-0 (FIG. 5F). In line with this, roots of UBQ10::mCitrine-IMA1 plants showed less MAPK phosphorylation upon flg22 treatment compared to Col-0 under iron sufficient conditions (FIG. 5G). Overall, these data suggest that IMA1 and its effect on rhizosphere acidification are important for root responses to flg22. Iron availability has been shown to mediate root colonization by rhizobacterium Bacillus velezensis SQR9. As the above data suggest that IMA1 might be a central player in mediating iron and immune responses, whether IMA1 plays a role in mediating host-microbe interaction was then studied. The rhizobacterium Pseudomonas protegens CHA0 (a model commensal Pseudomonas protegens strain which produces flagellin) showed less colonization on the root of ima8x, whereas it colonized roots of the UBQ10::mCitrine-IMA1 line to a higher extent (FIG. 5H). Taken together, this suggests that IMA1 functions at the nexus of iron deficiency and root-microbe interactions and that higher levels of IMA can facilitate elevated bacterial growth on roots.

Since IMA1 is considered a mobile signal that relays information from the shoot to the root, it was next studied whether IMA1 also plays a role in coordinating iron and immune responses in the shoot. IMA1 protein level was low in the shoot under +Fe conditions. −Fe led to accumulation of IMA1 protein in the shoot in epidermal cells, mesophyll cells, and abundantly in in the vascular tissue (FIG. 15A). Treatment with flg22 under −Fe conditions decreased IMA1 protein in the epidermal cells and mesophyll cells but not in the vascular tissue (FIG. 15A), suggesting that like in the root, there are also cell-type specific regulatory mechanisms for IMA1 in the shoot. By analyzing IMA1ox dependent transcriptome changes in the shoot from a published dataset, it was found that besides iron-responsive genes, immune response and systemic acquired resistance genes were enriched among the upregulated genes (FIG. 15B). This suggests that IMA1 also plays a role in regulating immune programs in the shoot. However, no strong differences in flg22-elicited MAPK phosphorylation in the shoot were observed, when comparing UBQ10::mCitrine-IMA1 and Col-0 wildtype (FIG. 15C), suggesting that the perception of flg22 or PTI signaling activation is not affected in UBQ10::mCitrine-IMA1 in the shoot. To further explore this, several PTI markers were measured in shoot tissue using qPCR after the plants had been exposed to flg22 for a short time (1 hour). Immunity marker genes were slightly upregulated without flg22, and the activation was more robust with flg22 in UBQ10::mCitrine-IMA1 compared to Col-0. Moreover, ethylene and jasmonic acid biosynthesis and signaling pathways were not hyperactive in UBQ10::mCitrine-IMA1. While the effects in the shoot were moderate, one explanation for this is that overexpression of IMA1 in the shoot induces a sub-set of systemic defense responses, which is different from the root. Based on this idea, if the systemic defense responses were indeed induced in the shoot of the IMA1 overexpressor, these plants might then be more resistant to foliar pathogen attacks. Consistent with this idea, UBQ10::mCitrine-IMA1 plants were more resistant to foliar bacterial pathogen Pseudomonas syringae pv. tomato DC3000 (FIG. 15E). Taken together, IMA1 plays an important role to coordinate iron deficiency and immunity.

### Example 7

### Iron, Root Surface and Invading Bacteria

Bacterial populations can positively or negatively impact plant fitness through interactions that relate to iron. For example, commensal bacterial strains can aid roots with iron acquisition, whereas pathogens are thought to be in competition for available iron. Recently, a spatially defined gating mechanism for cell damage dependent immune receptor activation that contributed to the distinction of commensal/beneficial and pathogenic bacteria was discovered. The above data show that iron uptake and defense programs are connected by an IMA related mechanism in a spatially restricted manner. Thus, it was next studied whether the repression of the iron signaling by a bacterial MAMP is dependent on the location of the bacteria. For this, roots with CHA0-mcherry/gfp2 were inoculated under −Fe. Unlike with the flg22 treatment, the pIRT1 reporter as well as the IRT1 protein induction were not fully repressed by CHA0 colonization (FIGS. 6A-6B; FIG. 16A), showing that root colonization by this strain of commensal bacteria does not strongly repress iron deficiency responses. Moreover, CHA0 colonization did not repress, but it even enhanced IMA1 protein induction by −Fe, whereas flg22 partially repressed IMA1 protein induction (FIG. 6C; FIG. 16B). When CHA0 was colonizing solely the surface of the root, IMA1 remained present in the outer cell layer, whereas flg22 treatment confined IMA1 into the stele (FIGS. 6E-6F). This indicates that this commensal/beneficial bacterial strain colonization on the root surface does not necessarily repress iron deficiency responses and that the plant is still able to take up iron.

During our inoculations, we noticed that in some cases, the CHA0 (which is considered a non-pathogenic bacterial strain) entered the roots at lateral root primordia, a region where cracks can naturally occur. Consistent with previous findings, higher immune responses were detected when CHA0 colonized at lateral root primordia compared with growth on the surface of differentiation zone (FIGS. 16C-16D). When looking at such cases systematically, it was found that when CHA0 entered the primary root through the LRP, IMA1 accumulation in the primary root was strongly reduced (FIGS. 6G-6H). Altogether, these data suggest that locally gated MAMP responsiveness can lead to spatially confined IMA1 repression and thereby might contribute to allow roots to locally shut off iron deficiency responses when internally colonized or to continue with iron acquisition in the presence of non-invasive, surface-dwelling bacteria (FIG. 16E).

It will be apparent that the precise details of the methods or compositions described may be varied or modified without departing from the spirit of the described aspects of the disclosure. We claim all such modifications and variations that fall within the scope and spirit of the claims below.

We claim:

1. A method for increasing resistance against a plant pathogen in a plant, plant part, or plant cell, comprising:
increasing expression and/or activity of an IRON MAN (IMA), and/or reducing expression and/or activity of a BRUTUS-Like E3 ligase (BTSL) in the plant, plant part, or plant cell, thereby increasing resistance against a plant pathogen in the plant, plant part, or plant cell.

2. The method of claim 1, wherein the plant pathogen is a foliar bacterial pathogen.

3. The method of claim 1, wherein the plant pathogen is a pathogen that infects a shoot of the plant.

4. The method of claim 1, wherein the plant pathogen is Pseudomonas syringae, Ralstonia solanacearum, Xanthomonas oryzae, Xanthomonas campestris, Xanthomonas axonopodis, Erwinia amylovora, Xylella fastidiosa, Dickeya dadantii, or Pectobacterium carotovorum.

5. The method of claim 1, wherein the increasing expression and/or activity of an IMA comprises introducing one or more exogenous nucleic acid molecules that encode an IMA protein into the plant, plant part, or plant cell, thereby generating a gene-edited plant, gene-edited plant part, or gene-edited plant cell comprising the exogenous nucleic acid.

6. The method of claim 5, wherein the IMA protein is a full-length IMA protein, or a C-terminal fragment of a full-length IMA protein.

7. The method of claim 6, wherein the C-terminal fragment of the IMA protein is 7 to 20 amino acids.

8. The method of claim 6, wherein the C-terminal fragment of the IMA protein comprises G/N/D-D-D-D-x₍₁₋₆₎-D-x-A-P-A-A.

9. The method of claim 5, wherein the IMA protein is an IMA protein native to the plant, plant part, or plant cell, or an IMA1 protein ortholog.

10. The method of claim 5, wherein:
at least one of the one or more exogenous nucleic acid molecules that encode the IMA protein comprises at least 80%, 85%, 90%, 95%, 96%, 97%, 98%, 99%, or 100% sequence identity to any one of SEQ ID NOs: 135-138;
the IMA protein is a full-length IMA protein and comprises at least 80%, 85%, 90%, 95%, 96%, 97%, 98%, 99%, or 100% sequence identity to any one of SEQ ID NOs: 1-134; or
the IMA protein is a C-terminal fragment of a full-length IMA protein and comprises at least 10 of the C-terminal amino acids of any one of SEQ ID NOs 1-134.

11. The method of claim 1, wherein reducing expression and/or activity of a BTSL comprises introducing one or more exogenous nucleic acid molecules that reduce expression of a BTSL gene and/or reduce activity of a protein encoded by the BTSL gene into a plant, a plant part, or a plant cell, thereby generating a gene-edited or transformed plant, gene-edited or transformed plant part, or gene-edited or transformed plant cell comprising the exogenous nucleic acid.

12. The method of claim 11, wherein the introducing one or more exogenous nucleic acid molecules generates a deletion of or loss-of-function mutation in the BTSL gene.

13. The method of claim 11, wherein the one or more exogenous nucleic acid molecules comprise one or more guide nucleic acid molecules that can delete or mutate the BTSL gene.

14. The method of claim 1, wherein the method further comprises introducing one or more Cas proteins or one or more nucleic acid molecules encoding a Cas protein into the plant, plant part, or plant cell.

15. The method of claim 5, wherein the one or more exogenous nucleic acid molecules are operably linked to a promoter.

16. The method of claim 15, wherein the promotor is a tissue-specific promoter that is active in a shoot cell, epidermal cell, or vascular cell of the plant.

17. The method of claim 11, wherein:
the BTSL gene comprises at least 80%, 85%, 90%, 95%, 96%, 97%, 98%, 99%, or 100% sequence identity to any one of SEQ ID NOs: 152-153; and/or
the protein encoded by the BTSL gene comprises at least 80%, 85%, 90%, 95%, 96%, 97%, 98%, 99%, or 100% sequence identity to any one of SEQ ID NOs: 139-151.

18. The method of claim 1, wherein the plant is a pennycress, soybean, canola, rice, tomato, Zea mays, or sorghum plant; the plant part is from a pennycress, soybean, canola, rice, tomato, Zea mays, or sorghum plant; and/or the plant cell is from a pennycress, soybean, canola, rice, tomato, Zea mays, or sorghum plant.

19. A gene-edited plant, gene-edited plant part, or gene-edited plant cell made by the method of claim 1.

20. A method of producing a commodity plant product, comprising collecting or producing the commodity plant product from the gene-edited plant, gene-edited plant part, or gene-edited plant cell of claim 19.

21. A method of producing plant seed, comprising crossing the gene-edited plant of claim 19 with itself or a second plant.

22. A method for breeding a plant with increased disease resistance, comprising crossing the gene-edited plant of claim 19 with a second plant;
obtaining seed from the crossing;
planting the seeds and growing the seeds to plants; and
selecting from said plants those with increased disease resistance.

23. A recombinant nucleic acid molecule comprising a nucleic acid sequence operably linked to a heterologous promoter, wherein said nucleic acid molecule encodes (a) an amino acid sequence comprising at least 80% sequence homology to any one of SEQ ID NOs: 1-134, (b) an amino acid sequence comprising at least 10 contiguous C-terminal amino acids of any one of SEQ ID NOs: 1-134, or (c) an amino acid sequence comprising G/N/D-D-D-D-x₍₁₋₆₎-D-x-A-P-A-A; and wherein expression of the recombinant nucleic acid molecule in a plant results in increased disease resistance in the plant when compared to a plant of the same species lacking the recombinant nucleic acid molecule.
